(19) United States
(12) Patent Application Publication
(10) Pub. No.: US 2025/0223559 A1
(43) Pub. Date: Jul. 10, 2025

(54) AXIOLOID: A STEM CELL-BASED MODEL OF HUMAN AXIAL DEVELOPMENT

(71) Applicant: KYOTO UNIVERSITY, Kyoto-shi (JP)

(72) Inventors: Cantas ALEV, Kyoto-shi (JP); Yoshihiro YAMANAKA, Kyoto-shi (JP); Sofiane HAMIDI, Kyoto-shi (JP); Sirajam MUNIRA, Kyoto-shi (JP)

(73) Assignee: KYOTO UNIVERSITY, Kyoto-shi (JP)

(21) Appl. No.: 18/852,869

(22) PCT Filed: Mar. 31, 2023
(86) PCT No.: PCT/JP2023/013711

§ 371 Date: Sep. 30, 2024

Related U.S. Application Data
(60) Provisional application No. 63/326,611, filed on Apr. 1, 2022.

(51) Int. Cl. C12N 5/077; C12N 5/00; G01N 33/50

(52) U.S. Cl. CPC C12N 5/0652; C12N 5/0018; G01N 33/5023; G01N 33/5026; C12N 2501/115; C12N 2501/15; C12N 2501/385; C12N 2501/415; C12N 2501/727; C12N 2506/45; C12N 2513/00; C12N 2533/52; C12N 2533/54; C12N 2533/70; C12N 2533/90

(57) ABSTRACT

The present disclosure relates to a pluripotent stem cell (PSC)-based method to reconstitute axial development in vitro and to a method for producing the same. The present disclosure provides a three-dimensional cellular aggregate, termed ‘axioloids’, generated in vitro from pluripotent stem and composed of mesodermal cells wherein the cellular aggregate is polarized along its antero-posterior axis and its apical-basolateral axis. This cellular aggregate can reconstitute various aspects of somitogenesis and axial development, including axial elongation, segmentation, epithelial somite formation and patterning (formation of one or more somite-like structures), and oscillation of the segmentation clock under somitogenic culture conditions. Axioloids can also be used to derive various cellular lineages and functional cell types and can be used as a platform to model and reconstitute human embryo development, disease and evolution. Axioloids can be further utilized, among other things, for the assessment of the teratogenicity and toxicology of chemical compounds, the production and testing of cellular therapy products, the study of congenital and acquired human diseases and the evaluation of ongoing and future therapeutic approaches.

### TECHNICAL FIELD

The present disclosure relates to a three-dimensional cellular aggregate termed axioloid generated in vitro from a pluripotent stem cell and to a method for producing the same.

### BACKGROUND ART

The segmented body plan of vertebrates is established during somitogenesis, a well-studied process in model organisms, but remains largely elusive in humans due to ethical and technical limitations. Despite recent advances with pluripotent stem cell (PSC)-based approaches¹⁻⁵, a system that robustly recapitulates the human embryo somitogenesis process in vitro in both space and time, including its characteristic morphogenetic features including the oscillatory expression of segmentation clock genes and the related sequential formation of epithelial somites along the anterior to posterior axis of the embryo, remains largely missing.

### SUMMARY OF INVENTION

It is an object of the present disclosure to provide a new three-dimensional cellular aggregate.

In order to achieve the object above, the present disclosure provides a three-dimensional cellular aggregate generated in vitro from a pluripotent stem cell, comprising: a mesodermal cell, wherein the cellular aggregate has a polarity in an antero-posterior axis or a rostro-caudal axis and an apical-basolateral axis, and the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition.

The present disclosure can provide a new three-dimensional cellular aggregate.

### BRIEF DESCRIPTION OF DRAWINGS

FIG. 1 shows generation of Axioloids from human pluripotent stem cells. a, Schematic summary of human iPSC-derived Axioloid induction protocol. Different color code for the major steps in the induction protocol: yellow, represents small molecule treatment, blue, suspension culture and purple, the Matrigel (MG) embedding phase; used abbreviations WNT, FGF and TGFβi stand for CHIR99421, bFGF and SB431542 respectively. b, and c, Time-lapse live imaging of human Axioloid induction. b, Representative bright field images of elongating Axioloids at 24 h, 48 h, and 72 h, followed by images of Axioloids after MG embedding at 96 h and 120 h of culture. c, Serial images of a forming Axioloid at 5 h intervals, from 74 h to 119 h (extracted from Supplementary Video 1). Colored arrowheads highlight the process of segment formation at each shown time point with the yellow arrowheads pinpointing areas where somite segmentation is ongoing, whereas red arrowheads highlight the areas where segmentation is completed. d, Periodicity of somite segmentation based live imaging observations (N=4, n=9). e, Axioloid length (antero-posterior) at 24 h, 48 h, 72 h, and at 96 h and 120 h with or without MG embedding (N=3, n=18). f and g, Immunofluorescence staining and signal quantification of MG embedded Axioloids at 96 h and 120 h. f, Merged channel images of Axioloids stained for F-actine (Phalloidin) in gray, TBXT (BRA) in green, and MEOX1 in red (corresponding single channel images are shown in FIG. 6i). g, Corresponding quantification along the posterior to anterior axis of TBXT (green line) and MEOX1 (in red) signal intensity (N=3, n=9). h, and i, Immunofluorescence staining of MG embedded Axioloids at 96 h and 120 h, images shown are representative of 3 independent experiments. h, F-actine (Phalloidin) is in gray, TBX6 in blue and SOX2 in red. i, F-actine (Phalloidin) is in gray, TBXT in green and SOX2 in red. j and k, HCR staining of MG embedded Axioloids at 96 h, shown merged channel images are representative of 2 independent experiments. j, HCR staining of MSGN1 in cyan, TCF15 in magenta and RIPPLY2 in yellow. k, HCR staining of RIPPLY2 in blue, LFNG in green and HES7 in red, white arrowhead highlight the strip pattern staining of LFNG in the posterior half of each somite. 1, and m, HCR staining of MG embedded Axioloids at 96 h for MESP2 in yellow, UNCX in cyan and TBX18 in magenta, and corresponding signal intensity measurement along the posterior to anterior axis normalized to the position of the MESP2 signal peak. Numbered red arrowheads pinpoint the TBX18 strip pattern observed in the posterior part of each somite. n, to q, Time series of a HES7:Luciferase expressing hiPSC cell line (201B7 Luc) from 72 h to 112 h. n, Quantification of the total HES7:luciferase signal in Axioloids with or without MG (N=6, n=22) and o, Periodicity was measured as the time interval between the consecutive HES7:Luciferase signal peaks. p, Right, kymograph along the line shown in the left image of Axioloid embedded in MG. q, Periodicity of the HES7:Luciferase signal measured in p, (N=4, n=8). Scale bar is 200 μm.

FIG. 2 shows scRNA-seq characterization of human Axioloids. a-c, UMAP projection of scRNA-seq datasets of hAxioloids at 48 h, 72 h, 96 h and 120 h, colored by a, samples, and b, c, identified clusters. Both non-MG and MG samples are included for 96 h and 120 h. Arrows in c, show RNA velocity. d, Proportions of cell types in Axioloids over time. Regarding 96 h and 120 h, only MG-plus samples are shown. e, Expression levels of the selected genes are indicated on the UMAP plot. f, Single-cell expression profiles of identified marker genes for each cell cluster except for E-SM2, M-SM2, and presumably apoptotic cells. The top 50 (or less) genes of higher fold changes are shown. Presumably apoptotic cells are omitted. g-h, UMAP plots of Axioloids at 96 h with MG, colored by identified clusters in g and by pseudotime in h. Arrows in g show RNA velocity. In this analysis, two replicates are integrated. i, Marker gene expression patterns at 96 h with MG samples along pseudotime rank in h. IM-like and EC-like cells are omitted. Used abbreviations: TB (tail bud), E-TB (early tail bud), M-TB (mid tail bud), L-TB (late tail bud), PSM (presomitic mesoderm), E-PSM (early presomitic mesoderm), APSM (anterior presomitic mesoderm), E-APSM (early anterior presomitic mesoderm), N-SM (nascent somatic mesoderm), SM1 (somitic mesoderm 1), E-SM1 (early somitic mesoderm 1), SM2 (somitic mesoderm 2), E-SM2 (early somitic mesoderm 2), M-SM1 (mid somitic mesoderm 1), M-SM2 (mid somitic mesoderm 2), L-SM (late somitic mesoderm), IM-like (intermediate mesoderm-like), EC-like (endothelial cell-like), MG (Matrigel).

FIG. 3 shows signaling pathways and MG effect in Axioloids. a-d, Merged HCR staining images and signal quantification of MG embedded Axioloids at 96 h and 120 h. Shown images are representative of at least 3 independent experiments, a, HCR staining of MESP2 in blue, FGF8 in green and b, corresponding quantification along the posterior to anterior axis of the signal intensity normalized the position of the WNT3a signal peak (96 h: N=3, n=9 and 120 h: N=4, n=10). c, HCR staining of CYP26 A1 in cyan, ALDH1 A2 in magenta and RIPPLY2 in yellow and d, corresponding quantification along the posterior to anterior axis of the signal intensity normalized the position of the CYP26 A1 signal peak (96 h: N=3, n=8 and 120 h: N=3, n=9). e, UMAP plots of two replicates of 96 h Axioloids with and without MG after MNN-integration of the four samples. Note that EC-like cells only appear with MG, highlighted by black arrow head. f, Volcano plots in PSM and SM. Red and blue dots indicate up- and down-regulated genes by MG in both replicates, respectively. g, Log2 fold changes of differentially expressed genes that are up- or down-regulated by MG in SM consistently in both replicate experiments are shown. h, Expression levels of indicated genes in SM are compared between samples. Used abbreviations: TB (tail bud), PSM (presomitic mesoderm), aPSM (anterior presomitic mesoderm), N-SM (nascent somatic mesoderm), SM (somitic mesoderm), EC-like (endothelial cell-like), MG (Matrigel). Scale bar is 200 μm.

FIG. 4 shows RA signaling and HOX Code in Axioloids. a and b, Bright field images of Axioloids at 96 h and 120 h after embedding with b, MG and RAL or a, MG and RA. c, Immunofluorescence staining of an Axioloid embedded in MG and RA at 120 h stained for F-actine (Phalloidin) in gray, FN in green, MEOX1 in red, and TBXT (BRA) in blue; shown merged channel image has been isolated from the middle of a z-stack that has been denoised using an AI based software (extracted from the Supplementary Video 4). d, and e, HCR staining of MG embedded Axioloids at 96 h (left image) and 120 h (right image) for MESP2 in yellow, UNCX in cyan and TBX18 in magenta, and corresponding signal intensity measurement along the posterior to anterior axis normalized to the position of the MESP2 signal peak for 96 h (top) and 120 h (bottom). Green and red arrowheads pinpoint the TBX18 strip pattern observed in the posterior part of each somite at 96 h and 120 h respectively. f, and g, UMAP projection of Axioloids at 96 h and 120 h, colored by samples f, and by clusters annotated f. All four conditions (MG, MG+RAL, and MG+RA) are included for each time point. g, RNA velocity is shown by arrows. h-j, UMAP projection of integrated scRNA-seq profiles of Axioloids and human embryos. Axioloids with MG and Retinal at 96 h and 120 h and the embryo at CS12 are analyzed. h, Axioloid cell clusters are colored, i, Cell clusters in the embryo are colored. j, Origins of embryonic body parts are indicated. k, Pearson correlation coefficient was calculated between each of the Axioloid clusters and each of the embryo clusters based on the distribution of number of cells assigned in the defined clusters for the integrated dataset. 1, Visualization of the spatial distribution of the ACTB, TBXT, HES7, TBX6, MESP2, RIPPLY2, MEOX1, TCF15 transcripts in a section of an Axioloid embedded in MG+RAL at 96 h using HybISS and m, corresponding heatmap plot showing the average gene expression along the posterior to anterior axis normalized to the position of the MESP2 signal peak (n=3). n, Heatmap showing the distribution of HOXC gene expression based on the pseudotime analysis of the RNA sequencing of Axioloids embedded in MG. o, and p, HOXC cluster analysis in Axioloids embedded in MG+RAL at 96 h. o, Top panel, analysis of the epigenetic landscape at the HOXC locus profiled by CUT&Tag using antibodies against H3K4me3 (green) and H3K27me3 (red). Bottom panel, visualization of the spatial distribution of the HOXC transcripts using HybISS analysis of the HOXC cluster. p, Heatmap plot of the HybISS data shown in s, showing the average HOXC cluster gene expression along the posterior to anterior axis normalized to the position of the MESP2 signal peak (n=3). Scale bar is 200 μm.

FIG. 5 shows molecular and functional characterization of patient-like Axioloids. Panels a-g, show data for HES7 KO1, panels h, to n, show data for HES7^{R25W} MT1 and panels o-u, show data for MESP2 KO1. a, h, and o, Serial brightfield images of a forming patient-like Axioloid at 72 h, 96 h and 120 h extracted from Supplementary Video 9, 11, and 13 respectively (check if it corresponds if not change the images). b, i, and p, Axioloid length along the posterior to anterior axis at 24 h, 48 h, 72 h, and at 96 h and 120 h for the corresponding cell lines HES7 KO1 (N=3, n=18), HES7 KO2 (N=2, n=12), HES7^{R25W} MT1 (N=3, n=18), HES7^{R25W} MT2 (N=3, n=18), MESP2 KO1 (N=3, n=17), MESP2 KO2 (N=3, n=15). c, j, and q, Immunofluorescence staining and signal quantification of MG+RAL embedded Axioloids at 120 h. Top panel, merged channel images of Axioloids stained for F-actine (Phalloidin) in gray, FN in green, and MEOX1 in red, TBXT (BRA) in Blue. Bottom panel, corresponding quantification along the posterior to anterior axis of TBXT (green line) and MEOX1 (in red) signal intensity for HES7 KO1 (N=3, n=10), HES7^{R25W} MT1 (N=4, n=9), MESP2 KO1 (N=3, n=12). d, k, and r, HCR staining and signal quantification of MG+RAL embedded Axioloids at 120 h. Top panel, MESP2 in yellow, UNCX in cyan and TBX18 in magenta, and bottom panel, corresponding signal intensity measurements along the posterior to anterior axis normalized to the position of the MESP2 signal peak for HES7 KO1 (N=3, n=9), HES7^{R25W} MT1 (N=3, n=8), MESP2 KO1 (N=3, n=7). e, 1, and s, Time series of the HES7:Luciferase expressing hiPSC cell line (209B7 Luc) from 72 h to 112 h for HES7 KO1 (N=4, n=12), HES7 KO2 (N=4, n=12), HES7^{R25W} MT1 (N=3, n=9), HES7^{R25W} MT2 (N=3, n=9), MESP2 KO1 (N=4, n=12), MESP2 KO2 (N=4, n=12). f, m, and t, Kymographs of f, HES7 KO1, m, HES7^{R25W} MT1, and t, MESP2 KO1 Axioloids embedded in MG+RAL. g, n, and u, Average measured HES7:Luciferase signal over time for HES7 KO1 (N=4, n=8), HES7 KO2 (N=5, n=9), HES7^{R25W} MT1 (N=6, n=12), HES7^{R25W} MT2 (N=6, n=11), MESP2 KO1 (N=4, n=9), MESP2 KO2 (N=4, n=9). Scale bar is 200 m.

FIG. 6 shows morphological and molecular characterization of human Axioloids. a-f, Bright field images of a, elongating Axioloids at 24 h, 48 h, and 72 h, followed by images of Axioloids after MG embedding at 96 h and 120 h or b, c, without MG embedding. d, and e, Representative bright field images reflecting the different morphology-based categories (Cat #1: straight, minimal curvature, clear segments; Cat #2: curved but clear segments; Cat #3: very curved, segment borders are still distinguishable; Cat #4: not properly elongated or completely collapsed; segment borders not clear) of MG embedded Axioloids at 96 h and 120 h in 2 different cell lines d, 409B2 (N=3, n=128 Axioloids) and e, 201B7 Luc (N=3, n=60 Axioloids). f, Serial images of a forming Axioloid at 5 h intervals, from 74 h to 119 h (extracted from Supplementary Video 1). Colored arrowheads highlight the process of segment formation at each shown time point with the yellow arrowheads pinpointing areas where somite segmentation is ongoing whereas green arrowheads highlight the areas where segmentation is completed. g, Periodicity of somite segmentation based on live-cell imaging observations (N=3, n=9). h, Axioloid length (antero-posterior) at 24 h, 48 h, 72 h, and at 96 h and 120 h with or without MG embedding (N=3, n=18). i-q, Immunofluorescence staining and corresponding quantifications. i, j, m, n, p, Representative images of F-actine (Phalloidin) in gray, TBXT in green, and MEOX1 in red stained Axioloids at m, 72 h, i, j, 96 h and 120 h with MG embedding and n, p, 96 h and 120 h without MG embedding. k, o, p, Corresponding quantification along the posterior to anterior axis of TBXT (green line) and MEOX1 (in red) signal intensity at k, 96 h (N=3, n=14) and 120 h (N=4, n=16) with MG embedding and o, q, 96 h (409B2 N=3, n=9 and 201B7 N=3, n=15) and 120 h (409B2 N=3, n=9 and 201B7 N=3, n=11) without MG embedding. 1, Somite length was measured based on Phalloidin staining done in i, and j, in both cell lines 409B2 (N=3, n=9) and 201B7 Luc (N=3, n=9). Scale bar is 200 μm.

FIG. 7 shows assessment of apicobasal polarity, developmental protein- & gene expression patterns, rostrocaudal patterning, traveling wave front of HES7 oscillatory activity & segmentation in human Axioloids embedded in MG. a-d, Immunofluorescence staining of MG embedded Axioloids. a, and b, High magnification images (X63) of a single segment at 120 h with F-actine (Phalloidin) in gray, a PKC and FN in green, and MEOX1 in red in a, 409B2 and b, 201B7 Luc iPS cell line-derived Axioloids; images are representative of 2 independent experiments. c, Images of F-actine (Phalloidin) in gray, TBX6 in blue and SOX2 in red and d, Images of F-actine (Phalloidin) in gray, TBXT in green and SOX2 in red. e-p, HCR staining and corresponding signal quantification of e, and f, HCR staining of MSGN1 in cyan, TCF15 in magenta and RIPPLY2 in yellow and g, and h, of RIPPLY2 in blue, LFNG in green and HES7 in red; the purple arrowhead highlights the stripe-like staining pattern of LFNG in the posterior half of each somite, shown images are representative of 2 independent experiments. i-p, HCR staining of MESP2 in yellow, UNCX in cyan and TBX18 in magenta, and corresponding signal intensity measurements along the posterior to anterior axis normalized to the position of the MESP2 signal peak of Axioloids embedded in MG at 96 h and 120 h respectively in 2 different cell lines i, j, 409B2 (N=4, n=10 and N=3, n=9) and k, l, 201B7 Luc (N=3, n=9 and N=3, n=10) and Axioloids without MG embedding at 96 h in 2 cell lines m, n, 409B2 (N=3, n=12) and o, p, 201B7 Luc (N=3, n=11). Single channel images shown in c-e, g, and i, top panel, corresponds to the merged channel images shown in FIG. 1 h-j. q, and r, Annotated serial images of a forming Axioloid with HES7:Luciferase signal overlayed in green (extracted from Supplementary Video 2). Colored dotted lines mark the furthermost anterior position reached by each HES7 oscillation wave of gene expression, identical colored arrowheads mark this position overtime, yellow first oscillation, red second, blue third, orange forth. r, Image at 24 h shows that each HES7:Luciferase expression wavefront position corresponds to the area of a formed segment. s, Average HES7:Luciferase intensity measurements overtime (N=4, n=8). Scale bars in a, and b, 50 m others are 200 μm.

FIG. 8 shows single cell RNA-seq analysis of human Axioloids. a, UMAP projection of scRNA-seq datasets of Axioloids at 48 h, 72 h, 96 h and 120 h, colored by inferred cell cycle phases (G1, G2M, S). b, G2M.Score and S.Score of the cells in each cluster of FIG. 2b. c, Proportions of cell types in Axioloids with and without MG for both 96 h and 120 h timepoints. d, Averaged expression levels of ribosomal protein genes in each cluster of FIG. 2b. e, Transition of TB marker gene expression along the time course. f, Expression levels of TBXT and SOX2 in each cell are plotted for the three TB clusters (E-TB, M-TB and L-TB, standing for early, mid and late tailbud respectively).

FIG. 9 shows expression gradients of FGF, WNT and RA signaling pathway members in human Axioloids embedded in MG. a-c, Pseudotime representation of expression of FGF, WNT and RA signaling pathway associated transcripts in human MG exposed Axioloids at 96 h of culture (24 h after embedding into MG); gene expression patterns arranged along pseudotime rank. Pseudotime expression patterns for effectors and negative regulators of all three pathways are included. d-i, HCR staining images and signal quantification of MG embedded Axioloids at 96 h and 120 h derived from d, and g, 409B2 and e, f, h, and i, 201B7 Luc iPSC lines. Shown images are representative of 3 independent experiments. d, and e, HCR staining of MESP2 in blue, FGF8 in green and WNT3a in red. f, corresponding quantification along the posterior to anterior axis of the signal intensity normalized the position of the WNT3a signal peak (96 h: N=3, n=8 and 120 h: N=3, n=7). g, and h, HCR staining of CYP26 A1 in cyan, ALDH1 A2 in magenta and RIPPLY2 in yellow and i, corresponding quantification along the posterior to anterior axis of the signal intensity normalized the position of the CYP26 A1 signal peak (96 h: N=3, n=6 and 120 h: N=3, n=7). Scale bar is 200 m.

FIG. 10 shows single cell RNA-seq analysis: identification of DEGs associated exposure of Axioloids to MG. a, UMAP projection of the integrated two replicates of Axioloids at 96 h with MG, colored by the clusters of FIG. 2g. b, UMAP projection of the integrated two replicates of Axioloids at 96 h with MG, colored by the clusters of FIG. 2b. Note that b, includes only replicate 1. c, Expression level of indicated genes on the same UMAP plot in a. d, Averaged expression levels of identified EC-like marker genes in each cluster of FIG. 2b. e, Enrichment analysis of upregulated genes in SM in the presence of MG. Hallmark gene sets and KEGG datasets are used. f, and g, differentially expressed genes between Axioloids with and without MG at 96 h in PSM and TB.

FIG. 11 shows assessing the morphogenetic effects of retinoid signaling on human Axioloids. a, and b, Bright field images of Axioloids at 96 h and 120 h after embedding in MG (Matrigel) only, MG+RAL (Retinal), MG+ROL (Retinol) or MG+RA (Retinoic Acid) for a, 409B2 and b, 201B7 Luc. c, Serial images of an elongating Axioloid at 5 h intervals, from 74 h to 119 h (extracted from Supplementary Video 3). Colored arrowheads highlight the process of segment formation at each shown time point with the yellow arrowheads pinpointing to areas where somite segmentation is ongoing whereas red for 409B2 or green for 201B7 Luc arrowheads highlight the areas where segmentation is completed. d, and e, Immunofluorescence high magnification images (X63) of a single somite of Axioloids embedded in MG+RAL at 120 h with F-actine (Phalloidin) in gray, and from top to bottom aPKC, CDH2, LAMC1, FN in green, and MEOX1 in red in d, 409B2 and e, 201B7 Luc iPS cell lines; images are representative of 2 different experiments. f, Bright field images of Axioloids at 120 h without MG embedding, after addition of RAL only, ROL only and RA only (no MG in all cases) in 409B2, top panel and 201B7 Luc, bottom panel. g, and h, Representative bright field images reflecting the different morphology-based categories (Cat #1: straight, minimal curvature, clear somites; Cat #2: curved but clear segments & somites; Cat #3: very curved, somites & segment borders are still distinguishable; Cat #4: not properly elongated or completely collapsed; somites & segment borders not clear) of MG+RAL embedded Axioloids at 96 h and 120 h in 2 different cell lines g, the 409B2 (N=5, n=128 Axioloids) and h, the 201B7 Luc (96 h N=3, n=91 Axioloids and 120 h N=3, n=92 Axioloids). i, and j, Length measurement of Axioloids at 96 h and 120 h after embedding in MG only, MG+RAL, MG+ROL or MG+RA for i, 409B2 and j, 201B7 Luc. k, and l, Periodicity of somite segmentation based on live-cell imaging observations k, for 409B2 (N=3, n=11) and l, 201B7 Luc (N=3, n=9) respectively. m, and n, Total number of somites in Axioloids embedded in m, MG+RAL or MG+ROL or MG+RA at 120 h in 409B2 and n, MG+RAL or MG+RA at 120 h in 201B7 Luc. Scale bar in d, and e, is 50 μm, others are 200 μm.

FIG. 12 shows molecular characterization of human Axioloids exposed to agonists or inhibitors of Retinoic Acid (RA) signaling. a-h, Representative images of immunofluorescence staining of F-actine (Phalloidin) in gray, TBXT in green, and MEOX1 in red and corresponding quantification of signal intensity of Axioloids embedded in MG+RAL a, b, 409B2 at 96 h (N=4, n=10) and 120 h (N=4, n=9) and c, d, 201B7 Luc at 96 h (N=4, n=13) and 120 h (N=4, n=13). Immunofluorescence data of Axioloids embedded in MG+RA shown in e, f, 409B2 at 96 h (N=4, n=15) and 120 h (N=3, n=12) and g, h, 201B7 Luc at 96 h (N=3, n=15) and 120 h (N=4, n=16). i-p, Representative images of HCR staining of MESP2 in yellow, UNCX in cyan and TBX18 in magenta, and corresponding signal intensity measurement along the posterior to anterior axis normalized to the position of the MESP2 signal peak of Axioloids embedded in MG+RAL i, j, 409B2 at 96 h (N=3, n=9) and 120 h (N=3, n=9) and k, l, 201B7 Luc at 96 h (N=3, n=9) and 120 h (N=3, n=9). In situ hybridization data of Axioloids embedded in MG+RA m, n, 409B2 at 96 h (N=3, n=9) and 120 h (N=3, n=5) and o, p, 201B7 Luc at 96 h (N=2, n=5) and 120 h (N=2, n=5). q, Immunofluorescence staining of F-actine (Phalloidin) in gray, TBXT in blue, and SOX2 in red of 96 h (top), and 120 h (bottom), 409B2 Axioloids embedded in MG+RAL. Images shown are representative of 3 independent experiments. Scale bar is 200 μm. r, and s, Representative images of immunofluorescence staining of F-actine (Phalloidin) in gray, TBXT in green, and MEOX1 in red and corresponding quantification of signal intensity of Axioloids embedded in MG+RAL+BMS493 (201B7 Luc) at 120 h of culture (N=3, n=11). t, and u, Representative images of HCR staining of MESP2 in yellow, UNCX in cyan and TBX18 in magenta, and corresponding signal intensity measurement along the posterior to anterior axis normalized to the position of the MESP2 signal peak of Axioloids embedded in MG+RAL+BMS493 (201B7 Luc) at 120 h of culture (N=2, n=6).

FIG. 13 shows single cell RNA-seq based assessment of RA signaling effect on MG embedded human Axioloids. a, Integrated UMAP projection of single-cell transcriptome profiles of Axioloids with all the four conditions (control, MG only, MG+RAL (Retinal) and MG+RA (Retinoic Acid) at both 96 h and 120 h. b, and c, Expression changes by MG+RAL (Retinal) and MG+RA (Retinoic Acid) compared to the MG only conditions for consistently up- or down-regulated genes at both 96 h and 120 h in SM b, and TB c. d, Enrichment analysis of down-regulated genes in SM by addition of Retinal (RAL) to MG. Hallmark and KEGG datasets were employed. e, Expression changes compared to the control (without MG) samples are indicated for the different conditions (RAL or RA addition) at both 96 h and 120 h. Genes indicated here are DEGs identified in FIG. 3g (96 h MG). f-h, Expression levels of indicated genes across different conditions in SM for both 96 h and 120 h Axioloids.

FIG. 14 shows midline and bilateral somite formation in human Axioloids. a, and e, Serial images of elongating human Axioloids (409B2) at 8-10 h intervals, from 72 h to 120 h (extracted from Supplementary Video 5). a, Red dotted arrow shows formation and extension of superficial midline along the posterior to anterior axis of an Axioloid embedded in MG+RAL. e, Red arrowhead shows the initiation of somite division into 2 bilateral structures highlighted and circled with dotted lines, Axioloid embedded in MG+ROL. b-d, Left, brightfield images of Axioloids (409B2) at 120 h, red dotted square encompasses the area enlarged in the right image. b, MG+RAL embedded Axioloid showing a noticeable midline highlighted by a dotted double arrow in the right image. c, and d, respectively MG+RAL and MG+RA embedded Axioloids showing in c, a single bilateral somite and in d, multiple bilateral somites highlighted by red dotted lines. f, h, i, k, Immunofluorescence staining of F-actine (Phalloidin) in gray, TBXT (BRA) in blue, FN in green and MEOX1 in red. g, and j, HCR staining of MESP2 in yellow, UNCX in cyan and TBX18 in magenta of human Axioloid (409B2) at 120 h. Axioloids in f, and g, present a single bilateral somite and in h, and k, multiple bilateral somites. i, j, and k Correspond to an enlarged view of the merged channel images of f, g, and h, respectively. Scale bar is 200 μm.

FIG. 15 shows morphometric and molecular (scRNA-seq) comparison of human Axioloids with human CS11 and CS12 embryos. a, and b, Phalloidin staining and z-stack image-based 3D model creation and somite volume measurement of a, 409B2 and b, 201B7 Luc human Axioloids embedded in MG+RAL at 120 h. c, and d, OPT stack single image and image stack-based 3D model creation and somite volume measurement of the 8 posterior somites of a CS11 human embryo (OPT data of human embryo obtained from the Human Developmental Biology Resource (HDBR)). e-f, UMAP projection of scRNA-seq datasets of CS12 human embryo. e, Colored based on the clustering f, on cell annotations by Xu et. al³⁵ and g, on the sample origins. h, and i, Averaged expression levels of indicated genes in each cluster. h, Shown genes are marker genes of annotated clusters of Xu et. al and i, marker genes of Axioloids. j-m, UMAP projection of integrated scRNA-seq profiles of Axioloids and human embryos, colored by j, their origins (Axioloid or embryo), k, defined clusters l, Axioloid samples (96 h or 120 h) and m, cell types (annotated by Xu et al).

FIG. 16 shows assessment of the HOX Code in human Axioloids. a-c, Pseudotime representation of expression of HOXA, HOXB and HOXD cluster associated genes in human MG exposed Axioloids at 96 h of culture (24 h after embedding into MG); gene expression patterns arranged along pseudotime rank. d, f, and h, Top panels, analysis of the epigenetic landscape at the HOXA, HOXB and HOXD loci profiled by CUT&Tag using antibodies against H3K4me3 (green) and H3K27me3 (red). Bottom panels, visualization of the spatial distribution of the HOXA, HOXB and HOXD transcripts using HybISS analysis of all the members of the respective HOX clusters using sections of 96 h and 120 h Axioloids cultured in MG+RAL. e, g, and i, Heatmap plots of the HybISS data shown in d, f, and h, showing the average HOXA, HOXB and HOXD cluster gene expression along the posterior to anterior axis of 96 h and 120 h MG+RAL Axioloids normalized to the position of the MESP2 signal peak (n=3).

FIG. 17 shows expression gradients of FGF, WNT and RA signaling pathway members in human Axioloids visualized and quantified via HCR and HybISS. a-h, HCR whole mount in situ hybridization images and signal quantification of MG+RAL embedded Axioloids at 96 h and 120 h derived from a, b, e, f, 409B2 and c, d, g, h, 209B7 Luc iPSC lines. Shown images are representative of 3 independent experiments, a, and c, HCR staining of FGF8 in green, WNT3a in red and MESP2 in blue. b, and d, corresponding quantification along the posterior to anterior axis of the signal intensity normalized the position of the WNT3a signal peak (409B2 96 h: N=4, n=7 and 120 h: N=3, n=6, 201B7 Luc 96 h: N=3, n=9 and 120 h: N=3, n=8,). e, and g, HCR staining of CYP26 A1 in cyan, ALDH1 A2 in magenta and RIPPLY2 in yellow. f, and h, corresponding quantification along the posterior to anterior axis of the signal intensity normalized to the position of the CYP26 A1 signal peak (409B2 96 h: N=4, n=10 and 120 h: N=3, n=7, 201B7 Luc 96 h: N=3, n=8 and 120 h: N=3, n=10,). Scale bar 200 m. i-l, HybISS based visualization and quantification of the spatial distribution of FGF/WNT and RA signaling pathway transcripts in human Axioloids at 96 h (top) and 120 h (bottom) of culture in MG+RAL. i, and j, HybISS images and quantification of spatial expression of FGF3, FGF4, FGF8, FGF17, WNT3a and WNT5b. k, and l, HybISS images and quantification of spatial expression of ALDH1 A2, CYP26 A1 and RDH10.

FIG. 18 shows modulating RA, FGF, WNT and Notch signaling in human Axioloids. a-r, Quantification of the total HES7:Luciferase signal over time and corresponding period measurements as the time interval between the consecutive HES7:Luciferase signal peaks in Axioloids+/−MG in a, b, +RAL (Retinal) (N=5, n=17 and N=5, n=20), c, d, +ROL (Retinol) (N=3 n=12 and N=5, n=20), e, f, +RA (Retinoic Acid) (N=6, n=22 and N=5, n=20). Kymographs of the HES7:Luciferase signal for Axioloids embedded in g, MG+RAL (N=4, n=6 data identical to the one showed in FIG. 4 g, n, u), h, MG+RA (N=4, n=8), and corresponding i, average signal and j, periodicity measurement, k, MG+RAL+DMSO (N=3, n=6), l, MG+RAL+BMS493 (N=3, n=6) and corresponding m, average signal and n, periodicity measurement. o, Effect of the addition of BMS493 (N=3, n=9), r, of DAPT (N=3, n=9), PD173074 (N=3, n=9), and XAV939 (N=3, n=9), compared to DMSO (N=3, n=9) on the total HES7:Luciferase signal over time in MG+RAL Axioloids. p, Comparison of the total number of somites of Axioloids embedded in MG+RAL only or supplemented with DMSO or BMS493 or DAPT, PD173074, XAV939 (N=3, n=9 for all), and length of the Axioloids at 96 h and 120 h after addition of DMSO (N=3, with n=24) in p, and (N=3, n=9) in t, BMS493 (N=3, n=9) or DAPT or PD173074 and XAV939 (N=3, n=9). s, Representative brightfield images of Axioloids embedded in MG+RAL supplemented with DMSO or BMS493 or DAPT or PD173074 and XAV939. Scale bar is 200 μm.

FIG. 19 shows morphological, molecular and functional characterization of patient-like iPSC-derived Axioloids with mutations in HES7 and MESP2. Panels a-d, show data for HES7 KO2, panels e-h, show data for HES7^{R25W} MT2 and panels i-l, show data for MESP2 KO2. a, h, and o, Serial brightfield images of a forming Axioloid at 72 h, 96 h and 120 h extracted from the Supplementary Videos 9, 11, and 13 respectively. b, f, and j, Immunofluorescence staining and signal quantification of MG+RAL embedded Axioloids at 120 h. Top, merged channel images of Axioloids stained for F-actine (Phalloidin) in gray, FN in green, and MEOX1 in red, TBXT (BRA) in Blue. Bottom, corresponding quantification along the posterior to anterior axis of TBXT (green line) and MEOX1 (in red) signal intensity for HES7 KO2 (N=3, n=9), HES7^{R25W} MT2 (N=3, n=9), MESP2 KO2 (N=3, n=12). c, g, and k, HCR staining and signal quantification of MG+RAL embedded Axioloids at 120 h, MESP2 in yellow, UNCX in cyan and TBX18 in magenta, and corresponding signal intensity measurements along the posterior to anterior axis normalized to the position of the MESP2 signal peak for HES7 KO2 (N=3, n=8), HES7^{R25W} MT2 (N=3, n=8), MESP2 KO2 (N=3, n=9). d, h, and l, Kymograph of HES7 oscillatory activity for d, HES7 KO2, h, HES7^{R25W} MT2, and 1, MESP2 KO2 Axioloids embedded in MG+RAL. Scale bar is 200 m.

FIG. 20 shows morphogenetic characterization of axioloids embedded in +MG only and scRNA-seq expression profiles of human axioloids. a, Representative bright field images reflecting the different morphology-based categories (Cat #1: straight, minimal curvature, clear segments; Cat #2: curved but clear segments; Cat #3: very curved, segment borders are still distinguishable; Cat #4: not properly elongated or completely collapsed; segment borders not clear) of MG embedded axioloids at 96 h and 120 h derived using the 201B7 Luc cell line (3 independent experiments, n=60 axioloids). Cat #1 corresponds to FIG. 7b, 96 h, CTL. b, scRNA-seq expression profiles of identified marker genes for each cell cluster described in FIG. 2b, except for E-SM2, M-SM2, and presumably apoptotic cells. The top 50 (or less) genes of higher fold changes are shown. Scale bar is 200 μm.

FIG. 21 shows characterization of retinoid treated human axioloids. a, Bright field images of axioloids at 120 h without MG embedding, after addition of RAL only, ROL only and RA only (no MG in all cases) in 409B2, top panel and 201B7 Luc, bottom panel. b, and c, Representative bright field images reflecting the different morphology-based categories (Cat #1: straight, minimal curvature, clear somites; Cat #2: curved but clear segments & somites; Cat #3: very curved, somites & segment borders are still distinguishable; Cat #4: not properly elongated or completely collapsed; somites & segment borders not clear) of +MG+RAL embedded axioloids at 96 h and 120 h in 2 different cell lines b, the 409B2 (5 independent experiments, n=128 axioloids, mean±SD) and c, the 201B7 Luc hiPSC line (96 h, 3 independent experiments, n=91 axioloids and 120 h, 3 independent experiments, n=92 axioloids, mean±SD). d-i, Representative images of immunofluorescence staining of F-actin (Phalloidin) in gray, TBXT in cyan, and MEOX1 in red and corresponding quantification of signal intensity of 409B2 derived axioloids embedded in d-e, +MG+ROL at 96 h (3 independent experiments, n=9 axioloids) and 120 h (3 independent experiments, n=9 axioloids), f-g, in +MG+RAL at 96 h (4 independent experiments, n=10 axioloids) and 120 h (4 independent experiments, n=9 axioloids), h-i, in +MG+RA 409B2 at 96 h (4 independent experiments, n=15 axioloids) and 120 h (4 independent experiments, n=12 axioloids). j-m, Representative images of HCR staining of UNCX in cyan, TBX18 in magenta and MESP2 in yellow, and corresponding signal intensity measurements along the posterior to anterior axis normalized to the position of the MESP2 signal peak of axioloids embedded in +MG+RAL 409B2 at 96 h (3 independent experiments, n=9 axioloids) and 120 h (3 independent experiments, n=9 axioloids) shown in j-k, and of axioloids embedded in +MG+RA at 96 h (3 independent experiments, n=9 axioloids) and 120 h (4 independent experiments, n=8 axioloids) shown in l-m. Single channel images shown in j correspond to the merged channel image shown in FIG. 4f. Lines in e, g, i, k and m correspond to mean values, error bands represent the 95% confidence interval, of which the top and bottom show the 2.5 and 97.5 percentiles for each data point. Scale bar is 200 m. a.u., arbitrary units.

FIG. 22 shows midline and bilateral somite formation in human axioloids. a, and b, Serial images of elongating human axioloids (409B2) at 8-10 h intervals, from 72 h to 120 h (extracted from Supplementary Video 5). a, Red dotted arrow shows formation and extension of superficial midline along the posterior to anterior axis of an axioloid embedded in +MG+RAL. b, Red arrowheads show the initiation of bilateral somite formation in an axioloid embedded in +MG+ROL. c-e, Left, brightfield images of axioloids (409B2) at 120 h, red dotted square encompasses the area enlarged on the right. c, +MG+RAL embedded axioloid showing a noticeable midline highlighted by an arrowhead in the right image. c, and d, +MG+RAL and +MG+RA embedded axioloids respectively showing in c, a single bilateral somite and in d, multiple bilateral somites highlighted by red arrowheads. f-k, Immunofluorescence and in situ-based assessment of +MG+RAL and +MG+RA treated axioloids with bilateral somites. f, h, i, k, Immunofluorescence staining of F-actin (Phalloidin) in gray, TBXT (BRA) in cyan, MEOX1 in red and FN1 in yellow. g, and j, HCR staining of UNCX in cyan, TBX18 and MESP2 in yellow in magenta of human axioloid (409B2) at 120 h. Axioloids in f and g present a single bilateral somite and in h and k multiple bilateral somites. i, j, and k, Correspond to an enlarged view of the merged channel images of f, g and h respectively. Images shown in c-h are representative of 3 independent experiments. Scale bar is 200 m.

FIG. 23 shows HOX gene expression in axioloids in +MG only supplemented medium. a, Visualization of the spatial distribution of the ACTB, TBXT, HES7, TBX6, MESP2, RIPPLY2, MEOX1, TCF15 transcripts in a section of a 409B2-derived axioloid embedded in +MG+RAL at 96 h using HybISS and b, corresponding heatmap plot showing the average gene expression along the posterior to anterior axis normalized to the position of the MESP2 signal peak (n=3 axioloids). c-j, HybISS-based visualization and quantification of HOX loci. c, e, g, and i, Visualization of the spatial distribution of the HOXA, HOXB, HOXC and HOXD transcripts using HybISS analysis of all the members of the respective HOX clusters using sections of 96 h and 120 h axioloids cultured in MG only. d, f, h, and j, Heatmap plots of the HybISS data shown in c, e, g and i showing the average HOXA, HOXB, HOXC and HOXD cluster gene expression along the posterior to anterior axis of 96 h and 120 h axioloids cultured in MG only (n=3 axioloids).

FIG. 24 shows expression gradients of FGF, WNT and RA signaling pathway members in human axioloids visualized and quantified via HCR and HybISS. a-h, HCR whole mount in situ hybridization images and signal quantification of +MG +RAL embedded axioloids at 96 h and 120 h derived from a, b, e, f, 409B2 and c, d, g, h, 209B7 Luc iPSC lines. Shown images are representative of 3 independent experiments, a, and c, HCR staining of FGF8 in green, WNT3 A in red and MESP2 in blue. b, and d, Corresponding quantification along the posterior to anterior axis of the signal intensity normalized to the position of the WNT3a signal peak (409B2 96 h: 4 independent experiments, n=7 axioloids and 120 h: 3 independent experiments, n=6 axioloids, 201B7 Luc 96 h: 3 independent experiments, n=9 axioloids and 120 h: 3 independent experiments, n=8 axioloids). e, and g, HCR staining of CYP26 A1 in cyan, ALDH1 A2 in magenta and RIPPLY2 in yellow. f, and h, Corresponding quantification along the posterior to anterior axis of the signal intensity (409B2 96 h: 4 independent experiments, n=10 axioloids and 120 h: 3 independent experiments, n=7 axioloids, 201B7 Luc 96 h: 3 independent experiments, n=8 axioloids and 120 h: 3 independent experiments, n=10 axioloids). Lines in b, d, f and h correspond to mean values, error bands represent the 95% confidence interval, of which the top and bottom show the 2.5 and 97.5 percentiles for each data point. Scale bar is 200 m. i-l, HybISS based visualization and quantification of the spatial distribution of FGF/WNT and RA signaling pathway transcripts in human 409B2-derived axioloids at 96 h (top) and 120 h (bottom) of culture in +MG+RAL. i, and j, HybISS images and quantification of spatial expression of FGF3, FGF4, FGF8, FGF17, WNT3 A and WNT5B. k, and l, HybISS images and quantification of spatial expression of ALDH1 A2, CYP26 A1 and RDH10. Lines in j and l correspond to mean values and error bands represent the SD for each data point (n=3 axioloids). a.u., arbitrary units.

FIG. 25 shows effect of alternative culture media on axioloid morphogenesis. ac Bright field images of axioloids embedded in 10% MG+RAL at 96 h and 120 h, generated in a, NDiff227 or b, RMPI media supplemented with B27 with (RPMI+) or without (RPMI-) retinol. Scale bar 200 μm.

FIG. 26 shows effect of bFGF, CHIR99421 (WNT agonist) & SB431542 (TGFβ-inhibitor) concentrations on axioloid induction & morphogenesis. a-c Bright field images of axioloids embedded in 10% MG+RAL at 96 h and 120 h generated with different concentrations of a, bFGF. b, CHIR99421 in 2 cell lines 409B2 (top panel) and 201B7 Luc (bottom panel). c, SB431542. Scale bar 200 μm.

FIG. 27 shows effect of FGF8b and A-83-01 (TGFβ inhibitor) on axioloid morphogenesis. a-b Bright field images of axioloids embedded in 10% MG+RAL at 96 h and 120 h generated with different concentrations of a, FGF8b and b, A-83-01. Scale bar 200 μm.

FIG. 28 shows effect of ECM rich components and TTNBP (retinoid agonist) on axioloid morphogenesis. a-b Bright field images of axioloids at 96 h and 120 h embedded in a medium containing a, RAL and 5% MG or 10% MG or 10% Cultrex or 10% Geltrex or 10% ECMGel. b, 10% MG and ROL or RAL or RA or TTNPB. Scale bar 200 μm.

### DESCRIPTION OF EMBODIMENTS

### Detailed Description

The present disclosure relates to a three-dimensional cellular aggregate termed axioloid generated in vitro from a pluripotent stem cell and to a method for producing the same.

The present disclosure also relates to a three-dimensional cellular aggregate generated in vitro from a pluripotent stem cell, comprising: a mesodermal cell, wherein the cellular aggregate has a polarity in an antero-posterior axis or a rostro-caudal axis and an apical-basolateral axis, and the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition.

The present disclosure also relates to a three-dimensional cellular aggregate generated in vitro from a pluripotent stem cell, comprising: a mesodermal cell and/or progenitor cell wherein the cellular aggregate has a polarity in an antero-posterior axis or a rostro-caudal axis and an apical-basolateral axis, and the cellular aggregate can reconstitute various aspects of somitogenesis and axial development, including e.g. axial elongation, segmentation, epithelial somite formation and patterning (formation of one or more somite like structures), and oscillation of the segmentation clock under a somitogenic culture condition.

The present disclosure also relates to a three-dimensional cellular aggregate generated in vitro from a pluripotent stem cell, comprising: a mesodermal cell, wherein the cellular aggregate has a polarity in an antero-posterior axis or a rostro-caudal axis and an apical-basolateral axis, and a proportion of the mesodermal cell in the cellular aggregate is at least 50%, at least 55%, at least 60%, at least 65%, at least 70%, at least 75%, at least 80%, at least 85%, at least 90%, at least 95%, at least 96%, at least 97%, at least 98%, or at least 99%, based on the number of cells.

The present disclosure also relates to a method for producing a three-dimensional cellular aggregate generated in vitro from a pluripotent stem cell, comprising the steps of: (a) culturing a pluripotent stem cell to induce a three-dimensional cellular aggregate comprising a mesodermal cell; and (b) culturing the cellular aggregate comprising the mesodermal cell to induce a three-dimensional cellular aggregate, wherein the three-dimensional cellular aggregate is the cellular aggregate.

The present disclosure also relates to a method for producing a progenitor cell or a differentiated cell, comprising the step of: culturing the cellular aggregate to induce the progenitor cell or the differentiated cell selected from the group consisting of the following (a) to (f): (a) neuro-mesodermal cell or a progenitor cell thereof; (b) a muscle cell or a progenitor cell thereof; (c) an osteocyte or a progenitor cell thereof; (d) a chondrocyte or a progenitor cell thereof; (e) a tenocyte or a progenitor cell thereof; and (f) an endothelial or hemogenic cell or a progenitor cell thereof.

The present disclosure also relates to a method for producing a progenitor cell or a differentiated cell, comprising the step of: culturing the cellular aggregate to induce the progenitor cell or the differentiated cell selected from the group consisting of the following (a) to (i): (a) neuro-mesodermal cell or a progenitor cell thereof; (b) a muscle cell or a progenitor cell thereof; (c) an osteocyte or a progenitor cell thereof; (d) a chondrocyte or a progenitor cell thereof; (e) a tenocyte or a progenitor cell thereof; and (f) an endotome, endothelial or hemogenic cell or a progenitor cell thereof, (g) an adipocyte cell including white, beige and brown cell or a progenitor cell thereof; (h) a dermis cell or a progenitor cell thereof; and (i) a neural tube cell or a progenitor cell thereof.

The present disclosure also relates to a method for evaluating a test substance, comprising the steps of: culturing a test substance in the presence of a three-dimensional cellular aggregate; and evaluating the three-dimensional cellular aggregate after the culture, wherein the three-dimensional cellular aggregate is the cellular aggregate.

The present disclosure also relates to a method for evaluating gene function or genome function, comprising the steps of: preparing a pluripotent stem cell in which a test gene or a test genome is modified; generating a three-dimensional cellular aggregate from the pluripotent stem cell; and evaluating a three-dimensional cellular aggregate after the culture, wherein the generation of the three-dimensional cellular aggregate is carried out by the method.

The present disclosure also relates to a method for evaluating gene function or genomic sequence function, comprising the steps of: preparing a pluripotent stem cell in which a test gene or a test genomic sequence is modified; generating a three-dimensional cellular aggregate from the pluripotent stem cell; and evaluating a three-dimensional cellular aggregate after the culture, wherein the generation of the three-dimensional cellular aggregate is carried out by the method.

In the present disclosure, the “lower” is used to intend a group having less number and/or amount of a subject compared with criterion, unless otherwise provided.

In the present disclosure, the “higher” is used to intend a group having more number and/or amount of a subject compared with criterion, unless otherwise provided.

In the present disclosure, suitable example of “one or more” may be the number of 1 to 6, in which the preferred one may be the number of 1 to 3.

In the present disclosure, the “marker” means a nucleic acid, a gene, a polypeptide, or a protein that is expressed to a different extent in a target cell. When the marker is a positive marker, the different extent means increased expression compared to the cells to be compared with. When the marker is a negative marker, the different extent means reduced expression compared to the cells to be compared with.

In the present disclosure, the “pluripotent cell” means a cell capable of differentiating into ectodermal, mesodermal, and endodermal cells. The pluripotent cell may also be referred to as a pluripotent stem cell when the pluripotent cell is capable of self-replication.

In the present disclosure, the “ectodermal cell” means a cell destined to be capable of differentiating into neural tissues such as nerves; epithelial tissues such as epidermis; and the like if there is a developmentally appropriate stimulation, and is a cell expressing an ectodermal cell marker to be described below.

In the present disclosure, the “mesodermal cell” means a cell destined to be capable of differentiating into connective tissue, such as bones, cartilages, blood vessels, lymphatic vessels and the like; muscle tissues; and the like if there is a developmentally appropriate stimulation, and is a cell expressing a mesodermal cell marker to be described below.

In the present disclosure, the “mesodermal cell” means a cell destined to be capable of differentiating into connective tissue, such as bones, cartilages, fat, blood vessels, lymphatic vessels and the like; muscle tissues; and the like if there is a developmentally appropriate stimulation, and is a cell expressing a mesodermal cell marker to be described below.

In the present disclosure, the “endodermal cell” means a cell destined to be capable of differentiating into thymus; digestive organs such as stomach, intestine, liver, and the like; respiratory organs such as trachea, bronchi, lungs, and the like; and urinary organs such as bladder, urethra, and the like; and the like if there is a developmentally appropriate stimulation, and is a cell expressing an endodermal cell marker to be described below.

In the present disclosure, the “three-dimensional cellular aggregate” means a structure in which cells are aggregated in three dimensions. The three-dimensional culture aggregate is different from, for example, a two-dimensional cellular aggregate (cell sheet) obtained in a planar culture, and forms a three-dimensional structure by, for example, accumulating cells in a thickness direction.

In the present disclosure, the sequence information of a protein or a nucleic acid encoding the same (e.g., DNA or RNA) described herein is available from Protein Data Bank, UniProt or Genbank.

The present disclosure refers to a cellular aggregate (hereinafter also referred to as “axioloid”) generated in vitro from one or more pluripotent stem cells, a method for producing such cellular aggregate, and the cell derived, obtained or obtainable from the cellular aggregate.

Certain aspect of the cellular aggregate (cell aggregate) described in the present disclosure includes being a three-dimensional cellular aggregate generated in vitro from pluripotent stem composed of mesodermal cells (including primitive streak and presomitic mesoderm cells), wherein the cellular aggregate has a polarity along its antero-posterior and apical-basolateral axis or can obtain, induce, or acquire a polarity along its antero-posterior and apical-basolateral axis, and the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition. The antero-posterior axis may also be referred to as a rostro-caudal axis.

Certain aspect of the cellular aggregate (cell aggregate) described in the present disclosure includes being a three-dimensional cellular aggregate generated in vitro from pluripotent stem cell-derived mesodermal cells (including primitive streak and presomitic mesoderm cells), wherein the cellular aggregate has a polarity along its antero-posterior and apical-basolateral axis or can establish, induce, or acquire a polarity along its antero-posterior and apical-basolateral axis, and the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition. The antero-posterior axis may also be referred to as a rostro-caudal axis.

In one another aspect, the cellular aggregate described in the present disclosure includes being a polarized three-dimensional cellular aggregate generated in vitro from pluripotent stem composed of mesodermal cell, wherein the cellular aggregate has a polarity along its antero-posterior and apical-basolateral axis or can obtain, induce, or acquire a polarity along its antero-posterior and apical-basolateral axis, and the cellular aggregate can form one or more somites or somite-like structures under a somitogenic culture condition.

In another aspect, the cellular aggregate described in the present disclosure includes being a polarized three-dimensional cellular aggregate generated in vitro from pluripotent stem cell-derived mesodermal cells and progenitor cells, wherein the cellular aggregate has a polarity along its antero-posterior and apical-basolateral axis or can obtain, induce, or acquire a polarity along its antero-posterior and apical-basolateral axis, and the cellular aggregate can form one or more somites or somite-like structures under a somitogenic culture condition.

In another aspect, a cellular aggregate of the present disclosure is a three-dimensional cellular aggregate generated in vitro from a pluripotent stem cell, composed of mesodermal cells, wherein the cellular aggregate has a polarity along its anteroposterior and apical-basolateral axis or can obtain, induce, or acquire a polarity along its antero-posterior and apical-basolateral axis, and a proportion of the mesodermal cells in the cellular aggregate is at least 50%, at least 55%, at least 60%, at least 65%, at least 70%, at least 75%, at least 80%, at least 85%, at least 90%, at least 95%, at least 96%, at least 97%, at least 98%, or at least 99%, based on the number of cells. The proportion is preferably at least 50%, more preferably at least 90%.

In the present disclosure, the antero-posterior axis may be defined by an anterior (rostral) region and a posterior (caudal) region. An anterior region cell has, for example, a higher or lower expression of one or more markers as compared to a posterior region cell.

In the present disclosure, the anterior region cell may have a lower expression of one or more markers as compared to the posterior region cell. In this case, for example, the one or more markers are selected from the group consisting of TBXT, SOX2, CYP26 A1, FGF3, FGF4, FGF8, FGF17, WNT3a, WNT5a, WNT5b, TBX6, HES7, MSGN1, MEOX1, TCF15, HOXD13, HOXB, HOXA9, HOXA10 and CDX2, preferably TBXT, SOX2, TBX6, HES7, MEOX1 and TCF15, more preferably TBXT. One type or two or more types of them may be used as the marker.

In the present disclosure, the anterior region cell may have a higher or lower expression of one or more markers as compared to the posterior region cell. In this case, for example, the one or more markers are selected from the group consisting of LFNG, MEOX1, TCF15, UNCX, TBX18, ALDH1 A2 and RDH10. The anterior region may include somitic mesoderm (SM) and head mesoderm-like cells.

In the present disclosure, the posterior (caudal) region may include tailbud (TB) like-cells.

In the present disclosure, the apical-basolateral axis may be defined by an apical region and a basolateral region. In this case, for example, an apical region of a cell has a higher or lower expression of one or more markers comprising aPKC, CDH2, Ezrin and ZO1 as compared to a basolateral region of a cell.

In the present disclosure, the apical region of a cell may have a lower expression of one or more markers as compared to the basolateral region of a cell. In this case, for example, the one or more markers are selected from the group consisting of Fibronectin, Collagen and Laminin. One type or two or more types of them may be used as the marker.

In the present disclosure, the apical region of a cell may have a higher expression of one or more markers as compared to the basolateral region of a cell. In this case, for example, the one or more markers are selected from the group consisting of CDH2, aPKC, Ezrin, ZO1 and F-actin, and is preferably, CDH2 and/or aPKC. One type or two or more types of them may be used as the marker.

In the present disclosure, for example, a proportion of the mesodermal cell in the cellular aggregate is at least 50%, at least 55%, at least 60%, at least 65%, at least 70%, at least 75%, at least 80%, at least 85%, at least 90%, at least 95%, at least 96%, at least 97%, at least 98%, or at least 99%, based on the number of cells. The proportion is preferably at least 50%, more preferably at least 90%.

The mesodermal cell expresses one or more markers (mesodermal cell marker) selected from the group consisting of BRA, MIXL1, NODAL, WNT3a, WNT5a, WNT5b, DLL1, CYP26 A1, TBX6, HES7, MSGN1, RIPPLY1, RIPPLY2, MESP1, MESP2, MEOX1, TCF15, TBX18, UNCX, ALDH1 A2, RDH10 and FLK1/KDR, for example. One type or two or more types of them may be used as the marker.

The mesodermal cell expresses one or more markers (mesodermal cell marker) selected from the group consisting of BRA, SOX2, MIXL1, NODAL, WNT3a, WNT5a, WNT5b, DLL1, CYP26 A1, TBX6, HES7, MSGN1, RIPPLY1, RIPPLY2, MESP1, MESP2, MEOX1, TCF15, TBX18, UNCX, ALDH1 A2, RDH10 and FLK1/KDR, for example. One type or two or more types of them may be used as the marker.

The cellular aggregate of the present disclosure can reconstitute various aspects of somitogenesis and axial development, including axial elongation, segmentation, epithelial somite formation and patterning (formation of one or more somite like structures), and oscillation of the segmentation clock under a somitogenic culture condition, for example. The somite and somite-like structure can be defined as the expression of one or more markers selected from the group consisting of MEOX1, TCF15, FOXC2, TBX18, UNCX, ALDH1 A2 and RDH10.

The cellular aggregate of the present disclosure can reconstitute various aspects of somitogenesis and axial development, including axial elongation, segmentation, epithelial somite formation and patterning (formation of one or more somite like structures), and oscillation of the segmentation clock under a somitogenic culture condition, for example. The somite and somite-like structure can be defined as the expression of one or more markers selected from the group consisting of MEOX1, TCF15, FOXC2, PAX3, TBX18, UNCX, ALDH1 A2 and RDH10.

The cellular aggregate of the present disclosure can form a somite or a somite-like structure under a somitogenic culture condition, for example. The somite and somite-like structure can be defined as the expression of one or more markers selected from the group consisting of MEOX1, TCF15, FOXC2, TBX18, UNCX, ALDH1 A2 and RDH10.

The cellular aggregate of the present disclosure can form a somite or a somite-like structure under a somitogenic culture condition, for example. The somite and somite-like structure can be defined as the expression of one or more markers selected from the group consisting of MEOX1, TCF15, FOXC2, PAX3, TBX18, UNCX, ALDH1 A2 and RDH10.

The somitogenic culture condition is, for example, a presence of a gel and/or a matrix and a retinoic acid, a retinoic acid precursor or its derivative and/or a retinoic acid receptor (RAR) agonist.

The somitogenic culture condition is, for example, a presence of a gel and/or a matrix and a retinoid, including retinoic acid, a retinoic acid precursor or its derivative and/or a retinoic acid receptor (RAR) agonist.

The RAR agonist includes, for example, Vitamin A, retinol, retinal, 9-cis retinoic acid, all-trans type retinoic acid (ATRA), TTNPB, AM580, AM80, LGD1550, E6060, AGN193312, AM555S, CD2314, AGN193174, LE540, CD437, CD666, CD2325, SR11254, SR11363, SR11364, AGN193078, TTNN(Ro19-0645), CD270, CD271, CD2665, SR3985, AGN193273, Ch55, 2AGN190521, CD2366, AGN193109 and/or Re80, preferably, retinal or retinol.

The retinoid includes, for example, Vitamin A, retinol, retinal, 9-cis retinoic acid, 13-cis-retinoic acid, all-trans type retinoic acid (ATRA), TTNPB, AM580, AM80, LGD1550, E6060, AM555S, CD2314, CD437, CD666, CD2325, SR11254, SR11364, TTNN(Ro19-0645), CD-270, CD271, SR3985, and/or Ch55 preferably, retinal or retinol.

The cellular aggregate is, for example, embedded in the gel or the matrix, or disposed inside the gel or the matrix.

The matrix includes, for example, an extracellular matrix. The extracellular matrix includes, for example, collagen, laminin, fibronectin, vitronectin, gelatin and/or entactin. The matrix can be used at various concentrations (e.g., 1%, or 5%, or 10% or 20%) per volume of utilized embedding media. The concentration is preferably 5%, more preferably 10%.

One type or two or more types of them used as the extracellular matrix.

The gel includes, for example, a hydrogel. The gel includes, for example, a basement membrane matrix. The basement membrane matrix includes, for example, one or more group comprising laminin, collagen, fibronectin, gelatin, vitronectin, heparan sulphate proteoglycan and/or entactin. One type or two or more types of them used as the basement membrane matrix. The gel includes, for example, an acrylamide gel, an arginine gel, an agarose gel and/or a polyethylene glycol hydrogel with various biomechanical properties.

In the present disclosure, the somite or the somite-like structure may include an anterior (rostral) portion and/or a posterior (caudal) portion in the antero-posterior axis. An anterior portion cell has, for example, a higher or lower expression of one or more markers as compared to a posterior portion cell.

The anterior (rostral) portion cell may have a higher expression of one or more markers than the posterior portion cell. In this case, for example, the one or more markers are selected from the group consisting of TBX18 and ALDH1 A2, and is preferably TBX18. One type or two or more types of them may be used as the marker.

The anterior portion cell may have a lower expression of one or more markers as compared to the posterior portion cell. In this case, for example, the one or more markers are selected from the group consisting of UNCX and LNFG, and is preferably UNCX. One type or two or more types of them may be used as the marker.

The cellular aggregate of the present disclosure may include anterior paraxial/presomitic mesoderm (aPSM). The aPSM means an area on the posterior (caudal) side of the axioloid located between PSM and SM. The aPSM can be defined by the expression of one or more makers selected from a group of markers consisting of MESP2, RIPPLY2, RIPPLY1 and PCDH8, preferably MESP2.

In the present disclosure, the somite is formed, for example, in a cycle of 3 to 7 hours, a cycle of 3.5 to 6.6 hours or a cycle of 4 to 6 hours. The somite is formed preferably in a cycle of 3.5 to 6.6 hours, more preferably in a cycle of 4 to 6 hours.

In the present disclosure, the length of the somite in the antero-posterior axis is, for example, 30 to 200 μm, 50 to 150 μm, 60 to 140 μm, 70 to 130 μm or 80 to 120 μm. The length of the somite is preferably 30 to 200 m, more preferably 80 to 120 m.

The cellular aggregate may include pluripotent stem cells.

In the present disclosure, the pluripotent stem cell expresses one or more markers (pluripotent stem cell marker) selected from the group consisting of OCT4, SOX2, NANOG, ABCG2, CRIPTO, FOXD3, Connexin43, Connexin45, hTERT, UTF1, ZFP42, SSEA-3, SSEA-4, TRA-1-60, TRA-1-81, LIN28 and REX1, for example. One type or two or more types of them (preferably OCT4, more preferably NANOG) may be used as the marker.

In the present disclosure, the pluripotent stem cell expresses one or more markers (pluripotent stem cell marker) selected from the group consisting of OCT4, SOX2, NANOG, ABCG2, CRIPTO, FOXD3, Connexin43, Connexin45, hTERT, UTF1, ZFP42, SSEA-3, SSEA-4, TRA-1-60, TRA-1-81, LIN28 and REX1. One type or two or more types of them (preferably OCT4, more preferably NANOG) may be used as the marker.

The proportion of the pluripotent stem cell in the cellular aggregate is, for example, 10% or less, 9% or less, 8% or less, 7% or less, 6% or less, 5% or less, 4% or less, 3% or less, 2% or less, or 1% or less, based on the number of cells. The proportion is preferably 1%, more preferably less than 1%.

The proportion of the pluripotent stem cell defined by the expression of NANOG in the cellular aggregate is, for example, 10% or less, 9% or less, 8% or less, 7% or less, 6% or less, 5% or less, 4% or less, 3% or less, 2% or less, or 1% or less, based on the number of cells. The proportion is preferably 1%, more preferably less than 1%.

The cellular aggregates do not substantially include, for example, an endodermal cell and/or an ectodermal cell.

The proportion of the endodermal cell in the cellular aggregate is 10% or less, 9% or less, 8% or less, 7% or less, 6% or less, 5% or less, 4% or less, 3% or less, 2% or less, or 1% or less, based on the number of cells. The proportion is preferably 1%, more preferably less than 1%.

The endodermal cell expresses one or more markers (endodermal cell marker) selected from the group consisting of GATA6, GSC, CDX2, NEDD9, PYY, SHH, SORCS2, CER1, SOX17, FOXA2, TRH1 and FOXA1, and preferably GATA6 and/or SHH, for example. One type or two or more types of them (preferably FOXA2, more preferably GATA6) may be used as the marker.

The proportion of the ectodermal cell in the cellular aggregate is, for example, 10% or less, 9% or less, 8% or less, 7% or less, 6% or less, 5% or less, 4% or less, 3% or less, 2% or less, or 1% or less, based on the number of cells. The proportion is preferably 1%, more preferably less than 1%.

The ectodermal cell expresses one or more markers (ectodermal cell marker) selected from the group consisting of OTX2, GBX2, SIX1, SIX3, SOX1, SOX3, DLXS, EYA2 and BARX1, and preferably OTX2, for example. One type or two or more types of them (preferably SOX1, more preferably OTX2) may be used as the marker.

In the cellular aggregate, an expression of a segmentation clock gene may be subjected to gene oscillation. The gene oscillation means, for example, that the expression level of the target gene is periodically oscillating in space and time. The gene oscillation is also referred to as, for example, a gene expression oscillation.

The segmentation clock gene is a gene selected from the group consisting of LFNG, DKK1, DLL1, DLL3 and HES7, and is preferably HES7. One type or two or more types of them (preferably LFNG, more preferably HES7) may be used as the segmentation clock gene.

The cycle of the gene oscillation is, for example, a cycle of 3 to 7 hours, preferably a cycle of 3.5 to 6.6 hours or more preferably a cycle of 4 to 6 hours.

The pluripotent stem cell used for derivation of axioloids is, for example, a human pluripotent stem cell or a non-human animal pluripotent stem cell. Examples of the non-human animal include amniotes such as a mouse, a rat, a rabbit, a dog, a cat, a cow, a horse, a pig, a monkey, an ape, a dolphin, an elephant, a sea lion, a snake, a gecko, a chicken and the like. The pluripotent stem cell is, for example, an embryonic stem cell or an artificial pluripotent stem cell.

The pluripotent stem cell used for derivation of axioloids is, for example, a human pluripotent stem cell or a non-human animal pluripotent stem cell. Examples of the non-human animal include amniotes such as a mouse, a rat, a rabbit, a dog, a cat, a cow, a horse, a pig, a monkey, an ape, a dolphin, a whale, an armadillo, a tenrec, an elephant, a sea lion, a snake, a gecko, a chicken and the like. Non-human animal pluripotent stem cells also include monotreme species, such as a platypus or an echidna and marsupial species, such as an opossum, a kangaroo, a wombat or the like. The pluripotent stem cell is, for example, an embryonic stem cell or an artificially engineered pluripotent stem cell.

The pluripotent stem cell may be, for example, a pluripotent stem cell in which a gene or genome is modified. Examples of the modification include introduction of a mutation into a coding or non-coding gene or genome region, repair of a coding or non-coding (regulatory) gene or genome mutation, introduction of a foreign gene or non-coding (regulatory) genome region, and knockout of a gene or genome region. The modification of the gene or genome can be performed using, for example, genome editing techniques such as ZFN, TALEN, CRISPR/Cas systems and the like; gene recombination techniques; and the like.

The pluripotent stem cell may be, for example, a pluripotent stem cell in which a gene or genomic sequence is modified. Examples of the modification include introduction of a mutation into a coding or non-coding gene or genome region, repair of a coding or non-coding (regulatory) gene or genome mutation, introduction of a foreign gene or non-coding (regulatory) genome region, and knockout of a gene or genome region. The modification of the gene or genomic sequence can be performed using, for example, genome editing techniques such as ZFN, TALEN, CRISPR/Cas systems and the like; gene recombination techniques; and the like.

In the present disclosure, the cellular aggregate includes, for example, at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000 cells, at least 2500 cells, at least 5000 cells, at least 10000 cells, at least 15000 cells, at least 20000 cells, at least 30000 cells, at least 40000 cells or at least 50000 cells. The number of the cellular aggregate is, for example, 100 to 100000 cells, 200 to 100000 cells, 300 to 100000 cells, 400 to 500000 cells, 600 to 100000 cells, 700 to 100000 cells, 800 to 100000 cells, 900 to 100000 cells, 1000 to 90000 cells, 1500 to 80000 cells, 2000 to 70000 cells, 2500 to 60000 cells, 5000 to 50000 cells, 10000 to 50000 cells, 15000 to 50000 cells, 20000 to 50000 cells, 30000 to 50000 cells or 40000 to 50000 cells. The number of the cellular aggregate is preferably at least 50 cells, more preferably 100 to 1000 cells.

In the present disclosure, the cellular aggregate has, for example, a length of at least 0.05 mm, at least 0.1 mm, at least 0.2 mm, at least 0.3 mm, at least 0.4 mm, at least 0.5 mm, at least 0.6 mm, at least 0.7 mm, at least 0.8 mm, at least 0.9 mm or at least 1 mm. The length of the cellular aggregate is, for example, 0.05 to 10 mm, 0.1 to 9 mm, 0.2 to 8 mm, 0.3 to 7 mm, 0.4 to 6 mm, 0.5 to 5 mm, 0.6 to 4 mm, 0.7 to 3 mm, 0.8 to 2 mm or 0.9 to 1 mm. The length of the cellular aggregate is preferably 0.05 mm to 10 mm, more preferably at least 1 mm.

In a further aspect, the method of the present disclosure is a method for producing a three-dimensional cellular aggregate generated in vitro from a pluripotent stem cell, including the steps of:
(a) culturing a pluripotent stem cell to induce a three-dimensional cellular aggregate including a mesodermal cell; and
(b) culturing the cellular aggregate including the mesodermal cell to induce a three-dimensional cellular aggregate, wherein
the three-dimensional cellular aggregate is the cellular aggregate according to the present disclosure.

In a further aspect, the present disclosure is a method for producing a three-dimensional cellular aggregate generated in vitro from a pluripotent stem cell, including the steps of:
(a) culturing a pluripotent stem cell under two dimensional or three dimensional culture conditions to induce a three-dimensional cellular aggregate including a mesodermal cell; and
(b) culturing the cellular aggregate including the mesodermal cell to induce a three-dimensional cellular aggregate, wherein
the three-dimensional cellular aggregate is the cellular aggregate (‘axioloid’) according to the present disclosure.

In the method of the present disclosure, the “culture” can be carried out, for example, using a medium optionally supplemented with a factor. In the method of the present disclosure, the medium may be replaced during the culture period.

The medium can be prepared using a medium used for culturing animal cells as a basal medium. Examples of the basal medium include IMDM, Medium199, Eagle's Minimum Essential Medium (EMEM), aMEM, Dulbecco's modified Eagle's Medium (DMEM), Ham's F12 medium, RPMI1640, Fischer's medium, a Neurobasal Medium (manufactured by Thermo Fisher Scientific), a stem cell culture medium (e.g., mTeSR-1 (manufactured by STEMCELL Technologies), TeSR-E8 (manufactured by STEMCELL Technologies), CDM-PVA, StemFit (registered trademark), AK02N (manufactured by ReproCELL Inc.), StemPRO (registered trademark) hESC SFM (manufactured by Life Technologies), E8 (manufactured by Life Technologies), Essential 6 (manufactured by Thermo Fisher scientific) and mixed media thereof. The medium may be supplemented with serum or may be serum-free. The medium may contain, for example, serum substitutes such as albumin, transferrin, Knockout Serum Replacement (KSR) (serum substitute for ES-cell culture), N2 supplement (manufactured by Invitrogen), B27 supplement (manufactured by Invitrogen), fatty acids, insulin, collagen progenitor, trace elements, 2-mercaptoethanol, 3′-thiol glycerol and the like. Further, the medium may contain additives such as lipids, amino acids, L-glutamine, Glutamax (manufactured by Invitrogen), non-essential amino acids (NEAA), vitamins, growth factors, low molecular weight compounds, antibiotics, antioxidants, pyruvate, buffers, inorganic salts and the like. When a growth culture is performed using the structure, the medium is preferably a stem cell culture medium to which NEAA, glutamic acid and an antibiotic are added.

The culture conditions in the culture may adopt, for example, common conditions of cell culture. As a specific example, the culture temperature is, for example, 25° C. to 40° C., preferably 30° C. to 40° C. or more preferably about 37° C. The carbon dioxide concentration during the culture is 1 to 10%, preferably 3 to 7%, or more preferably about 5%. The culture is carried out, for example, in a wet environment.

The culture conditions implemented during the culture may adopt, for example, common conditions of cell culture. As a specific example, the culture temperature is, for example, 25° C. to 40° C., preferably 30° C. to 40° C. or more preferably about 37° C. The carbon dioxide concentration during the culture is 1 to 10%, preferably 3 to 7%, or more preferably about 5%. The culture is carried out, for example, in a wet environment.

The method of the present disclosure includes, for example, the steps of:
(a1) culturing the pluripotent stem cell in a medium containing a WNT agonist such as GSK3β inhibitor and FGF agonist such as basic-FGF (bFGF) to initiate their commitment toward primitive streak and mesodermal fate, and/or induce the mesodermal cell, preferably in two-dimensional culture;
(a2) culturing the cells derived, obtained or obtainable from step (al) in a medium containing the Wnt agonist such as GSK3β inhibitor and FGF agonist such as bFGF and a TGFβ inhibitor such as SB-431542 and a ROCK inhibitor to induce the three-dimensional cellular aggregate including mesodermal cells; and optionally
(b2) culturing the three-dimensional cellular aggregate including the mesodermal cell in a medium containing a retinoic acid, a retinoic acid precursor or its derivative and/or a retinoic acid receptor (RAR) agonist in the presence of a gel or a matrix to induce the three-dimensional cellular aggregate, and/or, a morphogenesis and/or a self-organization of the three-dimensional cellular aggregate.

The method of the present disclosure includes, for example, the steps of:
(a1) culturing the pluripotent stem cell in a medium containing a Wnt agonist such as GSK3β inhibitor and FGF agonist such as bFGF to initiate their commitment toward a primitive streak and mesodermal fate, and/or induce the mesodermal cell, preferably in two-dimensional culture (planar culture);
(a2) culturing the cells derived, obtained or obtainable from step (a1) in a medium containing the Wnt agonist such as GSK3β inhibitor and FGF agonist such as bFGF and a TGFβ inhibitor such as SB-431542 and a ROCK inhibitor to induce the three-dimensional cellular aggregate including the mesodermal cell;
(b1) culturing the three-dimensional cellular aggregate including the mesodermal cell in a medium not containing the Wnt agonist such as GSK3β inhibitor, FGF agonist such as bFGF, a TGFβ inhibitor such as SB-431542 and a ROCK inhibitor; and optionally
(b2) culturing the three-dimensional cellular aggregate including the mesodermal cell (primitive streak and paraxial/presomitic mesoderm) in a medium containing a retinoic acid, a retinoic acid precursor or its derivative and/or a retinoic acid receptor (RAR) agonist in the presence of a gel or a matrix to induce the three-dimensional cellular aggregate, and/or, a morphogenesis and/or a self-organization of the three-dimensional cellular aggregate.

The method of the present disclosure includes, for example, the steps of:
(a1) culturing the pluripotent stem cell in a medium containing a WNT agonist such as GSK3β inhibitor and FGF agonist such as bFGF to initiate their commitment toward a primitive streak and mesodermal fate, and/or induce the mesodermal cell, preferably in two-dimensional culture (planar culture);
(a2) culturing the cells derived, obtained or obtainable from step (a1) in a medium containing the WNT agonist such as GSK3β inhibitor and FGF agonist such as bFGF and a TGFβ inhibitor such as SB-431542 and a ROCK inhibitor to induce the three-dimensional cellular aggregate including the mesodermal cell;
(b1) culturing the three-dimensional cellular aggregate including the mesodermal cell in a medium not containing the WNT agonist such as GSK3β inhibitor, FGF agonist such as bFGF, a TGFβ inhibitor such as SB-431542 and a ROCK inhibitor; and optionally
(b2) culturing the three-dimensional cellular aggregate including the mesodermal cell (primitive streak and paraxial/presomitic mesoderm) in a medium containing a retinoid, retinoic acid, a retinoic acid precursor or its derivative and/or a retinoic acid receptor (RAR) agonist in the presence of a gel or a matrix to induce the three-dimensional cellular aggregate, and/or, a morphogenesis and/or a self-organization of the three-dimensional cellular aggregate.

The method of the present disclosure includes, for example, the step of: culturing the three-dimensional cellular aggregate including the mesodermal cell, which is embedded in the gel or the matrix, or disposed inside the gel or the matrix, in a medium containing a retinoic acid, a retinoic acid precursor or its derivative and/or a retinoic acid receptor (RAR) agonist to induce the three-dimensional cellular aggregate.

The method of the present disclosure includes, for example, the step of: culturing the three-dimensional cellular aggregate including the mesodermal cell, which is embedded in the gel or the matrix, or disposed inside the gel or the matrix, in a medium containing a retinoid, retinoic acid, a retinoic acid precursor or its derivative and/or a retinoic acid receptor (RAR) agonist to induce the three-dimensional cellular aggregate.

In the steps (a1), (a2) and/or (b1), the FGF is, for example, bFGF (FGF2).

The concentration of bFGF in the medium is, for example, 1 to 1000 ng/ml or preferably 10 to 1000 ng/ml.

In the steps (a1), (a2) and/or (b1), the GSK3β inhibitor may be, for example, a substance that inhibits the kinase activity of GSK3β protein (e.g., phosphorylation ability to β catenin), specifically, an indirubin derivative such as BIO (GSK-3 P inhibitor IX: 6-bromoindirubin 3′-oxime) or the like; a maleimide derivative such as SB216763 (3-(2,4-dichlorophenyl)-4-(1-methyl-1H-indol-3-yl)-1H-pyrrole-2,5-dione), SB415286 (3-[(3-chloro-4-hydroxyphenyl)amino]-4-(2-nitrophenyl)-1H-pyrrole-2,5-dione or the like; a phenyl a bromomethylketone compound such as SK-3β inhibitor VII (4-dibromoacetophenone) or the like; a cell membrane penetrating phosphorylated peptide such as L803-mts (GSK-3p peptide inhibitor; Myr-N-GKEAPPAPPQSpP-NH2) or the like; CHIR99021 (6-[2-[4-(2,4-Dichlorophenyl)-5-(4-methyl-1H-imidazol-2-yl)pyrimidin-2-ylamino]ethylamino]pyridine-3-carbonitrile); an expression suppressing the nucleic acid molecule of GSK3β protein (siRNA, shRNA, antisense or the like) and the like. The GSK3β inhibitor is preferably CHIR99021, because of its high selectivity to GSK3β. The GSK3β inhibitor is commercially available, for example, from Calbiochem, Biomol and the like. In the present disclosure, a WNT agonist including recombinant WNT proteins such as WNT3a as well as other canonical and non-canonical WNT agonists can be used instead of the GSK3β inhibitor.

The concentration of the GSK3β inhibitor in the medium is, for example, 1 nmol/l (also referred to as “M” hereinafter) to 1000 μmol/l, preferably 10 nmol/l to 100 μmol/l or more preferably 100 nmol/l to 100 μmol/l.

In the steps (a2) and/or (b1), the TGFβ inhibitor is a substance that inhibits SMAD mediated signaling caused by binding of TGFβ to a receptor. Examples of the TGFβ inhibitor include a substance that inhibits binding to the ALK family, which is a TGFβ acceptor, and a substance that inhibits phosphorylation of SMAD by the ALK family. Specific examples of the TGFβ inhibitor include Lefty-1 (NCBI Accession Number: NM-010094 (mouse), NM-020997 (human) SB431542 (4-(4-(benzo[d][1,3]dioxol-5-yl)-5-(pyridine-2-yl)-1H-imidazol-2-yl)benzamide), SB202190 (4-(4-Fluorophenyl)-2-(4-hydroxyphenyl)-5-(4-pyridyl)-1H-imidazole), SB505124 (2-(5-Benzo1,3dioxol-5-yl-2-tert-butyl-3H-imidazol-4-yl)-6-methylpyridine), NPC30345, SD093, SD908, SD208 (Scios), LY2109761, LY364947, LY580276 (Lilly Research Laboratories) and A-83-01 (WO2009/146408), and SB431542 is preferred.

The concentration of the TGFβ inhibitor in the medium is, for example, 1 nmol/l to 1000 μmol/l, preferably 10 nmol/l to 100 μmol/l or more preferably 100 nmol/l to 100 μmol/1.

In the steps (a2) and/or (b1), the ROCK inhibitor is a substance that can suppress the function of the Rho-kinase (ROCK). Examples of the ROCK inhibitor include Y27632 ((+)-(R)-trans-4-(1-aminoethyl)-N-(4-pyridyl)cyclohexanecarboxamide dihydrochloride), Fasudil/HA1077 (5-(1, 4-Diazepane-1-sulfonyl)isoquinoline), H-1152 ((S)-(+)-2-Methyl-1-[(4-methyl-5-isoquinolinyl)sulfonyl]homopiperazine), Wf-536 ((+)-(R)-4-(1-Aminoethyl)-N-(4-pyridyl)benzamide) and an expression suppressing the nucleic acid molecule of ROCK protein (siRNA, shRNA, antisense, etc.), and Y27632 is preferred.

The concentration of the ROCK inhibitor in the medium is, for example, 1 nmol/l to 50 μmol/l, 10 nmol/l to 40 μmol/l, 50 nmol/l to 30 μmol/l, 100 nmol/l to 25 μmol/l, 500 nmol/l to 20 μmol/l or 750 nmol/l to 15 mol/l, and is preferably 1 nmol/l to 40 mol/l, more preferably 1 nmol/l to 15 mol/l.

In the step (b2), the RAR agonist is, for example, Vitamin A, retinol, retinal, 9-cis retinoic acid, all-trans type retinoic acid (ATRA), TTNPB, AM580, AM80, LGD1550, E6060, AGN193312, AM555S, CD2314, AGN193174, LE540, CD437, CD666, CD2325, SR11254, SR11363, SR11364, AGN193078, TTNN(Ro19-0645), CD270, CD271, CD2665, SR3985, AGN193273, Ch55, 2AGN190521, CD2366, AGN193109 and/or Re80, preferably, retinal or retinol.

In the step (b2), the retinoid includes, for example, Vitamin A, retinol, retinal, 9-cis retinoic acid, 13-cis-retinoic acid, all-trans type retinoic acid (ATRA), TTNPB, AM580, AM80, LGD1550, E6060, AM555S, CD2314, CD437, CD666, CD2325, SR11254, SR11364, TTNN(Ro19-0645), CD-270, CD271, SR3985, and/or Ch55 preferably, retinal or retinol.

The concentration of the retinoic acid, the retinoic acid precursor or derivative and/or the retinoic acid receptor (RAR) agonists in the medium is, for example, 1 nmol/l to 1000 μmol/l, preferably 10 nmol/l to 100 μmol/l or more preferably 100 nmol/l to 100 μmol/1.

The concentration of the retinoid, retinoic acid, the retinoic acid precursor or derivative and/or the retinoic acid receptor (RAR) agonists in the medium is, for example, 1 nmol/l to 1000 μmol/l, preferably 10 nmol/l to 100 μmol/l or more preferably 100 nmol/l to 100 μmol/l.

In the methods of the present disclosure, for example, the GSK3β inhibitor is CHIR99021, the FGF is bFGF, the TGFβ inhibitor is SB431542 and/or the ROCK inhibitor is Y-27632.

In the step (b2), for example, the matrix includes an extracellular matrix.

The matrix includes, for example, an extracellular matrix. The extracellular matrix includes, for example, collagen, laminin, fibronectin, vitronectin, gelatin and/or entactin. One type or two or more types of them may be used as the extracellular matrix.

In the step (b2), the gel includes, for example, a hydrogel. The gel includes, for example, a basement membrane matrix. The basement membrane matrix includes, for example, fibronectin, laminin, collagen, vitronectin, gelatin, heparan sulphate proteoglycan and/or entactin. One type or two or more types of them may be used as the basement membrane matrix. The gel includes an acrylamide gel, an arginine gel, an agarose gel and/or a polyethylene glycol hydrogel. The gel includes preferably an agarose gel, more preferably a polyethylene glycol hydrogel.

In the step (b2), the concentration of the gel and/or the matrix in the medium is, for example, a concentration capable of three-dimensional culture, and can be appropriately set according to the type of the gel and the matrix. As a specific example, the concentration of the gel and/or the matrix in the medium is, for example, 0.01 to 50% (v/v), 0.1 to 25% (v/v), 1 to 20% (v/v) or 5 to 10% (v/v). The concentration is preferably 0.01 to 50% (v/v), more preferably 5 to 10% (v/v).

In the step (b2), the concentration of the gel and/or the matrix in the medium is, for example, a concentration enabling three-dimensional culture, and can be appropriately adjusted according to the type of the gel and the matrix. As a specific example, the concentration of the gel and/or the matrix in the medium is, for example, 0.01 to 50% (v/v), 0.1 to 25% (v/v), 1 to 20% (v/v) or 5 to 10% (v/v). The concentration is preferably 0.01 to 50% (v/v), more preferably 5 to 10% (v/v).

In the method of the present disclosure, for example, a proportion of the mesodermal cell in the cellular aggregate including the mesodermal cell is at least 50%, at least 55%, at least 60%, at least 65%, at least 70%, at least 75%, at least 80%, at least 85%, at least 90%, at least 95%, at least 96%, at least 97%, at least 98% or at least 99%, based on the number of cells. The proportion is preferably at least 50%, more preferably at least 90%.

The mesodermal cell expresses one or more markers (mesodermal cell marker) selected from the group consisting of TBXT, SOX2, CYP26 A1, FGF3, FGF4, FGF8, FGF17, WNT3a, WNT5a, WNT5b, TBX6, HES7, MSGN1, MEOX1, TCF15, HOXD13, HOXB, HOXA9, HOXA10 and CDX2, for example. One type or two or more types of them may be used as the marker.

In the method of the present disclosure, for example, the cellular aggregate including the mesodermal cell substantially does not include an endodermal cell and/or an ectodermal cell.

The proportion of the endodermal cell in the cellular aggregate including the mesodermal cell is, for example, 10% or less, 9% or less, 8% or less, 7% or less, 6% or less, 5% or less, 4% or less, 3% or less, 2% or less, or 1% or less, based on the number of cells. The proportion is preferably 10% or less, more preferably 1% or less.

The endodermal cell expresses one or more markers (endodermal cell marker) selected from the group consisting of GATA6, GSC, CDX2, NEDD9, PYY, SHH, SORCS2, CER1, SOX17, FOXA2, TRH1 and FOXA1, and preferably GATA6 and/or SHH, for example. One type or two or more types of them may be used as the marker.

The proportion of the ectodermal cell in the cellular aggregate including the mesodermal cell is, for example, 10% or less, 9% or less, 8% or less, 7% or less, 6% or less, 5% or less, 4% or less, 3% or less, 2% or less, or 1% or less, based on the number of cells. The proportion is preferably 10% or less, more preferably 1% or less.

The ectodermal cell expresses one or more marker (ectodermal cell marker) selected from the group consisting of OTX2, GBX2, SIX1, SIX3, SOX1, SOX2, SOX3, DLXS, EYA2 and BARX1, and preferably OTX2, for example. One type or two or more types of them may be used as the marker.

The ectodermal cell expresses one or more marker (ectodermal cell marker) selected from the group consisting of OTX2, GBX2, SIX1, SIX3, SOX1, SOX3, DLXS, EYA2 and BARX1, and preferably OTX2, for example. One type or two or more types of them may be used as the marker.

The number of days of culture of the step (a) is, for example, 0.1 to 10 days, 0.25 to 6 days, 0.25 to 4 days, 0.5 to 3 days or 0.5 to 2 days. The number is preferably 0.1 to 10 days, more preferably 0.5 to 2 days.

The number of days of culture of the step (a1) is, for example, 0.1 to 5 days, preferably 0.25 to 3 days or more preferably 0.5 to 2 days.

The number of days of culture of the step (a2) is, for example, 0.1 to 5 days, preferably 0.25 to 3 days or more preferably 0.5 to 2 days.

The number of days of culture of the step (b) is, for example, 0.1 to 10 days, 1 to 9 days, 2 to 8 days or 3 to 7 days. The number is preferably 0.1 to 10 days, more preferably 2 to 8 days.

The number of days of culture of the step (b1) is, for example, 0.1 to 7 days preferably 0.25 to 5 days, or more preferably 0.5 to 4 days.

The number of days of culture of the step (b2) is, for example, 0.1 to 7 days preferably 0.25 to 5 days, or more preferably 0.5 to 4 days.

In the step (a1), for example, the pluripotent stem cell is one or more suitable dissociated pluripotent stem cell(s) or one or more suitable cell(s) suspension containing any pluripotent stem cell(s).

In the step (a1), the pluripotent stem cell is, for example, a human pluripotent stem cell or a non-human animal pluripotent stem cell. Examples of the non-human animal include amniotes such as a mouse, a rat, a rabbit, a dog, a cat, a cow, a horse, a pig, a monkey, an ape, a dolphin, an elephant, a sea lion, a snake, a gecko, a chicken, and the like. The pluripotent stem cell is, for example, an embryonic stem cell or an artificial pluripotent stem cell.

In the step (a1), the pluripotent stem cell is, for example, a human pluripotent stem cell or a non-human animal pluripotent stem cell. Examples of the non-human animal include amniotes such as a mouse, a rat, a rabbit, a dog, a cat, a cow, a horse, a pig, a monkey, an ape, a dolphin, a whale, an armadillo, a tenrec, an elephant, a sea lion, a snake, a gecko, a chicken and the like. Non-human animal pluripotent stem cells also include monotreme species, such as a platypus or an echidna and marsupial species, such as an opossum, a kangaroo, a wombat or the like. The pluripotent stem cell is, for example, an embryonic stem cell or an artificially engineered pluripotent stem cell.

The cellular aggregate including the mesodermal cell includes, for example, at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000 cells, at least 2500 cells, at least 5000 cells, at least 10000 cells, at least 15000 cells, at least 20000 cells, at least 30000 cells, at least 40000 cells or at least 50000 cells. The cellular aggregate includes preferably at least 50 cells, more preferably at least 100 to 1000 cells.

The cellular aggregate including the mesodermal cell is made of, for example, at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000 cells, at least 2500 cells, at least 5000 cells, at least 10000 cells, at least 15000 cells, at least 20000 cells, at least 30000 cells, at least 40000 cells or at least 50000 cells. The cellular aggregate includes preferably at least 50 cells, more preferably at least 100 to 1000 cells.

The cellular aggregate including the mesodermal cell has, for example, a length of at least 0.05 mm, at least 0.1 mm, at least 0.2 mm, at least 0.3 mm, at least 0.4 mm, at least 0.5 mm, at least 0.6 mm, at least 0.7 mm, at least 0.8 mm, at least 0.9 mm or at least 1 mm. The cellular aggregate has preferably at least 0.05 mm, more preferably at least 0.5 mm.

In another aspect, the present disclosure provides a cell obtained from the three-dimensional cellular aggregate of the present disclosure.

In yet another aspect, the present disclosure provides a method for producing a progenitor cell or a differentiated cell, including the step of:
culturing the three-dimensional cellular aggregate of the present disclosure to induce the progenitor cell or the differentiated cell selected from the group consisting of the following (a) to (f):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endothelial or hemogenic cell or a progenitor cell thereof.

In yet another aspect, the present disclosure provides a method for producing a progenitor cell or a differentiated cell, including the step of:
culturing the three-dimensional cellular aggregate of the present disclosure to induce the progenitor cell or the differentiated cell selected from the group consisting of the following (a) to (i):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endotome or endothelial or hemogenic cell or a progenitor cell thereof;
(g) an adipocyte cell including white, beige and brown cell or a progenitor cell thereof;
(h) a dermis cell or a progenitor cell thereof; and
(i) a neural tube cell or a progenitor cell thereof.

The method for inducing the differentiated cell and the progenitor cell thereof of (a) to (f) can be carried out in the same manner as, for example, a method for inducing each differentiated cell and a progenitor cell thereof from the mesodermal cell.

The method for inducing the differentiated cell and the progenitor cell thereof of (a) to (i) can be carried out in the same manner as, for example, a method for inducing each differentiated cell and a progenitor cell thereof from the mesodermal cell.

The production method of the present disclosure may include, for example, the step of inducing a three-dimensional cellular aggregate from a pluripotent stem cell prior to the inducing. In this case, for example, the inducing of the three-dimensional cellular aggregate may be performed by the method for producing the three-dimensional cellular aggregate of the present disclosure.

The production method of the present disclosure may include, for example, the step of forming a three-dimensional cellular aggregate from a pluripotent stem cell prior to the induction. In this case, for example, the formation of the three-dimensional cellular aggregate may be performed by the method for producing the three-dimensional cellular aggregate of the present disclosure.

In another aspect, the present disclosure provides a method for evaluating a test substance, including the steps of: culturing a test substance in the presence of a three-dimensional cellular aggregate; and evaluating the three-dimensional cellular aggregate through and/or after the culturing, wherein the three-dimensional cellular aggregate is the cellular aggregate of the present disclosure.

The type of the test substance is not particularly limited, and examples thereof include a protein, an antibody, a peptide, a nucleic acid molecule, a sugar chain, a lipid, an organic low molecular weight compound, an inorganic low molecular weight compound, a bacterial releasing substance, a fermentation product, a cell extract, a vacuole culture supernatant, a plant extract and an animal tissue extract. One type or two or more types of them (preferably an organic low molecular weight compound, more preferably a protein) may be used as the test substance.

In the evaluation, for example, a test substance that changes a polarity of the cellular aggregate, a shape of the cellular aggregate and/or a size of the cellular aggregate is selected as a candidate substance that modifies, promotes or suppresses the polarity of the cellular aggregate, the shape of the cellular aggregate and/or the size of the cellular aggregate.

The culture is, for example, a culture under a segmentation culture condition. The segmentation culture conditions can be referred to, for example, the above description. The culture conditions may adopt, for example, the common conditions of cell culture.

During the evaluation, for example, a test substance that changes somitogenesis of the cellular aggregate is selected as a candidate substance that modifies, promotes or suppresses the somitogenesis process of the cellular aggregate.

The culture is a culture inducing the progenitor cell or the differentiated cell selected from the group consisting of the following (a) to (f):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endothelial or hemogenic cell or a progenitor cell thereof.

The culture is a culture inducing the progenitor cell or the differentiated cell selected from the group consisting of the following (a) to (i):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endotome or endothelial or hemogenic cell or a progenitor cell thereof;
(g) an adipocyte cell including white, beige and brown cell or a progenitor cell thereof;
(h) a dermis cell or a progenitor cell thereof; and
(i) a neural tube cell or a progenitor cell thereof.

During the evaluation, for example, a test substance that promotes or suppresses induction of the progenitor cell or the differentiated cell selected from the group consisting of (a) to (f) is selected as a candidate substance that promotes or suppresses induction of the progenitor cell or the differentiated cell selected from the group consisting of (a) to (f).

During the evaluation, for example, a test substance that promotes or suppresses induction of the progenitor cell or the differentiated cell selected from the group consisting of (a) to (i) is selected as a candidate substance that promotes or suppresses induction of the progenitor cell or the differentiated cell selected from the group consisting of (a) to (i).

In the evaluation method, the evaluation is an evaluation using a control in which the test substance is not present as a reference.

During the evaluation, the method of evaluation uses a control in which the test substance is not present as a reference.

The culture is generation of a three-dimensional cellular aggregate from a pluripotent stem cell, and the generation of the three-dimensional cellular aggregate is carried out by the method for producing the three-dimensional cellular aggregate of the present disclosure.

The culture refers to the generation of a three-dimensional cellular aggregate from a pluripotent stem cell, where the generation of the three-dimensional cellular aggregate is carried out by the method for producing the three-dimensional cellular aggregate described in the present disclosure.

In another aspect, the present disclosure provides a method for evaluating gene function or genome function, including the steps of: preparing a pluripotent stem cell which a test gene or a test genome is modified; generating a three-dimensional cellular aggregate from the pluripotent stem cell; and evaluating a three-dimensional cellular aggregate through and/or after the culturing, wherein the generation of the three-dimensional cellular aggregate is carried out by the method for producing the three-dimensional cellular aggregate of the present disclosure.

In another aspect, the present disclosure provides a method for evaluating gene function or genomic sequence function, including the steps of: engineering a pluripotent stem cell in which a test gene or a test genomic sequence is modified; generating a three-dimensional cellular aggregate from the pluripotent stem cell; and evaluating a three-dimensional cellular aggregate through and/or after the culturing, wherein the generation of the three-dimensional cellular aggregate is carried out by the method for producing the three-dimensional cellular aggregate of the present disclosure.

During the evaluation, for example, a test gene or a test genome (coding and noncoding genome regions) that changes a polarity of the cellular aggregate, a shape of the cellular aggregate and/or a size of the cellular aggregate is evaluated as a candidate gene or a candidate genome (coding and non-coding genome regions) that modifies, promotes or suppresses the polarity of the cellular aggregate, the shape of the cellular aggregate and/or the size of the cellular aggregate.

During the evaluation, for example, a test gene or a test genomic sequence (coding and non-coding genome regions) that changes a polarity of the cellular aggregate, a shape of the cellular aggregate and/or a size of the cellular aggregate is evaluated as a candidate gene or a candidate genomic sequence (coding and non-coding genome regions) that modifies, promotes or suppresses the polarity of the cellular aggregate, the shape of the cellular aggregate and/or the size of the cellular aggregate.

The function evaluation method of the present disclosure includes, for example, culturing in the presence of a three-dimensional cellular aggregate under a somitogenic culture condition. In this case, during the evaluation, a test gene or a test genome (coding and non-coding genome regions) that changes somitogenesis, including axial elongation, segmentation, epithelialization or oscillation of the segmentation clock of the cellular aggregate is evaluated as a candidate gene or a candidate genome (coding or non-coding) that modifies, promotes or suppresses the somitogenesis, including axial elongation, segmentation, epithelialization or oscillation of the segmentation clock of the cellular aggregate.

The method to evaluate the function in the present disclosure includes, for example, culturing in the presence of a three-dimensional cellular aggregate under a somitogenic culture condition. In this case, during the evaluation, a test gene or a test genomic sequence (coding and non-coding genome regions) that changes somitogenesis, including axial elongation, segmentation, epithelialization or oscillation of the segmentation clock of the cellular aggregate is evaluated as a candidate gene or a candidate genomic sequence (coding or non-coding) that modifies, promotes or suppresses the somitogenesis, including axial elongation, segmentation, epithelialization or oscillation of the segmentation clock of the cellular aggregate.

In the present disclosure, the gene may be any gene and the example thereof is a gene related to human diseases (e.g., HES7). The test genes may be a gene having gene mutation related to human diseases (e.g., HES7^{R25W} and spondylocostal dysostosis).

In the function evaluation method of the present disclosure, the genome is an exon region, an intron region, a promoter region, an enhancer region and/or a non-coding region of the genome.

In the function evaluation method of the present disclosure, the genomic sequence is an exon region, an intron region, a promoter region, an enhancer region and/or a noncoding region of the genome.

The following Preparations and Examples are given for the purpose of illustrating the present invention in more detail. However, the scope of the present invention is not limited to the following.

### EXAMPLES

The present disclosure will be described specifically below with reference to examples. It is to be noted, however, that the present disclosure is by no means limited to embodiments described in the following examples.

Abstract The segmented body plan of vertebrates is established during somitogenesis, a well-studied process in model organisms, but remains largely elusive in humans due to ethical and technical limitations. Despite recent advances with pluripotent stem cell (PSC)-based approaches¹⁻⁵, a system that robustly recapitulates human somitogenesis in both space and time remains missing. Here, a PSC-derived mesoderm-based 3D model of human segmentation and somitogenesis is introduced, which is termed Axioloids, that captures accurately the oscillatory dynamics of the segmentation clock as well as the morphological and molecular characteristics of segmentation and sequential somite formation in vitro. Axioloids show proper rostrocaudal patterning of forming segments and robust anterior-posterior FGF/WNT signaling gradients and Retinoic Acid (RA) signaling components. An unexpected critical role of RA signaling in the stabilization of forming segments is identified, indicating distinct, but also synergistic effects of RA and extracellular matrix (ECM) on the formation and epithelialization of somites. Importantly, comparative analysis demonstrates striking similarities of Axioloids to the human embryo, further validated by the presence of the HOX code in Axioloids. Lastly, the utility of the Axioloid system is demonstrated to study the pathogenesis of human congenital spine diseases, by using patient-like iPSC cells with mutations in HES7 and MESP2, which revealed disease-associated phenotypes including loss of epithelial somite formation and abnormal rostrocaudal patterning. These results suggest that Axioloids represent a promising novel platform to study axial development and disease in humans.

### Introduction

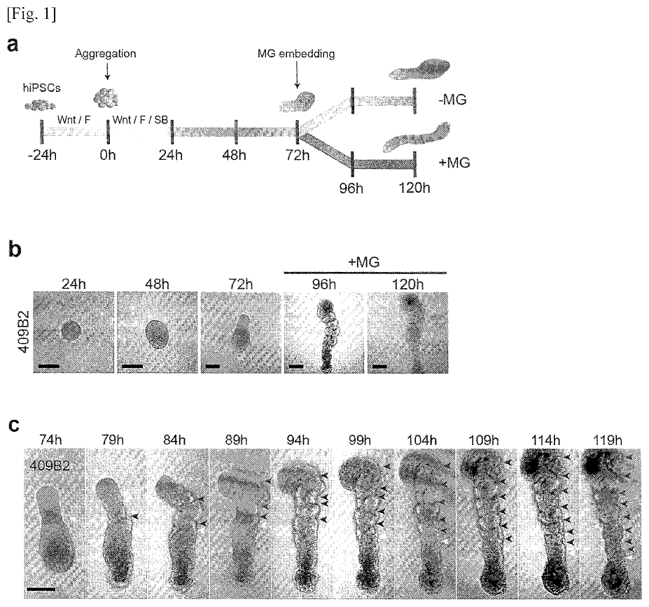

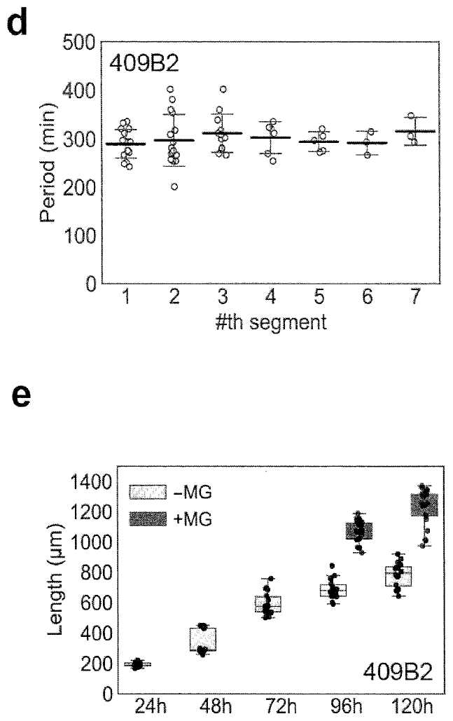

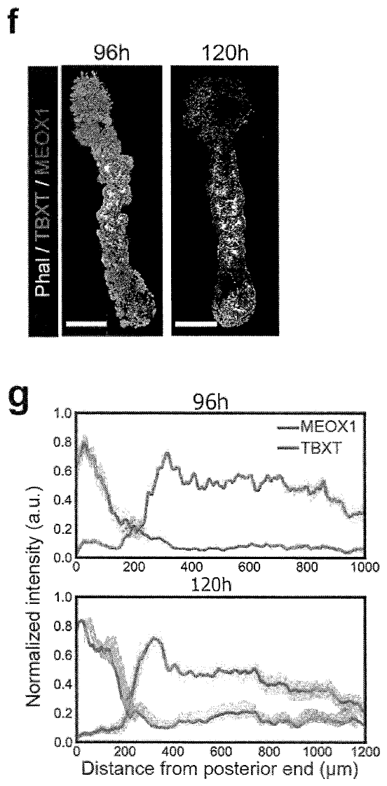

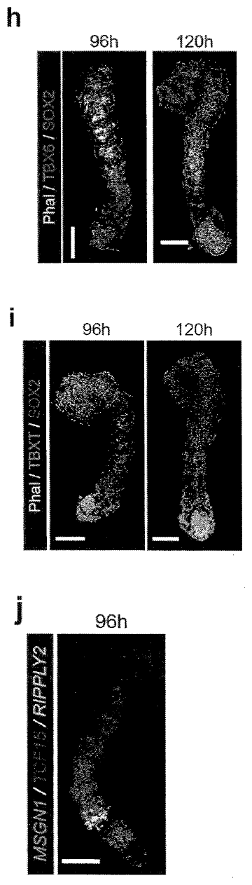

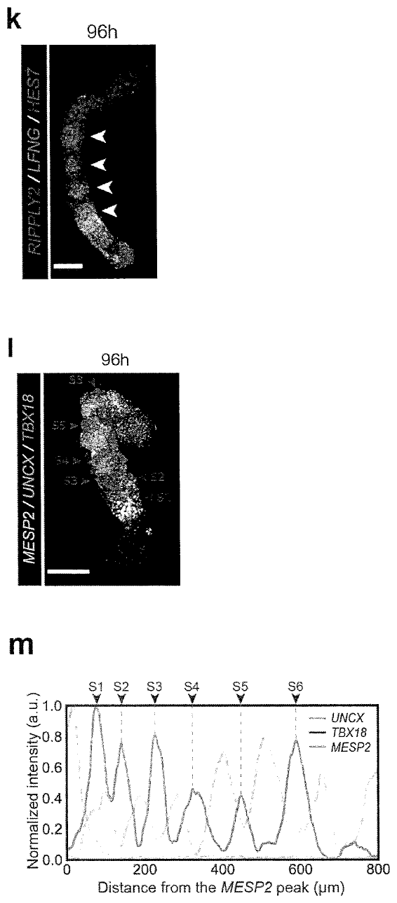

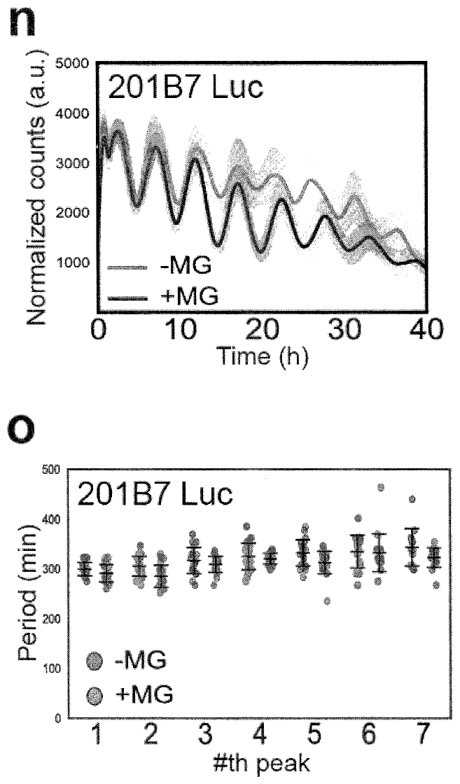

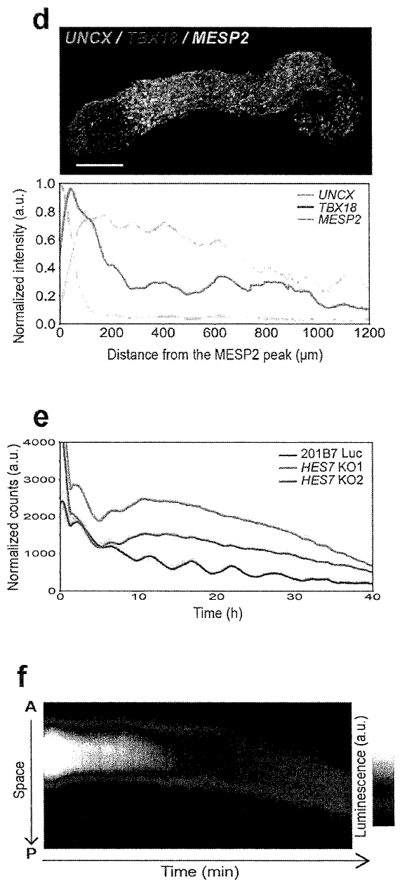

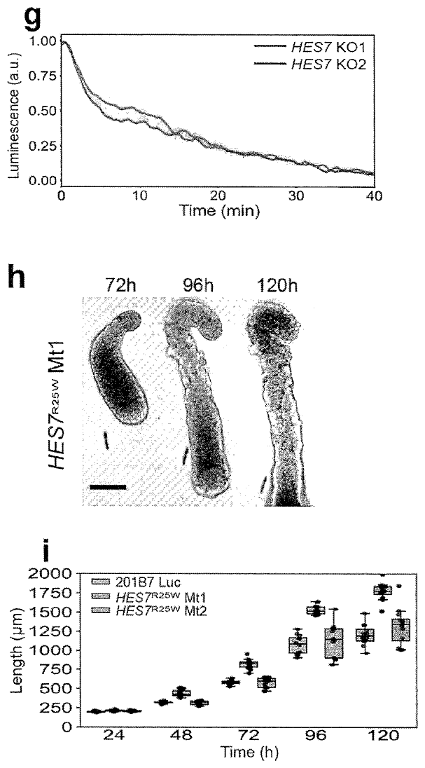

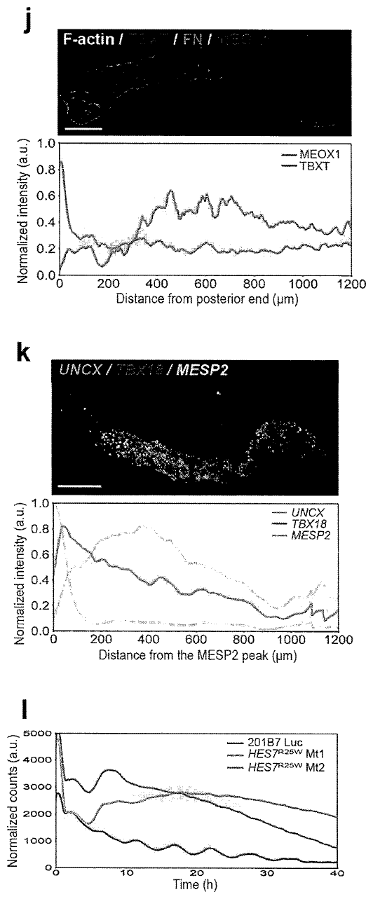

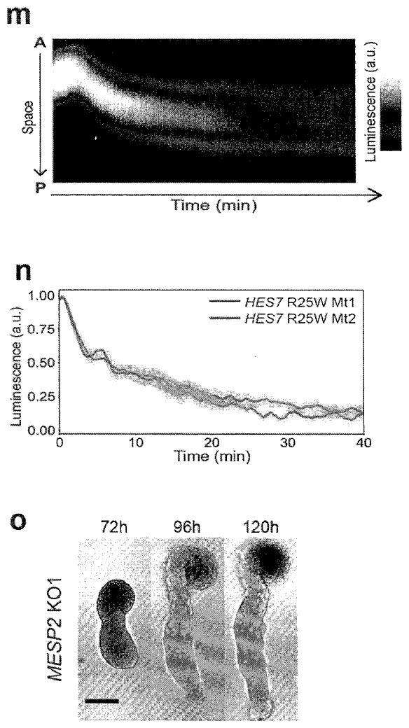

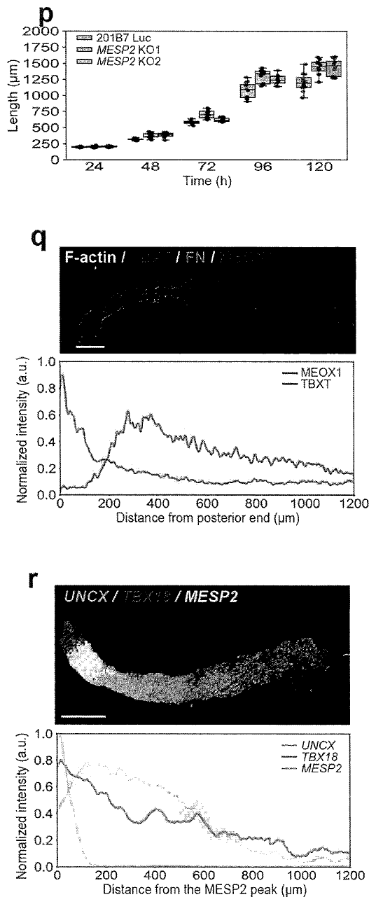

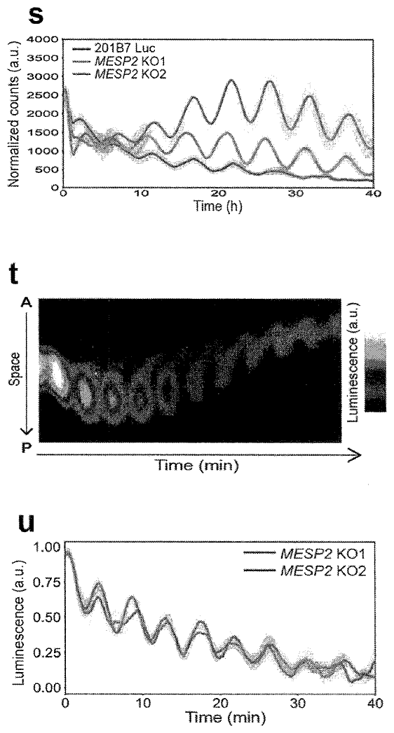

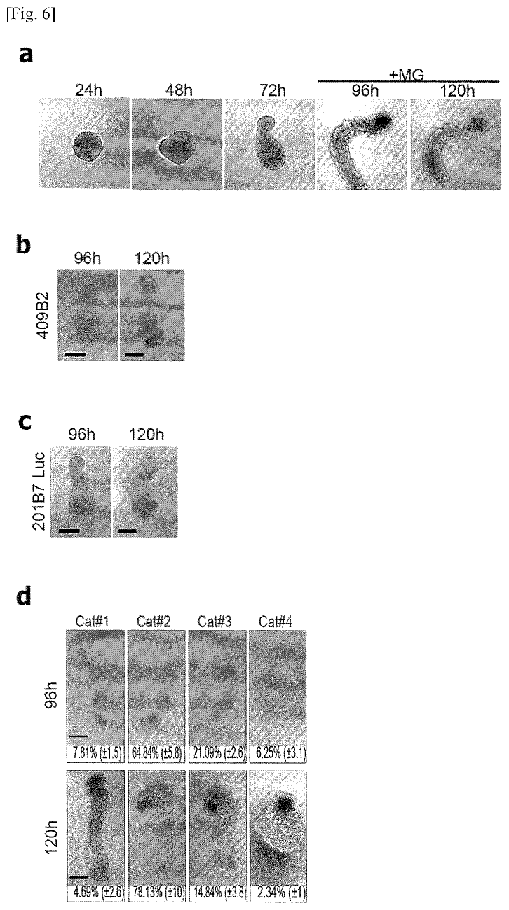

Previously, the human segmentation clock in vitro^{1,4,6} was able to be reconstructed. However, these systems lacked the ability to form proper axial segmental organization—a central feature of all vertebrates—limiting their utility to understand how higher-order tissue organization and more advanced stages of human embryonic development occur. As supplementation of extracellular matrix (ECM) molecules in vitro, have been shown to facilitate the formation of higher-order tissue structures in organoids as well as help mimic morphogenetic processes in mouse pluripotent stem cell-derived gas-truloids² and trunk-like structures³, a single-germ layer, mesoderm-based 3D model of human axial development using human induced pluripotent stem cells (iPSCs) and ECM was set out to be established. iPSCs were exposed in a step-wise manner to signals promoting primitive streak (PS) and presomitic mesoderm (PSM) fates (FIG. 1a and experimental procedures). The spontaneously symmetry-breaking and elongating mesodermal aggregates (FIG. 1b, FIG. 6a, b) were then embedded into 10% Matrigel (MG), an ECM-rich culture supplement, which led to the spatiotemporally coordinated emergence of segments along the anterior-posterior axis of these growing and usually curved structures, which were termed Axioloids (FIG. 1b, FIG. 6a-e). Axioloids were derived from two different human iPSC lines to ensure reproducibility and assessed for their morphological, molecular and functional features.

### Results and Discussion

### Axioloids Display Morphological and Molecular Features of the Vertebrate Embryonic Axis and Tail

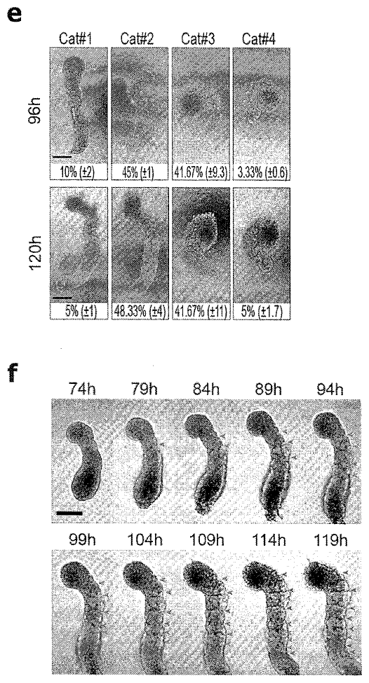

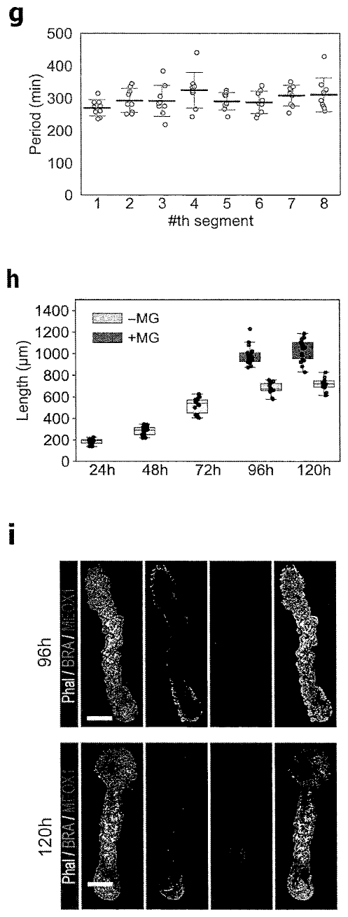

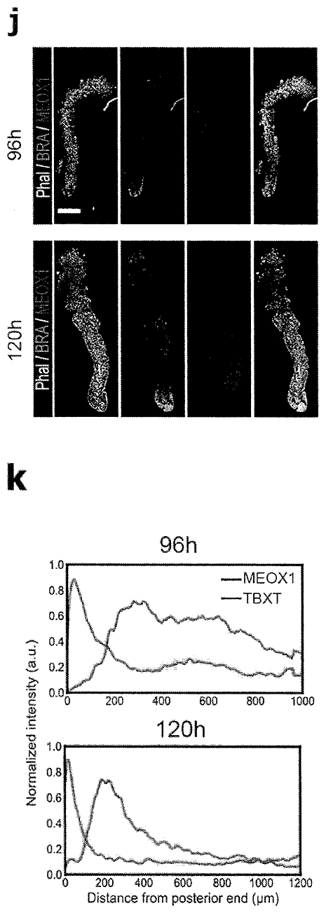

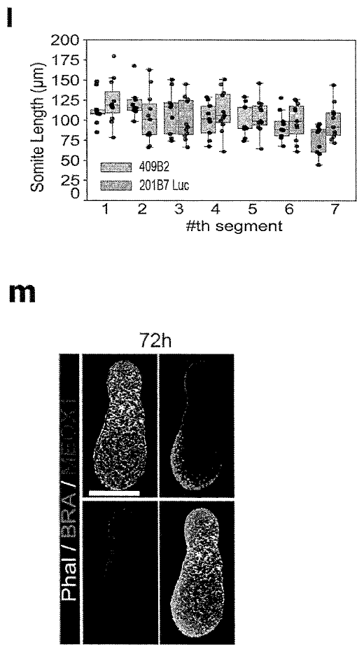

To determine the similarities of Axioloids to the vertebrate embryonic axis and tail, their morphological and molecular features were first assessed. In Axioloids, segments appeared with a periodicity of about 4-5 hours (FIG. 1c, d, FIG. 6f, g, Supplementary Video 1) and exposure to MG lead to a significant increase in convergent extension-based elongation, with Axioloid reaching total lengths of about 1000-1400 m at 120 h of culture (FIG. 1e, FIG. 6h). These polarized axial structures could be further divided into a TBXT (also known as Brachyury) expressing posterior terminal domain, and an anterior MEOX1 positive somitic mesoderm (SM) region (FIG. 1f, g, FIG. 6i-k). Segments formed every 80-140 m in the SM region of each Axioloid (FIG. 6l). The very end of the Axioloid tail was strongly positive for TBXT, with its expression reaching into and decreasing along the axis of the adjacent PSM, reminiscent of its reported expression in the tail bud (TB) and PSM of growing amniote embryos?. Interestingly, even in the absence of MG, this polarized pattern of expression of TBXT and MEOX1 could still be observed in Axioloids at 72, 96 and 120 hours of culture, suggesting that MG is not required for the initial establishment of this polarized expression pattern (FIG. 6m-q). Despite the regular nature of initial segment formation, fully epithelialized well-defined somite-like structures with proper apical-basal polarity and a central somitocoel-like cavity as seen in the developing embryo, were rarely observed in Axioloids (FIG. 7a, b). The segments in the MEOX1+SM area of human Axioloids, which formed upon exposure to MG, were characterized by apical accumulation of actin, indicating the initiation of an epithelialization process (FIG. 1f) but the epithelialization process within each segment appeared to be incomplete and largely disorganized (FIG. 7a, b).

Further assessment of gene and protein expression patterns of MG embedded Axioloids revealed a striking similarity to the anatomically and functionally regionalized molecular features described for the growing tail of vertebrate embryos. A posterior-most TB region positive for TBXT and SOX2 could be clearly distinguished (FIG. 1h, FIG. 7c), neighboring with a presomitic mesoderm (PSM) domain clearly demarcated by regionalized expression of TBX6, HES7 and MSGN1 (FIG. 1i-k, FIG. 7d-h). Further comparison of TBXT, SOX2 and TBX6 stained Axioloids revealed the presence of TBXT+ and SOX2⁺ double positive cells in the TB, which could be distinguished from TBX6⁺ but SOX2⁻ negative PSM cells (FIG. 1h, i, FIG. 7c, d). It was further found that the size of the TBX6⁺ PSM in Axioloids got reduced between 96 h to 120 h of Axioloid culture, while the SOX2⁺ population of the TB increased in size, which might be related to the observed slowing down of axial elongation and segment formation in Axioloids after 120 h of culture (FIG. 1h, i, FIG. 7c, d).

Besides the TB and PSM a narrow RIPPLY2 expressing anterior-PSM (aPSM) region (FIG. 1j, k, FIG. 7e-g) could also be distinguished, which delineated a further rostrally localized MEOX1⁺ and TCF15 expressing segmenting somitic mesoderm (SM) region, followed by a rostral-most MEOX1⁺ region of unsegmented mesoderm (FIG. 1f, j, FIG. 7e, f). LFNG (lunatic fringe), a major modulator of Notch signaling, was also expressed in the PSM, aPSM and the SM region of human Axioloids, with stripe-like expression pattern in SM similar to its expression reported for vertebrate embryos⁸⁻¹⁰ (FIG. 1k, FIG. 7g, h). Emerging segments in the SM aspect of Axioloids, anterior to the MESP2 expressing aPSM, showed also stripe-like expression of the rostrocaudal polarity genes UNCX¹¹ and TBX18¹², indicating the establishment of proper anterior-posterior identity within forming segments of Axioloids (FIG. 1l, m, FIG. 7i-l). Interestingly, UNCX and TBX18 were expressed in the SM region of Axioloids even in the absence of MG, albeit disorganized and without a clear rostrocaudal pattern (FIG. 7m-p). Together, this demonstrates that Axioloids, albeit lacking a notochord and neural tube, share both morphological and molecular features of the vertebrate embryonic axis and tail.

### Axioloids Recapitulate Traveling-Wave Like Oscillatory Activity of the Segmentation Clock

A universal key feature of somitogenesis is the oscillatory activity of the segmentation clock, a molecular oscillator and gene regulatory network centered around Notch signalling active across the growing tail of the vertebrate embryo, and believed to control the pace and size of forming segments¹³⁻¹⁵. To determine dynamics of oscillatory activity within human Axioloids, a reporter system was utilized, which had previously been used to characterize the human segmentation clock in vitro¹. Reproducible oscillatory activity of HES7, a well-studied segmentation clock gene^{1,4,16,17}, with a periodicity of about 4-5 hours in Axioloids could be clearly observed regardless of the presence of MG (FIG. 1n, o). Furthermore, HES7 displayed robust traveling-wave like expression from posterior-most TB and PSM region in an anterior direction along the PSM with a periodicity of 4-5 hours (FIG. 1p, q, FIG. 7q-s, Supplementary Video 2). In contrast to earlier in vitro models of the human segmentation clock^{1,4,6}, the clear formation of segments in human Axioloids happening in striking synchrony with the segmentation clock was observed. Segments formed every 4-5 hours in the anterior PSM region of Axioloids, a region, which overlaps and coincides with the wave front of HES7 oscillatory activity (FIG. 7q-s, Supplementary Video 2). The data thus indicates that both processes, segmentation and traveling wave-like oscillatory activity of the segmentation clock, are coupled in human Axioloids and happen in a spatiotemporally coordinated manner, a tight association that has so far only been reported in the embryonic tail of vertebrate model organisms^{18,19}.

### Single Cell RNA-Seq Analysis of Human Axioloids

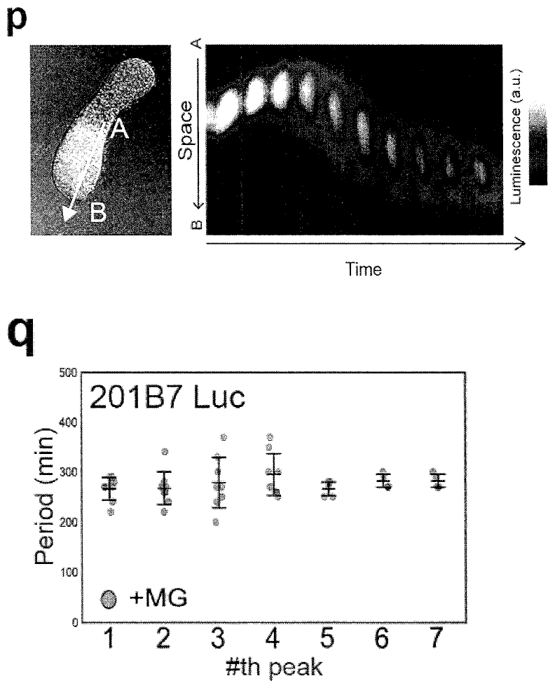

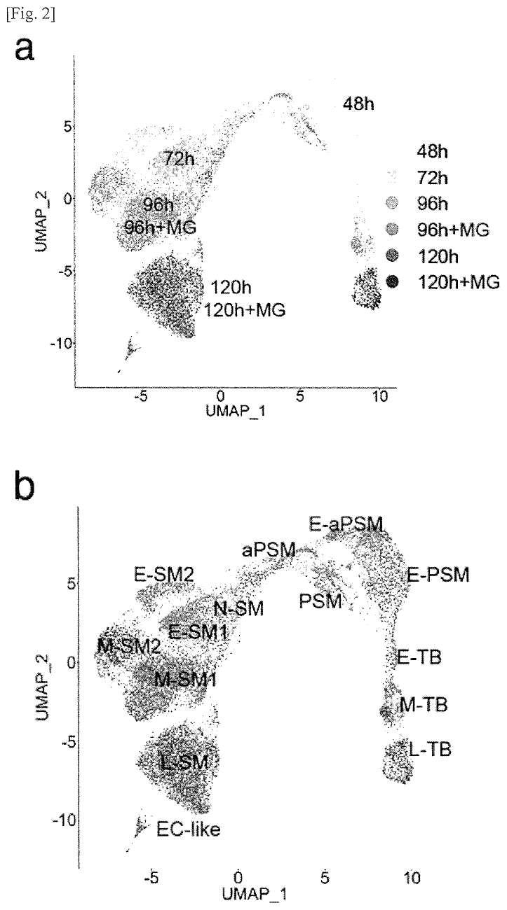

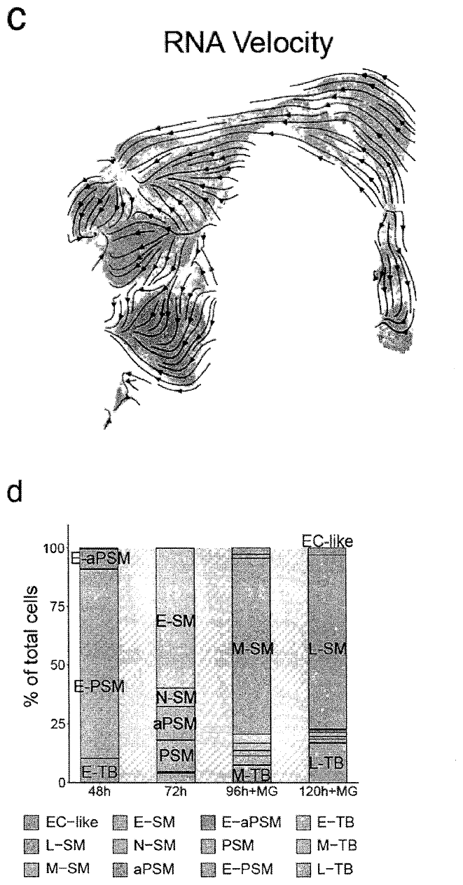

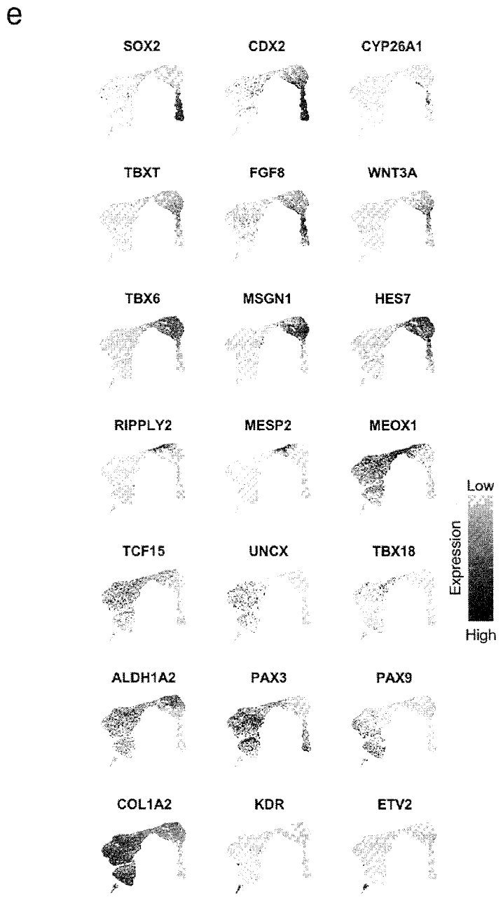

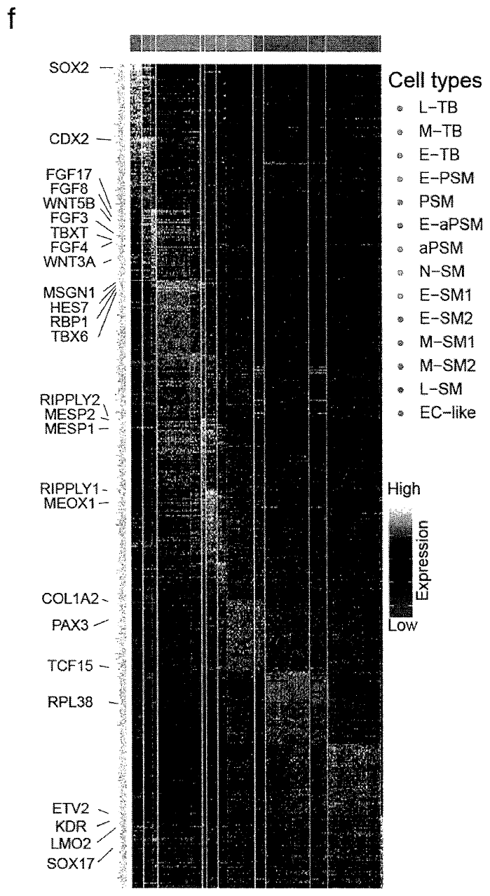

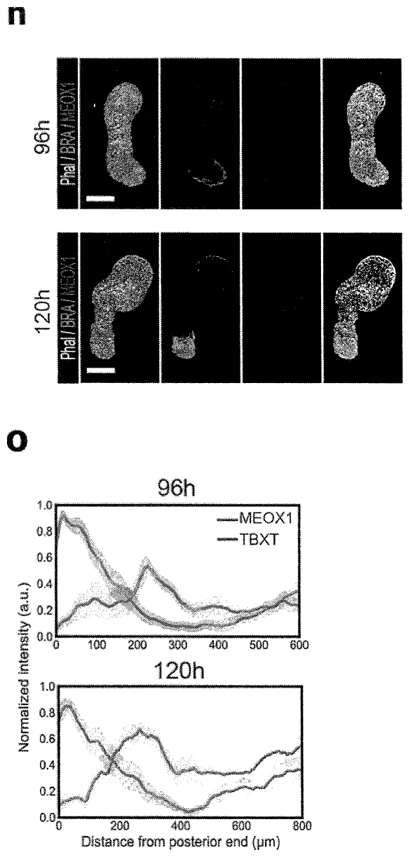

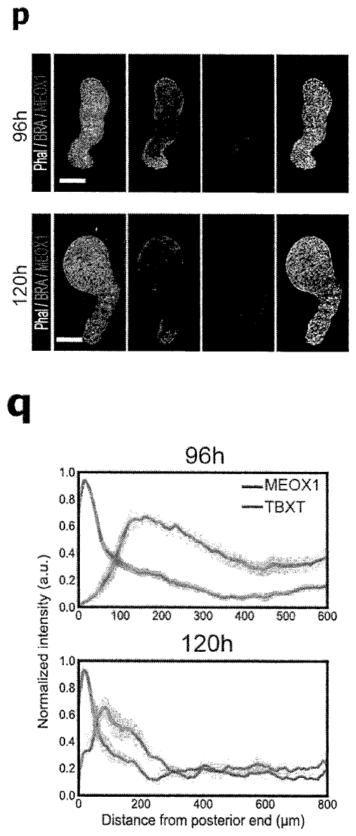

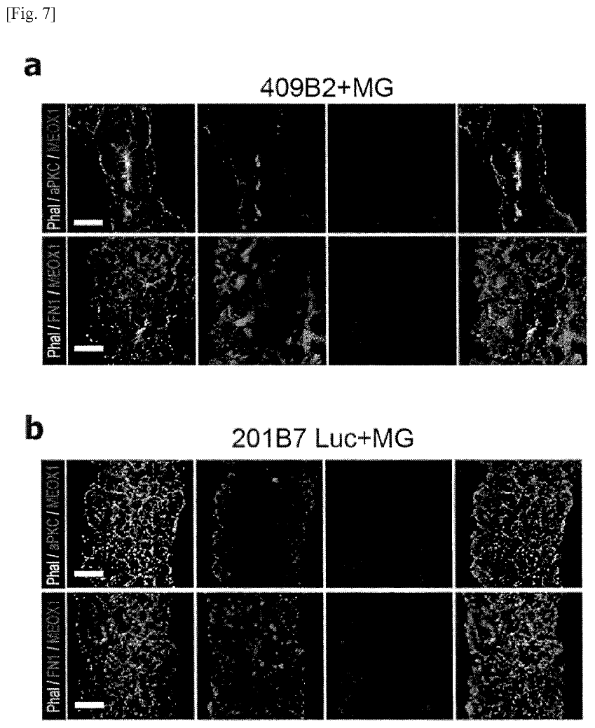

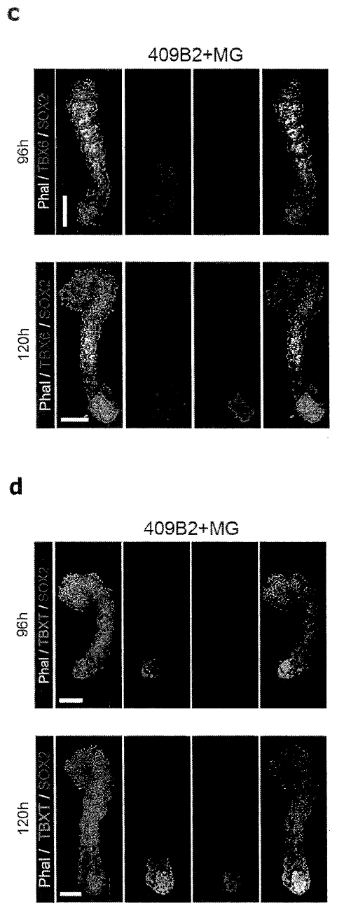

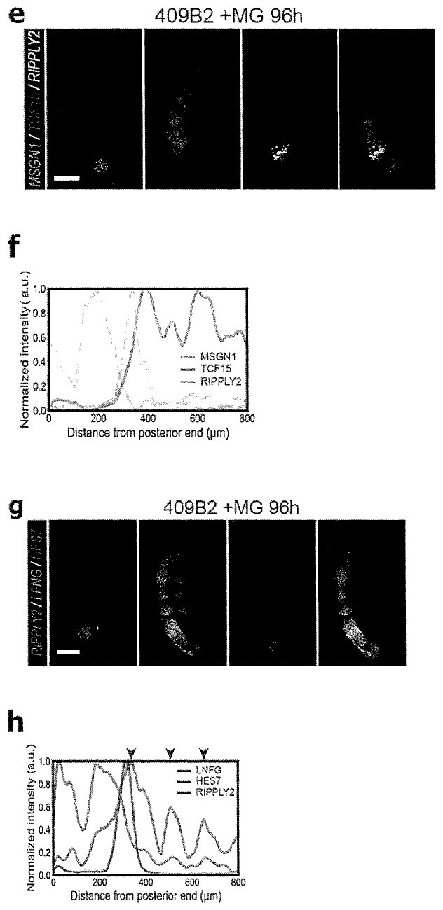

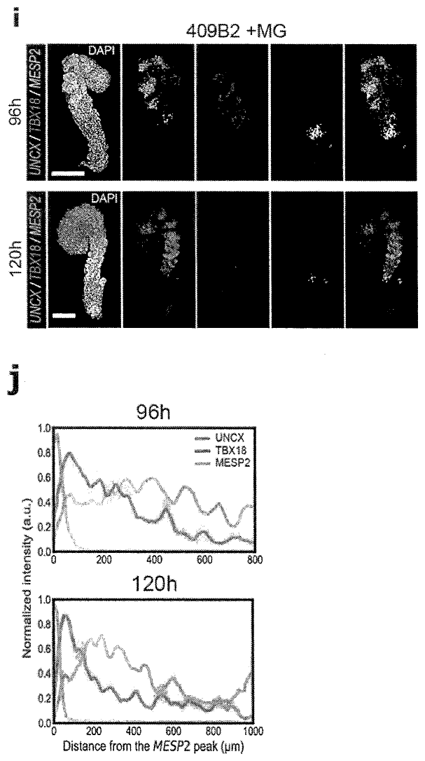

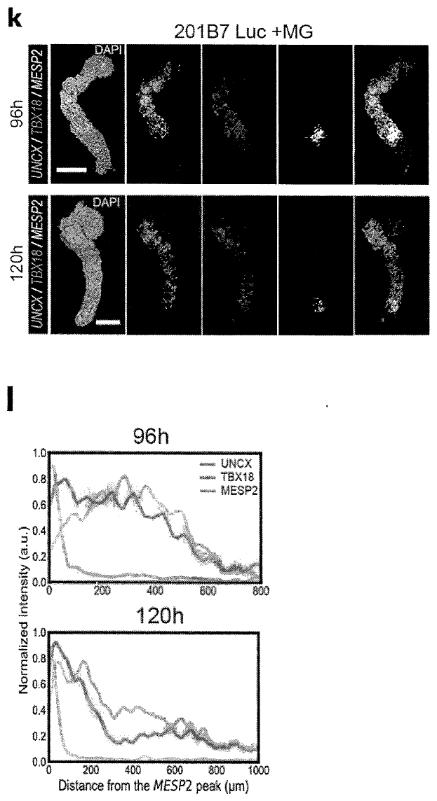

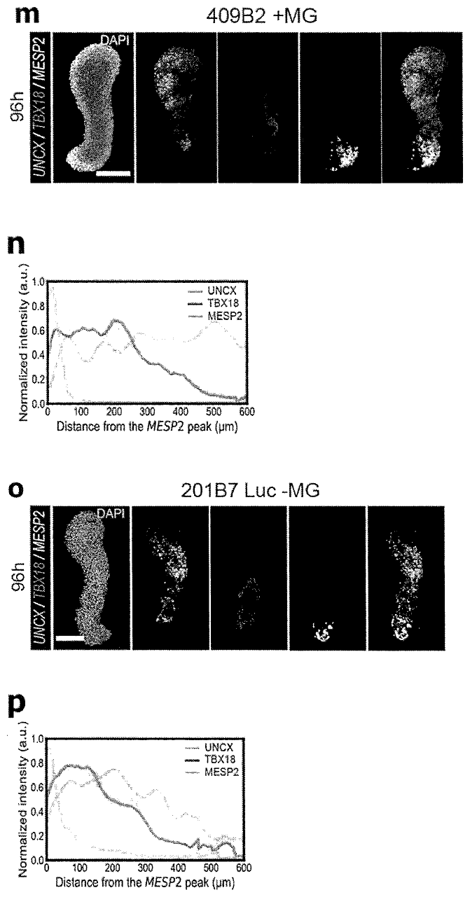

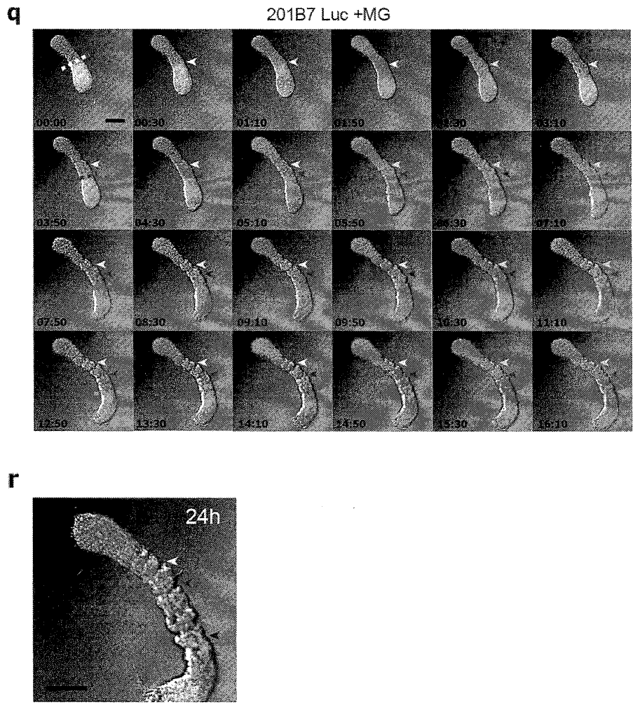

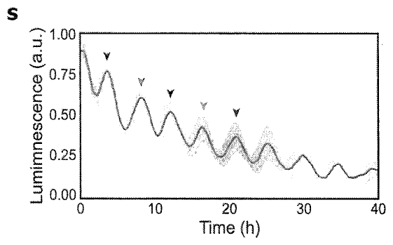

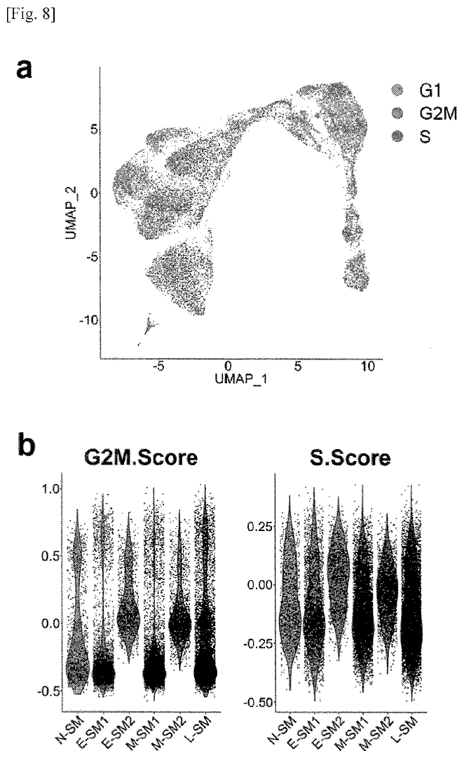

To further understand the functional features of the model system and to characterize the prospective dynamic changes in the cellular composition and molecular complexity of human Axioloids, temporally-resolved single cell RNA-seq (scRNA-seq) analysis of various stages (48 h, 72 h, 96 h & 120 h) and conditions (+/−MG) of the system was performed. The analysis revealed the presence of multiple dynamically changing cell clusters, which could be matched to different mesodermal cell populations present in the developing axis and tail of vertebrate embryos, including tail bud (TB), presomitic mesoderm (PSM), anterior presomitic mesoderm (aPSM), somitic mesoderm (SM) and angioblast/endothelial-like (EC-like) cells (FIG. 2a, b). These cell clusters could be further divided into putative subpopulations, based on their cell cycle state or time of emergence or respective maturation state during in vitro culture (FIG. 8a, b). RNA velocity analysis revealed a major differentiation trajectory, with early TB and early PSM cells dominating at 48 h of culture, and giving rise to SM populations, which make up the majority of cells at 96 h and 120 h of culture (FIG. 2b-d). Interestingly presence or absence of MG did not have a large effect on the differentiation trajectory or distribution of existing or emerging cell populations within Axioloids (FIG. 2d, FIG. 8c). Many genes previously reported to be specifically expressed in the anatomical compartments and cell populations making up the mesodermal aspect of the embryonic tail, were identified and shown to also match the specific expression profiles in human Axioloids (FIG. 2e, f). SM cells could be divided into six distinct clusters based on their expression profiles, with several of them showing specific high-level expression of various ribosomal proteins, including that of RPL38, for which mutations in mice were shown to result in homeotic transformations of the axial skeleton²⁰ (FIG. 2f, FIG. 8d). It was observed that several genes, including TBXT, CYP26 A1, WNT3a and FGF8 that are initially highly expressed in early TB, showed reduced expression at later stages in culture, while SOX2 remained strongly expressed in late TB. This suggests a possible change in number and function of putative somitogenic TBXT and SOX2 double positive neuromesodermal progenitor cells (NMPs) present in the TB of human Axioloids, consistent with the earlier observation of decreased TBXT staining in SOX2⁺ TBs of 120 h human Axioloids (FIG. 2f, FIG. 8e, f, see also FIG. 1h, i, FIG. 7c, d). RNA velocity and pseudotime analysis of scRNAseq data from MG embedded, sequentially segmenting Axioloids at 96 h of culture, revealed the presence of a major differentiation trajectory originating from TB and going over PSM and aPSM to SM cells, that matches with the posterior-to-anterior spatial distribution of these cell populations within human Axioloids (FIG. 2g-i).

### Axioloids Establish Proper FGF/WNT Gradients and Express Retinoic Acid Signaling Components

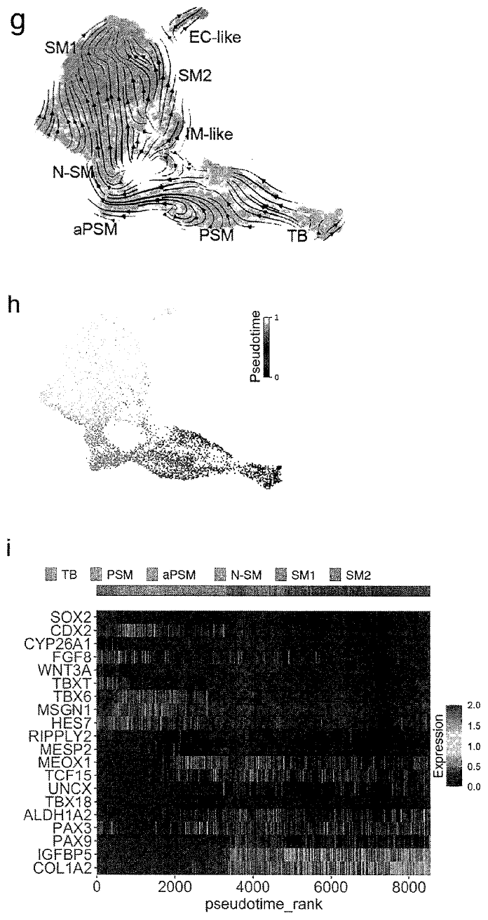

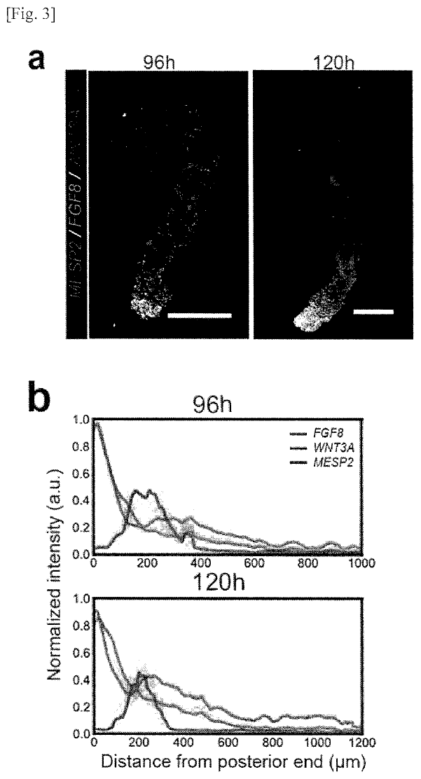

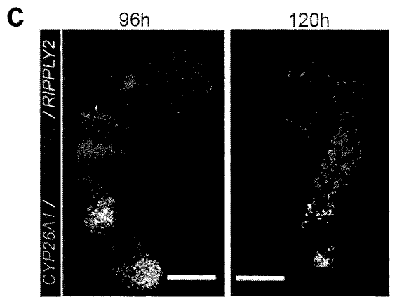

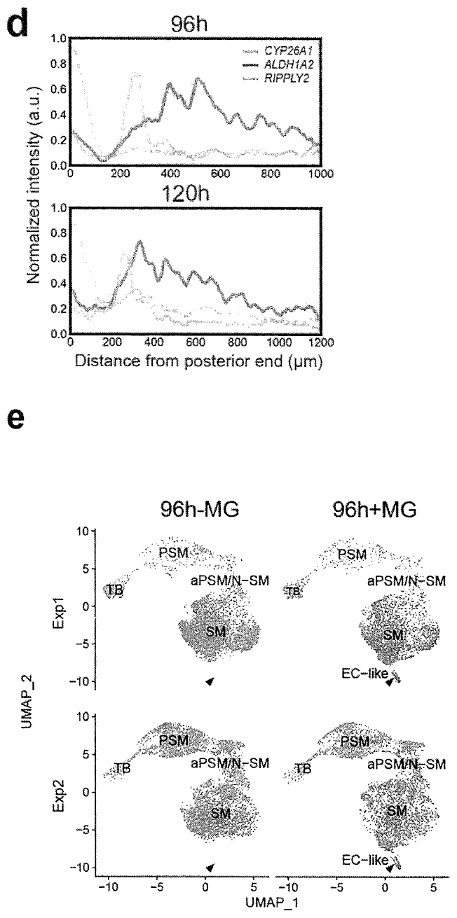

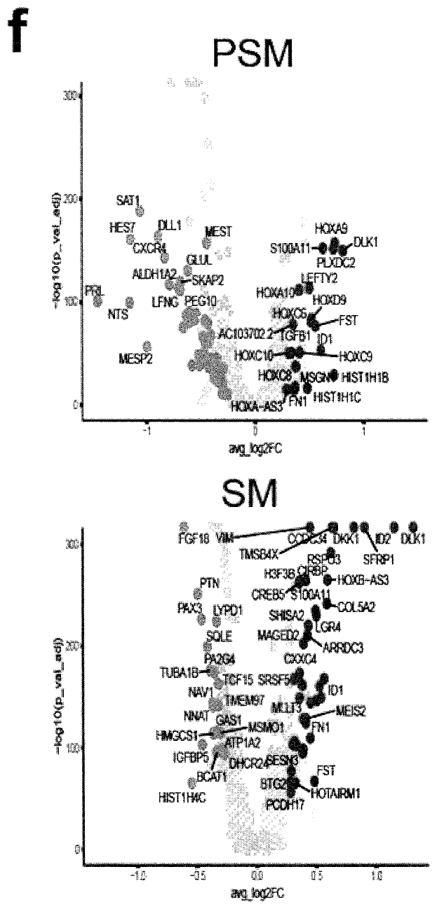

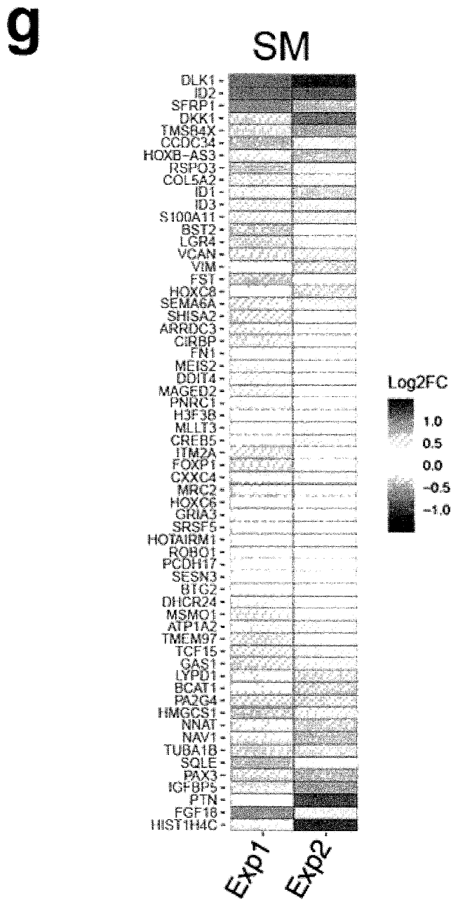

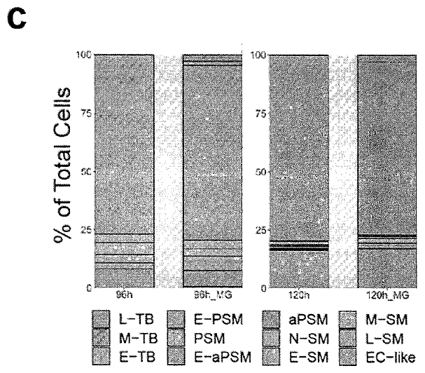

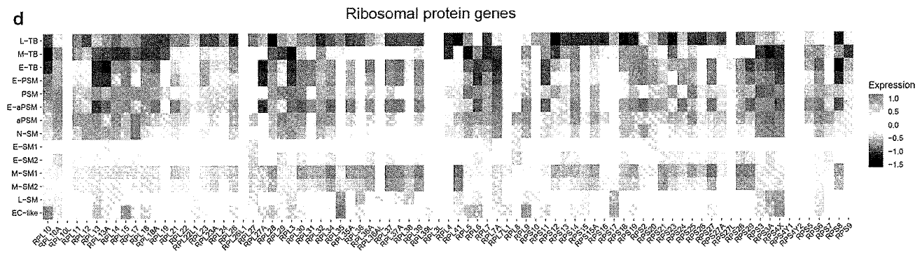

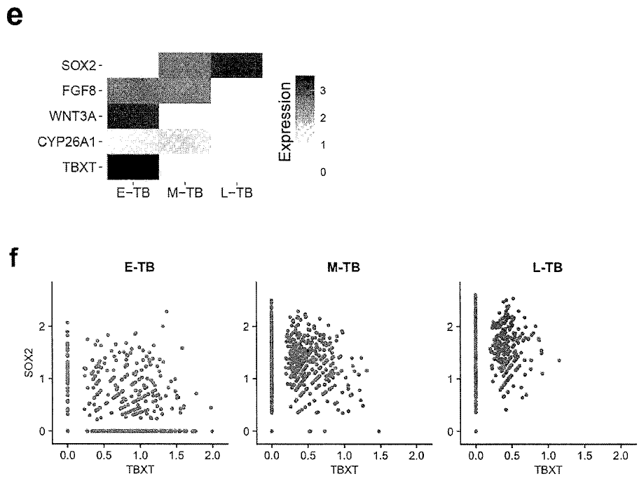

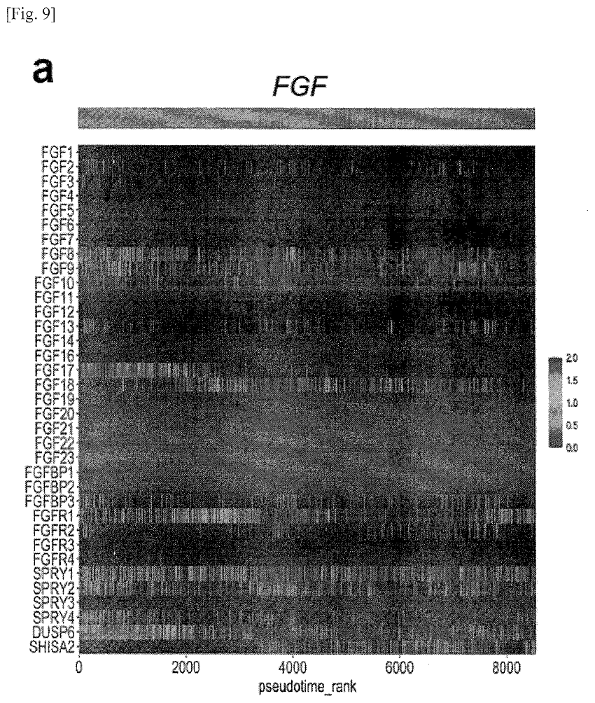

As the embryonic tail of vertebrates is characterized by opposing gradients of FGF/WNT and Retinoic Acid (RA), believed to be involved in the establishment of the “wave front” during somitogenesis²¹⁻²³, it was next asked whether similar gradients are also present within the human Axioloids. Pseudotime analysis of the scRNA-seq data, which matched well with the anterior-to-posterior organization and spatial distribution of the major cell populations found in Axioloids, was used to predict the expression patterns of multiple FGF, WNT and RA signaling associated transcripts within Axioloids (FIG. 9a-c). HCR based in situ hybridization was used to validate the predicted spatial expression patterns of FGF8 and WNT3a, two genes reported to be associated with the wave front during somitogenesis²⁴⁻²⁶, and revealed a clear posterior-to-anterior gradient of their expression in human Axioloids. Both genes were expressed strongest in the posterior TB and PSM region and their expression gradually decreased throughout the PSM up to the aPSM area marked by the expression of MESP2 (FIG. 3a, b, FIG. 9d-f). The expression of RA signaling associated molecules in Axioloids, focusing on ALDH1 A2, an enzyme involved in the synthesis of RA from Retinal (RAL), and CYP26 A1, a RA-metabolizing enzyme was then validated. High level localized expression of CYP26 A1 in the TB was detected, while ALDH1 A2 expression was high in the SM region of human Axioloids, showing highest expression rostral to the aPSM specific expression of RIPPLY2 and slowly decreasing along the posterior to anterior axis (FIG. 3c, d, FIG. 9g-i). Furthermore, ALDH1 A2 also formed a stripe-like expression pattern, showing higher levels of expression in the rostral halves of forming segments, similar to that described in the mouse²⁷ (FIG. 3c, d, FIG. 9g-i).

Matrigel Only Potentiates In Vitro Segmentation but does not Stabilize them

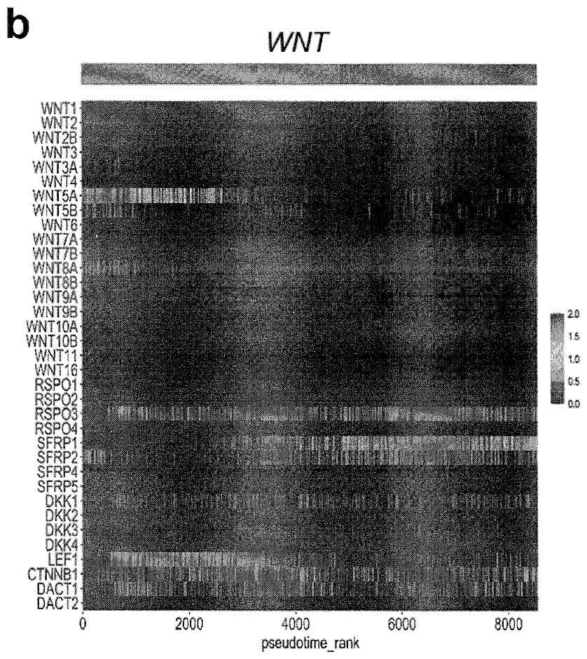

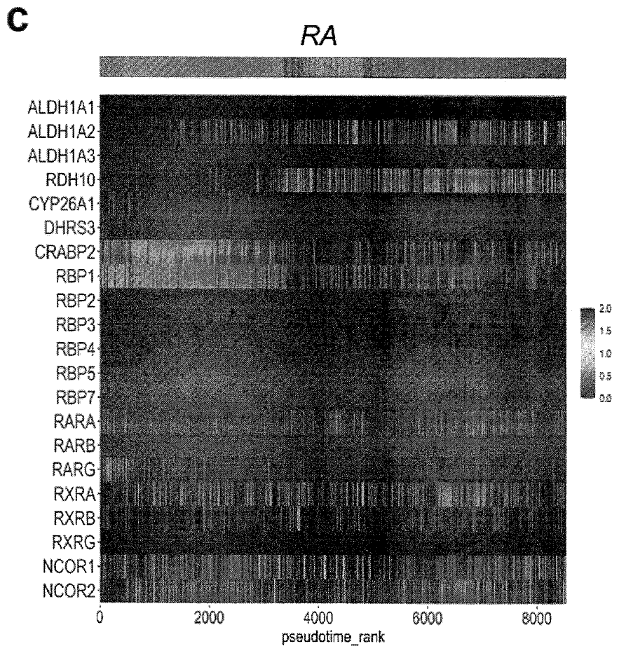

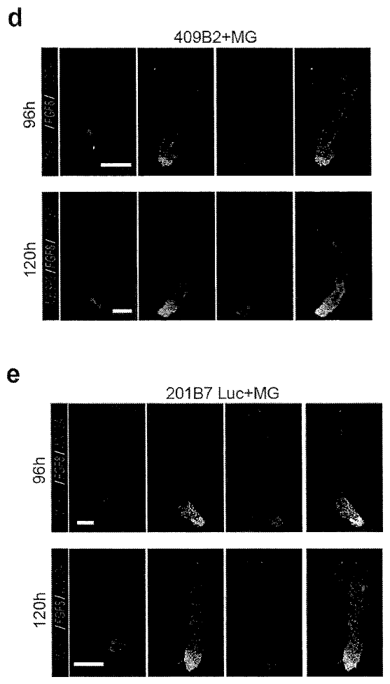

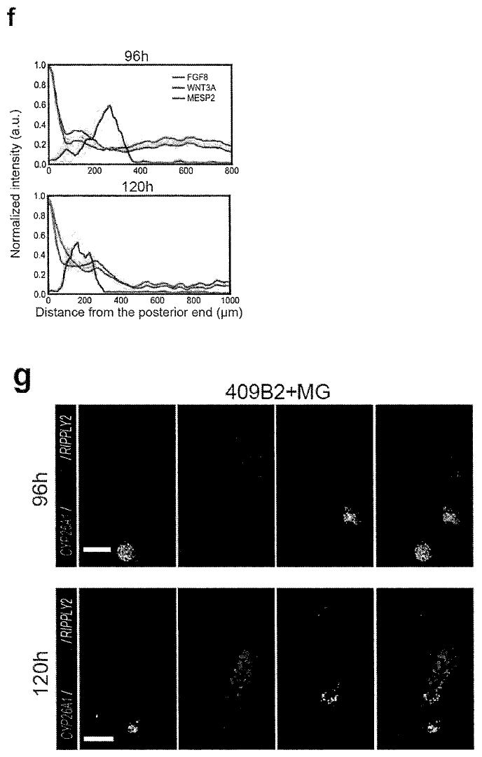

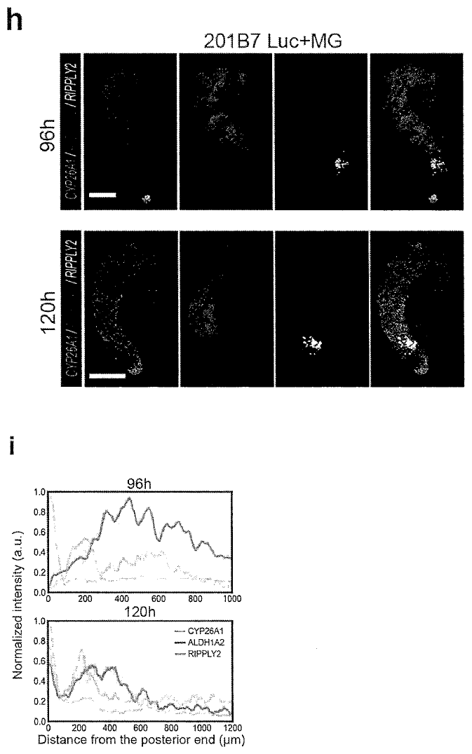

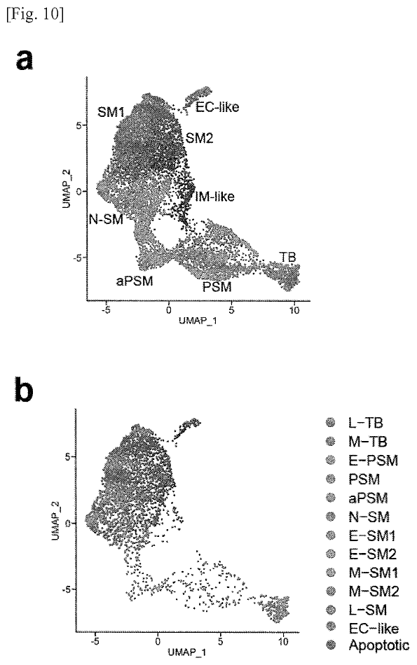

The data indicated that while MG promotes axial elongation and the initial formation of segments, it is unlikely to be sufficient to maintain or stabilize these segments. To better understand the role of MG in the elongation and segmentation of human Axioloids, the scRNA-seq data of MG-containing and -lacking cultures of Axioloids were compared. The analysis revealed the MG-dependent emergence of an angioblast and endothelial cell-like (EC-like) population of cells at 96 h of Axioloid culture (FIG. 3e, FIG. 10a-d). A similar angioblast or EC-like population has been also described in trunk-like structures generated from murine pluripotent stem cells in the presence of Matrigel³, suggesting an evolutionary conserved role of ECM components on the genesis of vascular progenitor cells known to emerge during somitogenesis²⁸.

The scRNA-seq data, focusing on differentially expressed genes (DEGs) in PSM and SM cells (FIG. 3f, g) were next further analyzed. Gene enrichment analysis identified up-regulation of genes associated with epithelial-to-mesenchymal transition (EMT) in SM cells of MG exposed Axioloids, while conversely PSM cells exposed to MG showed down-regulation of another distinct set of EMT associated genes (FIG. 3f, g, FIG. 10e, f, Supplementary Table 2)). MG-exposed SM cells furthermore showed an increase in the expression of negative regulators of WNT and TGFβ signaling pathways, DKK1, SFPR1 and FST, ID2 respectively (FIG. 3g, h, FIG. 10e). Basement membrane associated ECM components, including FN1 and VIM1, reported to be involved in the development of somitic mesoderm and known to be expressed in migratory mesenchymal cells²⁹⁻³¹, were also up-regulated in SM cells of Axioloids cultured in the presence of MG (FIG. 3g, h). MG exposure also changed gene expression in TB, albeit to a smaller extent than the changes in the PSM and SM of human Axioloids (FIG. 10f, g). These results, especially the identified MG-associated up-regulation of EMT signatures in SM cells, coupled with the down-regulation of epithelialization associated genes such as TCF15³² and PAX3³³ indicate that the effect of MG on human Axioloids is not straightforward and seems to both promote and counteract molecular processes related to segmentation and epithelialization. This matches well with the observation that MG exposure leads to enhanced axial elongation and initial clear formation of segments, but then fails to stabilize these segments and is not sufficient to establish well-organized and fully epithelialized somites in Axioloids.

### RA Signaling is Required for Segment Stability and Epithelialization of Somites

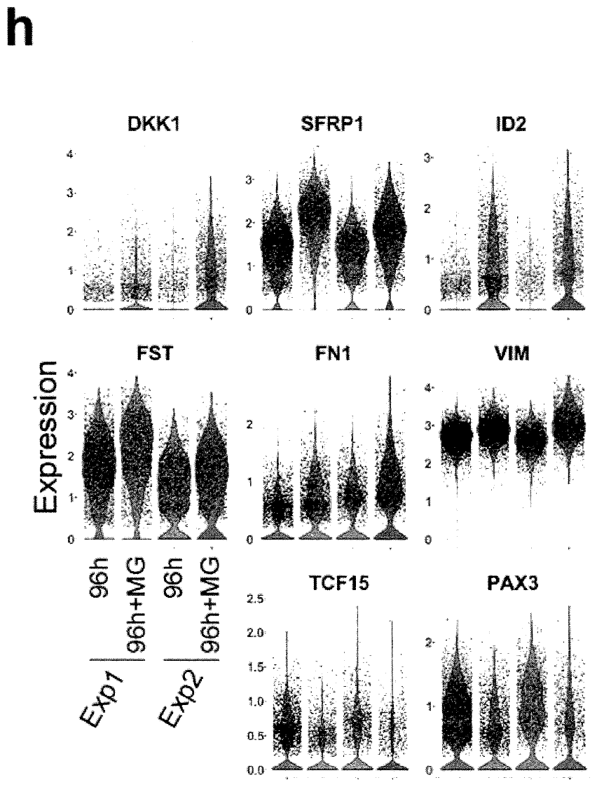

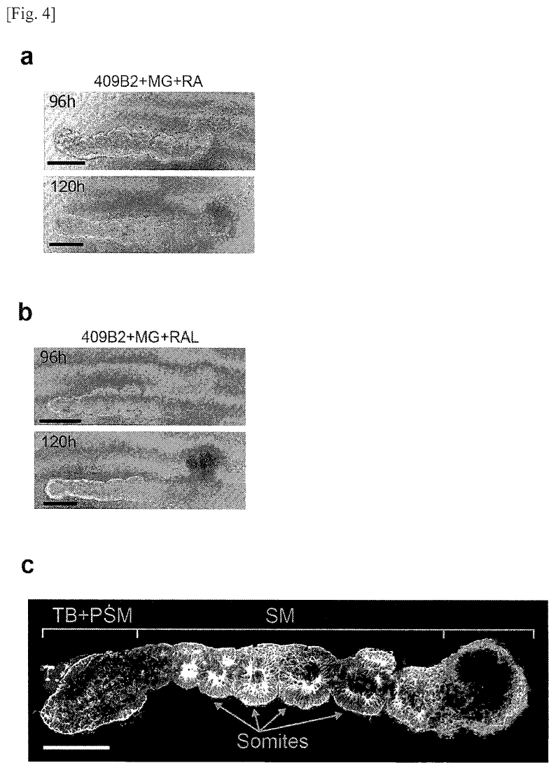

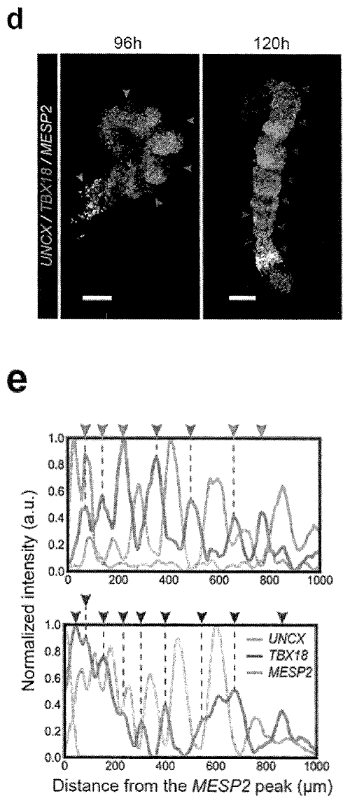

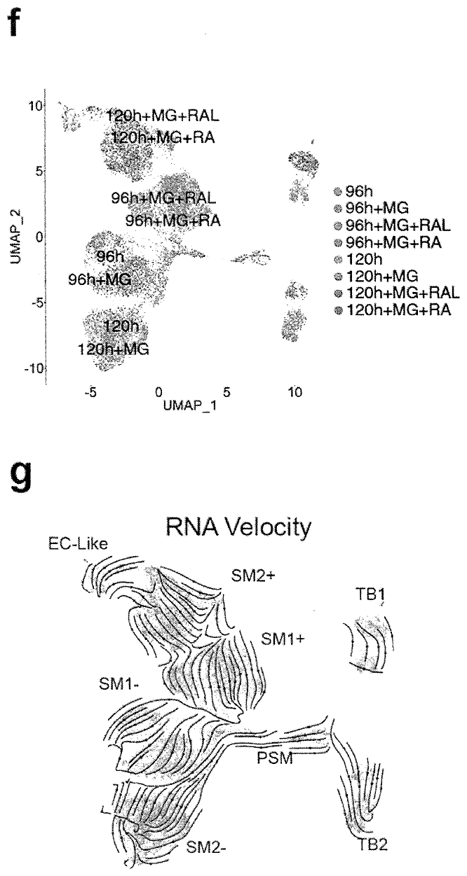

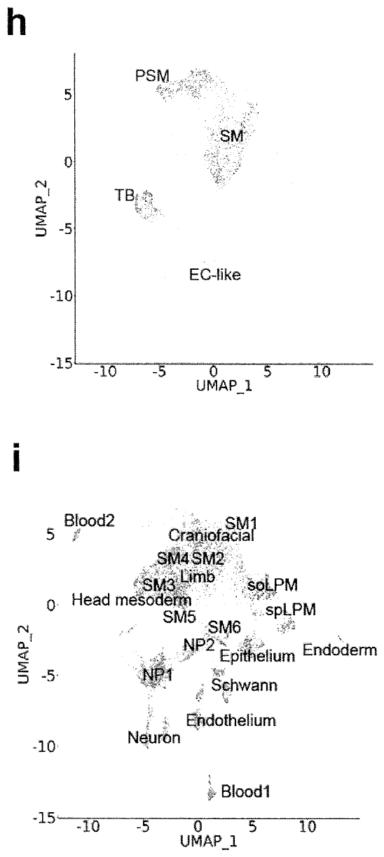

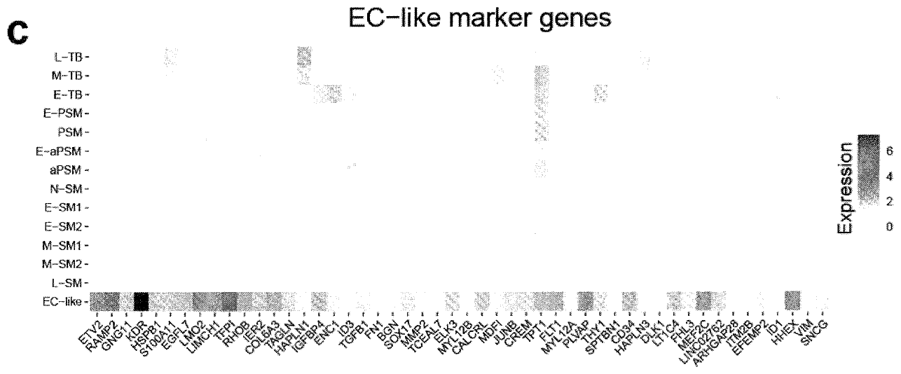

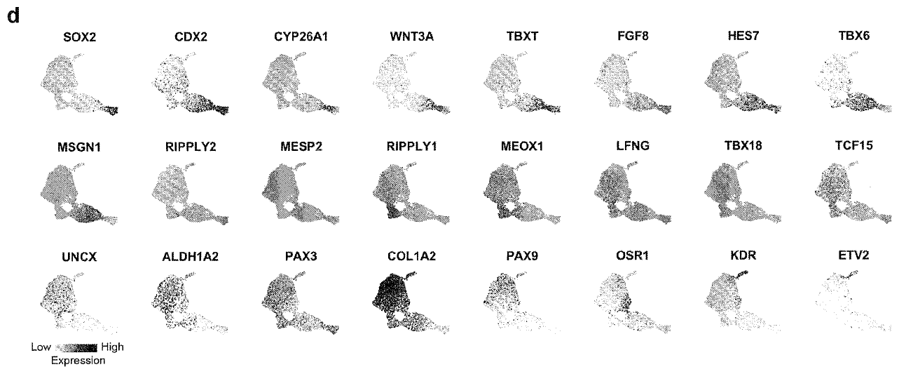

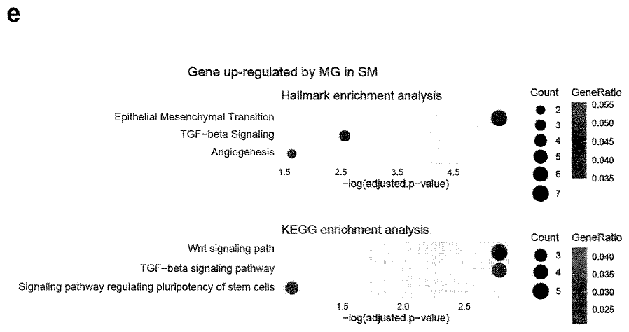

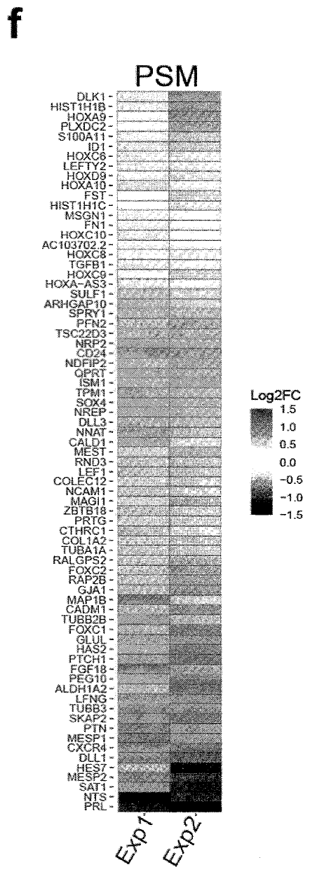

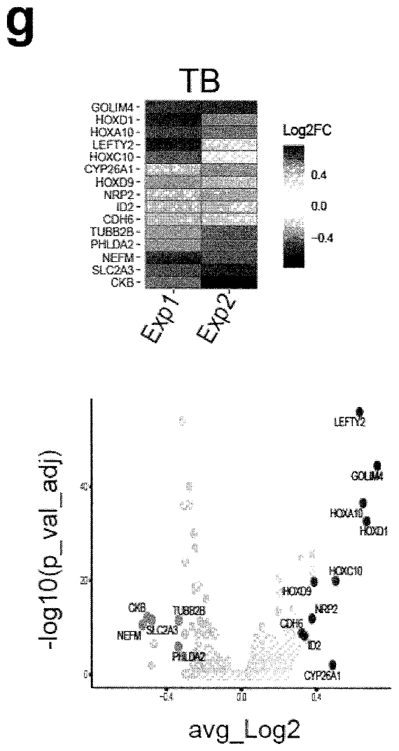

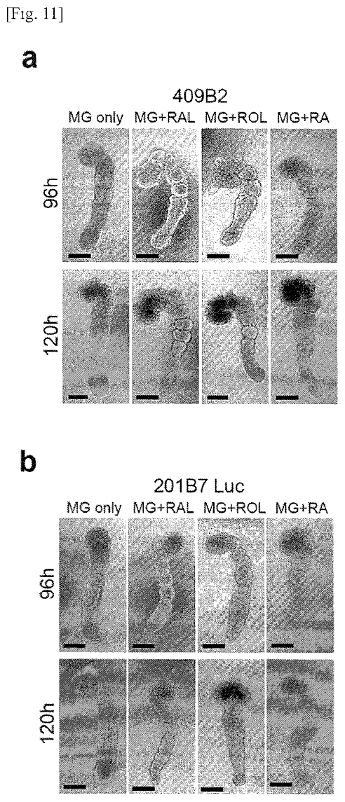

Potential factors that may contribute to stabilizing the segmentation process and increase epithelialization of somites were next looked for. Although ALDH1 A2 and molecules associated with synthesizing RA, such as RDH10, are specifically expressed in the system, the precursors Retinol (ROL) and Retinal (RAL) were not present in the culture conditions. Axioloids are thus unable to generate RA de novo, raising the question as to the function of RA signaling in Axioloids. To address this question, either directly RA or its precursors ROL or RAL was added into the in vitro system, during the MG embedding phase between 72 h-120 h. Surprisingly, it was observed that presence of RA molecules, led to a dramatic improvement of the stability and epithelialization of forming segments within MG-embedded Axioloids at 96 h and 120 h. (FIG. 4a, b, FIG. 11a-c, Supplementary Video 3). MG+RA Axioloids gave robustly rise to sequentially forming fully epithelialized somites with proper apical-basal polarity and central somitocoels, which MG or RA alone could not achieve (FIG. 4c, FIG. 11d, e). Despite a clear effect on epithelialization, which could not be achieved by RA signaling alone (FIG. 11f), the features of these Axioloids, including overall morphology, total length, periodicity of segment formation or number of segments formed within 48 h post-embedding, did not change upon addition of RA into the system (FIG. 11g-n). Assessing the effect of RA signaling on major protein and gene expression patterns within Axioloids, including that of TBXT and MEOX1, or UNCX and TBX18, it was found that these molecular features were largely unchanged or even improved in RA & RAL treated Axioloids showing clear rostrocaudal identity of forming epithelial somites (FIG. 4d, e, FIG. 12a-p, Supplementary Video 4). MG+RA Axioloids revealed a similar change in size and expression of the TBXT⁺ SOX2⁺ TB as seen for MG only Axioloids (FIG. 12q, see also FIG. 1h, FIG. 7c). Furthermore, it was found that simultaneous inhibition of RA signaling via BMS493, a pan-RAR inverse agonist, while permitting RAL-mediated synthesis of RA in Axioloids, could reverse the RA effect, strongly inhibiting somite formation and epithelialization (FIG. 12r-u, Supplementary Video 5).

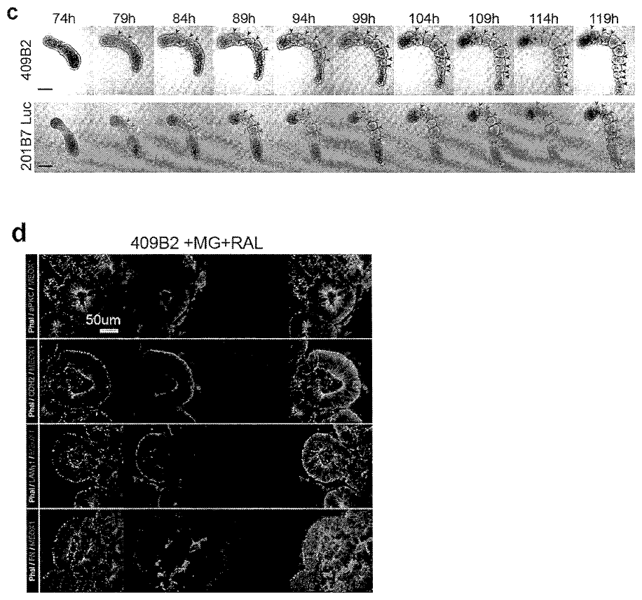

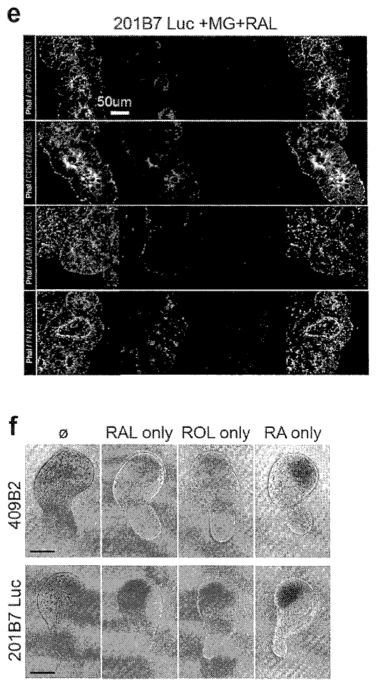

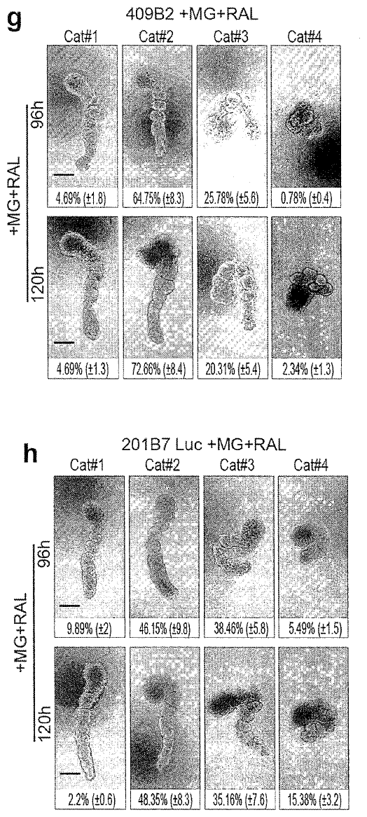

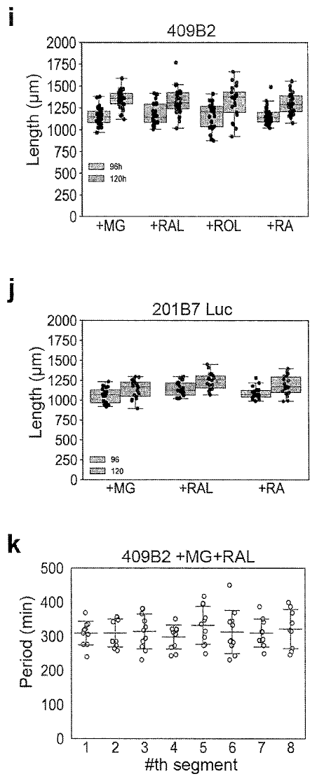

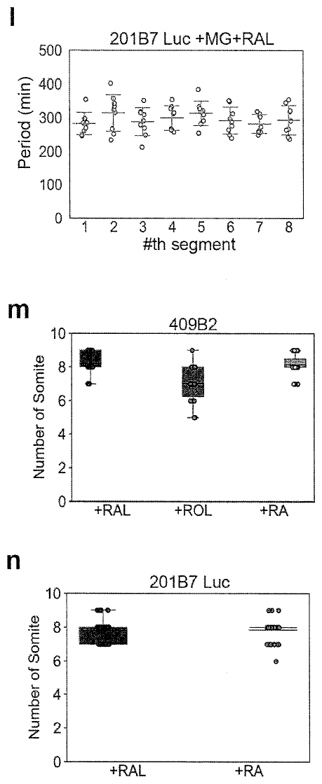

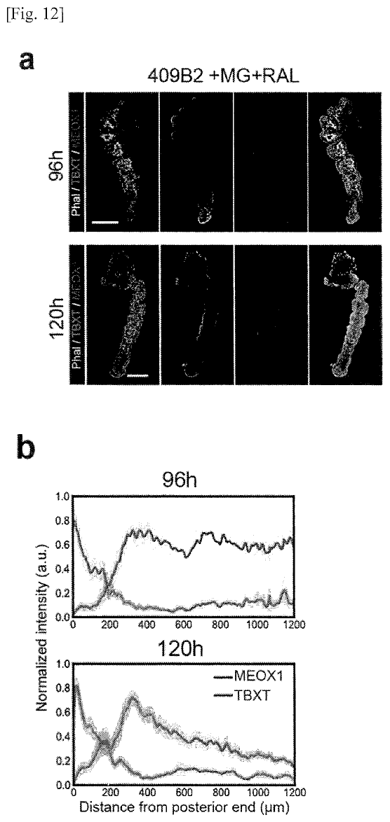

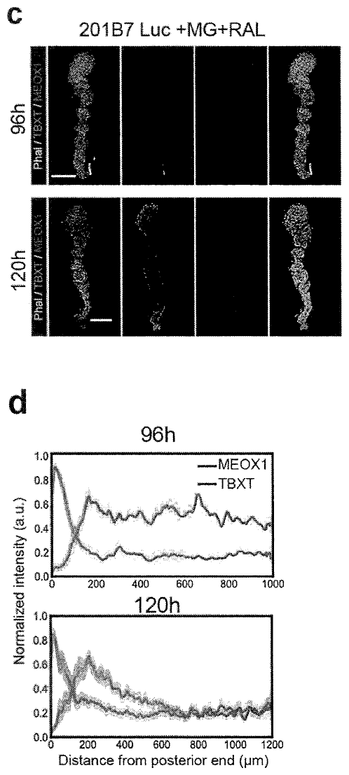

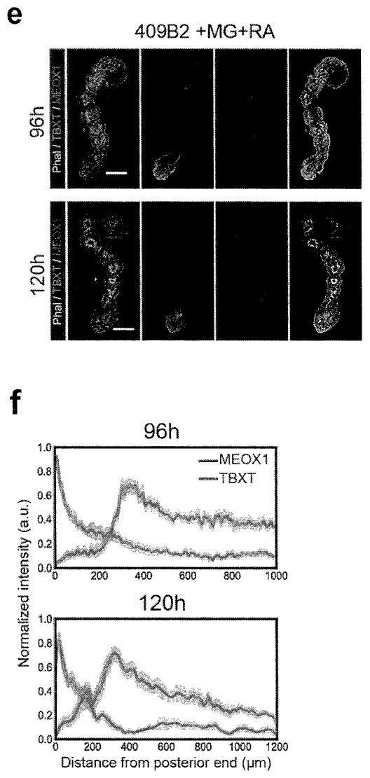

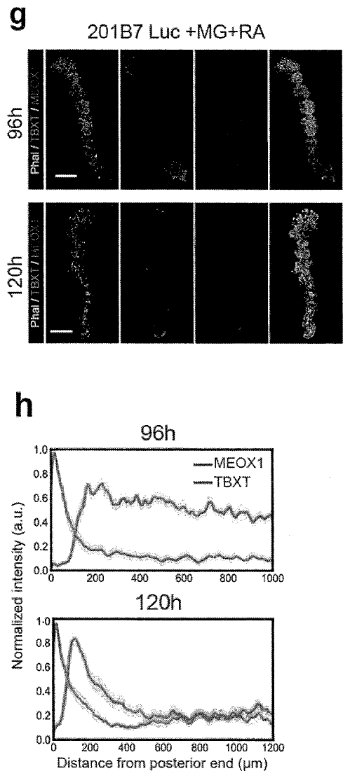

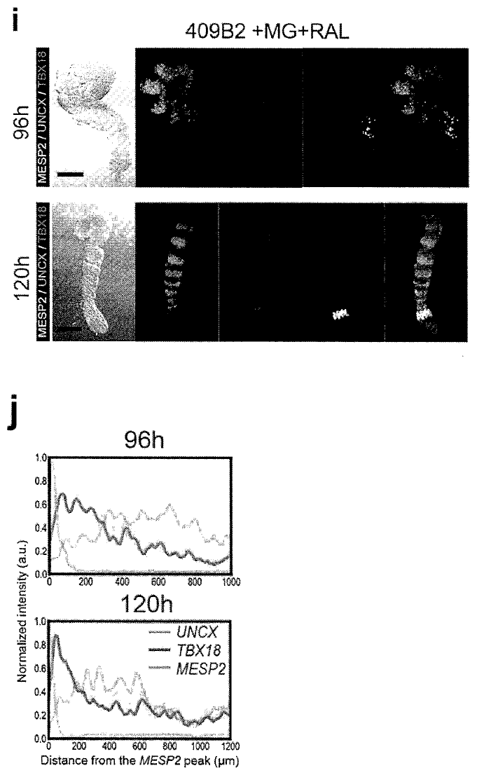

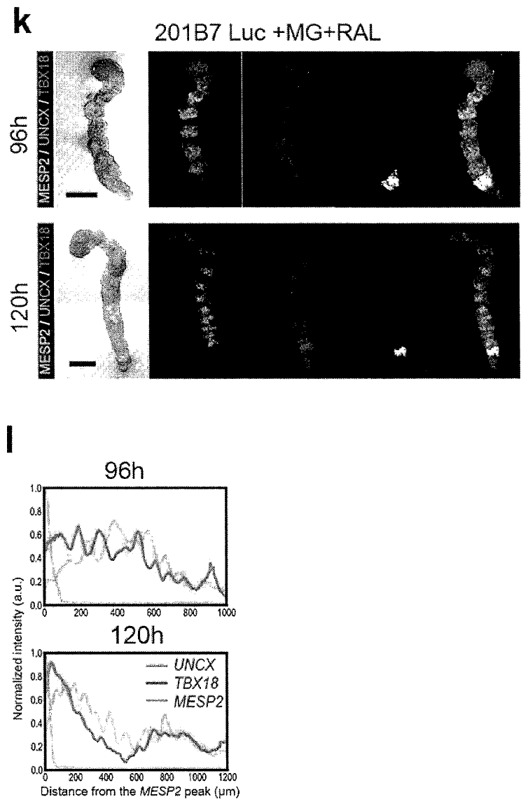

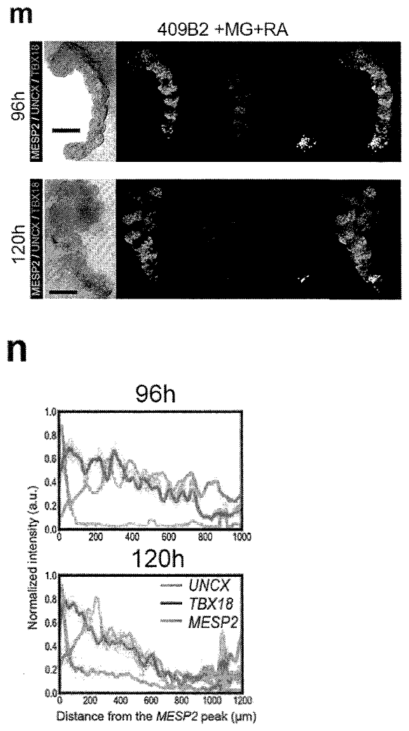

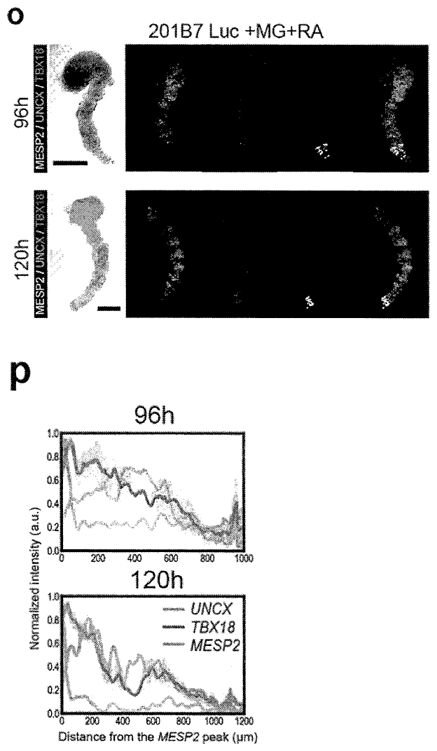

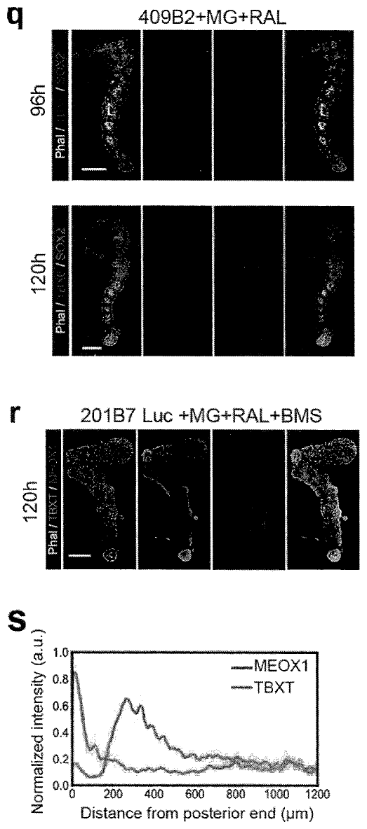

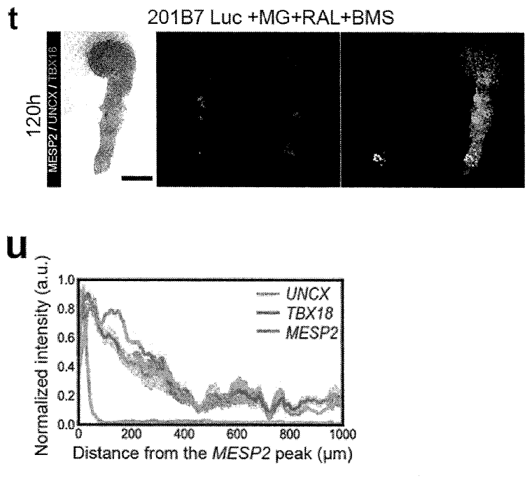

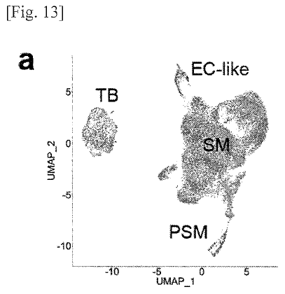

To further dissect how RA mediates its function and alters somite formation in Axioloids, Axioloid samples that were supplemented with RA and RAL at 96 h and 120 h using scRNA-seq analysis was analyzed. a clear segregation of the SM and TB clusters based on the presence or absence of RA and RAL was observed, while overall identities of the cell clusters remained stable and could still be matched with each other (FIG. 4f, g, FIG. 13a). Comparing the different conditions, it was found that many of the DEGs, which were observed in Axioloids upon addition of MG alone, including upregulation of EMT associated genes and of negative regulators of WNT (SFRP1, DKK1) or TGFβ (FST, ID2) signaling as well as the down-regulation of epithelialization associated transcription factors TFC15 and PAX3 observed in SM, were attenuated upon the addition of RA or RAL (FIG. 13b-f). This suggests the intriguing possibility that the presence of RA activity in the system is balancing the effect of MG, and that both, MG-mediated axial elongation and initiation of segmentation and RA-mediated stabilization and epithelialization of forming segments are required for the proper establishment and progression of somitogenesis within human Axioloids. Furthermore, many known, as well as novel targets of RA signaling in the context of human segmentation and somitogenesis, including multiple RA targets in the TB and SM were identified (FIG. 13b, c). Retinoid/RA signaling homeostasis related genes such as RBP1, DHRS3, CYP26 A1 or CRABP2 were also upregulated by RA both in TB and SM, suggesting the presence of a negative feedback loop regulating the production and availability of RA in the system (FIG. 13e, g). Multiple transcription factors including TCF15, MEOX1, PAX3, PBX1, ZIC3, NR2F1 and MEIS2, known to be expressed in somites and important for axial development of model organisms were also up-regulated in MG-embedded human Axioloids exposed to RAL/RA (FIG. 13e, h).

The findings, showing that RA has a critical role in somite formation and epithelialization, are surprising, as the loss of RA in embryos has generally been reported to cause smaller somites or asymmetric formation of bilateral somites^{21,27,34}, rather than leading to a strong epithelialization-related phenotype. Furthermore, regarding symmetry and bilaterality in the system, in MG and RA treated Axioloids a single axis of sequentially forming epithelial somites with single central somitocoels was typically observed. Intriguingly, Axioloids also frequently displayed a superficial groove or midline-like structure, starting in the PSM and going through most of the forming segments (FIG. 14a, b), though it typically remained superficial, without separating the segments into two fully segregated somites. (FIG. 14a, b, Supplementary Video 5a). True bilateral, fully segregated somites, each with their respective central somitocoel, were nevertheless occasionally present in Axioloids, sometimes as an isolated fully segregated bilateral single pair of somites and in some rare cases also as a sequence of two or more somite pairs along the AP-axis of an Axioloid (FIG. 14c-e, Supplementary Video 5b). These bilateral somites showed usually normal protein and gene expression patterns, including proper rostrocaudal polarity (FIG. 14f-k).

### Human Axioloids Mimic Characteristics of Human Embryos

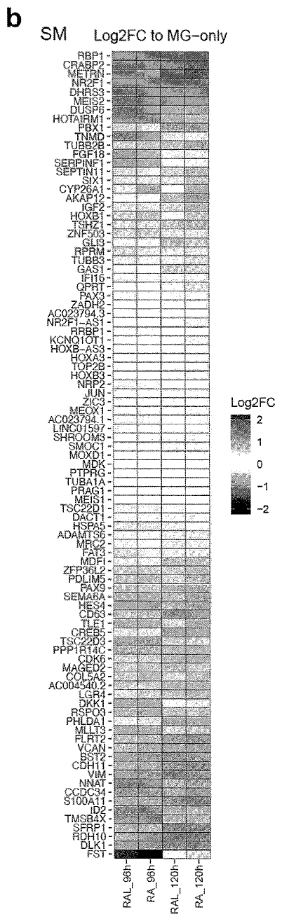

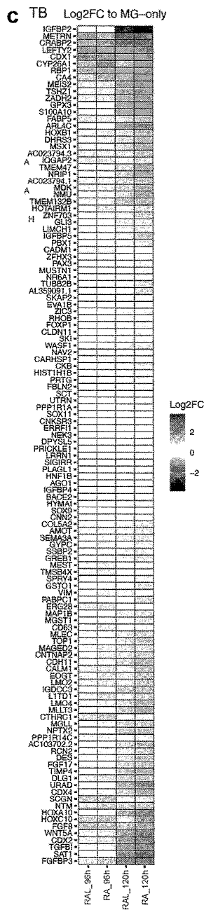

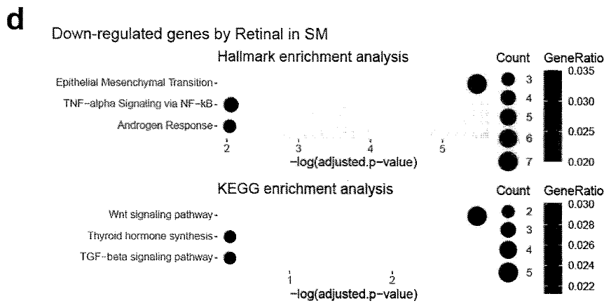

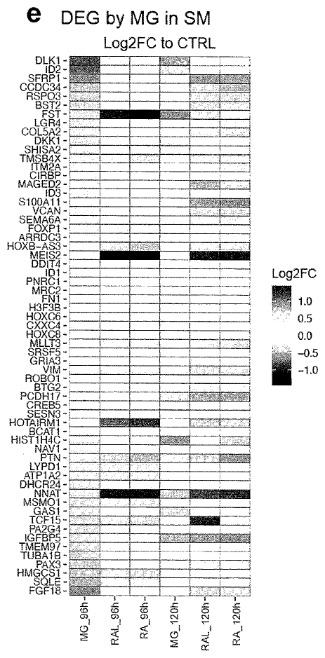

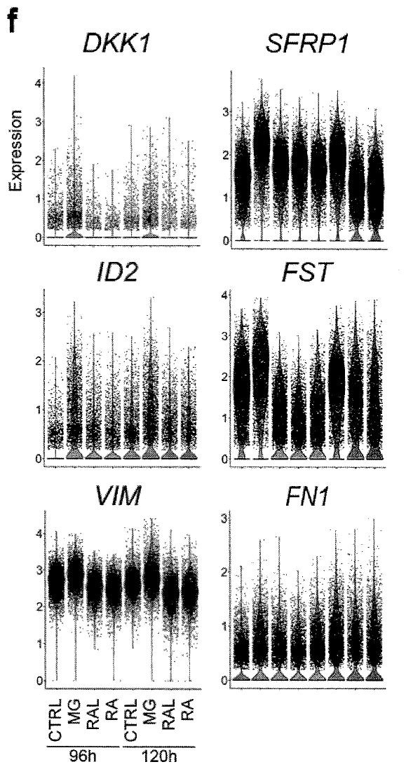

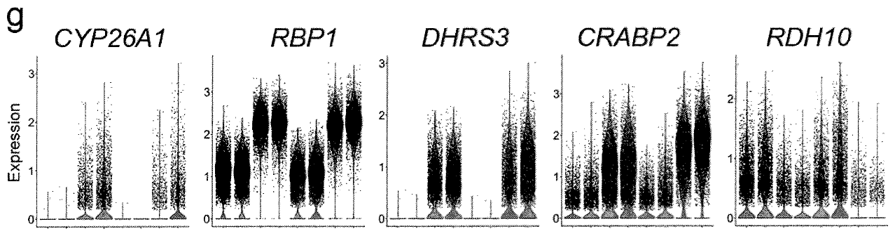

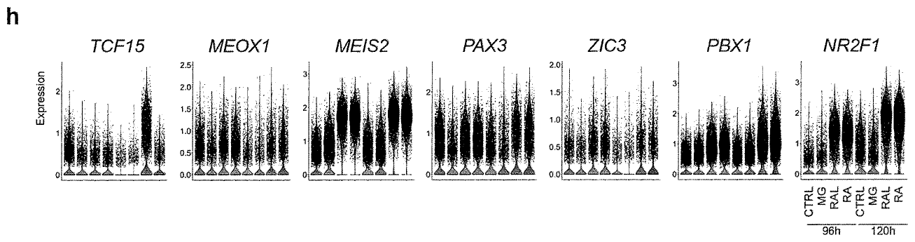

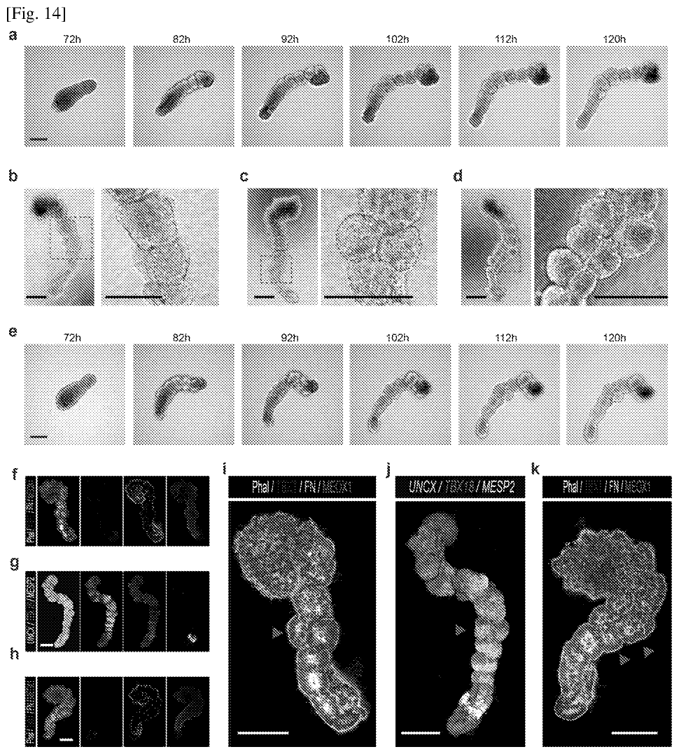

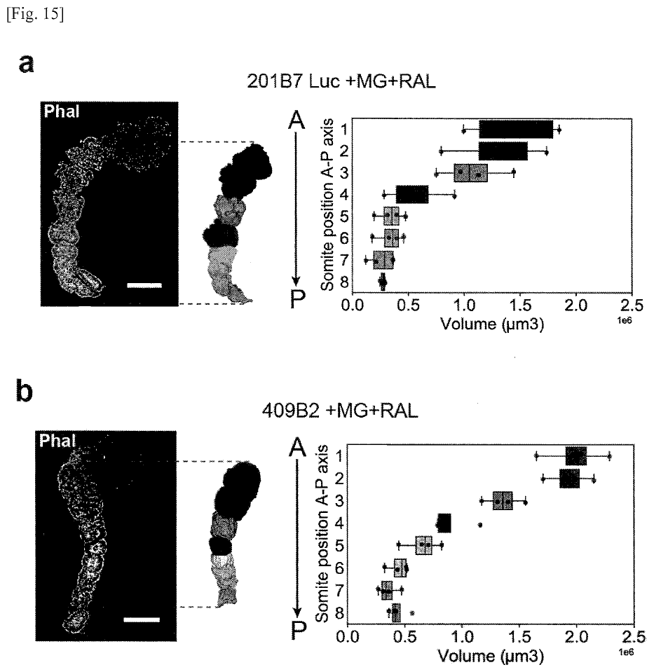

Somitogenesis is a distinctive feature of vertebrate embryos and the number of somites allows an approximate assessment of the age of an embryo. Carnegie stage (CS) 10 human embryos are characterized by 4 to 12 pairs of somites, suggesting that 96 h and 120 h old Axioloids are, at least, in an equivalent stage. Comparison of the dimensions of the Axioloid somites with those of CS10 & 11 embryos suggests that they are similar in shape and size (FIG. 15a-d). It was furthermore found in both, Axioloids and human embryos, that earlier formed (older) somites closer to the anterior rostral region, are larger and increase in size, while newly forming somites in the caudal region have smaller volumes. The decrease in size of the posterior somites is especially evident in human Axioloids (FIG. 15a, b, Supplementary Video 6). This level of high morphometric similarity observed between in vitro-derived Axioloids and in vivo human embryos, thus strikingly suggests, that even in the absence of other germ layers, the mesoderm alone can robustly self-organize and give rise to properly sized somites making up the metameric axis of the early embryo.

To further benchmark the morphogenetic and molecular features of Axioloids with that of actual human embryos, recently uploaded scRNA-seq data set of CS12 to CS16 human embryos was utilized 35. The CS12 embryo data for comparison was reanalyzed, and mutual nearest neighbors (MNN)-based integration analysis³⁶ (FIG. 15e-m) revealed, that the majority of cells present in human Axioloids matched well with the cells identified for the CS12 human embryo (FIG. 4h-k, FIG. 15h-m). As expected, endodermal, neuroectodermal and other non-related mesodermal cell populations (e.g. lateral plate mesoderm (LPM), blood) identified in the human embryo, were not matching with human Axioloids (FIG. 4i, k). On the other hand, Axioloids showed strong overlap with paraxial mesoderm and axial development related mesodermal cell populations found in the CS12 human embryo including populations labeled as somites or head mesoderm (FIG. 4i, k, FIG. 15h-m).

It was also found that a portion of Axioloid-derived angioblast/EC-like cells matched with cells marked as endothelium in the human CS12 data set (FIG. 4i, k), while TB cells of Axioloids overlapped with cells labeled as neural progenitor cells (FIG. 4h-k, FIG. 15e, f, h, i). As the latter population mainly matched with cells derived from the lower half of the embryo, they may represent a population similar to SOX2/TBXT double positive neural mesodermal progenitor cells (NMPs) found in the tail of the developing human embryo³⁷ (FIG. 4j).

### Presence of the HOX Code in Human Axioloids

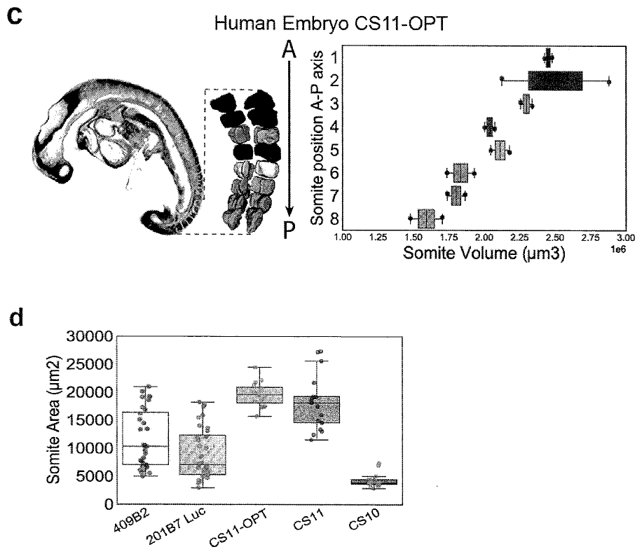

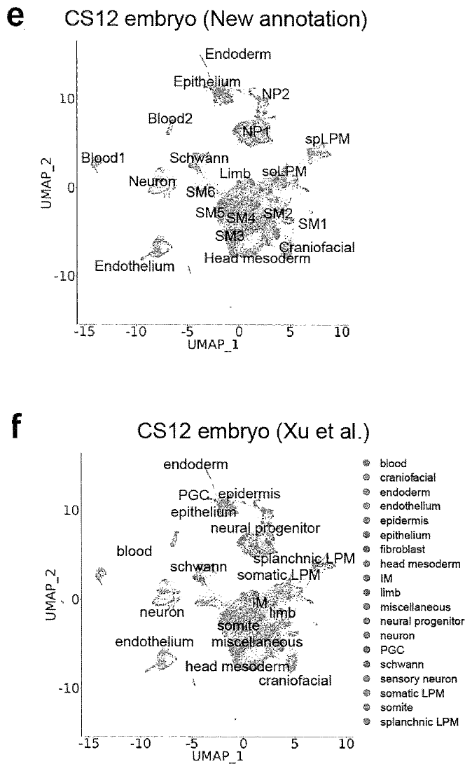

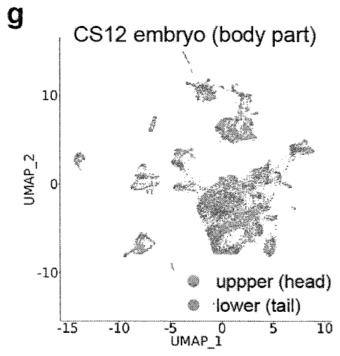

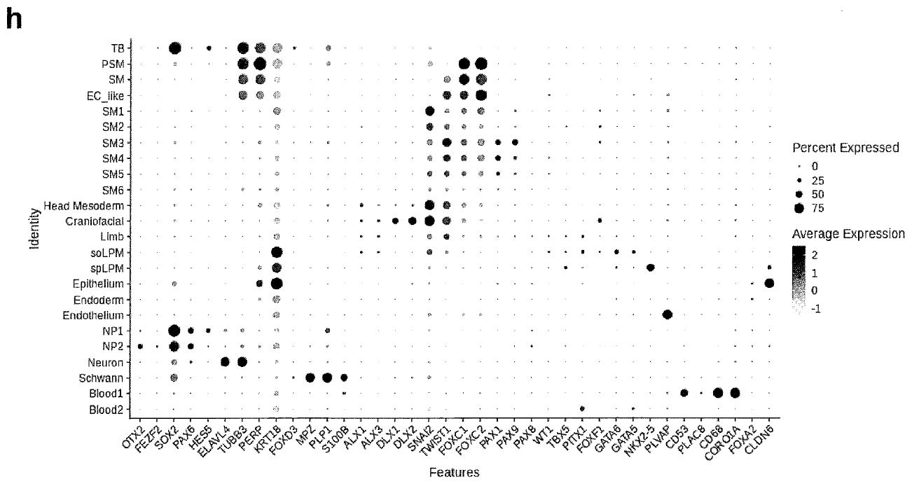

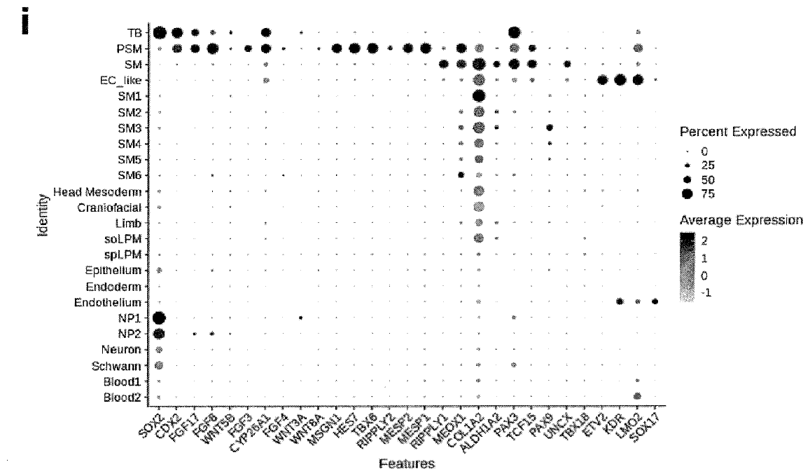

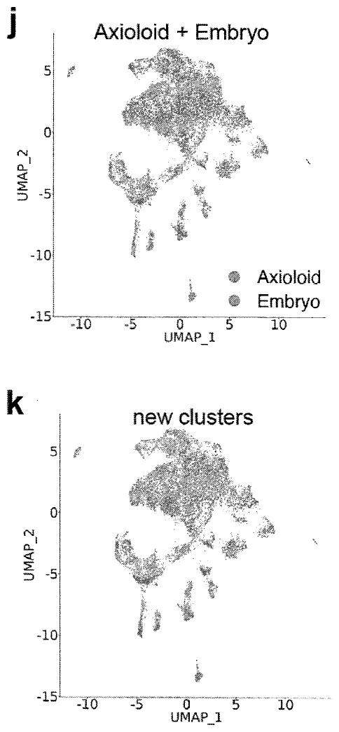

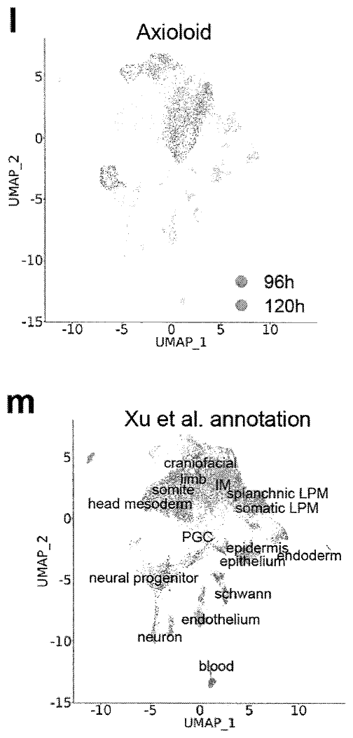

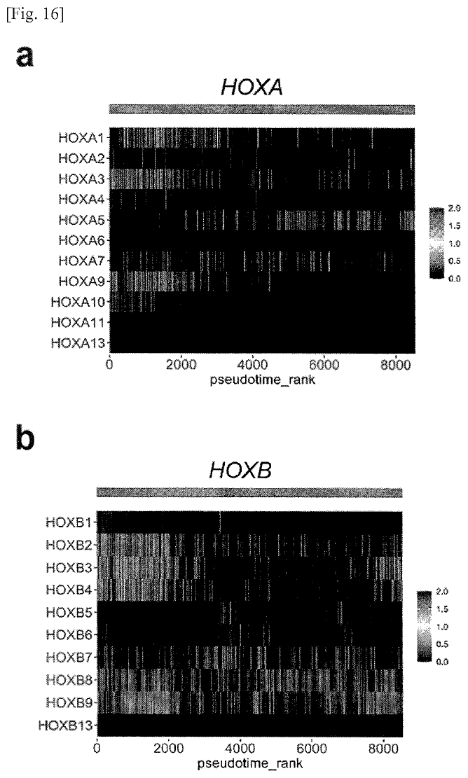

Having established a robust in vitro system to model axial development, with the use of both MG and RA, next stage was to see whether there is a HOX Code, i.e. the spatiotemporally controlled expression of HOX genes, in human Axioloids. Combining the scRNA-seq data with HybISS-based spatial transcriptomics³⁸, the spatial expression of major TB, PSM and SM markers was successfully recapitulated (FIG. 4l, m) and the expression of all four HOX clusters and associated HOX genes in human Axioloids were evaluated. Pseudotime analysis-based prediction of the HOX Code in Axioloids matched well with the actual spatial distribution of HOX gene expression along the anterior-posterior axis of human Axioloids (FIG. 4n, FIG. 16a-c). Especially for the HOXC cluster caudal regression of HOXC genes could be observed, in line with results obtained for mouse embryos (FIG. 4n-p). Combining these data sets with CUT & Tag experiments, the conducive and inhibitory epigenetic land scape of Axioloids was assessed, and a clear link between the switching of HOX gene expression in Axioloids and the active and repressive chromatin marks within the respective four HOX clusters was observed, supporting the notion of a HOX Code in human Axioloids, similar to earlier findings in human and mouse gastruloids^{5,39} (FIG. 4n-p, FIG. 16d-i).

### Modulating Signaling Pathways in Human Axioloids

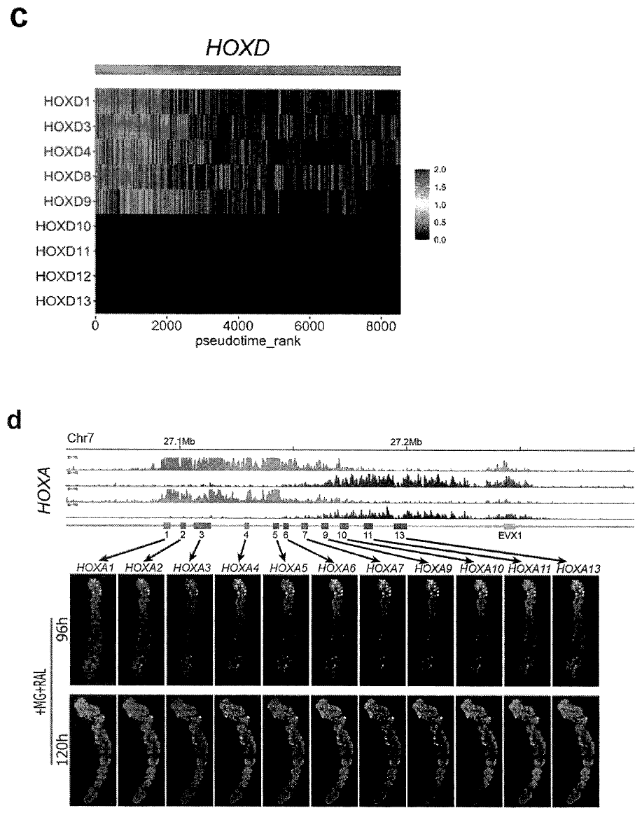

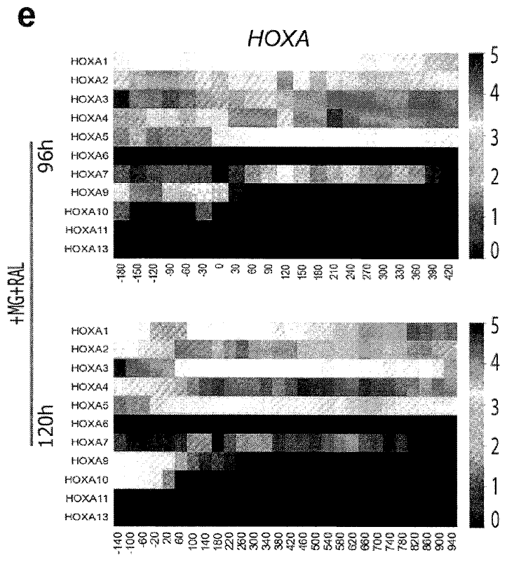

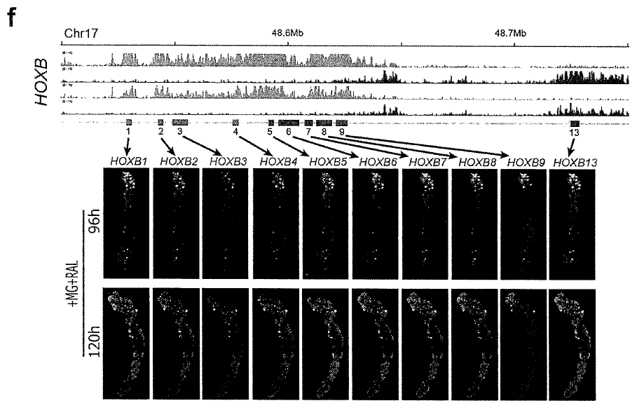

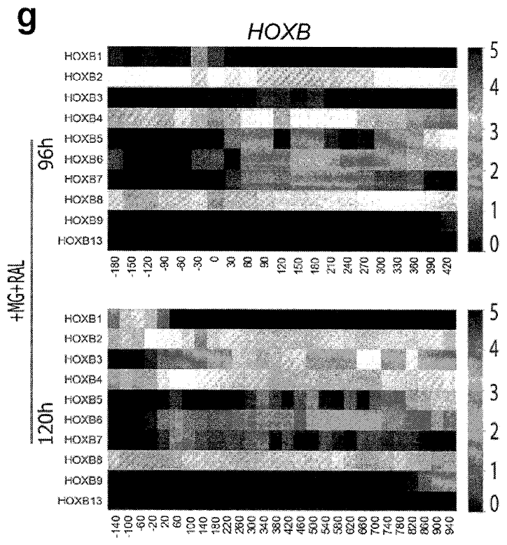

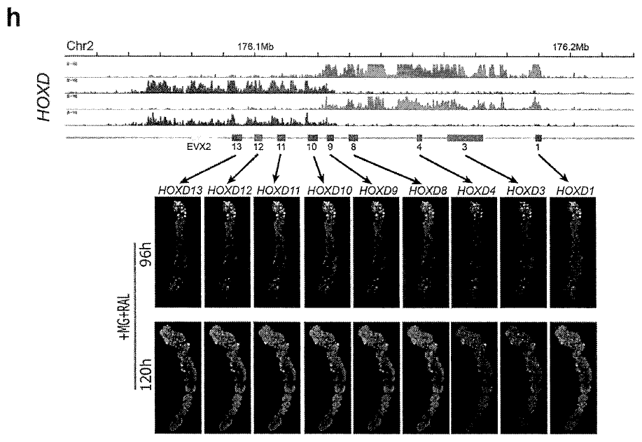

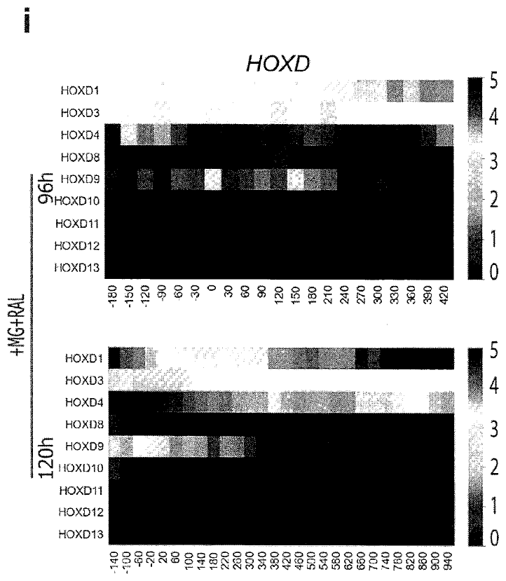

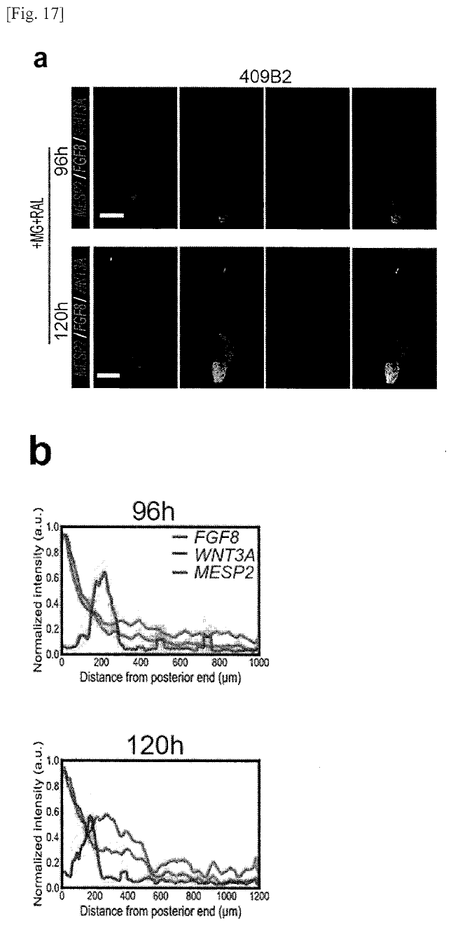

Based on the morphogenetic and molecular similarities between Axioloids and actual human embryos, it was then asked whether the latest iteration of the model system (MG+RAL) could be used to investigate the role of signaling pathways during human somitogenesis. Using HCR-based in situ hybridization, it was observed that Axioloids cultured in the presence of MG+RAL, still showed clear expression gradients of FGF8 and WNT3a in their TB and PSM region similar to Axioloids cultured in the presence of Matrigel only (FIG. 17a-d). RA signaling associated genes ALDH1 A2 and CYP26 A1 were also specifically expressed in their SM and TB regions respectively (FIG. 17e-h). To visualize and validate the spatial expression profiles of these and additional FGF/WNT and RA signaling pathway members predicted to exist in Axioloids from the pseudotime analysis of the scRNAseq data, HybISS-based spatial transcriptomics was applied (FIG. 17i-1). It was found that FGF3, FGF4, FGF17, WNT5a, and WNT5b showed graded expression in Axioloids similar to that of FGF8 & WNT3a while RDH10 was expressed similarly to ALDH1 A2 in the SM region of MG+RAL treated Axioloids, which is consistent with previous reports in vertebrate embryos (FIG. 17i-1).

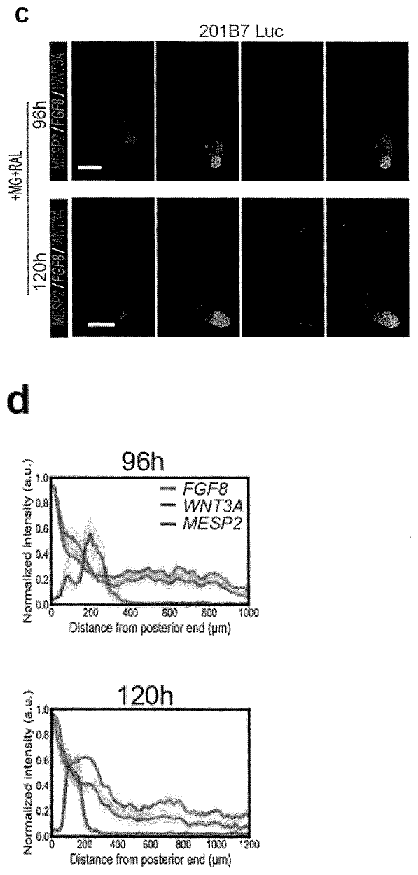

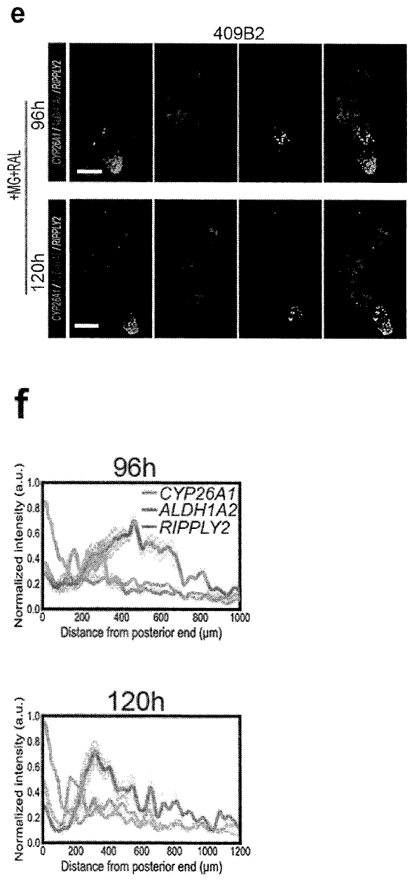

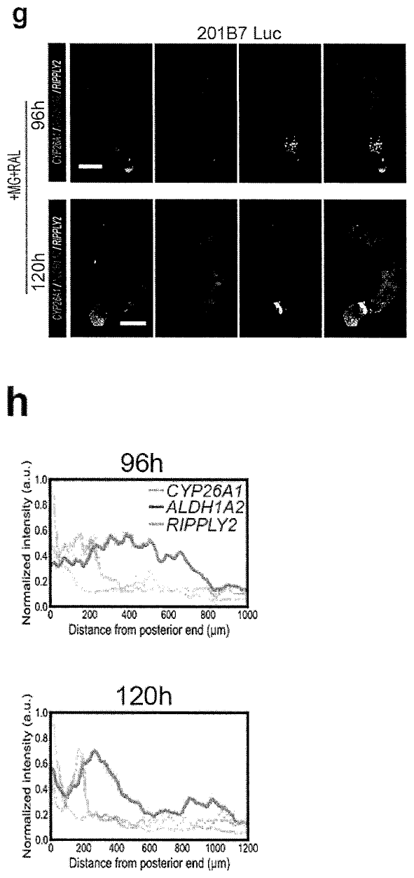

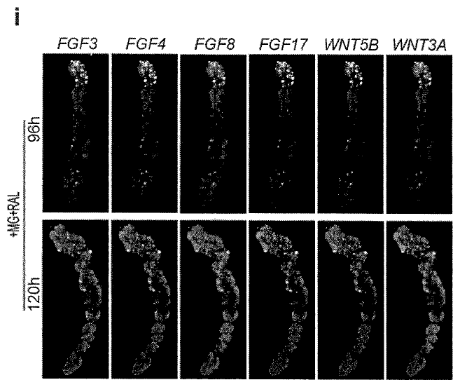

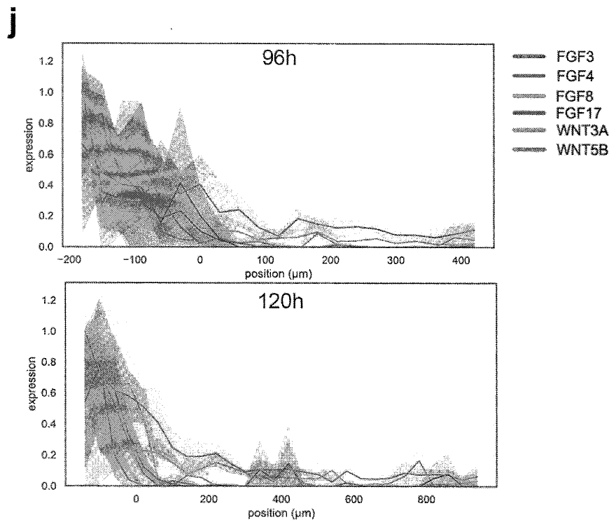

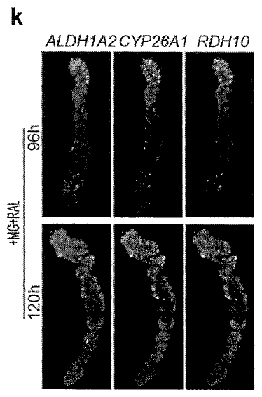

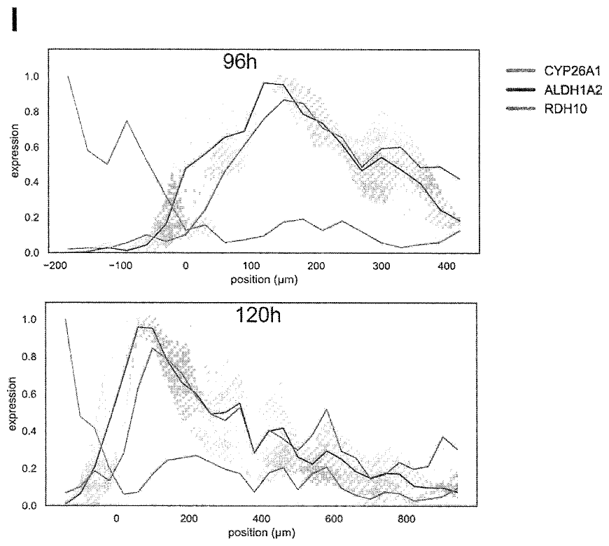

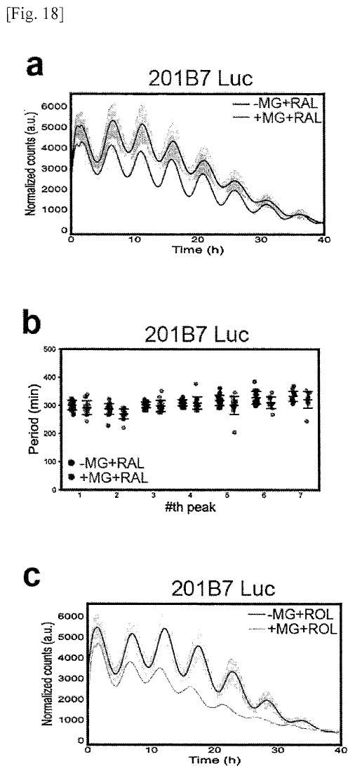

As RA signaling has a strong effect on somite formation and epithelialization, it was next asked whether it also influenced the oscillation and traveling wave-like activity of the segmentation clock within Axioloids. It was found that RA, RAL and ROL regardless of the presence or absence of MG in the system had largely no effect on the oscillatory activity including periodicity of the segmentation clock gene HES7, including robust presence of traveling wave-like expression in all treated Axioloids (FIG. 18a-j, Supplementary Video 7a). Inhibition of RA signaling via BMS493 had also no effect on the oscillatory activity of the segmentation clock gene HES7 (FIG. 18k-o, Supplementary Video 7b). Despite the clear detrimental effect of BMS493 on epithelialization and somite formation, the overall length of BMS493 treated Axioloids remained stable (FIG. 18p-r). These findings suggest that the segmentation clock and axial elongation can be uncoupled from RA-dependent epithelial somite formation.

Then next stage was to modulate via small molecules also the FGF, WNT and Notch signaling pathways in human Axioloids. The observed alterations of the segmentation clock were similar to what had been previously reported in vitro^{1,4-40} and in vivo⁴¹. As expected, Notch inhibition with DAPT led to a quick damping and loss of oscillatory activity in Axioloids, while FGF and especially WNT inhibition had less severe effects on the segmentation clock (FIG. 18s). The morphology of the treated Axioloids was affected and somite numbers were reduced at 120 h in all three cases albeit to a smaller extent than RA inhibition (FIG. 18p, t, u). Surprisingly, FGF inhibition had the most dramatic effect on overall length of Axioloids at 120 h (FIG. 18p, u, Supplementary Video 8), suggesting a role of FGF signaling on axial elongation of Axioloids, similar to what has been observed in classical embryo models^{42,43}.

Modeling Diseases of the Human Spine with Axioloids

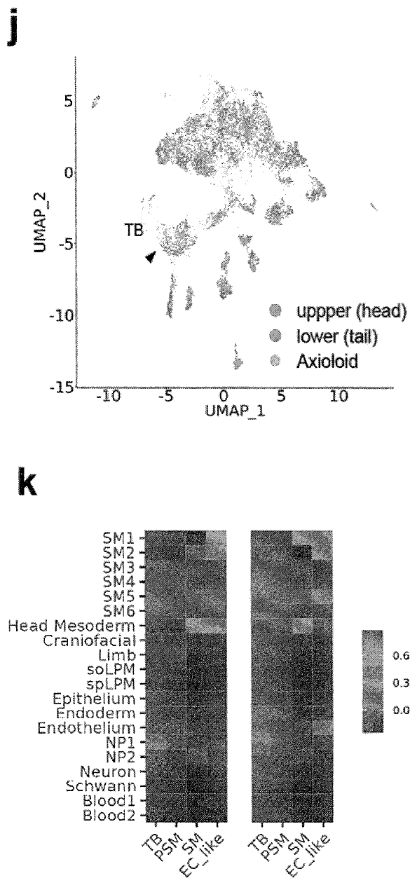

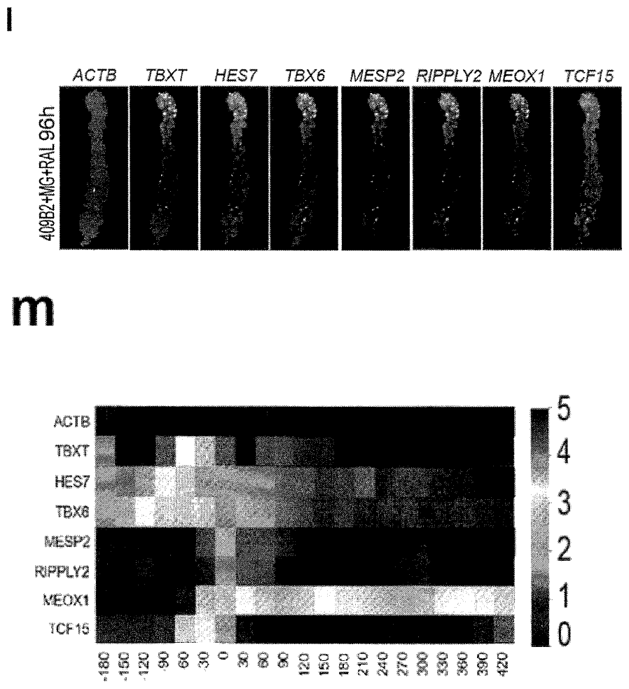

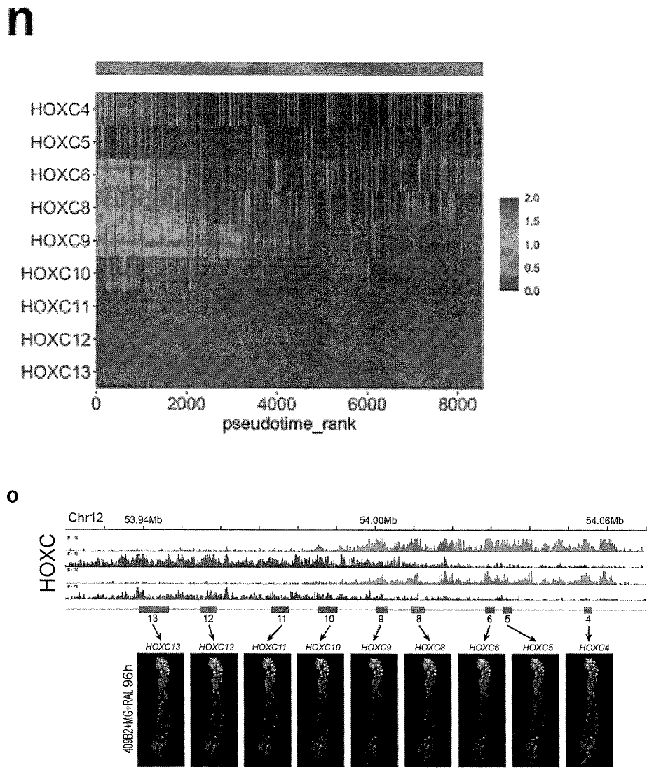

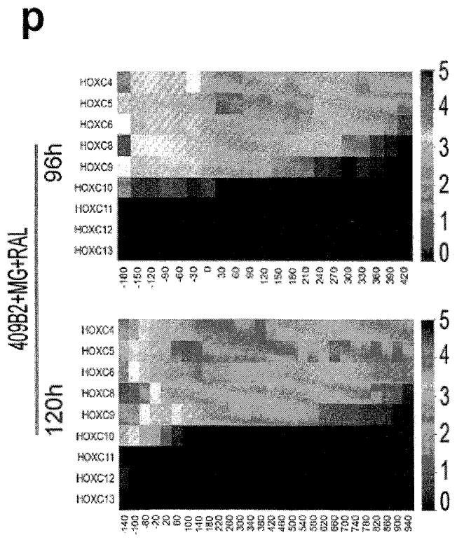

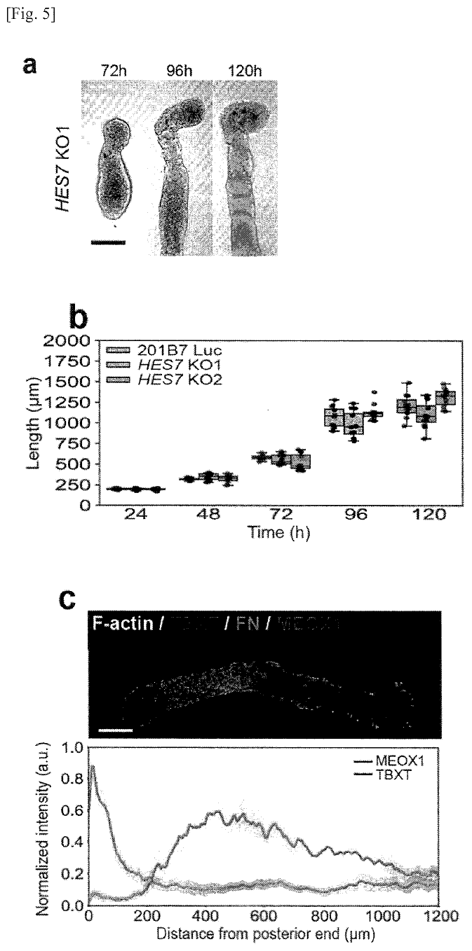

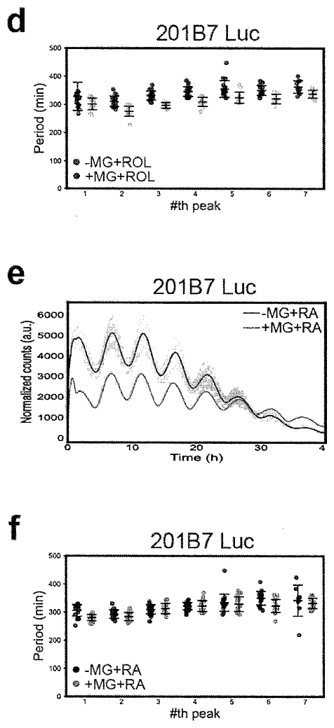

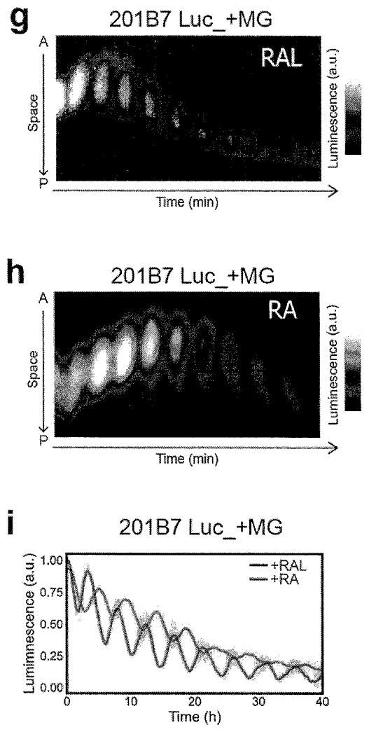

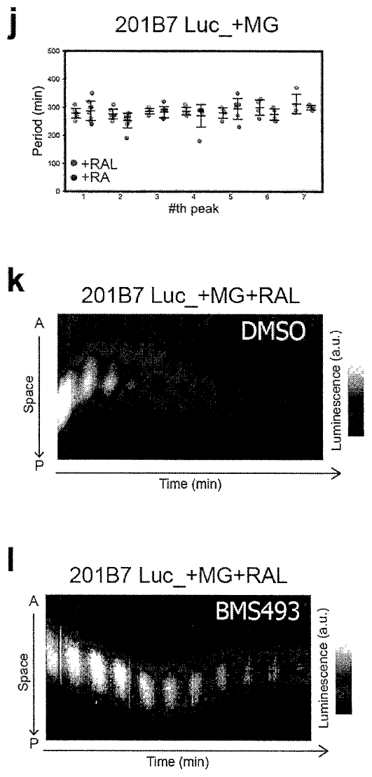

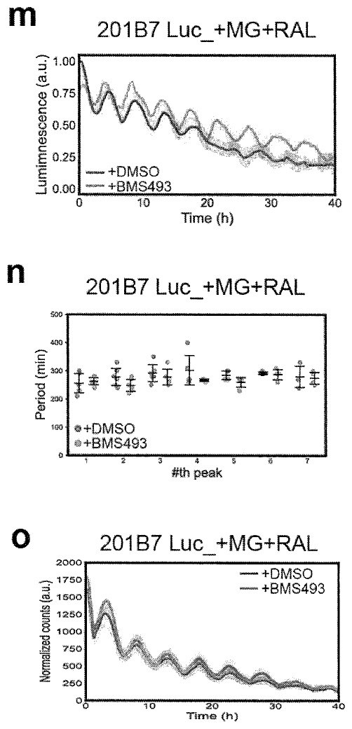

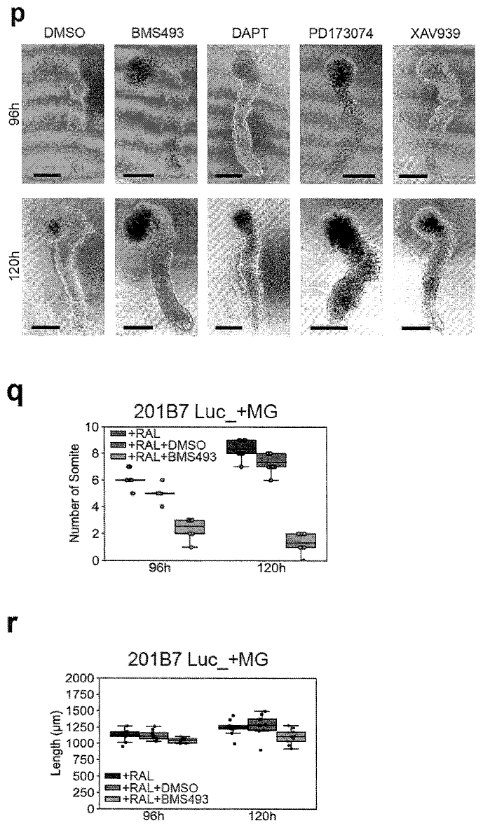

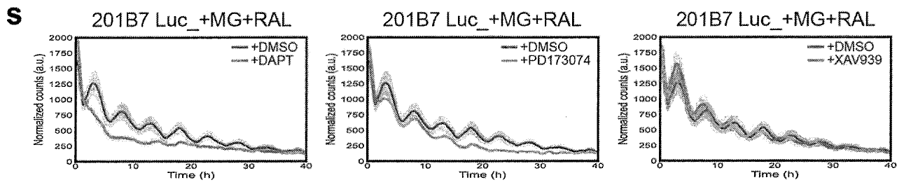

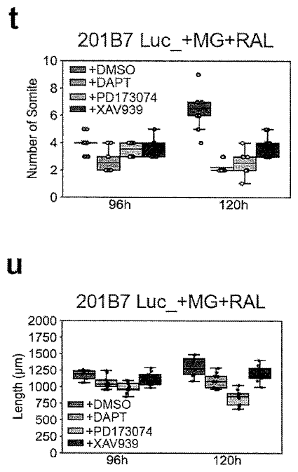

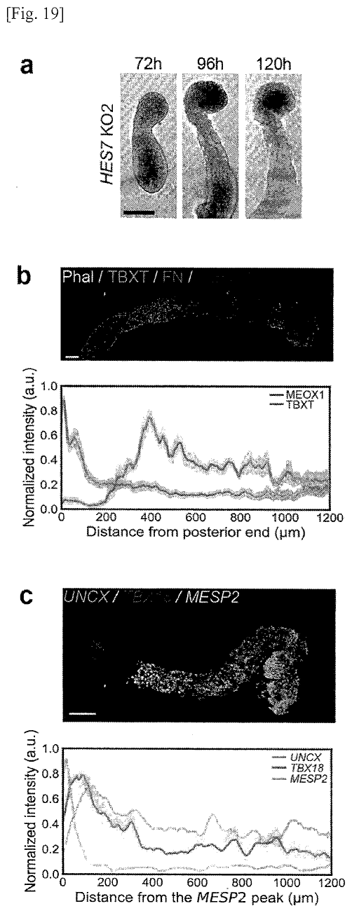

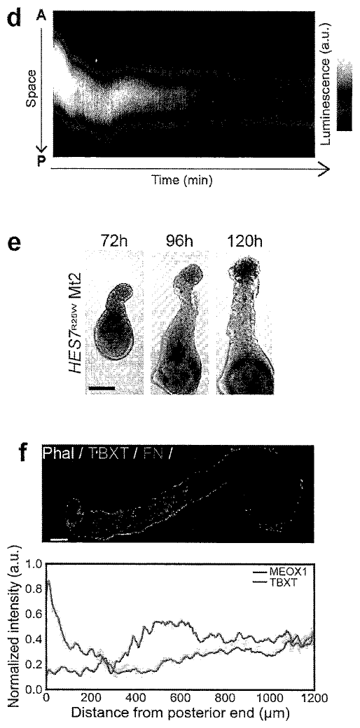

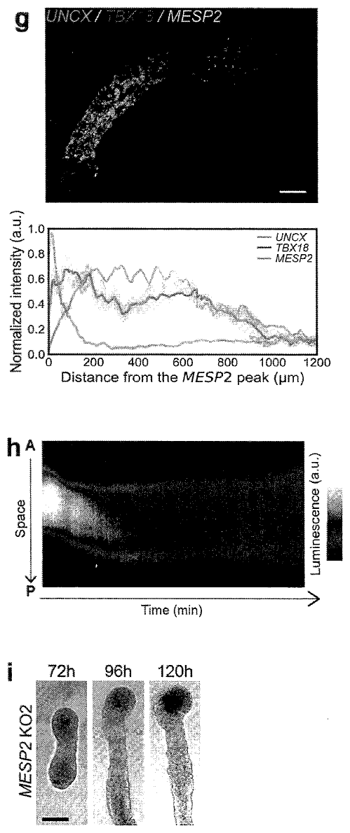

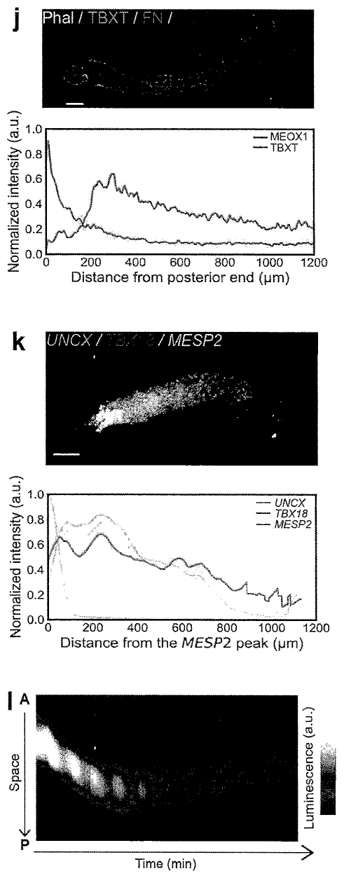

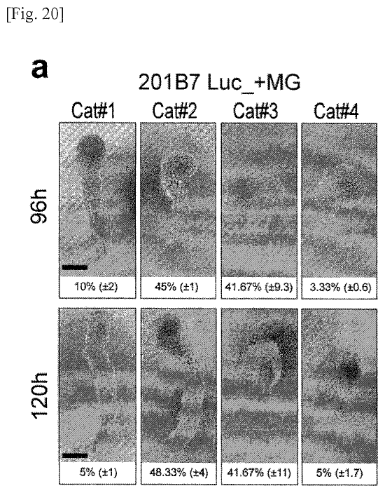

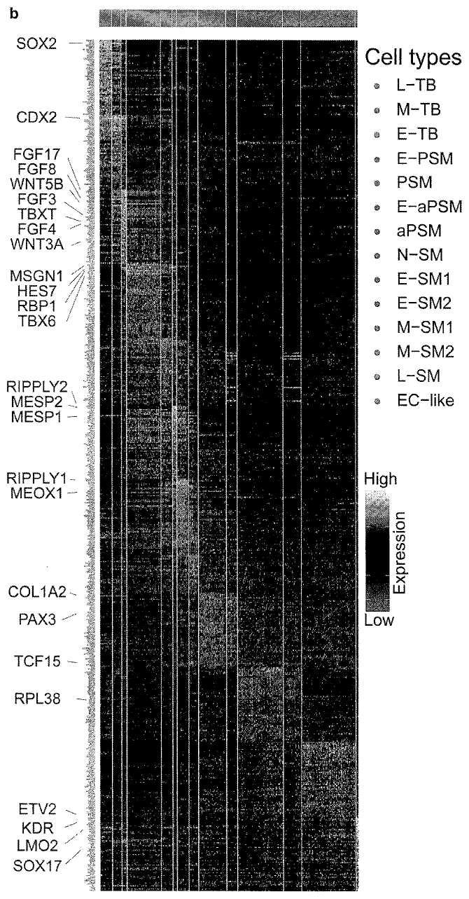

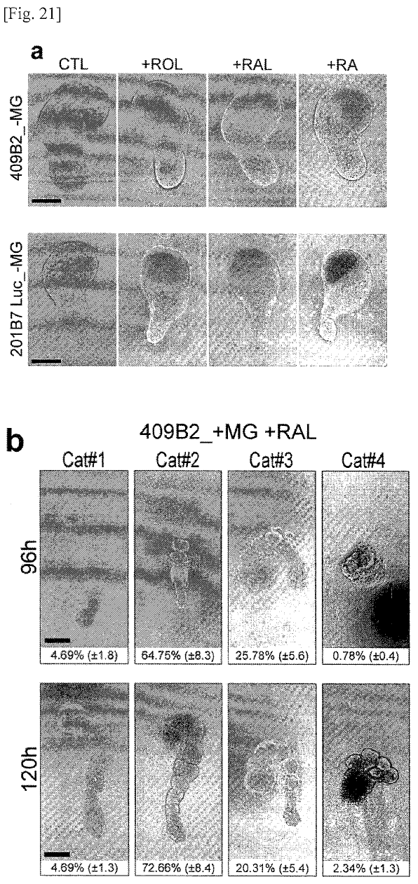

Lastly, it was investigated whether Axioloids can be used to model genetically associated diseases of the human spine. Using patient-like iPSC-lines harboring loss-of-function mutations in the coding regions of genes known to be associated with segmentation defects of the vertebrae (SDV), focusing on HES7⁴⁴ or MESP2⁴⁵, Axioloids was generated and their morphological, molecular and functional features were assessed. Two different HES7 knock-out iPSC-lines were used, which showed a similar phenotype: a conspicuous loss of segments and epithelial somite formation despite evident axial elongation (FIG. 5a, b, FIG. 19a). Notwithstanding the obvious absence of segments and somites, the polarized protein expression of TBXT and MEOX1 remained largely normal (FIG. 5c, FIG. 19b, Supplementary Video 9). A manifest loss of rostrocaudal patterning in HES7 KO Axioloids indicated by the loss of stripe-like expression pattern of UNCX and TBX18 was furthermore observed (FIG. 5d, FIG. 19c). Then the oscillatory activity of the segmentation clock in these patient-like Axioloids lacking HES7 was assessed and a clear loss of HES7 oscillation was observed, similar to the oscillatory phenotype, which was observed for HES7 KO PSM cells in the previous model system¹ (FIG. 5e-g, FIG. 19d, Supplementary Video 10).

A similar range of phenotypes in Axioloids derived from patient-like iPSC lines containing a point mutation (rs113994160: c.73C>T) in HES7 was observed, resulting in a pathogenic missense mutation R25W in the helix-loop-helix domain of HES7 reported to cause segmentation defects of the vertebrae (SDV)⁴⁴ (FIG. 5h-n, FIG. 19e-h, Supplementary Videos 11 & 12). Axial elongation in these HES7^{R25W} Axioloids appeared to be slightly increased as compared to the healthy donor control iPSC-derived Axioloids (FIG. 5h, i, FIG. 19e). HES7^{R25W} MT1 & MT2 derived HES7^{R25W} Axioloids exhibited normal expression of TB & SM markers TBXT and MEOX1 (FIG. 5j, FIG. 19f, Supplementary Video 11) combined with clear loss of rostrocaudal patterning (FIG. 5k, FIG. 19g), and loss of HES7 oscillation, again matching the findings for HES7 KO Axioloids as well as the previously reported oscillatory phenotype for HES7^{R25W} iPSC-derived PSM cells¹ (FIG. 5l-n, FIG. 19h, Supplementary Video 12).

Next, the effects of the loss of MESP2 were assessed, an aPSM associated transcription factor for which pathogenic mutations in patients with SDV have been previously reported⁴⁵. Using MESP2 knock-out iPSC lines (MESP2 KO1 & MESP2 KO2) patient-like Axioloids was derived and various morphological, molecular and functional features were again assessed. MESP2 KO Axioloids elongated normally but were devoid of segments or epithelial somites (FIG. 5o, p, FIG. 19i). In contrast to the largely normal expression pattern of TBXT and MEOX1, MESP2 KO Axioloids (FIG. 5q, FIG. 19j, Supplementary Video 13) exhibited severely impaired rostrocaudal patterning, characterized by the lack of stripe-like expression of UNCX coupled with low-level expression of TBX18 (FIG. 5r, FIG. 19k). This in vitro “human phenotype” resembled strikingly the one reported for MESP2 KO mice^{46,47}, indicating that Axioloids can reconstitute complex genetic phenotypes in vitro. In contrast to HES7, loss of MESP2 did not lead to a loss of HES7 oscillatory activity in Axioloids (FIG. 5s-u, FIG. 19l, Supplementary Video 14). Together, this data shows that Axioloids can provide not only invaluable insights into normal human axial development but also contribute to a better understanding of the pathogenesis of diseases of the human spine.

In summary, a pluripotent stem cell-derived 3D mesodermal model of human axial development have been established and characterized in-depth, which could reconstitute various aspects of human somitogenesis and axial development in vitro. Axioloids recapitulated a range of complex developmental processes including axial elongation, segmentation, epithelialization to oscillation of the segmentation clock, while also sharing molecular and morphometric features with the tail and axis of the developing human embryo. The bottom-up approach revealed the remarkable self-organization potential of primitive and paraxial mesoderm, which can give rise to the metameric basic body plan of the human embryo even in the absence of other germ layers. A crucial role of RA signaling on the morphogenetic processes associated with segmentation and somite formation within Axioloids was also uncovered, suggesting that RA supplementation especially in combination with ECM components might also improve the morphogenetic features of other in vitro model systems of human and non-human early embryonic development.

The bottom-up experimental approach demonstrates that complex developmental events such as somitogenesis, can be deconstructed and dissected into discrete “building blocks” of developmental principles which are usually intricately connected and cannot be easily uncoupled in vivo. Axioloids, a self-organizing in vitro model of human axial development allowed us to individually assess and manipulate such building blocks at the molecular, cellular and morphogenetic level. Further iterations of this model system will likely incorporate still “missing” anatomical structures such as notochord or neutral tube, which will allow assessment of subsequent stages of somitic development and differentiation including compartmentalization of somites into sclerotome, dermomyotome and other functional derivatives. Axioloids, a surrogate model of the human embryonic tail and forming axis, are capable of recapitulating core features of human somitogenesis, and represent an exciting new platform to investigate axial development and disease in a human context.

### Methods

### Culture of Human iPS Cells

Two human induced pluripotent stem cell (hiPSC) lines derived from healthy donors, 409B2⁴⁸ and 201B7⁴⁹, were used in this study, with the latter used mainly in the form of a HES7 reporter line described previously¹. For disease modeling, patient-like iPSCs with pathogenic mutations in HES7 and MESP2 generated via CRISPR-Cas9-based genetic editing were used¹. Human iPS cells were maintained in StemFit AK02N (Reprocell) medium supplemented with 50 U penicillin and 50 mg ml⁻¹ streptomycin (Gibco) on iMatrix-511 silk (Nippi) coated dishes. StemFit AK02N (Reprocell) medium contains three components, A, B and C, all three of which were mixed and used for standard maintenance culture of hiPSCs in humidified incubators at 37° C. and 5% CO₂. Utilized iPS cell lines were regularly tested and reported negative for mycoplasma contamination.

### Derivation of Human Axioloids

Human iPS cells were seeded on iMatrix-511 silk (Nippi) coated dishes at a density of 1.3×10⁴ cells/well into 6 well plates 5 days prior to Axioloid induction and used at 60% confluency. All subsequent induction steps were performed in AK02N (Reprocell) medium without component C (AK02N-C). Initially, hiPSCs were pulsed with a strong mesoderm and primitive streak fate inducing combination of bFGF (20 ng ml-1) and WNT agonist CHIR99021 (5 μM) for 24 h. CHIR99021 concentration may need to be adjusted depending on the used iPSC line, but remains usually in the range of 3-5 μM. 24 h after the initial pulse, cells were dissociated with Accutase (Thermo Fisher Scientific) and cultured for 24 h in 96-well U-bottom low attachment plates (Sumilon) at 500 cells/well in 50 μl of AK02N—C based aggregation medium supplemented with CHIR99021 (5 μM), basic FGF (20 ng ml¹), TGFβ inhibitor SB431542 (10 μM) and ROCK inhibitor Y27632 (10 μM). 24 h after aggregation, 150 μl of AK02N-C medium was added into each well and exchanged again after 24 h with 150 μl of fresh AK02N-C medium. 48 h after aggregation Axioloids were transferred one-by-one into BSA-treated 96-well flat-bottom low attachment plates (Watson) and embedded into 80 μl of AK02N-C medium containing 10% growth factor-reduced Matrigel (Corning) and cultured further at 37° C. and 5% CO₂ for 24 to 48 h. Depending on the experimental setup Retinoic Acid signaling molecules, i.e. Retinoic Acid (RA) (100 nM), Retinol (ROL) (10 μM) or all trans-Retinal (RAL) (1 μM) were added to the MG containing medium throughout the embedding phase. Small molecule inhibitors of FGF (PD173074 (250 nM)), WNT (XAV939 (2 μM)), RA (BMS493 (2.5 μM)) and Notch (DAPT (25 μM)) signaling were also added during the embedding phase and their effects on Axioloids assessed accordingly. For details of the used recombinant human proteins, small molecule agonists and inhibitors, please see Supplementary Table 1.

### Human Embryo Data

Digital data of human embryos were obtained from the MRC/Wellcome-Trust funded Human Developmental Biology Resource (HDBR, www.www.hdbr.org) with appropriate maternal written informed consent and approval from the Newcastle and North Tyneside NHS Health Authority Joint Ethics Committee (REC reference 18/NE/0290 & 08/H0906/21+5). HDBR is regulated by the UK Human Tissue Authority (license #12534) and operates in accordance with the relevant HTA Codes of Practice. The embryos were staged as Carnegie Stage (CS) 10 (n=1) or CS11 (n=4) based on features that were visible externally in the unfixed sample (https://hdbratlas.org/staging-criteria/carnegie-staging.html). One CS11 embryo (N662) was imaged using Optical Projection Tomography (OPT)⁵⁰. The remaining CS10 (13446) and CS11 (CS11-1021, 1177 and 1053) embryos were sectioned at a thickness of 4 μm (interval 20 im) for CS10 and 7 μm (interval 56 m for 1021 and 1053, and 35 μm for 1177) for CS11, stained with Hematoxylin and Eosin dye and imaged. The data related to the embryos utilized in this manuscript have been published^{51,52} and/or are publicly available on the HDBR Atlas website (https://hdbratlas.org/). OPT acquired image series of the CS11 embryo was 3D reconstructed and used for somite volume measurements, and scaled images of stained sections were used to measure the area of several visible and identifiable somite using ImageJ software.

### Immunohistochemistry (IHC)

Human Axioloid samples were washed twice with 0.1% BSA (Nacalai) in PBS (Takara), fixed with 4% paraformaldehyde (PFA) for 20 minutes at room temperature and then washed again twice with 0.1% BSA in PBS. Samples were permeabilized in 0.2% Triton X-100 (Nacalai) in PBS for 15 minutes at room temperature and blocked in 5% BSA in PBS for 1 hour at room temperature. Axioloid samples were stained with primary antibodies diluted in 0.5% BSA in PBST (1% Tween-20 (Nacalai) in PBS) for overnight (12-16 hours) at 4° C. Primary antibodies used in this study were: goat anti-TBXT (TBXT) (1:500, R&D Systems), mouse anti-Fibronectin (1:10, DSHB), mouse anti-Laminin (γ1) (1:10, DSHB), rabbit anti-MEOX1 (1:500, ATLAS), mouse anti-N-Cadherin (1:200, BD Biosciences), mouse anti-PKCζ (1:100, Santa Cruz), rabbit anti-SOX2 (1:400, Cell Signaling), and goat anti-TBX6 (1:500, R&D Systems). Samples were then washed twice with PBST and stained with secondary antibodies and Alexa Fluor 647 conjugated Phalloidin (Invitrogen) diluted in 0.5% BSA in PBST for 3 hours at room temperature, washed twice with PBST and counterstained with DAPI for 5 min at room temperature. Secondary antibodies used in this study (all diluted 1:500) were: Alexa Fluor 405 donkey anti-goat (Abcam), Alexa Fluor 405 donkey anti-mouse (Abcam), Alexa Fluor 488 donkey anti-goat (Invitrogen), Alexa Fluor 488 donkey anti-mouse (Invitrogen), and Alexa Fluor 555 donkey anti-rabbit (Invitrogen). Prior to imaging, stained Axioloid samples were submerged in and treated with Scale S4 clearing solution⁵³ for overnight at 4° C. Images were taken using LSM980 (Carl Zeiss), Ti2 (Nikon) equipped with Dragonfly (Andor) or Nikon A1R MP (Multiphoton+N-STORM) fluorescence microscopes. For further details of used primary and secondary antibodies see Supplementary Table 2.

### Whole-Mount In Situ Hybridization (HCR)

All probes, HCR amplifiers and buffers (hybridization, wash and amplification buffers) were purchased from Molecular Instruments and whole-mount in situ hybridization chain reaction (HCR) was performed as previously described⁵⁴. Briefly, human Axioloids were collected in microtubes coated with 1% BSA in PBS, washed once with 1% BSA in PBS and then fixed with 4% PFA in PBS for 1 h at room temperature. Samples were then washed three times with PBST (0.1% Tween-20 in PBS) at room temperature 5 min each, post-fixed with 100% Methanol and stored at −30° C. until further use. Samples were rehydrated by washing with a series of graded 500 μL Methanol/PBST wash steps (75%, 50%, and 25%) for 5 min each at room temperature. Human Axioloid samples were then incubated for 5 min with hybridization buffer at room temperature, then for 30 min at 37° C. A mixture of probes (8 nM final for each) in hybridization buffer was incubated for 30 min at 37° C. prior to use. Samples were incubated for 12-16 h at 37° C. with probe-containing hybridization buffer. Samples were then washed with probe wash buffer 4 times 15 min each at 37° C. followed by washing with 5×SSCT 3 times at room temperature. Next, samples were pre-amplified by incubating at least 30 min in probe amplification buffer at room temperature. Amplifier hairpins were prepared by snap-cooling; heating each hi and h2 hairpins separately at 95° C. for 90 sec and then cool down at room temperature for 30 min in the dark. Hairpin mixtures were prepared at 6 nM each by adding h1 and h2 in 250 μL of amplification buffer. Axioloid samples were then incubated with the amplifier hairpin-containing solution for 12-16 h at room temperature in the dark. Finally, samples were washed with 5×SSCT and PBST, followed by counter staining with DAPI. During each wash and after addition of buffers, probes and hairpin mixtures into samples, tubes were inverted several times (5-20 times) to mix properly. HCR stained Axioloids were stored in 1% BSA in PBST at 4° C. not more than two weeks before imaging. HCR probe design and associated hairpins was as follows: ALDH1 A2 (Accession NM_003888.4, hairpin 514-B5); CYP26 A1 (Accession NM_000783.4, hairpin 594-B4); FGF8 (Accession NM_033163.5, hairpin 546-B2); HES7 (Accession NM_001165967.2, hairpin 594-B4); LFNG (Accession NM_001040167.2, hairpin 488-B1); MESP2 (Accession NM_001039958.2, hairpin 647-B3); MSGN1 (Accession NM_001105569.3, hairpin 514-B5); PCDH8 (Accession NM_002590.4, hairpin 647-B3); RIPPLY2 (Accession NM_001009994.3, hairpin 647-B3); TBX6 (Accession NM_004608.4, hairpin 647-B5); TBX18 (Accession NM_001080508.3, hairpin 546-B2); TCF15 (Accession NM_004609.3, hairpin 594-B4); UNCX (Accession NM_001080461.3, hairpin 488-B1); WNT3 A (Accession NM_033131.4, hairpin 488-B1).

### Hybridization-Based In Situ Sequencing (HybISS)

Human Axioloids were washed with 0.1% BSA (Nacalai) in PBS twice and fixed with 0.4% PFA for 20 minutes at room temperature and then washed twice with 0.1% BSA in PBS. Axioloid samples were then transferred to a cryomold (Sakura Finetek), embedded in frozen section compound (Leica) and stored in −80° C. until sectioning. Tissues were cryosectioned at 8 m thickness and collected on a slide glass (MAS-01, MATSUNAMI). HybISS was performed as described previously³⁸ with slight modifications. Briefly, slides were fixed with 3% formaldehyde for 5 minutes, washed with PBS twice, permeabilized with 0.1 M HCl for 5 minutes, and washed with PBS twice. After passing through 70% and 100% ethanol for dehydration, gaskets (SecureSeal Hybridization chambers, Grace Bio-Labs) were glued onto the sides enclosing the tissue. The mRNA was in situ reverse-transcribed to complementary DNA through following steps: Tissues were treated with random decamer (5 μM, obtained from IDT) for 5 minutes at 65° C. and cooled down on ice for 5 minutes. Then tissues were incubated with in-situ reverse transcription mix including superscript IV (20 U/μl, Thermo Fisher Scientific) and random decamer (5 μM) for 10 minutes RT and then overnight at 42° C. After reverse transcription, tissues were fixed again with 3% formaldehyde for 40 minutes at RT, followed by degradation of the mRNA with RNaseH (0.4 U/μl, NEB) for 30 minutes at 37° C., hybridization of padlock probes (PLPs) to the remaining cDNA (20 nM for each PLPs in 20% formamide), and ligation of padlock probes with Tth ligase (0.5 U/μl, BURT) for 90 minutes at 45° C. For the padlock probe design, 5 target sequences were selected per gene using Python padlock design software package (https://github.com/Moldia/multi_padlock_design) with the following parameters: arm length, 15 (if targets were not found, 20); Tm, low 65, high 75; space between targets, 15. Every set of padlock probes for a given gene carries a unique 20 nucleotide (nt) ID sequence as well as 20 nt sequences that is common among all of PLPs. After padlock probe hybridization and ligation, cDNA was digested with exonuclease I (0.5 U/μl, Thermo Fisher Scientific) for 3 hours at 37° C. Then rolling circle amplification (RCA) was performed with phi29 polymerase (1 U/μl, Monserate) overnight at 30° C. Resulting RCA products (RCPs) were subjected to sequencing by hybridization. Each round of sequencing contains the process of bridge probe hybridization (0.2 μM each) in 1× hybridization buffer (2×SSC, 20% formamide), detection probe hybridization (0.2 μM each) with Hoechst staining (1 μg/mL) in 1× hybridization buffer, imaging, and stripping with stripping solution (2×SSC, 65% formamide). The sequences of PLPs, bridge probes, and detection probes are shown in Supplementary Table 3.

### Imaging and Sequencing by Hybridization

Imaging was performed using a standard epifluorescence microscope (Nikon Ti2-E) connected to LED light source (Lumencor SPECTRA X light engine). Images were obtained with a CMOS camera (ORCA-Flash4.0V3, Hamamatsu) with CFI Plan Apochromat Lambda objective 40× (1.3 NA, oil). Filter cubes for wavelength separation were as follows: Chroma 89402X (Hoechst, Cy5), Chroma 89403X (AlexaFluor750), Semrock GFP-A-Basic (AlexaFluor488), Semrock Cy3-4040C (Cy3), and Semrock CFP-2432C (Atto425). For multiplex analyses, unique 4-digit code was assigned to each gene, in which each digit corresponds to one of four different fluorophore-conjugated detection probes (1: AlexaFluor750, 2: AlexaFluor488, 3: Cy3, 4: Cy5). Every digit code was reconstituted through 4 rounds of hybridization and imaging in situ. Several genes were imaged separately to avoid optical crowding: ACTB, HOXB9, HOXA3, HOXB3, and HOXD4 for tissue 1, 2, and 3 of 96 h and tissue 4 of 120 h. ACTB and HOXB9 for tissue 1 and 3 of 120 h. Multispectral image was obtained for multiple cycles. Each image consists of multiple tiles that together cover the tissue section (10% overlap), and each field of view consists of Z-stacks stepping 0.8 m through entire tissue thickness. Tiles were stitched and Z-stacks were merged to maximum-intensity projections (NIS-Elements). 8-bit TIF image of each channel from each round was exported for data analyses.

### Analyses of HybISS Data

Data analyses of HybISS and subsequent quantification were performed with homemade Python code and Fiji. For decoding gene spot, firstly, images were roughly aligned to first round images using Hoechst staining. Then images were top-hat filtered and split into multiple smaller images, which will be referred as tiles hereafter. For each tile in each round, composite images of the four detection probe channels were created, aligned to the first-round composite images, and split into each channel again. Then each tile is stitched to create the whole image. Gene spots were detected using Laplacian of Gaussian Filter in the first-round image, and signal intensity of each channel at the spots was calculated in the rest of rounds. Spot intensity was normalized by dividing the intensity by the 99^{th} percentile in the channel. Following spots were removed from the analysis due to the low quality: the maximum intensity in the set of channels is less than 0.15. The maximum intensity in the set of channels divided by the sum of the all of the channels is less than 0.5. Spots passed through the quality control were assigned to the gene according to their reconstituted 4-digit code. Density of gene spots along anterior-posterior axis was quantified as follows: firstly, anterior-posterior axis was manually drawn and tissues were divided so that each segment have the same area. Then density of gene spots, which was represented as number of spots/1,000 μm², was calculated in each segment for each gene, and the center of mass of the segment whose density of MESP2 is highest was set as reference position. Distance between the center of mass of adjacent segments was calculated, and sum of distance from the reference position was shown in x-axis, in which posterior-to-anterior is minus-to-plus direction. To produce line graph, gene density was normalized so that maximum density was set to one. To produce heatmap, the value of gene density plus 1 was log-transformed (base e). All of the python code used in the analyses above is available via GitHub (xxxxx) or upon request from the corresponding author.

### Live-Cell Imaging Using Ti2

Human Axioloids were transferred into 1% BSA (Nacalai) treated 96 well flat-bottom low attachment plates (Watson) with one Axioloid per well in 80 μl of AKO2-N—C based embedding medium (+/−MG, +/−RA, RAL or ROL, +/−small molecule modulators of signaling pathways) and were cultured in a stage top incubator (Tokai Hit) which was set to 37° C. and 5% CO₂. Brightfield live-cell imaging was performed with an inverted Ti2 microscope system (Nikon) using PlanApo λ 10× objective, with the microscope running in autofocus mode, focusing at 10 μm intervals over a total range of 190 μm. Images were taken every 3 minutes and processed with Fiji.

### Bioluminescence Live-Cell Imaging

Bioluminescence live-cell imaging of HES7-reporter (201B7Luc) iPSC-derived human Axioloids (201B7Luc) was performed and signal was quantified as previously described^{55,56} Briefly, one day before imaging glass-bottom 96-well plates (Iwaki) were coated with 50 μl of 1.5% PVA (Nacalai), the PVA solution was hereby aspirated and the plates dried on top of a clean bench overnight at room temperature. Prior to imaging, Axioloids were set onto coated glass-bottom 96-well plates with 80 μl of AK02N-C medium containing 100 mM D-Luciferin and 10% Matrigel. Small molecule agonists and antagonists of signaling pathways were added according to the experimental setup. Bioluminescence signals of Axioloids induced from the HES7-reporter cell line were recorded on IX83 (Olympus) equipped with iXon EMCCD camera (Andor) cultured in a stage top incubator (Tokai Hit). Signal was acquired with 2×2 binning and 1 min exposure. Cosmic rays were removed from the raw images by applying spike noise filter, then smoothed by median filter. Temporal fluctuation of signal baseline was corrected by background subtraction. Kymograph was generated by averaging luminescence intensity values along the lateral axis of Axioloids and resulted values were aligned in temporal order.

### Imaging of Fixed Samples

For confocal imaging of stained Axioloids, samples were set onto glass-bottom 35 mm dishes or 8-well chamber covers (Matsunami) in BSA-containing PBS and imaged. Confocal microscopy was performed on LSM980 (Carl Zeiss Microscopy), Dragonfly (Andor) equipped Ti2 fluorescent microscope (Nikon) or Nikon A1R MP (Multiphoton+N-STORM) fluorescence microscopes. For HCR-stained samples, images were taken as tiled z-stacks with z-intervals of 10 μm. For immunostained samples, a center z-plane of each Axioloid was observed. For 3D reconstruction of immunostained Axioloids, samples were cleared and mounted in ScaleS4 clearing solution⁵³ and tiled multi-stack images with z-intervals of 1.4-5 μm were acquired. 3D reconstruction Videos of immunostained samples were generated using NIS-Elements and the 3D Slicer software.

### Somite Volume Measurements

Measurement of somite volume was done using the 3D Slicer software (https://discourse.slicer.org/), based on z-stack images of immunostained Axioloids and CS11 human embryo data acquired previously using confocal microscope and OPT technic respectively. Using the segment editor, each somite was individually highlighted based on manual selections on several images of each stack, then global structure of each somite across each image was extrapolated automatically by the software using the fill between slices function. This updated selection was then manually cured before being used to recreate a 3D view of the highlighted structures. Voxel number and volume of each individual segment was then extracted automatically using the segment statistics function.

### Period Measurement (Oscillation)

Periodicity of HES7 oscillation was quantified from time-series luminescence intensity data obtained from either live imaging or Kronos HT (Atto) measurements using HES7 reporter-derived Axioloids. For Kronos HT based oscillation measurements, Axioloids were transferred at 72 h (48 h after aggregation) into 24-well film-bottom plates (Eppendorf) covered in 400 μl of embedding medium (+/−MG) supplemented with D-Luciferin (100 μM) (bioWORLD). Axioloid containing culture plates were cultured at 37° C. and 5% CO₂. Oscillations were measured for 48 hours and each well was measured for 10 seconds with 8 min intervals. Intensity values were processed using Matlab. Temporal trend was obtained by subtracting moving average (window size of 10 h), and the detrended signal was smoothed by Savitzky-Golay filter (window size of 3 h). Instantaneous oscillation phase was calculated by applying Hilbert transform, then peak detection was performed on cosine values of instantaneous oscillation phase. Peak-to-peak period was then quantified on each n-th oscillation.

### Period Measurement (Ti2 Brightfield Live Imaging)

Periodicity of segmentation was quantified from brightfield live-cell imaging data obtained with an inverted Ti2 microscope system (Nikon) using PlanApo λ 10× objective. All images were processed with Fiji and segments were assigned and corresponding time points of segment formation noted for the 24 h and 48 h periods following Axioloid embedding into MG.

### Quantification of Imaging Data

Quantification of length or intensity based on imaging data was performed using Fiji⁵⁷. Rostrocaudal length of formed segments was quantified from a center plane in z-stack images of Phalloidin-stained Axioloids. The longitudinal axes of Axioloids were measured on bright field images using Segmented Line tool. For quantifying fluorescent intensity of HCR or IHC staining, first, maximum intensity z projection images were created from multi-channel z-stack confocal images, then intensity of each channel was quantified along the longitudinal axis of Axioloids by Segmented Line tool, with a line width which corresponds to approximately 80% of the lateral length of Axioloids. Intensity values were further processed and plotted by using custom Python codes. Savitzky-Golay filter was applied on raw intensity values and then normalized. For HCR datasets including MESP2 staining, positional values were normalized using MESP2 peak before calculating average intensity of multiple samples.

pA-Tn5 Transposome Preparation

The recombinant Protein A-conjugated Tn5 transposase (pA-Tn5) was extracted and purified from bacterial cell lysates of T7 Express lysY/Iq Competent E. coli (NEB) harboring 3λFlag-pA-Tn5-Fl plasmid (a gift from Prof. Steven Henikoff, Addgene, #124601) using the columns filled with chitin slurry resin (NEB) after sonication-mediated solubilization as described before⁵⁸. The transposase was assembled with a quarter of equimolar amount of the two types of oligo DNA adaptors (5′-TCGTCGGCAGCGTCAGATGTGTATAAGAGACAG-3′ (SEQ ID NO: 1) and 5′-GTCTCGTGGGCTCGGAGATGTGTATAAGAGACAG-3′ (SEQ ID NO: 2)), each of which were pre-annealed with 5′-[PHO]CTGTCTCTTATACACATCT-3′ (SEQ ID NO: 3).

### CUT&Tag Library Preparation

Axioloids were pooled and dissociated into single cell suspension in the same way as for the single-cell RNA-seq library preparation. Then, the cells were pelleted and snap-frozen in liquid nitrogen. Later on, the cells were processed according to previous literature⁵⁸ with slight modifications. Briefly, 50,000 cells per experiment were first washed with wash buffer (20 mM HEPES pH 7.5, 150 mM NaCl, 0.5 mM spermidine, 1× Protease Inhibitor (Roche)), and then resuspended again in the wash buffer. The cell suspension was then mixed with Concanavalin A-coated beads (Bangs Laboratories) resuspended in binding buffer (20 mM HEPES pH 7.9, 10 mM KCl, 1 mM CaCl₂, 1 mM MnCl₂). Next, cells were resuspended in 100 μL of ice-cold wash buffer, supplemented with 0.05% digitonin, 0.1 mM EDTA, and 0.1% BSA together with each 0.5 μl primary antibody. The antibodies used were mouse monoclonal IgG1 antibodies against H3K4me3 (MAB Institute, #MA304B) and H3K27me3 (MAB Institute, #MA323B). After incubation at room temperature for 2 hours, the buffer containing the primary antibody was replaced with 100 μl of ice-cold wash buffer, supplemented with 0.05% digitonin, together with 1 μl secondary rabbit anti-mouse IgG antibody (abcam, ab46540), and was incubated at 4° C. for overnight. After washing the beads with Dig-300 buffer (20 mM HEPES pH 7.5, 300 mM NaCl, 0.5 mM spermidine, 0.01% digitonin, 1× Protease Inhibitor (Roche)) twice, 100 μl of the Dig-300 buffer containing 0.5 femtomol pA-Tn5 transposome assembled above was applied and incubated for 1 hour at room temperature. After washing the beads with Dig-300 buffer twice, the beads in 50 μl of Dig-300 buffer supplemented with 10 mM MgCl₂ were resuspended and incubated at 37° C. for 1 hour. Then the supernatant was removed, and the beads were resuspended in 20 μl of 10 mM TAPS buffer (pH 8.5). Then thermolabile proteinase K (NEB) was added, and the mixture was incubated at 37° C. for 30 minutes, followed by incubation at 55° C. for 20 minutes to inactivate the proteinase. PCR-amplification of the library was directly performed from the tube after adding the reaction mixture (KAPA BIOSYSTEMS, #KK2502) using the primers listed in Supplementary Table 7.

The amplified libraries were purified with 1.3 volumes of AMPure XP beads. The libraries were sequenced with NovaSeq 6000 Sequencing System (Illumina) using NovaSeq 6000 SP Reagent Kit v1.5 (100 Cycles) (Illumina, #20028401) with the paired-end mode.

### CUT&Tag Data Processing

After the removal of the tagmentation adapter sequences with Cutadapt (version 3.4)⁵⁹, paired-end reads were aligned to the human genome reference hg38 using Bowtie2 (version 2.2.4)⁶⁰ with options—very-sensitive—X 2000. From mapped reads, properly paired (Samtools flag 0×2), MAPQ>=20, and non-mitochondrial chromosomal reads were extracted using Samtools (version 1.12)⁶¹. PCR duplicate reads were removed with Picard (version 2.25.2) (http://broadinstitute.github.io/picard/), and then reads mapped to blacklist (http://mitra.stanford.edu/kundaje/akundaje/release/blacklists/hg38-human/hg38.blacklist.bed.gz) were removed with bedtools (version 2.30)⁶². Bam files were converted to bigwig format with deepTools (version 3.5)⁶³ and visualized with Integrative Genome Viewer (IGV) (version 2.12.2).

RNA Library Preparation for scRNA-Seq Analysis

All plastic materials used during the dissociation process of human Axioloids such as round bottom 96 well cell suspension plates, tubes, tips and cell strainers, were precoated with 0.1% BSA (SIGMA) in HBSS(−) (Wako). Axioloids were collected into wells of a 96-well plate and washed three times by transferring to another well filled with 0.1% BSA (SIGMA) in HBSS(−). Axioloids of the desired stage and number were then transferred to another well filled with 100 μl of enzyme P solution of the Neural Tissue Dissociation Kit (P) (Miltenyi Biotec) and incubated at 37° C. for 10 minutes. After adding 100 μl of 0.1% BSA in HBSS(−) Axioloids were dissociated by pipetting with an uncut P200 tip 20 times. The dissociated cells were filtered through Flowmi Cell Strainers with 40 m porosity (Merck) into a BSA-coated 2 ml tube (Eppendorf). Cells were washed twice in 1% BSA in HBSS(−) with centrifugation steps at 400×g for 6 minutes at room temperature. Cells were then resuspended in 1% BSA in HBSS(−) using a BSA-coated uncut-P200 tip and the concentration of the cell suspension was determined with hemocytometer. The cell suspension was subjected to library preparation using Chromium Next GEM Single Cell 3′ Kit v3.1 (10× Genomics), aiming for a target cell recovery of 5000-10000. Library preparation was preformed following the instructions provided by the manufacturer 10X Genomics (CG000315 Rev B).

scRNA-Seq Library Sequencing

The libraries were sequenced with NovaSeq 6000 Sequencing System (Illumina) using NovaSeq 6000 SP Reagent Kit v1.5 (100 Cycles) (Illumina, #20028401) and NovaSeq 6000 S1 Reagent Kit v1.5 (100 Cycles) (Illumina, #20028319) with the paired-end mode as described in the instructions by 10X Genomics (CG000315 Rev B).

scRNA-Seq Data Processing

scRNA-seq data were first mapped to the human reference genome (hg38) to make matrices of UMI count for each gene and each cell using Cell Ranger (10X Genomics, version 6.0.1). Putative doublet clusters were removed using Scrublet (version 0.2.3) with the expected_doublet_rate set as 0.06⁶⁴. Then, the count data were imported into the Suerat package (version 4.0.3)⁶⁵ for the downstream analysis. Cells with nfeature>1.500, nCount between 2,500 and 50,000, and proportions of mitochondrial gene counts between 2% and 12% were only considered for further analyses. The raw counts were normalized using the log-normalization method. The cell cycle scores and cell cycle phases were then determined as described before⁶⁶.

### UMAP Analysis, Clustering, and Markers Identification

Count matrices of different samples were first merged to be simultaneously projected on a UMAP plot and normalized variations due to differences of total UMI counts as well as the cell cycle phases with the SCTransform function employing the vars.to.regress option⁶⁷. Principal component analysis (PCA) using the RunPCA function in the Seurat package was next performed. Then, the RunUMAP function was ran with default parameters except for dim=1:30 and the FindClusters function with a resolution of 0.5 for FIG. 2b and 0.15 for FIG. 3k in the Seurat package. To identify marker genes for each cluster, the Seurat FindAllMarkers function was used with the min.pct and logfc.threshold parameters set as 0.15 and 0.3, respectively, with a Wilcoxon rank sum test.

### Batch Correction and Integration Analysis

the two replicates of single-cell transcriptome data of MG-embedded Axioloids at 96 h were integrated with the mutual nearest neighbor algorithm after reciprocal principal component analysis using the Seurat package to correct batch effects³⁶. For this, the SCTransform normalization was first performed as described above and 3,000 integration features with SelectIntegrationFeatures function were selected. Next, the RunPCA function was ran with the selected features. Then, integration anchor sets using FindIntegrationAnchors was obtained with the normalization method and the reduction method set as “SCT” and “rpca”, respectively. The k.anchor parameter was set as 5. Then, the two replicates were integrated using the obtained anchor sets. After running PCA using the integration data, UMAP and clustering analysis were performed as described above with dim=1:20 and a resolution of 0.25 for FIG. 2g and Extended FIG. 5a. The integration of the two replicate sets of MG-embedded and non-embedded Axioloids at 96 h was carried out in the same way as above. UMAP and clustering was performed with dim=1:20 and a resolution of 0.1 for FIG. 3e. Similarly, the data sets of non-embedded, MG-embedded, MG-embedded with retinal, and MG-embedded with retinoic acid at both 96 h and 120 h were integrated altogether in the same way as above. UMAP and clustering was performed with dim=1:30 and a resolution of 0.04 for Extended FIG. 8a.

### Differentially Expressed Genes Analysis

To call differentially expressed genes (DEGs) between different Axioloid culture conditions, the batch correction and cluster annotation were first performed as described above to define cell sets of the same cell types to be compared with each other condition. In each of the defined cell clusters, gene expression levels were compared based on the normalized count data using the FindMarkers function of Seurat. For the comparison between MG-plus and MG-minus, DEGs were first called with the log 2 fold changes threshold of 0.25 for each of the two replicate experiments. Then, genes that were commonly up- or down-regulated by MG in both replicates were listed. The expression changes with the two replicate data sets combined together using the FindMarkers function with a Wilcoxon rank sum test were also calculated to make the volcano plots in FIG. 3f and Extended FIG. 5g. Regarding the effect of the addition of Retinal and Retinoic Acid, gene expression changes between the different conditions for each time point in each annotated cluster defined after the integration mentioned above were similarly calculated. To call for DEGs with the addition of Retinal, genes that were commonly up- or down-regulated both at 96 h and 120 h with the log 2 fold changes threshold of 0.25 were listed. Enrichment analysis was performed using the commonly up- or down-regulated genes as queries, with the database of KEGG_2021_Human and MSigDB_Hallmark_2020 using the Enrichr package (version 3.0)⁶⁸.

### RNA Velocity Analysis and Pseudotime Analysis

To perform velocity analysis, the fastq sequence data were reanalyzed with Kallisto (version 0.46.0)⁶⁹ and loompy (version 3.0.6) to obtain count matrices for both spliced and unspliced transcripts, followed by filtering out cells that were not subject to the UMAP and clustering analysis above. RNA velocity was then analyzed using scVelo (version 0.2.3) with the stochastic mode⁷⁰. The parameters used were min_shared_counts=20 and n_top_genes=2000 for the scv.pp.filter_and_normalize function, and n_pcs=30 and n_neighbors=30 for the scv.pp.moments function. The velocities are projected onto the UMAP plot generated above. For the integrated data set of the two replicates of 96 h_MG, pseudotime analysis was subsequently performed based on the velocity graph, using the scv.tl.velocity_pseudotime function in the scVelo package. The cells except for those annotated as IM-like or EC-like cells according the to the rank of the pseudotime were ordered to have the heatmap plots in FIG. 2i, FIG. 4n, Extended FIGS. 4a-c and 11a-c.

Comparison with Human Embryo scRNA-Seq Data

To compare the Axioloids with human embryos, the single cell RNAseq data of a CS12 human embryo were used 35. From this dataset, Seurat object of the embryo was created using Seurat (version 4.0.6), and SCTransform was performed with options vars.to.regress=c(“S.Score”, “G2M.Score”), variable.features.n=5000. UMAP analysis and clustering were performed using the RunUMAP function with the option dims=1:60, and the FindClusters function with the resolution 0.65 for FIG. 4i. The cell clusters were basically named based on the annotated cell types by Xu et al.³⁵. The cluster termed “limb” mainly consists of cells annotated as limb previously, but also includes cells annotated as intermediate mesodermal cells. The same SCTransform function was used for the dataset consisting of MG-embedded Axioloids with Retinal addition sampled at 96 h and 120 h. The cell type annotation for the Axioloids is from Extended FIG. 8a. Integration of the Axioloid and embryo data was carried out in the same way as described above, with the exception of the number of the integration features to be 5000 and the k.anchor to be 10. UMAP analysis and clustering were performed in the same way, besides the dims set to be 40 and the resolution to be 1.0. Calculation of the Pearson correlation coefficient between cell groups of the Axioloids and the embryo was performed based on how many cells in each cell group are assigned to the clusters defined after integration in FIG. 15k.

### Representation of Gene Expression Levels

To represent expression levels of each gene on the UMAP plots in FIG. 2e and Extended FIG. 5d as well as in the scatter plots in Extended FIG. 3f and the violin plots in FIG. 3h and Extended FIG. 8f-h, the log-normalized UMI counts were used. The gene expression levels were also scaled so that the mean expression levels are zero and the variances are 1 across cells using the Seurat ScaleData function. The scaled values were used in heatmaps in FIGS. 2f and i, and 4n, and Extended FIGS. 4a-c and 11a-c. In Extended FIGS. 3d and e, 5c, and 10h and i, the averaged values of the scaled data were calculated and plotted across cells in each cluster.

### Data Availability

All single cell RNA sequencing data and CUT&Tag data used for this study have been deposited in the NCBI Gene Expression Omnibus under accession number GSE199576. To review the data please go to https://www.ncbi.nlm.nih.gov/geo/query/acc.cgi?acc=GSE199576 and enter reviewer token ktunygmklvclpwp into the box.

### Code Availability

Computational codes and scripts used in this study are available at GitHub (https://github.com/Alev-Lab) and upon request from the corresponding author.

### Supplementary Videos

### Video 1: Effect of Matrigel on Axioloid Morphology.

Live imaging of Axioloids derived from 409B2 (upper) and 201B7 Luc (lower) with +MG (right) or without −MG (left) embedding into Matrigel (MG) between 72 h and 120 h of culture. Data shown is representative of at least three independent experiments. Scale bar is 200 m.

### Video 2: Effect of Matrigel Embedding on HES7 Gene Expression Dynamics.

Live imaging of the spatiotemporal morphogenetic expression of the HES7 gene in a 201B7 Luc-derived Axioloid embedded in MG from 72 h to 120 h of culture. BF video (left) and HES7:Luciferase signal (right). Data shown is representative of at least three independent experiments. Scale bar is 200 m.

### Video 3: Effect of RA Signaling of Axioloid Growth and Morphology.

Live imaging of Axioloids derived from 409B2 (Top panels) and 201B7 Luc (bottom panels) after embedding in MG only (left) or in MG supplemented with Retinal (RAL) (middle) or Retinoic Acid (RA) (right) from 72 h to 120 h of culture. Data shown is representative of at least three independent experiments. Scale bar is 200 m.

### Video 4:3D Visualization of Axioloid Structure.

3D reconstruction of an Axioloid embedded in MG+RA at 120 h stained for F-actin (Phalloidin) in gray, TBXT (BRA) in blue, Fibronectin (FN) in green and MEOX1 in red.

### Video 5: Midline and Bilateral Somite Formation in Axioloids.

Visualization of midline formation in a 409B2-derived Axioloid embedded in MG+RAL (Top) and of formation of a single bilateral somite in a 409B2-derived Axioloid embedded in MG+ROL (Bottom). Live imaging was performed between 72 and 120 h of culture. Scale bar is 200 m.

### Video 6: 3D Reconstruction of Segments in Axioloids and Human CS11 Embryo.

3D reconstruction of Axioloid segments derived from 409B2 (Right top) and 201B7 Luc (Right bottom) and reconstruction of the 8 posterior-most somites of a CS11 human embryo (right). Each somite-like structure is highlighted by a different color depending on its position along the antero-posterior axis.

### Video 7: Effect of RAL. RA or BMS493 Supplementation on HES7 Gene Expression Dynamics.

Live imaging of the spatiotemporal morphogenetic expression of the HES7 gene in 201B7 Luc-derived Axioloids embedded in MG supplemented with either RAL (top left) or RA (top right) or RAL+DMSO (bottom left) or RAL+BMS493 (bottom right), from 72 h to 120 h of culture. BF video (left) and HES7:Luciferase signal (right) for each condition. Data shown is representative of at least three independent experiments. Scale bar is 200 m.

### Video 8: Effect of RA, NOTCH, FGF and WNT Pathway Inhibition on Axioloid Growth and Morphology

Live imaging of Axioloids derived from 201B7 Luc after embedding in MG+RAL supplemented with (from left to right) DMSO, BMS493, DAPT, PD173074 or XAV939 from 72 h to 120 h of culture. Data shown is representative of at least three independent experiments. Scale bar is 200 m.

### Video 9: Effect of HES7 Gene KO on Axioloid Growth and Morphology.

Top panel, live imaging of Axioloids embedded in MG+RAL derived from 201B7 Luc (top left), HES7 KO1 (top middle) and HES7 KO2 (top right) cell lines. Data shown is representative of at least three independent experiments. Scale bar is 200 m. Bottom panel, 3D reconstruction of Axioloids embedded in MG+RAL derived from HES7 KO1 (middle) and HES7 KO2 (bottom) stained with F-actin (Phalloidin) in gray, TBXT (BRA) in blue, Fibronectin (FN) in green and MEOX1 in red.

### Video 10: Effect of HES7 Gene KO on HES7 Gene Expression Dynamics.

Live imaging of the spatiotemporal expression of the HES7 gene in 201B7 Luc, (top), HES7 KO1 (middle) and HES7 KO2 (bottom)-derived Axioloids embedded in MG+RAL. BF video (left) and HES7:Luciferase signal (right) for each condition. Data shown is representative of at least three independent experiments. Scale bar is 200 m.

### Video 11: Effect of HES7 Point Mutation (Rs113994160: c.73C>T: HES7^{R25W}) on Axioloid Growth and Morphology.

Top panel, live imaging of Axioloids embedded in MG+RAL derived from 201B7 Luc (left), HES7^{R25W} MT1 (middle) and HES7^{R25W} MT2 (right) cell lines. Data shown is representative of at least three independent experiments. Scale bar is 200 m. Bottom panel, 3D reconstruction of Axioloids embedded in MG+RAL derived from HES7^{R25W} MT1 (top) and HES7^{R25W} MT2 (bottom) stained with F-actin (Phalloidin) in gray, TBXT (BRA) in blue, Fibronectin (FN) in green and MEOX1 in red.

### Video 12: Effect of HES7 Point Mutation (Rs113994160: c.73C>T: HES7^{R25W}) on HES7 Gene Expression Dynamics.

Live imaging of the spatiotemporal expression of the HES7 gene in 201B7 Luc, (top), HES7^{R25W} MT1 (middle) and HES7^{R25W} MT2 (bottom)-derived Axioloids. BF video (left) and HES7:Luciferase signal (right) for each condition. Data shown is representative of at least three independent experiments. Scale bar is 200 m.

### Video 13: Effect of MESP2 Gene KO on Axioloid Growth and Morphology.

Top panel, live imaging of Axioloids embedded in MG+RAL derived from 201B7 Luc (top left), MESP2 KO1 (top middle) and MESP2 KO2 (top right) cell lines. Data shown is representative of at least three independent experiments. Scale bar is 200 m. Bottom panel, 3D reconstruction of Axioloids embedded in MG+RAL derived from MESP2 KO1 (middle) and MESP2 KO2 (bottom) stained with F-actin (Phalloidin) in gray, TBXT (BRA) in blue, Fibronectin (FN) in green and MEOX1 in red.

### Video 14: Effect of MESP2 Gene KO on HES7 Gene Expression Dynamics.

Live imaging of the spatiotemporal expression of the HES7 gene in 201B7 Luc, (top), MESP2 KO1 (middle) and MESP2 KO2 (bottom)-derived Axioloids embedded in MG+RAL. BF video (left) and HES7:Luciferase signal (right) for each condition. Data shown is representative of at least three independent experiments. Scale bar is 200 m.

### Supplementary Discussion

### Midline and Bilateral Somite Formation in Axioloids

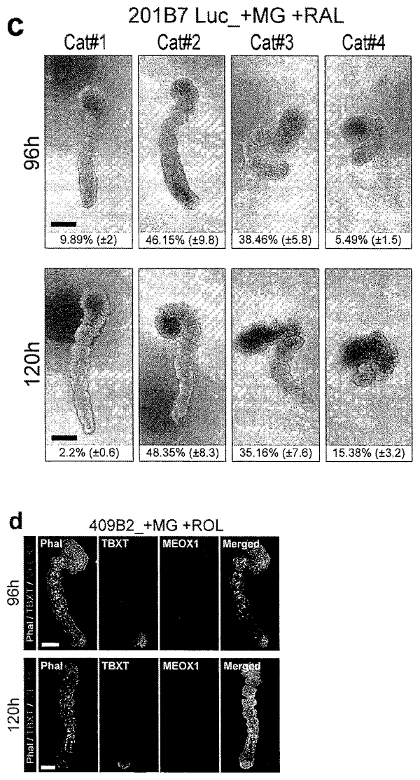

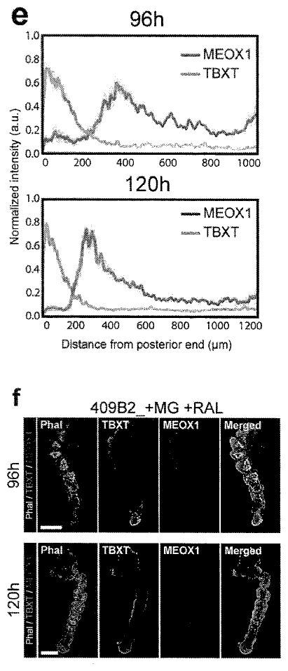

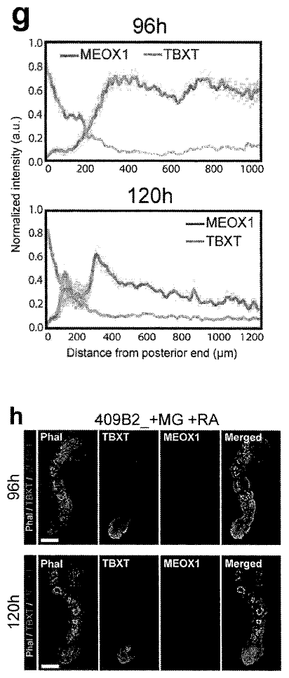

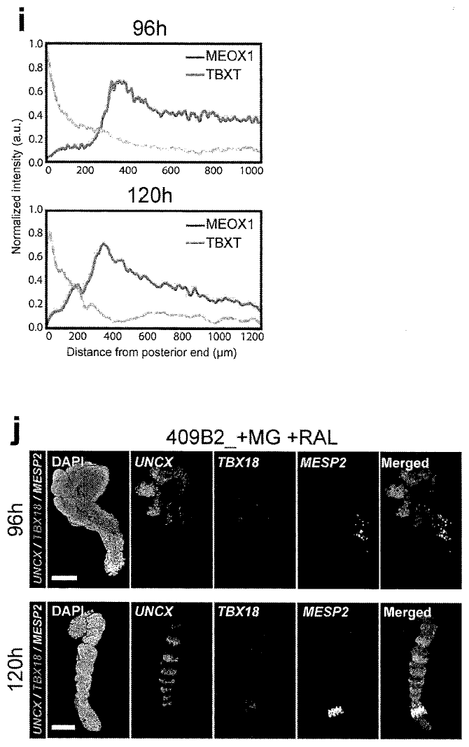

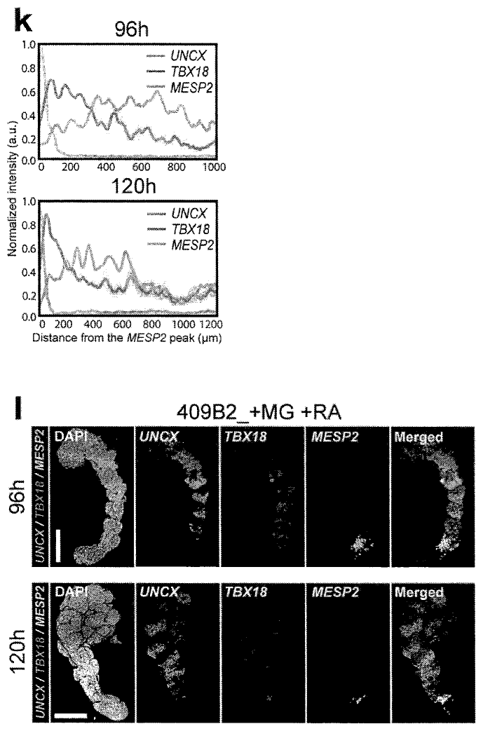

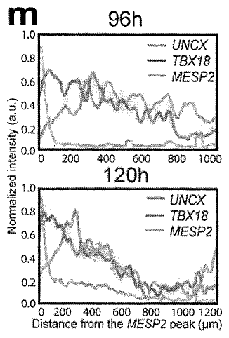

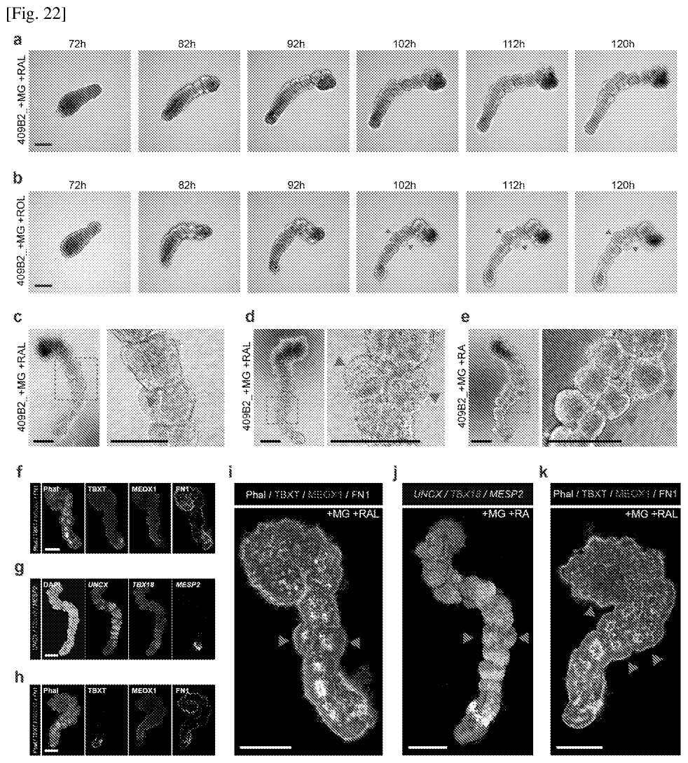

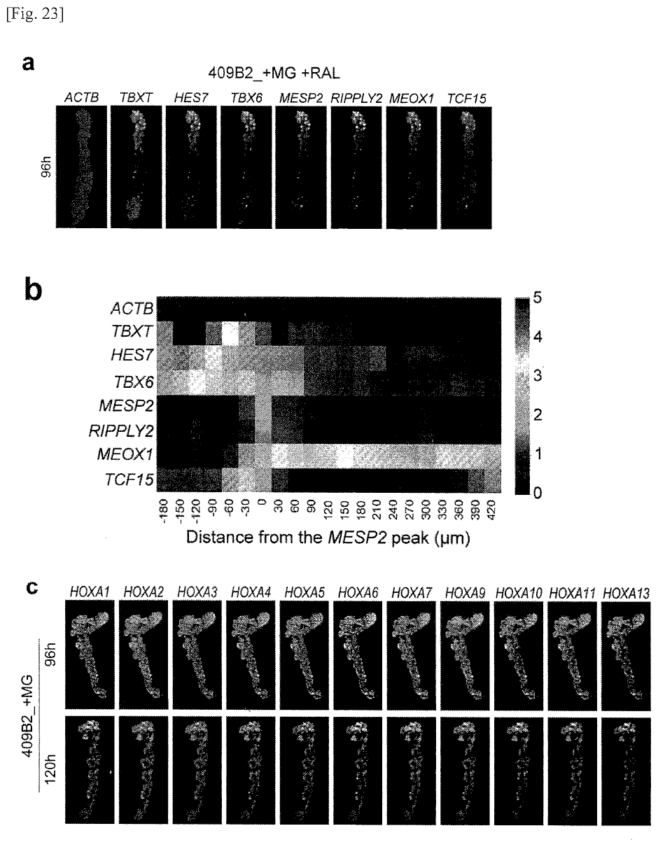

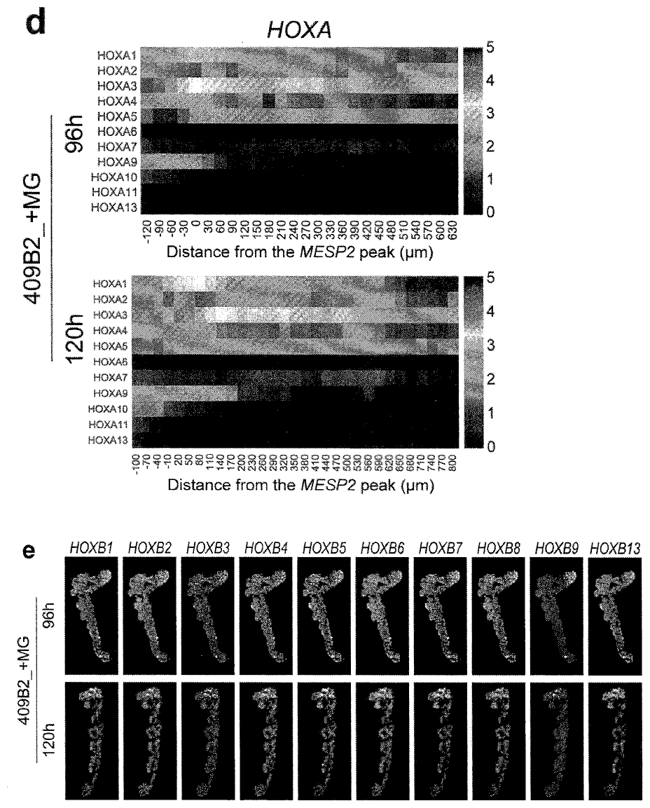

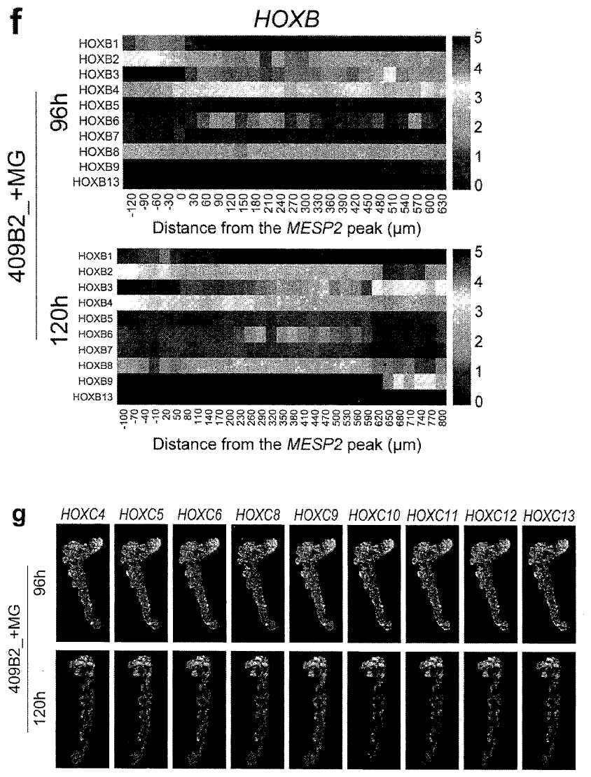

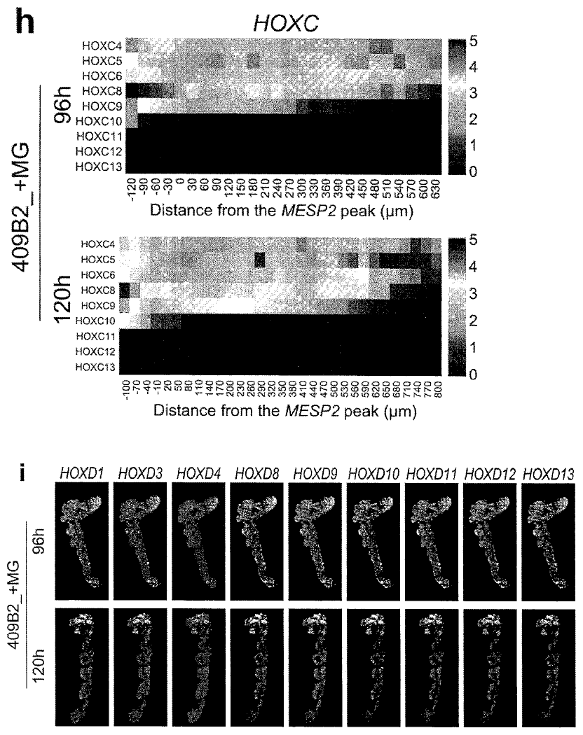

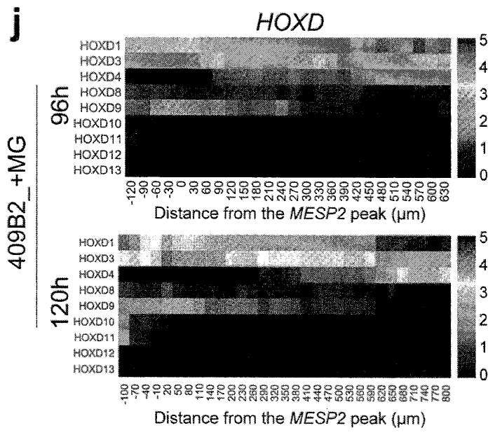

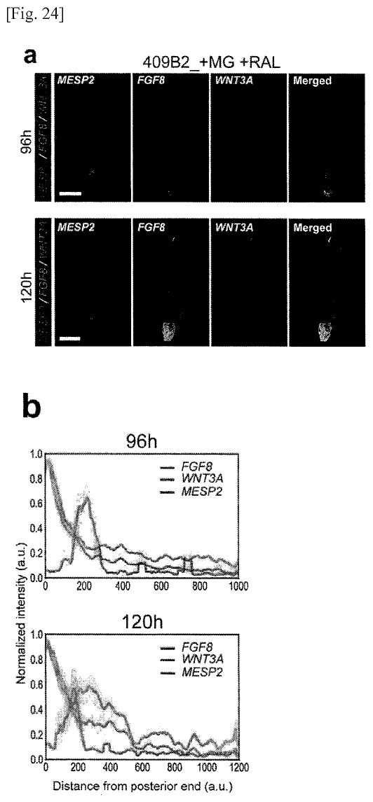

In MG and RA treated axioloids a single axis of sequentially forming epithelial somites with single central somitocoels was typically observed. Intriguingly, axioloids also frequently displayed a superficial groove or midlinelike structure, starting in the PSM and going through most of the forming segments (Supplementary FIG. 22a, b), though it typically remained superficial, without separating the segments into two fully segregated somites. (Supplementary FIG. 22a, b, Supplementary Video 8). True bilateral, fully segregated somites, each with their respective central somitocoel, were nevertheless occasionally present in retinoid treated axioloids, sometimes as an isolated fully segregated bilateral single pair of somites and in some rare cases also as a sequence of two or more somite pairs along the AP-axis of an axioloid (Supplementary FIG. 22c-e, Supplementary Video 8). These bilateral somites showed usually normal protein and gene expression patterns, including proper rostrocaudal polarity (Supplementary FIG. 22f-k). The exact molecular nature and possible mechanism which may underlie the formation of single vs. bilateral somites in axioloids remains to be elucidated.

### Supplementary Tables

Supplementary Table 1 (Table 1-A to 1I): Genes expressed in identified clusters of scRNA-seq data.

TABLE 1-A

| p_val | avg_log2FC | pct.1 | pct.2 | p_val_adj | cluster | gene |
|---|---|---|---|---|---|---|
| 0 | 2.297443543 | 0.982 | 0.054 | 0 | L-TB | HOXA10 |
| 0 | 2.234209826 | 0.976 | 0.169 | 0 | L-TB | TGFBI |
| 0 | 2.216774834 | 0.996 | 0.082 | 0 | L-TB | SOX2 |
| 0 | 2.057415581 | 0.981 | 0.046 | 0 | L-TB | HOXC10 |
| 0 | 1.813769658 | 0.982 | 0.23 | 0 | L-TB | FGFBP3 |
| 0 | 1.796539698 | 0.991 | 0.54 | 0 | L-TB | NRP2 |
| 0 | 1.743670154 | 0.965 | 0.104 | 0 | L-TB | AC103702.2 |
| 0 | 1.74311261 | 0.762 | 0.085 | 0 | L-TB | LEFTY2 |
| 0 | 1.696103252 | 0.99 | 0.42 | 0 | L-TB | HOXC8 |
| 0 | 1.635542867 | 0.973 | 0.131 | 0 | L-TB | URAD |
| 0 | 1.53120954 | 0.968 | 0.375 | 0 | L-TB | HOXD1 |
| 0 | 1.457173687 | 0.996 | 0.62 | 0 | L-TB | HOXC6 |
| 0 | 1.393913172 | 0.952 | 0.237 | 0 | L-TB | LINC02381 |
| 0 | 1.379258695 | 0.978 | 0.307 | 0 | L-TB | HOXC9 |
| 0 | 1.334439739 | 0.903 | 0.052 | 0 | L-TB | CDH6 |
| 0 | 1.262310078 | 0.916 | 0.502 | 0 | L-TB | CKB |
| 0 | 1.192472104 | 0.964 | 0.729 | 0 | L-TB | GOLIM4 |
| 0 | 1.158998457 | 0.593 | 0.025 | 0 | L-TB | SCGN |
| 0 | 1.142417493 | 0.925 | 0.273 | 0 | L-TB | PLAGL1 |
| 0 | 1.140980499 | 0.76 | 0.062 | 0 | L-TB | KCTD12 |
| 0 | 1.122919236 | 0.99 | 0.684 | 0 | L-TB | CCND1 |
| 0 | 1.116915885 | 0.856 | 0.127 | 0 | L-TB | HOXA9 |
| 0 | 1.107594071 | 0.921 | 0.455 | 0 | L-TB | JARID2 |
| 0 | 1.082559703 | 0.973 | 0.488 | 0 | L-TB | DUSP6 |
| 0 | 1.058731374 | 0.807 | 0.158 | 0 | L-TB | TMEM121 |
| 0 | 1.043582124 | 0.925 | 0.386 | 0 | L-TB | IL17RD |
| 0 | 1.018600862 | 1 | 0.951 | 0 | L-TB | IGDCC3 |
| 0 | 1.012431424 | 0.799 | 0.155 | 0 | L-TB | OAF |
| 0 | 0.982708557 | 0.905 | 0.354 | 0 | L-TB | TRIM2 |
| 0 | 0.963861915 | 0.909 | 0.369 | 0 | L-TB | RND2 |
| 0 | 0.946218181 | 0.867 | 0.255 | 0 | L-TB | RPRM |
| 0 | 0.924969219 | 0.916 | 0.393 | 0 | L-TB | ZIC2 |
| 0 | 0.917651077 | 0.961 | 0.539 | 0 | L-TB | WNT5A |
| 0 | 0.910806692 | 0.521 | 0.058 | 0 | L-TB | LIX1 |
| 0 | 0.889879234 | 0.813 | 0.117 | 0 | L-TB | NPTX2 |
| 0 | 0.88660335 | 0.796 | 0.12 | 0 | L-TB | ASXL3 |
| 0 | 0.872366793 | 0.941 | 0.555 | 0 | L-TB | HES6 |
| 0 | 0.867420002 | 0.883 | 0.265 | 0 | L-TB | HOXD9 |
| 0 | 0.838843005 | 0.713 | 0.055 | 0 | L-TB | TIMP4 |
| 0 | 0.82890873 | 0.763 | 0.112 | 0 | L-TB | NEDD9 |
| 0 | 0.814033048 | 0.876 | 0.348 | 0 | L-TB | KIF5C |
| 0 | 0.79747432 | 0.464 | 0.005 | 0 | L-TB | HOXA11-AS |
| 0 | 0.777166746 | 0.531 | 0.037 | 0 | L-TB | LEFTY1 |
| 0 | 0.763167043 | 0.874 | 0.333 | 0 | L-TB | ID2 |
| 0 | 0.759285316 | 0.751 | 0.295 | 0 | L-TB | FABP7 |
| 0 | 0.758807521 | 1 | 0.99 | 0 | L-TB | CALM2 |
| 0 | 0.752187783 | 0.817 | 0.224 | 0 | L-TB | ZBTB16 |
| 0 | 0.748291681 | 0.998 | 0.959 | 0 | L-TB | H2AFY |
| 0 | 0.730560735 | 0.998 | 0.948 | 0 | L-TB | CSRP2 |
| 0 | 0.729102921 | 0.815 | 0.247 | 0 | L-TB | AC004540.2 |
| 0 | 1.981073852 | 0.878 | 0.295 | 0 | M-TB | CDX4 |
| 0 | 1.902240375 | 0.972 | 0.302 | 0 | M-TB | MSX1 |
| 0 | 1.867731939 | 0.994 | 0.296 | 0 | M-TB | CDX2 |
| 0 | 1.806851935 | 0.995 | 0.539 | 0 | M-TB | HOXA-AS3 |
| 0 | 1.705180418 | 1 | 0.967 | 0 | M-TB | HOXB-AS3 |
| 0 | 1.315949745 | 0.98 | 0.612 | 0 | M-TB | LIN28A |
| 0 | 1.202902916 | 0.993 | 0.985 | 0 | M-TB | RCN2 |
| 0 | 1.196604732 | 0.877 | 0.16 | 0 | M-TB | L1TD1 |
| 0 | 1.17859994 | 0.922 | 0.228 | 0 | M-TB | C3orf52 |
| 0 | 1.130177096 | 0.91 | 0.26 | 0 | M-TB | CNTNAP2 |
| 0 | 1.000956014 | 0.813 | 0.083 | 0 | M-TB | UGT8 |
| 0 | 0.99677849 | 0.925 | 0.593 | 0 | M-TB | DLG1 |
| 0 | 0.981617863 | 0.85 | 0.177 | 0 | M-TB | GNG4 |

TABLE 1-B

| | | | | | | |
|---|---|---|---|---|---|---|
| 0 | 0.948381014 | 0.997 | 0.879 | 0 | M-TB | GTF3A |
| 5.09E−299 | 0.912627802 | 0.972 | 0.766 | 1.86E−294 | M-TB | ABRACL |
| 0 | 0.881592566 | 0.855 | 0.215 | 0 | M-TB | SPON1 |
| 2.87E−268 | 0.869364514 | 0.982 | 0.789 | 1.05E−263 | M-TB | MLLT3 |
| 6.50E−213 | 0.864077089 | 0.971 | 0.84 | 2.38E−208 | M-TB | PPP1R14B |
| 4.58E−217 | 0.854518834 | 0.952 | 0.653 | 1.68E−212 | M-TB | IGFBP2 |
| 0 | 0.852598763 | 0.75 | 0.093 | 0 | M-TB | LY6G6D |
| 4.89E−220 | 0.677577635 | 0.993 | 0.94 | 1.79E−215 | M-TB | TOP1 |
| 6.84E−292 | 0.647523305 | 0.828 | 0.336 | 2.50E−287 | M-TB | UGCG |
| 4.72E−295 | 0.632741207 | 0.786 | 0.289 | 1.73E−290 | M-TB | HOXC4 |
| 2.78E−237 | 0.624045528 | 0.787 | 0.346 | 1.02E−232 | M-TB | GREB1 |
| 1.51E−236 | 0.623250627 | 0.998 | 0.967 | 5.54E−232 | M-TB | SNRPG |
| 0 | 0.610605265 | 0.62 | 0.158 | 0 | M-TB | PDPN |
| 3.66E−166 | 0.606561204 | 0.942 | 0.715 | 1.34E−161 | M-TB | NPM3 |
| 6.65E−218 | 0.599736664 | 0.835 | 0.394 | 2.43E−213 | M-TB | TM7SF2 |
| 4.04E−286 | 0.593652657 | 0.771 | 0.286 | 1.48E−281 | M-TB | SPRY2 |
| 2.44E−191 | 0.593176775 | 0.744 | 0.344 | 8.94E−187 | M-TB | ADGRV1 |
| 2.93E−270 | 0.586942281 | 0 | 0.999 | 1.07E−265 | M-TB | HMGN2 |
| 0 | 0.582799703 | 0.657 | 0.061 | 0 | M-TB | LYPD6B |
| 0 | 0.570367021 | 0.656 | 0.143 | 0 | M-TB | PTPRZ1 |
| 0 | 0.567215433 | 0.606 | 0.054 | 0 | M-TB | MGLL |
| 3.15E−161 | 0.562662669 | 0.937 | 0.71 | 1.15E−156 | M-TB | MTIF3 |
| 3.61E−164 | 0.559547978 | 0.987 | 0.934 | 1.32E−159 | M-TB | NDUFA6 |
| 9.07E−175 | 0.557561595 | 0.98 | 0.869 | 3.32E−170 | M-TB | FAM136A |
| 2.50E−209 | 0.556332598 | 0.746 | 0.296 | 9.17E−205 | M-TB | PPP1R14C |
| 0 | 0.547034586 | 0.707 | 0.21 | 0 | M-TB | PHLPP1 |
| 1.48E−155 | 0.538979746 | 0.966 | 0.814 | 5.41E−151 | M-TB | C7orf50 |
| 0 | 0.529861047 | 0.562 | 0.041 | 0 | M-TB | GABRB2 |
| 2.51E−165 | 0.517354058 | 0.897 | 0.584 | 9.19E−161 | M-TB | CTNNBIP1 |
| 9.13E−159 | 0.513881562 | 0.829 | 0.475 | 3.34E−154 | M-TB | DUSP23 |
| 5.16E−168 | 0.513815043 | 0.995 | 0.973 | 1.89E−163 | M-TB | NDUFB2 |
| 1.67E−169 | 0.510336433 | 0.989 | 0.944 | 6.11E−165 | M-TB | GCSH |
| 4.60E−141 | 0.508476769 | 0.97 | 0.835 | 1.68E−136 | M-TB | SRM |
| 5.28E−157 | 0.504226304 | 0.997 | 0.971 | 1.93E−152 | M-TB | HNRNPAB |
| 6.01E−253 | 0.499432509 | 1 | 1 | 2.20E−248 | M-TB | SET |
| 1.13E−136 | 0.497701092 | 0.997 | 0.961 | 4.14E−132 | M-TB | FDPS |
| 3.70E−112 | 0.488746369 | 0.978 | 0.892 | 1.35E−107 | M-TB | SQLE |
| 6.34E−294 | 2.366874333 | 0.996 | 0.656 | 2.32E−289 | E-TB | KRT18 |
| 4.31E−296 | 2.236032359 | 1 | 0.56 | 1.58E−291 | E-TB | CRABP2 |
| 0 | 2.099044214 | 0.985 | 0.214 | 0 | E-TB | FGF17 |
| 1.26E−283 | 1.881015186 | 1 | 0.687 | 4.61E−279 | E-TB | KRT8 |
| 0 | 1.879664871 | 0.931 | 0.279 | 0 | E-TB | SLC2A3 |
| 5.60E−240 | 1.768874609 | 0.893 | 0.32 | 2.05E−235 | E-TB | NEFM |
| 0 | 1.764965898 | 0.944 | 0.297 | 0 | E-TB | FGF8 |
| 3.01E−292 | 1.743100683 | 0.97 | 0.424 | 1.10E−287 | E-TB | TUBB2A |
| 0 | 1.598094683 | 0.923 | 0.098 | 0 | E-TB | WNT5B |
| 0 | 1.518461428 | 0.985 | 0.309 | 0 | E-TB | CDX1 |
| 0 | 1.514269168 | 0.938 | 0.155 | 0 | E-TB | TTYH1 |
| 0 | 1.490877023 | 0.908 | 0.19 | 0 | E-TB | WFDC2 |
| 2.89E−209 | 1.44121644 | 0.983 | 0.7 | 1.06E−204 | E-TB | APOE |
| 0 | 1.417168882 | 0.94 | 0.24 | 0 | E-TB | SLC2A1 |
| 0 | 1.349099432 | 0.949 | 0.264 | 0 | E-TB | ETV4 |
| 9.315-255 | 1.301064997 | 0.968 | 0.448 | 3.41E−250 | E-TB | PDLIM1 |
| 8.39E−294 | 1.277739046 | 0.927 | 0.273 | 3.07E−289 | E-TB | SP5 |
| 0 | 1.264480834 | 0.722 | 0.079 | 0 | E-TB | FGF3 |
| 0 | 1.263732045 | 0.835 | 0.079 | 0 | E-TB | TMEM100 |
| 0 | 1.186605571 | 0.929 | 0.25 | 0 | E-TB | VAMP8 |
| 0 | 1.140510501 | 0.85 | 0.115 | 0 | E-TB | PHLDA2 |
| 0 | 1.119482746 | 0.844 | 0.083 | 0 | E-TB | TBXT |
| 0 | 1.082965137 | 0.6 | 0.02 | 0 | E-TB | FGF4 |
| 5.48E−189 | 1.076083507 | 0.989 | 0.911 | 2.01E−184 | E-TB | MLEC |
| 1.71E−169 | 1.039047727 | 0.916 | 0.508 | 6.26E−165 | E-TB | SERPINE2 |
| 5.69E−161 | 1.017926413 | 0.987 | 0.892 | 2.08E−156 | E-TB | TUBB2B |
| 3.13E−227 | 0.979323839 | 0.929 | 0.354 | 1.15E−222 | E-TB | TNNT1 |

TABLE 1-C

| | | | | | | |
|---|---|---|---|---|---|---|
| 8.45E−167 | 0.952419941 | 0.9791 | 0.787 | 3.09E−162 | E-TB | GSTO1 |
| 1.18E−172 | 0.921917683 | 0.946 | 0.556 | 4.34E−168 | E-TB | PGM1 |
| 7.37E−139 | 0.873969369 | 0.987 | 0.848 | 2.70E−134 | E-TB | PYURF |
| 0 | 0.869692881 | 0.672 | 0.101 | 0 | E-TB | MIXL1 |
| 8.49E−199 | 0.863138374 | 0.865 | 0.316 | 3.11E−194 | E-TB | PLP1 |
| 4.37E−127 | 0.861427526 | 0.985 | 0.889 | 1.60E−122 | E-TB | ACAT2 |
| 2.68E−206 | 0.852001214 | 1 | 0.999 | 9.81E−202 | E-TB | PABPC1 |
| 2.79E−159 | 0.848253963 | 0.998 | 0.954 | 1.02E−154 | E-TB | FKBP1A |
| 0 | 0.840492888 | 0.749 | 0.055 | 0 | E-TB | WNT3A |
| 0 | 0.839866738 | 0.747 | 0.099 | 0 | E-TB | CA4 |
| 4.20E−233 | 0.818279544 | 0.85 | 0.258 | 1.54E−228 | E-TB | ALPL |
| 2.13E−153 | 0.811207174 | 1 | 0.989 | 7.80E−149 | E-TB | CALR |
| 8.73E−136 | 0.804937297 | 0.985 | 0.82 | 3.20E−131 | E-TB | COTL1 |
| 0 | 0.794610511 | 0.563 | 0.073 | 0 | E-TB | GLIPR1 |
| 3.34E−140 | 0.790975821 | 0.989 | 0.851 | 1.22E−135 | E-TB | PERP |
| 2.11E−121 | 0.780610836 | 0.949 | 0.602 | 7.73E−117 | E-TB | EPHA4 |
| 4.82E−109 | 0.779445437 | 0.989 | 0.963 | 1.77E−104 | E-TB | HSPA5 |
| 0 | 0.779364631 | 0.715 | 0.058 | 0 | E-TB | CLDN6 |
| 9.29E−127 | 0.764648643 | 0.979 | 0.693 | 3.40E−122 | E-TB | DHCR24 |
| 3.90E−105 | 0.759317986 | 0.792 | 0.364 | 1.43E−100 | E-TB | MT1X |
| 0 | 0.741866031 | 0.557 | 0.077 | 0 | E-TB | STC1 |
| 2.79E−105 | 0.741221008 | 0.884 | 0.585 | 1.02E−100 | E-TB | CACHD1 |
| 7.57E−229 | 0.726516016 | 0.743 | 0.174 | 2.77E−224 | E-TB | DES |
| 0 | 1.911088168 | 0.899 | 0.097 | 0 | E-PSM | MSGN1 |
| 0 | 1.71597613 | 1 | 0.988 | 0 | E-PSM | MEST |
| 0 | 1.597717513 | 0.94 | 0.234 | 0 | E-PSM | HES7 |
| 0 | 1.566909216 | 0.999 | 0.939 | 0 | E-PSM | RBP1 |
| 0 | 1.49861739 | 0.953 | 0.153 | 0 | E-PSM | TBX6 |
| 0 | 1.250918702 | 0.915 | 0.308 | 0 | E-PSM | CITED1 |
| 0 | 1.234899383 | 0.893 | 0.123 | 0 | E-PSM | HOXB1 |
| 0 | 1.127609604 | 0.997 | 0.939 | 0 | E-PSM | GYPC |
| 0 | 1.125738294 | 0.979 | 0.789 | 0 | E-PSM | SMC6 |
| 0 | 1.122929922 | 0.953 | 0.744 | 0 | E-PSM | RSPO3 |
| 0 | 0.969924613 | 0.968 | 0.756 | 0 | E-PSM | ITM2A |
| 0 | 0.955686302 | 0.991 | 0.868 | 0 | E-PSM | CYCS |
| 0 | 0.924666953 | 0.999 | 0.959 | 0 | E-PSM | EIF5A |
| 0 | 0.903580041 | 0.991 | 0.909 | 0 | E-PSM | GLUL |
| 0 | 0.876032065 | 0.977 | 0.745 | 0 | E-PSM | AKAP12 |
| 0 | 0.862355876 | 0.998 | 0.995 | 0 | E-PSM | HSPA8 |
| 0 | 0.858198208 | 0.999 | 0.993 | 0 | E-PSM | CALM1 |
| 0 | 0.857445706 | 0.746 | 0.077 | 0 | E-PSM | VWA2 |
| 0 | 0.832205815 | 0.856 | 0.341 | 0 | E-PSM | CNTFR |
| 0 | 0.832194396 | 0.937 | 0.62 | 0 | E-PSM | TIMM50 |
| 0 | 0.80592839 | 0.943 | 0.672 | 0 | E-PSM | C20orf27 |
| 0 | 0.80566297 | 0.994 | 0.861 | 0 | E-PSM | UBE2S |
| 0 | 0.766134289 | 0.899 | 0.518 | 0 | E-PSM | YARS |
| 0 | 0.75614876 | 1 | 0.999 | 0 | E-PSM | HSP90AA1 |
| 0 | 0.706157063 | 0.98 | 0.851 | 0 | E-PSM | TOMM40 |
| 0 | 0.694488377 | 0.658 | 0.26 | 0 | E-PSM | DKK1 |
| 0 | 0.688170682 | 0.943 | 0.8 | 0 | E-PSM | IFT57 |
| 0 | 0.687521052 | 0.995 | 0.971 | 0 | E-PSM | ARPC2 |
| 0 | 0.685298361 | 0.791 | 0.548 | 0 | E-PSM | ATP1B1 |
| 0 | 0.672983556 | 0.999 | 0.996 | 0 | E-PSM | OAZ1 |
| 0 | 0.670626693 | 0.997 | 0.988 | 0 | E-PSM | TXN |
| 0 | 0.665209623 | 0.999 | 0.991 | 0 | E-PSM | HMGB3 |
| 0 | 0.663272218 | 0.999 | 0.994 | 0 | E-PSM | ENO1 |
| 0 | 0.662309758 | 0.797 | 0.376 | 0 | E-PSM | MLXIP |
| 0 | 0.660753903 | 0.949 | 0.77 | 0 | E-PSM | CHCHD10 |
| 0 | 0.660593259 | 0.952 | 0.717 | 0 | E-PSM | FASN |
| 0 | 0.657921646 | 0.971 | 0.815 | 0 | E-PSM | SEPTIN9 |
| 0 | 0.651059248 | 0.873 | 0.493 | 0 | E-PSM | PLXNA2 |
| 0 | 0.646194984 | 0.926 | 0.779 | 0 | E-PSM | LDHA |
| 0 | 0.637457349 | 0.997 | 0.962 | 0 | E-PSM | DBI |
| 0 | 0.633774957 | 0.755 | 0.273 | 0 | E-PSM | SERINC5 |

TABLE 1-D

| | | | | | | |
|---|---|---|---|---|---|---|
| 0 | 0.632970064 | 0.61 | 0.177 | 0 | E-PSM | ARL4D |
| 0 | 0.628136867 | 0.928 | 0.704 | 0 | E-PSM | ARHGDIA |
| 0 | 0.624812944 | 0.471 | 0.113 | 0 | E-PSM | EFHD1 |
| 0 | 0.624004947 | 0.997 | 0.982 | 0 | E-PSM | PRELID1 |
| 0 | 0.607427846 | 0.879 | 0.683 | 0 | E-PSM | COL6A1 |
| 0 | 0.59575578 | 0.993 | 0.989 | 0 | E-PSM | PSMB5 |
| 0 | 0.591677632 | 0.973 | 0.814 | 0 | E-PSM | CYP51A1 |
| 0 | 0.589370088 | 0.973 | 0.91 | 0 | E-PSM | PGK1 |
| 0 | 0.581882609 | 0.996 | 0.998 | 0 | E-PSM | PRDX1 |
| 0 | 1.064591514 | 0.859 | 0.303 | 0 | PSM | FOXB1 |
| 0 | 0.999779058 | 0.935 | 0.487 | 0 | PSM | WWC2 |
| 2.19E−295 | 0.846181133 | 0.956 | 0.672 | 8.00E−291 | PSM | RAP2B |
| 4.03E−303 | 0.778403163 | 0.8 | 0.349 | 1.47E−298 | PSM | S1PR3 |
| 4.11E−247 | 0.746066711 | 0.924 | 0.633 | 1.50E−242 | PSM | RALGPS2 |
| 1.91E−260 | 0.738737777 | 0.927 | 0.606 | 6.98E−256 | PSM | HOXB4 |
| 3.86E−238 | 0.717648312 | 0.95 | 0.706 | 1.41E−233 | PSM | HOXB3 |
| 5.25E−237 | 0.67794601 | 0.922 | 0.576 | 1.92E−232 | PSM | DSG2 |
| 6.02E−227 | 0.672105221 | 0.884 | 0.537 | 2.20E−222 | PSM | GREB1L |
| 1.00E−30 | 0.62843307 | 0.646 | 0.533 | 3.67E−26 | PSM | HISTIH1C |
| 1.97E−124 | 0.623612774 | 0.906 | 0.736 | 7.19E−120 | PSM | MT-ND6 |
| 1.16E−17 | 0.565223077 | 0.651 | 0.581 | 4.23E−13 | PSM | HIST1H1B |
| 3.52E−184 | 0.524042683 | 0.695 | 0.334 | 1.29E−179 | PSM | BAALC |
| 8.79E−16 | 0.519015432 | 0.956 | 0.959 | 3.22E−11 | PSM | HIST1H4C |
| 2.42E−234 | 0.514942044 | 1 | 1 | 8.87E−230 | PSM | MT-ND1 |
| 1.48E−126 | 0.513974015 | 0.962 | 0.865 | 5.43E−122 | PSM | SMC2 |
| 9.28E−163 | 0.511934008 | 1 | 1 | 3.40E−158 | PSM | MT-ND2 |
| 1.13E−188 | 0.506383422 | 0.64 | 0.291 | 4.13E−184 | PSM | MOB3B |
| 2.27E−90 | 0.492182446 | 0.957 | 0.888 | 8.31E−86 | PSM | PEG10 |
| 1.39E−211 | 0.491215941 | 0.632 | 0.257 | 5.08E−207 | PSM | SEMA4D |
| 8.80E−108 | 0.462058174 | 0.909 | 0.742 | 3.22E−103 | PSM | RERE |
| 0 | 0.460058636 | 0.448 | 0.086 | 0 | PSM | PDYN |
| 2.72E−294 | 0.440295905 | 0.549 | 0.153 | 9.96E−290 | PSM | TTC39B |
| 1.76E−101 | 0.436453439 | 0.864 | 0.685 | 6.45E−97 | PSM | CDV3 |
| 1.88E−101 | 0.433649012 | 0.754 | 0.53 | 6.89E−97 | PSM | FAM98A |
| 2.97E−118 | 0.431653188 | 0.885 | 0.663 | 1.09E−113 | PSM | TSPAN5 |
| 1.24E−187 | 0.426795233 | 1 | 1 | 4.54E−183 | PSM | MT-ND4 |
| 2.28E−102 | 0.426187623 | 0.914 | 0.718 | 8.36E−98 | PSM | COL5A2 |
| 9.53E−132 | 0.416761921 | 0.707 | 0.402 | 3.49E−127 | PSM | AC103702.1 |
| 9.67E−142 | 0.404727085 | 0.688 | 0.362 | 3.54E−137 | PSM | ACSS3 |
| 6.37E−94 | 0.401458871 | 0.958 | 0.865 | 2.33E−89 | PSM | APP |
| 2.59E−147 | 0.390310301 | 0.588 | 0.271 | 9.48E−143 | PSM | PCSK5 |
| 3.40E−12 | 0.386830938 | 0.624 | 0.572 | 1.24E−07 | PSM | HIST2H2AC |
| 3.01E−100 | 0.371691315 | 0.982 | 0.918 | 1.10E−95 | PSM | EIF5B |
| 3.85E−139 | 0.370442178 | 0.5 | 0.214 | 1.41E−134 | PSM | ITGB8 |
| 3.14E−87 | 0.36619268 | 0.815 | 0.582 | 1.15E−82 | PSM | SALL1 |
| 1.30E−77 | 0.363205306 | 0.782 | 0.591 | 4.75E−73 | PSM | SUCO |
| 5.94E−126 | 0.361657155 | 0.565 | 0.266 | 2.17E−121 | PSM | TRIL |
| 1.26E−88 | 0.348135361 | 0.753 | 0.518 | 4.63E−84 | PSM | CYYR1 |
| 7.55E−85 | 0.337373333 | 0.976 | 0.912 | 2.76E−80 | PSM | RBM3 |
| 5.20E−249 | 0.329717596 | 0.443 | 0.114 | 1.90E−244 | PSM | PRICKLE2 |
| 1.11E−32 | 0.323421634 | 0.484 | 0.34 | 4.05E−28 | PSM | HIST1H2AH |
| 5.68E−69 | 0.319046831 | 0.935 | 0.847 | 2.08E−64 | PSM | DDX1 |
| 5.01E−121 | 0.314640528 | 0.501 | 0.223 | 1.83E−116 | PSM | RAB30 |
| 2.61E−63 | 0.307706445 | 0.987 | 0.943 | 9.54E−59 | PSM | SMC3 |
| 0 | 3.416766697 | 0.976 | 0.082 | 0 | E-APSM | RIPPLY2 |
| 0 | 2.688722729 | 0.966 | 0.056 | 0 | E-APSM | MESP2 |
| 3.07E−259 | 2.394478114 | 0.942 | 0.277 | 1.12E−254 | E-APSM | NTS |
| 0 | 1.908316934 | 0.847 | 0.066 | 0 | E-APSM | CER1 |
| 9.95E−208 | 1.712305193 | 1 | 0.516 | 3.64E−203 | E-APSM | CXCR4 |
| 8.15E−211 | 1.617943106 | 0.992 | 0.499 | 2.98E−206 | E-APSM | DLL1 |
| 7.04E−148 | 1.613614672 | 1 | 0.748 | 2.58E−143 | E-APSM | SAT1 |
| 0 | 1.445842158 | 0.745 | 0.064 | 0 | E-APSM | MESP1 |
| 3.15E−190 | 1.388588051 | 0.997 | 0.471 | 1.15E−185 | E-APSM | APLNR |
| 5.07E−218 | 1.379815898 | 0.963 | 0.36 | 1.86E−213 | E-APSM | RND3 |

TABLE 1-E

| | | | | | | |
|---|---|---|---|---|---|---|
| 1.32E−179 | 1.330050142 | 0.974 | 0.476 | 4.82E−175 | E-APSM | PTCH1 |
| 4.91E−170 | 1.324212365 | 0.987 | 0.646 | 1.80E−165 | E-APSM | CITED2 |
| 1.81E−237 | 1.253400152 | 0.8 | 0.183 | 6.62E−233 | E-APSM | TTN |
| 2.11E−153 | 1.249268196 | 0.987 | 0.658 | 7.72E−149 | E-APSM | SKAP2 |
| 1.39E−188 | 1.141276441 | 0.971 | 0.371 | 5.08E−184 | E-APSM | DLL3 |
| 1.05E−184 | 1.129172491 | 0.966 | 0.429 | 3.85E−180 | E-APSM | MAGI1 |
| 1.74E−220 | 1.126192762 | 0.913 | 0.267 | 6.37E−216 | E-APSM | HAS2 |
| 2.21E−157 | 1.113217809 | 0.937 | 0.45 | 8.10E−153 | E-APSM | LFNG |
| 6.53E−249 | 1.093107953 | 0.884 | 0.239 | 2.39E−244 | E-APSM | ZBTB18 |
| 1.16E−130 | 0.981431549 | 0.953 | 0.517 | 4.25E−126 | E-APSM | ALDH1A2 |
| 1.56E−147 | 0.911923972 | 1 | 0.974 | 5.71E−143 | E-APSM | LAPTM4B |
| 4.09E−167 | 0.896189553 | 0.882 | 0.329 | 1.50E−162 | E-APSM | STRA6 |
| 5.72E−273 | 0.872879305 | 0.847 | 0.175 | 2.09E−268 | E-APSM | ABCG1 |
| 2.78E−159 | 0.852620156 | 0.868 | 0.303 | 1.02E−154 | E-APSM | ANOS1 |
| 4.11E−111 | 0.715314759 | 0.795 | 0.309 | 1.51E−106 | E-APSM | SNAI2 |
| 1.08E−57 | 0.705523519 | 0.982 | 0.866 | 3.94E−53 | E-APSM | HOTAIRM |
| 1.69E−89 | 0.70259339 | 0.782 | 0.392 | 6.17E−85 | E-APSM | DMRTA1 |
| 2.63E−71 | 0.669343441 | 0.984 | 0.886 | 9.63E−67 | E-APSM | LMO4 |
| 3.19E−102 | 0.653358008 | 0.792 | 0.333 | 1.17E−97 | E-APSM | MGST2 |
| 1.14E−89 | 0.648321947 | 0.674 | 0.258 | 4.17E−85 | E-APSM | PAPPA |
| 5.35E−91 | 0.646465885 | 0.887 | 0.472 | 1.96E−86 | E-APSM | ZIC3 |
| 5.91E−60 | 0.614645081 | 0.839 | 0.538 | 2.16E−55 | E-APSM | SPRY1 |
| 2.04E−68 | 0.581726386 | 0.887 | 0.552 | 7.46E−64 | E-APSM | COLEC12 |
| 1.63E−65 | 0.561954564 | 0.903 | 0.585 | 5.95E−61 | E-APSM | PPP3CA |
| 1.21E−52 | 0.555919874 | 0.918 | 0.659 | 4.41E−48 | E-APSM | FZD7 |
| 6.06E−56 | 0.554665339 | 0.984 | 0.782 | 2.22E−51 | E-APSM | FOXC1 |
| 2.03E−84 | 0.54584715 | 0.782 | 0.378 | 7.43E−80 | E-APSM | NDFIP2 |
| 6.02E−62 | 0.533656229 | 0.945 | 0.729 | 2.21E−57 | E-APSM | PCBP4 |
| 4.28E−53 | 0.531553205 | 0.763 | 0.448 | 1.57E−48 | E-APSM | SVIL |
| 1.97E−50 | 0.531360185 | 0.874 | 0.593 | 7.21E−46 | E-APSM | DACT1 |
| 3.73E−215 | 0.501826458 | 0.582 | 0.097 | 1.36E−210 | E-APSM | AFAP1L2 |
| 1.09E−28 | 0.487598572 | 0.745 | 0.505 | 4.00E−24 | E-APSM | LGALS1 |
| 1.56E−143 | 0.48190137 | 0.637 | 0.157 | 5.70E−139 | E-APSM | CD44 |
| 5.86E−122 | 0.481480527 | 0.632 | 0.179 | 2.15E−117 | E-APSM | ISM1 |
| 6.37E−54 | 0.474265698 | 0.979 | 0.924 | 2.33E−49 | E-APSM | ANXA5 |
| 1.80E−184 | 0.464111317 | 0.329 | 0.037 | 6.57E−180 | E-APSM | PENK |
| 1.77E−44 | 0.462024612 | 0.939 | 0.749 | 6.48E−40 | E-APSM | ARID3A |
| 4.69E−47 | 0.461650539 | 0.876 | 0.627 | 1.72E−42 | E-APSM | ADD3 |
| 1.94E−63 | 0.459294218 | 0.7 | 0.332 | 7.12E−59 | E-APSM | TNIK |
| 1.03E−98 | 0.448622363 | 0.611 | 0.193 | 3.79E−94 | E-APSM | TMEM108 |
| 0 | 3.762331336 | 0.917 | 0.427 | 0 | APSM | RIPPLY1 |
| 0 | 2.696992333 | 0.927 | 0.088 | 0 | APSM | PCDH8 |
| 0 | 2.382565672 | 0.981 | 0.258 | 0 | APSM | SSTR2 |
| 0 | 1.757021512 | 0.768 | 0.162 | 0 | APSM | PRL |
| 0 | 1.64713422 | 1 | 0.966 | 0 | APSM | CALD1 |
| 0 | 1.222645423 | 0.993 | 0.739 | 0 | APSM | LEF1 |
| 0 | 1.203799477 | 0.837 | 0.306 | 0 | APSM | PLK2 |
| 0 | 1.160503589 | 0.959 | 0.447 | 0 | APSM | MEOX1 |
| 0 | 1.135954622 | 0.946 | 0.511 | 0 | APSM | PCDH19 |
| 0 | 1.114750671 | 0.988 | 0.727 | 0 | APSM | FOXC2 |
| 0 | 1.097290258 | 0.935 | 0.476 | 0 | APSM | NOTCH1 |
| 1.62E−169 | 1.045988664 | 0.697 | 0.366 | 5.94E−165 | APSM | FGF18 |
| 0 | 1.003927338 | 1 | 0.99 | 0 | APSM | SOX4 |
| 0 | 1.003748109 | 0.925 | 0.522 | 0 | APSM | ADGRL2 |
| 0 | 0.990450962 | 0.992 | 0.727 | 0 | APSM | GJA1 |
| 0 | 0.957619102 | 0.415 | 0.063 | 0 | APSM | GADD45G |
| 0 | 0.883297769 | 0.675 | 0.118 | 0 | APSM | NAV2 |
| 0 | 0.858622917 | 0.874 | 0.264 | 0 | APSM | ENPP2 |
| 0 | 0.849561378 | 0.998 | 0.934 | 0 | APSM | PFN2 |
| 1.57E−258 | 0.826642907 | 0.647 | 0.252 | 5.76E−254 | APSM | SOX9 |
| 0 | 0.812536324 | 0.731 | 0.241 | 0 | APSM | PDE4DIP |
| 9.17E−259 | 0.7922868 | 0.876 | 0.54 | 3.36E−254 | APSM | ZNF608 |
| 4.18E−240 | 0.787692558 | 0.991 | 0.919 | 1.53E−235 | APSM | MAP1B |
| 0 | 0.777435428 | 0.898 | 0.504 | 0 | APSM | ARHGAP10 |

TABLE 1-F

| | | | | | | |
|---|---|---|---|---|---|---|
| 0 | 0.762505689 | 0.64 | 0.088 | 0 | APSM | TMEM132B |
| 9.12E−239 | 0.760658033 | 0.883 | 0.583 | 3.34E−234 | APSM | SLC5A3 |
| 9.93E−243 | 0.747194909 | 0.959 | 0.73 | 3.64E−238 | APSM | NCAM1 |
| 0 | 0.712146741 | 0.484 | 0.054 | 0 | APSM | MYOCD |
| 0 | 0.708961468 | 0.77 | 0.267 | 0 | APSM | STOX1 |
| 0 | 0.701171792 | 0.781 | 0.339 | 0 | APSM | FHOD3 |
| 8.40E−249 | 0.693695021 | 0.887 | 0.571 | 3.08E−244 | APSM | TMEM47 |
| 1.58E−216 | 0.68631519 | 0.988 | 0.903 | 5.76E−212 | APSM | FGFR1 |
| 1.19E−223 | 0.669104094 | 0.881 | 0.567 | 4.34E−219 | APSM | MYO10 |
| 8.12E−181 | 0.654215703 | 0.863 | 0.627 | 2.97E−176 | APSM | SDC2 |
| 1.82E−239 | 0.635392482 | 0.774 | 0.387 | 6.65E−235 | APSM | UPP1 |
| 4.01E−204 | 0.628762038 | 1 | 0.976 | 1.47E−199 | APSM | JPT1 |
| 3.84E−186 | 0.614042454 | 0.959 | 0.782 | 1.40E−181 | APSM | SALL4 |
| 1.39E−212 | 0.611007157 | 0.993 | 0.922 | 5.08E−208 | APSM | MRPS6 |
| 6.66E−262 | 0.600838164 | 0.744 | 0.315 | 2.44E−257 | APSM | NTM |
| 1.78E−208 | 0.596647215 | 0.723 | 0.355 | 6.53E−204 | APSM | FRMD6 |
| 0 | 0.595391367 | 0.618 | 0.134 | 0 | APSM | SYT6 |
| 6.23E−257 | 0.595335708 | 0.744 | 0.331 | 2.28E−252 | APSM | MYCN |
| 2.38E−241 | 0.582752718 | 0.759 | 0.377 | 8.72E−237 | APSM | TRMT5 |
| 6.09E−197 | 0.560832418 | 0.858 | 0.532 | 2.23E−192 | APSM | FAM13A |
| 4.39E−184 | 0.5514245 | 0.802 | 0.481 | 1.60E−179 | APSM | LPIN2 |
| 3.88E−107 | 0.538906778 | 0.989 | 0.903 | 1.42E−102 | APSM | CADM1 |
| 2.99E−264 | 0.533426241 | 0.526 | 0.164 | 1.09E−259 | APSM | SLAIN1 |
| 6.62E−178 | 0.532636359 | 0.802 | 0.485 | 2.42E−173 | APSM | SEC22B |
| 7.69E−159 | 0.52074163 | 0.794 | 0.495 | 2.82E−154 | APSM | CCDC66 |
| 0 | 0.520598913 | 0.474 | 0.062 | 0 | APSM | C6orf132 |
| 0 | 1.773954339 | 1 | 0.995 | 0 | N-SM | FABP5 |
| 0 | 1.587703473 | 0.94 | 0.611 | 0 | N-SM | HES4 |
| 7.57E−175 | 0.925298456 | 0.99 | 0.888 | 2.77E−170 | N-SM | DSP |
| 2.37E−245 | 0.857438977 | 0.728 | 0.306 | 8.68E−241 | N-SM | ADA |
| 1.51E−147 | 0.744085994 | 0.945 | 0.725 | 5.54E−143 | N-SM | TUBB3 |
| 2.35E−240 | 0.72655748 | 0.625 | 0.204 | 8.61E−236 | N-SM | GADD45B |
| 6.91E−161 | 0.708598103 | 0.907 | 0.629 | 2.53E−156 | N-SM | EFNB2 |
| 4.45E−109 | 0.707039708 | 0.873 | 0.673 | 1.63E−104 | N-SM | AL031058.1 |
| 7.92E−135 | 0.682010328 | 0.99 | 0.905 | 2.90E−130 | N-SM | PTN |
| 1.54E−225 | 0.633393456 | 0.635 | 0.223 | 5.62E−221 | N-SM | OLFM3 |
| 2.13E−146 | 0.548145101 | 0.69 | 0.339 | 7.79E−142 | N-SM | ZBTB38 |
| 1.81E−107 | 0.525002135 | 0.703 | 0.417 | 6.61E−103 | N-SM | TSPAN13 |
| 6.60E−247 | 0.523127248 | 0.546 | 0.15 | 2.42E−242 | N-SM | IL18 |
| 6.87E−108 | 0.516076682 | 0.818 | 0.574 | 2.52E−103 | N-SM | TCHP |
| 5.23E−126 | 0.500789116 | 0.988 | 0.902 | 1.91E−121 | IN-SM | FBLN1 |
| 2.72E−109 | 0.484672092 | 0.859 | 0.552 | 9.94E−105 | N-SM | MXRA5 |
| 1.52E−158 | 0.481139529 | 0.601 | 0.25 | 5.57E−154 | N-SM | FJX1 |
| 5.61E−50 | 0.465115164 | 0.769 | 0.625 | 2.05E−45 | N-SM | BEX1 |
| 3.61E−124 | 0.412924389 | 0.568 | 0.254 | 1.32E−119 | N-SM | PRSS23 |
| 2.99E−74 | 0.384827891 | 0.979 | 0.919 | 1.09E−69 | N-SM | BZW2 |
| 2.15E−136 | 0.359661535 | 1 | 0.999 | 7.87E−132 | N-SM | SERBP1 |
| 2.52E−70 | 0.358622984 | 0.959 | 0.856 | 9.24E−66 | N-SM | EIF4EBP1 |
| 2.85E−84 | 0.353847372 | 0.993 | 0.958 | 1.04E−79 | N-SM | TIMM8B |
| 5.11E−98 | 0.323172471 | 0.998 | 0.997 | 1.87E−93 | N-SM | PSMA7 |
| 5.51E−78 | 0.302679942 | 0.998 | 0.992 | 2.02E−73 | N-SM | SNRPD1 |
| 0 | 1.138259202 | 0.999 | 0.94 | 0 | E-SM1 | NNAT |
| 0 | 1.043862076 | 0.935 | 0.632 | 0 | E-SM1 | ALCAM |
| 0 | 0.992087163 | 0.996 | 0.866 | 0 | E-SM1 | COL1A2 |
| 0 | 0.92093589 | 0.771 | 0.313 | 0 | E-SM1 | COL14A1 |
| 0 | 0.85641532 | 0.914 | 0.714 | 0 | E-SM1 | ANXA2 |
| 0 | 0.757118826 | 0.792 | 0.457 | 0 | E-SM1 | RDH10 |
| 0 | 0.741606533 | 0.786 | 0.454 | 0 | E-SM1 | NAV1 |
| 0 | 0.70569367 | 0.886 | 0.698 | 0 | E-SM1 | LHFPL6 |
| 0 | 0.689194596 | 0.828 | 0.613 | 0 | E-SM1 | CNKSR3 |
| 0 | 0.687799124 | 0.917 | 0.65 | 0 | E-SM1 | BOC |
| 0 | 0.656658003 | 0.743 | 0.494 | 0 | E-SM1 | ANK3 |
| 0 | 0.644201532 | 0.802 | 0.527 | 0 | E-SM1 | PAX3 |
| 0 | 0.64270727 | 0.867 | 0.746 | 0 | E-SM1 | FBN2 |

TABLE 1-G

| | | | | | | |
|---|---|---|---|---|---|---|
| 0 | 0.639998767 | 0.784 | 0.558 | 0 | E-SM1 | ZNF704 |
| 0 | 0.631977241 | 0.963 | 0.897 | 0 | E-SM1 | CDH11 |
| 0 | 0.626536011 | 0.835 | 0.621 | 0 | E-SM1 | PDLIM5 |
| 1.35E−254 | 0.615929038 | 0.851 | 0.699 | 4.94E−250 | E-SM1 | CTHRC1 |
| 2.49E−295 | 0.575315756 | 0.704 | 0.469 | 9.10E−291 | E-SM1 | IL6ST |
| 1.01E−253 | 0.555453509 | 0.873 | 0.752 | 3.71E−249 | E-SM1 | PDGFRA |
| 4.19E−305 | 0.552895523 | 0.902 | 0.803 | 1.53E−300 | E-SM1 | FARP1 |
| 1.53E−238 | 0.550067784 | 0.674 | 0.488 | 5.59E−234 | E-SM1 | TENM4 |
| 0 | 0.538186196 | 0.976 | 0.944 | 0 | E-SM1 | QPRT |
| 8.46E−239 | 0.52562248 | 0.597 | 0.36 | 3.10E−234 | E-SM1 | KLHL4 |
| 1.01E−237 | 0.525482783 | 0.888 | 0.786 | 3.69E−233 | E-SM1 | SEMA6A |
| 2.66E−275 | 0.519061793 | 0.66 | 0.409 | 9.75E−271 | E-SM1 | CEP126 |
| 4.46E−236 | 0.518717102 | 0.723 | 0.536 | 1.63E−231 | E-SM1 | ATP1A2 |
| 2.32E−256 | 0.515590554 | 0.759 | 0.589 | 8.51E−252 | E-SM1 | ARID3B |
| 3.83E−157 | 0.515264957 | 0.908 | 0.842 | 1.40E−152 | E-SM1 | KCNQ1OT1 |
| 7.71E−204 | 0.513837447 | 0.921 | 0.858 | 2.82E−199 | E-SM1 | SYNE2 |
| 5.65E−175 | 0.502336563 | 0.58 | 0.393 | 2.07E−170 | E-SM1 | MIR503HG |
| 2.29E−190 | 0.484785981 | 0.881 | 0.817 | 8.36E−186 | E-SM1 | GOLGB1 |
| 1.54E−206 | 0.481583754 | 0.77 | 0.628 | 5.63E−202 | E-SM1 | CCDC80 |
| 8.07E−213 | 0.476949825 | 0.643 | 0.472 | 2.95E−208 | E-SM1 | C1orf21 |
| 1.38E−214 | 0.45771165 | 0.927 | 0.868 | 5.05E−210 | E-SM1 | COL4A2 |
| 1.10E−236 | 0.451994862 | 0.984 | 0.951 | 4.01E−232 | E-SM1 | TPM2 |
| 8.41E−215 | 0.442554122 | 0.568 | 0.355 | 3.08E−210 | E-SM1 | PLXDC2 |
| 2.86E−192 | 0.427123251 | 0.501 | 0.299 | 1.05E−187 | E-SM1 | NHS |
| 1.71E−143 | 0.42430546 | 0.789 | 0.704 | 6.25E−139 | E-SM1 | GABPB1-AS1 |
| 6.37E−140 | 0.417742334 | 0.971 | 0.926 | 2.33E−135 | E-SM1 | ZFP36L1 |
| 1.03E−262 | 0.415656652 | 0.424 | 0.182 | 3.77E−258 | E-SM1 | TMEM88 |
| 1.45E−186 | 0.41344267 | 0.984 | 0.984 | 5.29E−182 | E-SM1 | ARGLU1 |
| 2.78E−154 | 0.40770255 | 0.568 | 0.401 | 1.02E−149 | E-SM1 | AF117829.1 |
| 1.47E−185 | 0.406753393 | 0.555 | 0.367 | 5.39E−181 | E-SM1 | CBLB |
| 8.32E−135 | 0.404518813 | 0.748 | 0.651 | 3.05E−130 | E-SM1 | TXNIP |
| 3.78E−168 | 0.398024873 | 0.972 | 0.956 | 1.38E−163 | E-SM1 | SEPTIN11 |
| 1.05E−236 | 0.396997299 | 0.99 | 0.987 | 3.83E−232 | E-SM1 | RBMX |
| 2.81E−168 | 0.390729195 | 0.871 | 0.825 | 1.03E−163 | E-SM1 | KMT2E |
| 3.28E−204 | 0.384075183 | 0.544 | 0.314 | 1.20E−199 | E-SM1 | TCF15 |
| 2.01E−149 | 0.383691559 | 0.698 | 0.575 | 7.36E−145 | E-SM1 | MT-ATP8 |
| 1.20E−145 | 0.37856534 | 0.927 | 0.905 | 4.38E−141 | E-SM1 | BPTF |
| 0 | 1.311532049 | 0.96 | 0.712 | 0 | M-SM1 | FST |
| 0 | 1.076060257 | 0.935 | 0.529 | 0 | M-SM1 | PLCG2 |
| 0 | 0.771000449 | 0.974 | 0.84 | 0 | M-SM1 | BRI3 |
| 0 | 0.766157299 | 0.914 | 0.626 | 0 | M-SM1 | LINC01578 |
| 0 | 0.748465829 | 0.922 | 0.642 | 0 | M-SM1 | MAP2K2 |
| 0 | 0.672466293 | 1 | 1 | 0 | M-SM1 | RPS27 |
| 0 | 0.669701543 | 0.97 | 0.902 | 0 | M-SM1 | SFRP1 |
| 0 | 0.658550696 | 1 | 1 | 0 | M-SM1 | RPS29 |
| 0 | 0.658514654 | 1 | 1 | 0 | M-SM1 | RPS28 |
| 0 | 0.61597534 | 1 | 1 | 0 | M-SM1 | RPL37 |
| 0 | 0.613463237 | 1 | 1 | 0 | M-SM1 | RPL34 |
| 0 | 0.610210657 | 1 | 1 | 0 | M-SM1 | RPL39 |
| 0 | 0.58870973 | 1 | 1 | 0 | M-SM1 | RPS21 |
| 0 | 0.575078555 | 1 | 1 | 0 | M-SM1 | RPL36A |
| 0 | 0.569666447 | 1 | 1 | 0 | M-SM1 | RPL31 |
| 0 | 0.565838866 | 1 | 1 | 0 | M-SM1 | RPL41 |
| 0 | 0.563002584 | 1 | 1 | 0 | M-SM1 | RPL37A |
| 0 | 0.532720695 | 0.794 | 0.528 | 0 | M-SM1 | METRN |
| 0 | 0.527628934 | 1 | 1 | 0 | M-SM1 | RPS25 |
| 0 | 0.505160948 | 0.811 | 0.49 | 0 | M-SM1 | GAS1 |
| 0 | 0.492999142 | 1 | 1 | 0 | M-SM1 | RPS8 |
| 0 | 0.483230383 | 0.995 | 0.975 | 0 | M-SM1 | GPC3 |
| 0 | 0.48041295 | 1 | 1 | 0 | M-SM1 | RPL38 |
| 0 | 0.470299859 | 1 | 1 | 0 | M-SM1 | RPL30 |
| 0 | 0.468368171 | 0.999 | 0.997 | 0 | M-SM1 | UQCRB |
| 0 | 0.463988247 | 0.995 | 0.983 | 0 | M-SM1 | GAS5 |
| 0 | 0.461245933 | 0.999 | 0.997 | 0 | M-SM1 | COX7C |

TABLE 1-H

| | | | | | | |
|---|---|---|---|---|---|---|
| 0 | 0.460121206 | 1 | 1 | 0 | M-SM1 | RPL35A |
| 0 | 0.458912349 | 0.989 | 0.971 | 0 | M-SM1 | SNHG6 |
| 0 | 0.455902467 | 1 | 1 | 0 | M-SM1 | RPL36 |
| 0 | 0.455237906 | 0.996 | 0.991 | 0 | M-SM1 | ZFAS1 |
| 0 | 0.453801477 | 1 | 1 | 0 | M-SM1 | RPL28 |
| 0 | 0.453063349 | 0.448 | 0.144 | 0 | M-SM1 | SCX |
| 2.05E−191 | 0.440966275 | 0.912 | 0.876 | 7.50E−187 | M-SM1 | CCDC34 |
| 0 | 0.436978017 | 1 | 1 | 0 | M-SM1 | RPS15A |
| 0 | 0.429832669 | 0.996 | 0.986 | 0 | M-SM1 | TMEM258 |
| 0 | 0.424163522 | 0.978 | 0.941 | 0 | M-SM1 | COMMD6 |
| 0 | 0.422629735 | 1 | 0.999 | 0 | M-SM1 | ATP5MC2 |
| 0 | 0.42026579 | 1 | 1 | 0 | M-SM1 | RPS26 |
| 0 | 0.411388023 | 0.458 | 0.189 | 0 | M-SM1 | PRRX2 |
| 0 | 0.411246115 | 1 | 1 | 0 | M-SM1 | RPLP1 |
| 0 | 0.410636439 | 1 | 1 | 0 | M-SM1 | RPLP2 |
| 0 | 0.40806768 | 0.538 | 0.27 | 0 | M-SM1 | DDIT4L |
| 0 | 0.39701697 | 1 | 1 | 0 | M-SM1 | RPL23 |
| 1.52E−209 | 0.396259803 | 0.551 | 0.344 | 5.55E−205 | M-SM1 | HES1 |
| 0 | 0.391931434 | 1 | 1 | 0 | M-SM1 | RPS12 |
| 0 | 0.38621807 | 1 | 1 | 0 | M-SM1 | RPL32 |
| 0 | 0.384452187 | 1 | 1 | 0 | M-SM1 | RPS13 |
| 5.88E−285 | 0.379379761 | 0.976 | 0.951 | 2.15E−280 | M-SM1 | HMGA2 |
| 7.93E−300 | 0.37902944 | 0.978 | 0.964 | 2.90E−295 | M-SM1 | DANCR |
| 0 | 2.045059356 | 0.988 | 0.512 | 0 | L-SM | IGFBP5 |
| 0 | 1.100003946 | 0.803 | 0.323 | 0 | L-SM | BST2 |
| 0 | 1.029228868 | 0.71 | 0.175 | 0 | L-SM | NR2F1 |
| 0 | 1.002769151 | 0.731 | 0.205 | 0 | L-SM | FLRT3 |
| 0 | 0.970325569 | 0.643 | 0.103 | 0 | L-SM | SHISA2 |
| 0 | 0.969871867 | 0.909 | 0.418 | 0 | L-SM | PCOLCE |
| 0 | 0.886864093 | 0.797 | 0.372 | 0 | L-SM | AMOT |
| 0 | 0.842159182 | 0.888 | 0.569 | 0 | L-SM | SFRP2 |
| 0 | 0.830397913 | 0.957 | 0.792 | 0 | L-SM | SOX11 |
| 0 | 0.794168438 | 0.751 | 0.317 | 0 | L-SM | PHLDA1 |
| 0 | 0.791560528 | 0.938 | 0.615 | 0 | L-SM | SEPTIN6 |
| 0 | 0.711586979 | 0.701 | 0.299 | 0 | L-SM | DDIT4 |
| 0 | 0.703100373 | 0.941 | 0.73 | 0 | L-SM | FOXP1 |
| 0 | 0.671558274 | 0.744 | 0.343 | 0 | L-SM | MYLIP |
| 0 | 0.6668989 | 0.89 | 0.534 | 0 | L-SM | NR2F2 |
| 0 | 0.651371983 | 0.643 | 0.266 | 0 | L-SM | PCDH17 |
| 0 | 0.624709604 | 0.984 | 0.74 | 0 | L-SM | COL2A1 |
| 0 | 0.616784226 | 0.99 | 0.92 | 0 | L-SM | TSC22D1 |
| 0 | 0.607646545 | 0.925 | 0.594 | 0 | L-SM | MXRA8 |
| 7.78E−209 | 0.604710705 | 0.476 | 0.295 | 2.85E−204 | L-SM | COL3A1 |
| 0 | 0.602680429 | 0.417 | 0.029 | 0 | L-SM | DNM3OS |
| 0 | 0.597553885 | 0.802 | 0.492 | 0 | L-SM | FLRT2 |
| 0 | 0.594875476 | 0.999 | 0.997 | 0 | L-SM | STMN1 |
| 0 | 0.579401471 | 0.789 | 0.495 | 0 | L-SM | ARRDC3 |
| 0 | 0.566665565 | 0.856 | 0.592 | 0 | L-SM | LRIG3 |
| 0 | 0.557045879 | 1 | 0.999 | 0 | L-SM | MARCKS |
| 0 | 0.547689573 | 0.444 | 0.151 | 0 | L-SM | JUN |
| 0 | 0.546485447 | 0.75 | 0.493 | 0 | L-SM | PIK3R1 |
| 0 | 0.529036596 | 0.437 | 0.101 | 0 | L-SM | MAFB |
| 0 | 0.512490871 | 0.294 | 0.027 | 0 | L-SM | GASK1B |
| 0 | 0.499368144 | 0.432 | 0.073 | 0 | L-SM | MIR100HG |
| 0 | 0.494475741 | 0.556 | 0.14 | 0 | L-SM | EYA2 |
| 0 | 0.493201064 | 0.849 | 0.608 | 0 | L-SM | NRIP1 |
| 0 | 0.491814352 | 0.821 | 0.538 | 0 | L-SM | WFIKKN1 |
| 0 | 0.488509325 | 0.987 | 0.929 | 0 | L-SM | DDAH2 |
| 0 | 0.483729759 | 0.756 | 0.447 | 0 | L-SM | LGR4 |
| 0 | 0.480222729 | 0.615 | 0.341 | 0 | L-SM | DRAXIN |
| 0 | 0.478688132 | 0.994 | 0.978 | 0 | L-SM | PPDPF |
| 0 | 0.477642022 | 0.862 | 0.571 | 0 | L-SM | SIX1 |
| 0 | 0.473946712 | 0.966 | 0.888 | 0 | L-SM | CCND2 |
| 0 | 0.46826439 | 0.689 | 0.414 | 0 | L-SM | TENM3 |

TABLE 1-I

| | | | | | | |
|---|---|---|---|---|---|---|
| 0 | 0.464030382 | 0.524 | 0.242 | 0 | L-SM | CXXC4 |
| 0 | 0.450578701 | 0.688 | 0.39 | 0 | L-SM | TSHZ2 |
| 0 | 0.442715313 | 0.608 | 0.346 | 0 | L-SM | DLEU2 |
| 0 | 0.437319066 | 0.964 | 0.89 | 0 | L-SM | HOMER3 |
| 0 | 0.423318392 | 0.786 | 0.49 | 0 | L-SM | LTBP4 |
| 5.32E−290 | 0.416368331 | 0.891 | 0.782 | 1.95E−285 | L-SM | FZD2 |
| 1.60E−303 | 0.414036168 | 0.842 | 0.689 | 5.87E−299 | L-SM | TSC22D3 |
| 1.10E−234 | 0.407952516 | 0.816 | 0.685 | 4.03E−230 | L-SM | ZNF503 |
| 0 | 0.402492204 | 1 | 1 | 0 | L-SM | RPS4X |
| 1.20E−257 | 2.925860612 | 0.64 | 0.07 | 4.40E−253 | EC-like | ETV2 |
| 3.75E−226 | 2.401588704 | 0.901 | 0.19 | 1.37E−221 | EC-like | RAMP2 |
| 3.83E−127 | 2.400828996 | 0.928 | 0.373 | 1.40E−122 | EC-like | GNG11 |
| 0 | 2.327016584 | 0.914 | 0.024 | 0 | EC-like | KDR |
| 4.29E−95 | 2.316864579 | 0.955 | 0.726 | 1.57E−90 | EC-like | HSPB1 |
| 4.34E−130 | 2.263218842 | 0.995 | 0.969 | 1.59E−125 | EC-like | S100A11 |
| 1.45E−40 | 1.93189211 | 0.559 | 0.268 | 5.30E−36 | EC-like | EGFL7 |
| 7.06E−192 | 1.89795614 | 0.914 | 0.238 | 2.59E−187 | EC-like | LMO2 |
| 3.64E−167 | 1.897619067 | 0.932 | 0.267 | 1.33E−162 | EC-like | LIMCH1 |
| 0 | 1.772574314 | 0.815 | 0.072 | 0 | EC-like | TFPI |
| 2.26E−100 | 1.487226495 | 0.689 | 0.204 | 8.29E−96 | EC-like | RHOB |
| 1.10E−83 | 1.456205643 | 0.941 | 0.656 | 4.01E−79 | EC-like | IER2 |
| 2.05E−152 | 1.436435785 | 0.739 | 0.156 | 7.52E−148 | EC-like | COL6A3 |
| 1.58E−93 | 1.419504862 | 0.581 | 0.137 | 5.79E−89 | EC-like | TAGLN |
| 4.60E−42 | 1.37664091 | 0.896 | 0.768 | 1.68E−37 | EC-like | HAPLN1 |
| 3.74E−119 | 1.370203231 | 0.865 | 0.315 | 1.37E−114 | EC-like | IGFBP4 |
| 4.29E−85 | 1.356976507 | 0.937 | 0.499 | 1.57E−80 | EC-like | ENC1 |
| 9.99E−62 | 1.230522905 | 0.977 | 0.853 | 3.65E−57 | EC-like | ID3 |
| 1.69E−76 | 1.216081378 | 0.847 | 0.399 | 6.20E−72 | EC-like | TGFB1 |
| 1.64E−51 | 1.208472895 | 0.932 | 0.706 | 6.01E−47 | EC-like | FN1 |
| 4.19E−80 | 1.198699648 | 0.91 | 0.474 | 1.53E−75 | EC-like | BGN |
| 0 | 1.196803483 | 0.257 | 0 | 0 | EC-like | SOX17 |
| 1.29E−87 | 1.188055426 | 0.923 | 0.532 | 4.70E−83 | EC-like | MMP2 |
| 4.60E−58 | 1.183723515 | 0.833 | 0.533 | 1.68E−53 | EC-like | TCEAL7 |
| 1.81E−145 | 1.16839538 | 0.896 | 0.283 | 6.64E−141 | EC-like | ELK3 |
| 2.27E−57 | 1.161560994 | 0.973 | 0.916 | 8.30E−53 | EC-like | MYL12B |
| 4.81E−153 | 1.108525314 | 0.342 | 0.031 | 1.76E−148 | EC-like | CALCRL |
| 2.43E−84 | 1.103506448 | 0.973 | 0.632 | 8.89E−80 | EC-like | MDFI |
| 3.62E−117 | 1.078463891 | 0.721 | 0.177 | 1.32E−112 | EC-like | JUNB |
| 1.31E−56 | 1.073170058 | 0.806 | 0.485 | 4.81E−52 | EC-like | CREM |
| 1.01E−127 | 1.05611775 | 1 | 1 | 3.71E−123 | EC-like | TPT1 |
| 5.12E−166 | 1.055138823 | 0.622 | 0.097 | 1.87E−161 | EC-like | FLT1 |
| 6.50E−69 | 1.054125378 | 0.991 | 0.912 | 2.38E−64 | EC-like | MYL12A |
| 0 | 1.049909977 | 0.441 | 0.012 | 0 | EC-like | PLVAP |
| 2.13E−58 | 1.048123124 | 0.68 | 0.311 | 7.81E−54 | EC-like | THY1 |
| 2.59E−29 | 1.041692263 | 0.829 | 0.602 | 9.48E−25 | EC-like | SPTBN1 |
| 0 | 1.036928295 | 0.347 | 0.002 | 0 | EC-like | CD34 |
| 1.45E−63 | 1.031980641 | 0.865 | 0.494 | 5.32E−59 | EC-like | HAPLN3 |
| 2.20E−70 | 1.027941005 | 0.617 | 0.18 | 8.05E−66 | EC-like | DLK1 |
| 1.96E−124 | 1.021898969 | 0.694 | 0.168 | 7.18E−120 | EC-like | SULT1C4 |
| 1.71E−97 | 1.007996039 | 0.842 | 0.323 | 6.25E−93 | EC-like | FHL3 |
| 0 | 1.004482635 | 0.626 | 0.04 | 0 | EC-like | MEF2C |
| 4.63E−126 | 0.991323811 | 0.477 | 0.072 | 1.70E−121 | EC-like | LINC02762 |
| 6.29E−72 | 0.982005264 | 0.905 | 0.53 | 2.30E−67 | EC-like | ARHGAP28 |
| 4.58E−47 | 0.976392254 | 0.973 | 0.876 | 1.67E−42 | EC-like | ITM2B |
| 2.48E−72 | 0.941395907 | 0.797 | 0.375 | 9.09E−68 | EC-like | EFEMP2 |
| 1.06E−40 | 0.931248136 | 0.676 | 0.327 | 3.89E−36 | EC-like | ID1 |
| 0 | 0.918215676 | 0.518 | 0 | 0 | EC-like | HHEX |
| 5.45E−55 | 0.912050517 | 1 | 1 | 2.00E−50 | EC-like | VIM |
| 6.18E−55 | 0.901407831 | 0.599 | 0.234 | 2.26E−50 | EC-like | SNCG |

Supplementary Table 2 (Table 2-A to 2-B): NMP and NE module score genes. List of neuromesodenrmal progenitor (NMP) and neuroectodenrm (NE) associated genes used to calculate the module scores for FIG. 8g.

TABLE 2-A

| NMP genes | NE genes | NMP genes | NE genes | NMP genes | NE genes | NMP genes | NE genes |
|---|---|---|---|---|---|---|---|
| AKR1B1 | GTF3A | ANTXR1 | SRGAP3 | | CELSR2 | | TIMP3 |
| MPST | DUSP14 | SLIT3 | HSD17B8 | | DTX4 | | AKAP12 |
| B3GNT7 | IGFBP2 | ZNF300 | QPRT | | FHDC1 | | PKP2 |
| SNHG9 | HLA-A | CD40 | IGSF9B | | FAM20C | | JPH4 |
| TPCN2 | HLA-B | ANGPTL4 | SFN | | DEDD2 | | KCNAB3 |
| C16orf74 | HLA-C | SPRY4 | TCEA2 | | TCFL5 | | SLC4A8 |
| LPAR1 | HLA-E | RIPPLY2 | GGH | | GLI2 | | TANC2 |
| ENDOG | HLA-F | LAMTOR2 | UCHL1 | | ADARB2 | | LRP2 |
| RHBG | HLA-G | CDC42EP5 | SEMA5B | | SLC35F1 | | SLC31A2 |
| FSTL4 | FHOD3 | CLDN7 | PARD6A | | ABCC4 | | CERS4 |
| AVPR1B | BEX1 | NOTCH4 | CAMSAP3 | | TIMP4 | | BOC |
| ADAMTSL2 | BEX2 | ETS2 | NRP2 | | PLCG2 | | LRRC8D |
| PPP1R3C | RNF208 | C6orf47 | PDXK | | CNTFR | | TSPYL5 |
| ATP1A2 | EMID1 | HINT3 | MBP | | RFX4 | | RASSF2 |
| FGF3 | NAT8L | INSIG1 | ZGLP1 | | SCAMP5 | | UST |
| TRIM9 | GGCT | MSMP | RMRP | | POSTN | | LCAT |
| HLA-A | BOK | TRIM25 | NOG | | IRX2 | | PSD3 |
| HLA-B | SPIRE2 | MMP17 | MKRN1 | | TP53I11 | | N48P3 |
| HLA-C | FGFBP3 | TRIM36 | SOX3 | | YPEL1 | | MAFB |
| HLA-E | URAD | SBSN | CIB1 | | SOX2 | | SEMA3A |
| HLA-F | HPCAL1 | THEM4 | NDUFS5 | | PDGFA | | ARMC10 |
| HLA-G | PLA2G12A | TMPRSS2 | GALNT16 | | E2F2 | | ICA1 |
| CHST7 | KLHL8 | B4GALT7 | HSDL1 | | ZNF442 | | GDF10 |
| ZIC3 | BEX4 | EFCAB2 | CCDC181 | | TOX3 | | ALX3 |
| NPM3 | GCAT | ZFP28 | TUBB4A | | PCSK9 | | ZNF169 |
| SP5 | SLC37A4 | PCP4 | SOX21 | | SLITRK5 | | KLF3 |
| GAD1 | PIP5K1B | NPL | SEMA3E | | ADAM23 | | STK10 |
| TBXT | ACOT1 | GUCY1A2 | BRSK2 | | USP44 | | NEXN |
| SH3GL3 | ACOT2 | LTBR | FBXO36 | | TMSB15A | | VANGL1 |
| SLC2A6 | WSCD1 | SULF1 | OCIAD2 | | TMSB15B | | LENEP |
| PRICKLE2 | PVT1 | RPS6KA5 | SPSB4 | | SPON1 | | KIAA1671 |
| MSGN1 | GRIK5 | RAB8B | TSPAN7 | | SOX13 | | MAP1B |
| DDIT4 | ARL4A | ZNF557 | CCDC92 | | GBX2 | | CDH6 |
| CPA6 | TTC7A | ZNF558 | GDPD1 | | PROM1 | | PHYHIPL |
| LRRN1 | DDR1 | RSPO3 | SYT11 | | HNF1B | | PMAIP1 |
| PTK7 | HEXB | CACNG4 | IRX5 | | WNK2 | | PRRG3 |
| UAP1L1 | PRODH | ARL4D | PAX6 | | KCNG1 | | HES3 |
| RETSAT | ZIC2 | GSTK1 | PCGF5 | | GLI1 | | RAB6B |
| FGF8 | TM7SF2 | SP4 | PMEL | | SCG5 | | ESYT1 |
| IER3 | GATM | GPC3 | CMTM8 | | NINJ1 | | ELOVL2 |
| RBM24 | COL18A1 | FABP7 | NRCAM | | FBXL16 | | GABRB3 |
| HOXD11 | ASPHD2 | TLN2 | SLC35G1 | | CELSR1 | | SERPINE2 |
| TF | SLC25A23 | SCARA5 | MAPK12 | | RPRM | | ZNF493 |
| DKK 1.00 | DCLK2 | HAS2 | SLC2A14 | | COL2A1 | | ZNF595 |
| MBLAC1 | SLC29A1 | LMO7 | SLC2A3 | | MUC1 | | ZNF681 |
| FGF4 | GLT1D1 | PAM | UPK1A | | AJAP1 | | ZNF724 |
| ST14 | VSTM2B | IFITM5 | NPDC1 | | CD24 | | ZNF85 |
| TMEM151B | SLC7A3 | LRRC55 | MTMR11 | | LMLN | | MORC4 |
| BLNK | CHST15 | CEP70 | C1GALT1C1 | | SLC50A1 | | SOBP |
| RFTN1 | PTN | ARAP1 | SCRN1 | | HSPA4L | | DTX3L |
| DNAJC22 | SNURF | ST6GALNAC6 | IRX3 | | SSBP2 | | AHNAK |
| WNT3A | CHAC1 | LIMCH1 | SYN1 | | CD47 | | DOCK9 |
| COL13A1 | ZIC5 | APLNR | USP51 | | FGFR2 | | RB1 |
| OXCT1 | TOM1L1 | GDF11 | EPHA2 | | PCDH1 | | METTL18 |
| ISLR | MID1IP1 | TMEM176A | NUAK2 | | CPNE2 | | PLAGL1 |
| EPHA1 | PRADC1 | ADAMTS6 | APELA | | PCBD1 | | LETM2 |

TABLE 2-B

| | | | | | |
|---|---|---|---|---|---|
| GNAL | SLC2A1 | S100A16 | DAB1 | NUP210 | GADD45A |
| LGALSL | CERCAM | RASSF9 | LRRC4 | APOBEC3B | BACH2 |
| SLC39A11 | TDRKH | ADAM12 | CHRDL1 | APOBEC3C | BICD1 |
| PROKR2 | TUBB3 | CNRIP1 | TMEM108 | APOBEC3D | GLIPR1 |
| SGTB | RRAGB | FGF19 | FRMD5 | APOBEC3F | EMB |
| CA3 | NCAM1 | | NKX2-8 | APOBEC3G | RSC1A1 |
| SLCO2B1 | LGI2 | | SCUBE2 | GAS7 | FREM1 |
| SAT2 | DLX5 | | SFRP2 | HSD1182 | PPM1E |
| GABRB2 | ARHGEF26 | | GPRC5A | PDZK1 | NOVA1 |
| DPPA3 | RND2 | | CCDC160 | PRDM12 | SFT2D2 |
| TCP11L1 | NUDT12 | | IGFBP5 | PARD3B | FRAS1 |
| C11orf42 | CYB561 | | ELAVL4 | TOR3A | LONRF2 |
| RIPK4 | EMILIN2 | | GPR153 | THSD4 | ASXL3 |
| VWASA | IRX1 | | GKAP1 | LRRC8C | WHRN |
| ERMARD | CLDN3 | | SOX1 | STON2 | LRRC8B |
| DNAJC12 | C15orf61 | | GFRA3 | FZD9 | INAFM2 |
| DHRS3 | DACT2 | | SERAC1 | SYNRG | CHD3 |
| LIPG | C22orf39 | | CADM4 | C1QL1 | CORO2B |
| SGK1 | ATP6V0E2 | | NELL2 | PIK3CB | ID2 |
| SERHL2 | ZCCHC18 | | CPN1 | TRAK1 | SLC22A23 |
| GK5 | PERP | | SERINC2 | NRBP2 | PROX1 |
| ARC | FAHD2A | | MAP3K9 | NIN | ZNF45 |
| GSTM2 | FAHD2B | | NLGN3 | STEAP1 | OVGP1 |
| JPH3 | SNX10 | | TPD52 | STEAP1B | FAM184B |
| NPTX2 | KCNE5 | | B3GALNT1 | ALDH1L2 | DCLK1 |
| CDH1 | RBM38 | | TOX2 | SPRY1 | ZFHX2 |
| | | | | | BEGAIN |
| | | | | | PODXL |
| | | | | | KIF1A |
| | | | | | IFNGR2 |
| | | | | | PPP1R3E |
| | | | | | ITGA6 |
| | | | | | OLIG3 |
| | | | | | OMD |

Supplementary Table 3 (Table 3-A to 3-D) Recombinant proteins, small molecules, reagents & consumables/equipment used in this study. List of utilized recombinant proteins & small molecules (3.1), modulators of signaling pathways (3.2) reagents (3.3) and consumables/equipment (3.4).

TABLE 3-A
Supplementary Table 3.1: Human recombinant
proteins & small molecule modulators

| Small Molecule | Company | Catalog Number | Description |
|---|---|---|---|
| all trans-Retinal | Sigma-Aldrich | R2500 | RA precursor |
| basic FGF (bFGF) | PeproTech | 100-18B | FGF signaling |
| Retinoic acid | Sigma-Aldrich | R2625 | RAR agonist |
| Retinol | Sigma-Aldrich | R7632 | RA precursor |

TABLE 3-B
Supplementary Table 3.2: Small molecule modulators
(agonists or inhibitors) of signaling pathways

| | | Catalog | |
|---|---|---|---|
| Modulators | Company | Number | Description |
| AGN193109 | Tocris | 5758 | pan-RAR inhibitor |
| BMS493 | Sigma-Aldrich | B6688 | inverse pan-RAR agonist |
| DAPT | Sigma-Aldrich | D5942 | NOTCH inhibitor |
| ER50891 | Tocris | 3823 | RARα receptor inhibitor |
| IWP-2 | Selleck Chemicals | S7085 | WNT inhibitor |
| PD0325901 | WAKO | 162-25291 | MEK inhibitor |
| PD173074 | Tocris | 3044 | FGFR inhibitor |
| SB431542 | Selleck Chemicals | S1067 | TGFβ inhibitor |
| XAV939 | Tocris | 3748 | WNT inhibitor |
| CHIR99021 | Nacalai | 18764-44 | WNT agonist |
| Y-27632 | Nacalai | 18188-04 | ROCK inhibitor |

TABLE 3-C
Supplementary Table 3.3: Reagents

| | | Catalog |
|---|---|---|
| Reagents | Company | Number |
| 0.5 mmol/I-EDTA/PBS solution | Nacalai | 13567-84 |
| 1x Protease Inhibitor | Roche | 11873580001 |
| Bovine Serum Albumin (BSA) | Nacalai | 01863-77 |
| Bovine Serum Albumin (BSA) | Sigma-Aldrich | A1595 |
| Chitin Resin | NEB | S6651 |
| Chromium Next GEM Single Cell 3′ | 10X Genomics | 10000269 |
| Kit v3.1 for Dual Index | | |
| Concanavalin A-coated beads | Bangs | BP531 |
| | Laboratories | |
| D-Luciferin Potassium Salt | bioWORLD | 40400076-1 |
| Frozen sectioning compound | Leica | 3801481 |
| HBSS(−) without Phenol Red | Wako | 085-09355 |
| KAPA HiFi HotStart PCR Kit | KAPA | KK2502 |
| | BIOSYSTEMS | |
| Matrigel | Corning | 356231 |
| Neural Tissue Dissociation Kit (P) | Miltenyi Biotec | 130-092-628 |
| NovaSeq 6000 S1 Reagent Kit v1.5 | Illumina | 20028319 |
| (100 Cycles) | | |
| NovaSeq 6000 SP Reagent Kit v1.5 | Illumina | 20028401 |
| (100 Cycles) | | |
| Penicillin-Streptomycin | Thermo Fisher | 15140122 |
| | Scientific | |
| Phosphatase Inhibitor Cocktail | Nacalai | 07574-61 |
| Phosphate buffered saline (PBS) tablets | Takara | T900 |
| Poly vinyl alcohol (PVA) | Nacalai | 11738-62 |
| StemProTM AccutaseTM cell | Thermo Fisher | A11105-01 |
| dissociation reagent (AccutaseTM) | Scientific | |
| T7 Express lysY/Iq Competent E. coli | NEB | C3013 |
| (High Efficiency) | | |
| Thermolabile Proteinase K | NEB | P8111S |
| TritonX-100 | Nacalai | 12967-45 |
| TrypLE Select enzyme | Thermo Fisher | 12563-011 |
| | Scientific | |
| TWEEN20 | Nacalai | 28353-85 |

TABLE 3-D
Supplementary Table 3.4: Consumables & equipments

| | | Catalog |
|---|---|---|
| Consumables & equipments | Company | Number |
| 12 multichannel pipettes | Eppendorf | 3122000060 |
| 24 well cell imaging plates | Eppendorf | 30741005 |
| 6 well sterile cell culture plate | WATSON | 197-06CPS |
| 96 well flat glass bottom sterile cell | Iwaki | 5866-096 |
| culture plate | | |
| 96 well flat-bottom sterile cell culture | WATSON | 197-96CS |
| plate | | |
| 96 well flat-bottom sterile cell culture | WATSON | 197-96CIS |
| plate | | |
| 96 well U-bottom ultra-low attachment | Sumitomo | MS-9096U |
| plate | Bakelite | |
| Cryomold | Sakura | 4566 |
| | Finetek | |
| Electronic pipette | Eppendorf | 4861000040 |
| Flowmi cell strainer | Merck | BAH136800040 |

Supplementary Table 4 (Table 4-A to 4-D): Antibodies used in this study. List of utilized primary antibodies for immunohistochemistry (4.1), secondary antibodies for immunohistochemistry (4.2), primary antibodies for CUT&Tag library preparation (4.3), secondary antibodies for CUT&Tag library preparation (4.4).

TABLE 4-A
Supplementary Table 4.1: Primary antibodies used for immunocytochemistry

| Antibody | Company | Catalog Number | Lot Number | Species/Clone Number | Dilution |
|---|---|---|---|---|---|
| β-catenin | BD Biosciences | 610153 | 8240536 | Mouse/Clone 14 | 1:100 |
| ETV2 | abcam | ab181847 | GR3357579-1 | Rabbit/EPR5229(3) | 1:500 |
| Fibronectin | DSHB | III-15-s | 11/19/15 | Mouse/13G3B7 Fibronectin III-15 | 1:10 |
| KDR | R&D Systems | AF357 | CUE0621081 | Polyclonal Goat IgG | 1:20 |
| MEOX1 | ATLAS | HPA045214 | A115362 | Polyclonal Rabbit IgG | 1:500 |
| MEOX1 | ORIGENE | TA804716 | F001 | Mouse/OTI5D8 | 1:500 |
| Phospho-p44/42 MAPK (Erk 1/2) | Cell Signaling | 4370 | 28 | Rabbit/D13.14.4E | 1:200 |
| PKCζ | Santa Cruz | SC-17781 | F2520 | Mouse/H-1 | 1:100 |
| SOX2 | Cell Signaling | 3579 | 8 | Rabbit/D6D9 | 1:400 |
| TBX6 | R&D Systems | AF4744 | CAPT0217111 | Polyclonal Goat IgG | 1:100 |
| TBXT | R&D Systems | AF2085 | KQP0618021 | Polyclonal Goat IgG | 1:100 |

TABLE 4-B
Supplementary Table 4.2: Secondary antibodies used for immunocytochemistry

| | | Catalog | | | |
|---|---|---|---|---|---|
| Antibody | Company | Number | Lot Number | Species | Dilution |
| Alexa Fluor ® 647 Phalloidin | Invitrogen | A22287 | 2101947 | | 1:200 |
| Alexa Fluor ™ 405 Donkey Anti-Goat IgG H&L | Abcam | ab175665 | GR3267053-3 | Polyclonal Donkey IgG | 1:500 |
| Alexa Fluor ™ 405 Donkey Anti-Mouse IgG H&L | Abcam | ab175658 | GR3258168-7 | Polyclonal Donkey IgG | 1:500 |
| Alexa Fluor ™ 488 Donkey Anti-Goat IgG (H + L) | Invitrogen | A11055 | 2059218 | Polyclonal Donkey IgG | 1:500 |
| Alexa Fluor ™ 488 Donkey Anti-Mouse IgG (H + L) | Invitrogen | A21202 | 2018296 | Polyclonal Donkey IgG | 1:500 |
| Alexa Fluor ™ 488 Donkey Anti-Rabbit IgG (H + L) | Invitrogen | A21206 | 2072687 | Polyclonal Donkey IgG | 1:500 |
| Alexa Fluor ™ 555 Donkey Anti-Goat IgG (H + L) | Invitrogen | A21432 | 2026158 | Polyclonal Donkey IgG | 1:500 |
| Alexa Fluor ™ 555 Donkey Anti-Mouse IgG (H + L) | Invitrogen | A31570 | 2045336 | Polyclonal Donkey IgG | 1:500 |
| Alexa Fluor ™ 555 Donkey Anti-Rabbit IgG (H + L) | Invitrogen | A31572 | 2017396 | Polyclonal Donkey IgG | 1:500 |

TABLE 4-C
Supplementary Table 4.3: Primary antibodies
used for CUT&Tag library preparation

| | | Catalog | Lot | Species/Clone | |
|---|---|---|---|---|---|
| Antibody | Company | Number | Number | Number | Dilution |
| H3K4me3 | MAB | MA304B | 18032 | Mouse/ | 1:100 |
| | Institute | | | MABI0304 | |
| H3K27me3 | MAB | MA323B | 19014 | Mouse/ | 1:100 |
| | Institute | | | MABI0323 | |

TABLE 4-D
Supplementary Table 4.4: Secondary antibodies
used for CUT&Tag library preparation

| | | Catalog | | | |
|---|---|---|---|---|---|
| Antibody | Company | Number | Lot Number | Species | Dilution |
| Rabbit | Abcam | ab46540 | GR3286449-5 | Polyclonal | 1:100 |
| Anti- | | | | Rabbit | |
| Mouse | | | | IgG | |
| IgG HL | | | | | |

Supplementary Table 5 (Table 5): List of probes used for HCR. List of utilized probes for HCR-based whole-mount in situ hybridization analysis of human axioloid samples.

TABLE 5
Supplementary Table 5.1: Probes used
for HCR in situ hybridization

| Probe | Accession No | Amplifier |
|---|---|---|
| ALDH1A2 | NM_003888.4 | 514 (B5) |
| AXIN2 | NM_004655.4 | 514 (B5) |
| CRABP2 | NM_001878.4 | 594 (B4) |
| CYP26A1 | NM_000783.4 | 594 (B4) |
| DUSP6 | NM_001946.4 | 647 (B3) |
| ETV2 | NM_014209.4 | 647 (B3) |
| FGF8 | NM_033163.5 | 546 (B2) |
| HES7 | NM_001165967.2 | 594 (B4) |
| KDR | NM_002253.4 | 488 (B1) |
| LEF1 | NM_016269.5 | 647 (B3) |
| LFNG | NM_001040167.2 | 488 (B1) |
| MESP2 | NM_001039958.2 | 647 (B3) |
| MSGN1 | NM_001105569.3 | 514 (B5) |
| RARA | NM_000964.4 | 488 (B1) |
| RARG | NM_000966.6 | 594 (B4) |
| RDH10 | NM_172037 | 488 (B1) |
| RIPPLY2 | NM_001009994.3 | 647 (B3) |
| SPRY4 | NM_001127496.3 | 514 (B5) |
| TBX18 | NM_001080508.3 | 546 (B2) |
| TCF15 | NM_004609.3 | 594 (B4) |
| UNCX | NM_001080461.3 | 488 (B1) |
| WNT3A | NM_033131.4 | 488 (B1) |

Supplementary Table 6 (Table 6-A to 6-R): List of probe sequences used for HybISS analysis. List of utilized probe sequences used for HybISS-based spatial transcriptomic analysis of human axioloid samples.

TABLE 6-A

| Gene | | padlock probe sequence | |
|---|---|---|---|
| TCF15 | probe1 | GGCTGCTGCGAGGGCCCTTAACTCGCGTTCAAGTATGCGTCTATTTAGTGGAGCCTCGGACCAGTCGTTC | SEQ ID NO: 4 |
| | proba2 | GTGAACACGGCCTTCCCTTAACTCGCGTTCAAGTATGGGTCTATTTAGTGGAGCCGACCGCACTCAGAGC | SEQ ID NO: 5 |
| | probe3 | GCCGTCGTGACCTGGCCTTAACTCGCGTTCAAGTATGCGTCTATTTAGTGGAGCCAGCGCAAGGGGGGTG | SEQ ID NO: 6 |
| | proba4 | ATCCATTCAGCCTGCCCTTAACTCGCGTTCAAGTATGCGTCTATTTAGTGGAGCCTGGAGAGCTGTGAGG | SEQ ID NO: 7 |
| | probe5 | CAAACTTTCCTCAGTCCTTAACTCGCGTTCAAGTATGCGTCTATTTAGTGGAGCCTCCGGGCGCCATAAC | SEQ ID NO: 8 |
| TBX6 | probe 1 | TCCCTGGGGGCCGGCGGTTGCTACGATGACTCAGTTGCGTCTATTTAGTGGAGCCCGAGAATTGTACCCG | SEQ ID NO: 9 |
| | probe2 | ACATTCACCCCGACTGGTTGCTACGATGACTCAGTTGCGTCTATTTAGTGGAGCCTGCCTGACCGTGTCT | SEQ ID NO: 10 |
| | probe3 | TGGAGACATTCGTGAGGTTGCTACGATGACTCAGTTGCGTCTATTTAGTGGAGCCCGACTCCACCCTGGG | SEQ ID NO: 11 |
| | probe4 | ACTGAACCACTGCTGGGTTGCTACGATGACTCAGTTGCGTCTATTTAGTGGAGCCGCTCCAAACCCATGT | SEQ ID NO: 12 |
| | probe5 | CTGTTGTTCTGGGACGGTTGCTACGATGACTCAGTTGCGTCTATTTAGTGGAGCCGCAGGCCACACCAGT | SEQ ID NO: 13 |
| TBXT | probe1 | GACCACCTGCTGAGCATAGACTTAACGCACGATCCTGCGTCTATTTAGTGGAGCCCTGCAGTACCGAGTG | SEQ ID NO: 14 |
| | probe2 | CCCGTCTCCTTCAGCATAGACTTAACGCACGATCCTGCGTCTATTTAGTGGAGCCCACTGGATGAAGGCT | SEQ ID NO: 15 |
| | proba3 | CAACGGCGCCGTCACATAGACTTAACGCACGATCCTGCGTCTATTTAGTGGAGCCCCTGTGGTCTGTGAG | SEQ ID NO: 16 |
| | prob&4 | TGTCGTGGCAGCCAGATAGACTTAACGCACGATCCTGCGTCTATTTAGTGGAGCCGATGCAGTGACTTTT | SEQ ID NO: 17 |
| | proba5 | CTAGAATGTGTGTATATAGACTTAACGCACGATCCTGCGTCTATTTAGTGGAGCCTGACTGCTCTGCCCC | SEQ ID NO: 18 |
| MESP2 | probe 1 | CTGCGACGGCGCCCGCGGACTAAGGGCGACTAATATGCGTCTATTTAGTGGAGCCGTCGGGTTCGTGCCC | SEQ ID NO: 19 |
| | probe2 | GTCCCCTTGGGGCTGCGGACTAAGGGCGACTAATATGCGTCTATTTAGTGGAGCCGCGGGGGGACGGGGG | SEQ ID NO: 20 |
| | probe3 | TCGTCCCCAGAGCCCCGGACTAAGGGCGACTAATATGCGTCTATTTAGTGGAGCCTGTCGCTGGACGCAG | SEQ ID NO: 21 |
| | probe4 | CCCTGGATGGAGTCACGGACTAAGGGCGACTAATATGCGTCTATTTAGTGGAGCCGATAGCGGGTACCTT | SEQ ID NO: 22 |
| | probe5 | GTTGGCCAGAGCCCTCGGACTAAGGGCGACTAATATGCGTCTATTTAGTGGAGCCTGCTTGTTGGTGACA | SEQ ID NO: 23 |
| MEOX1 | probe1 | CCGGCGCAGGCCCAAGAACTATGCTGACAGTACCGTGCGTCTATTTAGTGGAGCCCCCTGTCTCAGACGC | SEQ ID NO: 24 |
| | probe2 | ACCCAACTACCCCCAGAACTATGCTGACAGTACCGTGCGTCTATTTAGTGGAGCCAGGACTGAGCCCCCT | SEQ ID NO: 25 |
| | probe3 | CGACTCGGAAAGTAAGAACTATGCTGACAGTACCGTGCGTCTATTTAGTGGAGCCTTTGGAACCAGCTGG | SEQ ID NO. 26 |
| | proba4 | GGCTTGACTGGGTGGGAACTATGCTGACAGTACCGTGCGTCTATTTAGTGGAGCCAGGCCCGTCCACACA | SEQ ID NO: 27 |
| | proba5 | GAAGGTTCTCCAGTGGAACTATGCTGACAGTACCGTGCGTCTATTTAGTGGAGCCTGGAACCCTGGAGTG | SEQ ID NO: 28 |
| RiPPLY2 | probe1 | GGCGATGCCCGATGGATATCTTTAACGTCGAGCGCTGCGTCTATTTAGTGGAGCCGAACCACGCCGCGGA | SEQ ID NO: 29 |
| | probe2 | TTACCAATTCAGGCAATATCITTAACGTCGAGCGCTGCGTCTATTTAGTGGAGCCAGCCTCAGGAAAGCT | SEQ ID NO: 30 |
| | probe3 | GAGGCAAGAAAGAAGAGGAGATATCTTTAACGTCGAGCGCTGCGTCTATTTAGTGGAGCCAGCCGTGACT | SEQ ID NO: 31 |
| | | ATCGACTGAGACCCTGGGTGGACGCCG | |
| | probe4 | CTGAAAAATTTTCCAATTCAATATCTTTAACGTCGAGCGCTGCGTCTATTTAGTGGAGCCAGCCGTGACT | SEQ ID NO: 32 |
| | | ATCGACTATCAAGAAGCAGAAGCTCTT | |
| | probe5 | ATCCAAAGCTTGTGGGGTTTATATCTTTAACGTCGAGCGCTGCGTCTATTTAGTGGAGCCAGCCGTGACT | SEQ ID NO: 33 |
| | | ATCGACTGATTAGCTACTTTTGATTAT | |
| HES7 | probe1 | AGCGAAGCCGGGTGGCGGTCAACTCTACCTAAGGATGCGTCTATTTAGTGGAGCCTGGGCTACTTGAGGG | SEQ ID NO: 34 |
| | probe2 | CGAGCAGGTGTTGGGCGGTCAACTCTACCTAAGGATGCGTCTATTTAGTGGAGCCACCCGGGCGTCCGGG | SEQ ID NO: 35 |
| | probe3 | TCCCTAGTGCATGGACGGTCAACTCTACCTAAGGATGCGTCTATTTAGTGGAGCCCAGTCCCTTCCCGAG | SEQ ID NO: 36 |
| | jprobe4 | AGAGCCACCCGACGACGGTCAACTCTACCTAAGGATGGGTCTATTTAGTGGAGCCTATCCATTGCGCCAC | SEQ ID NO. 37 |
| | proba5 | ATATAGTCCCACCCACGGTCAACTCTACCTAAGGATGCGTCTATTTAGTGGAGCCGCTCCCCACTCCCAG | SEQ ID NO: 38 |

TABLE 6-B

| | | | |
|---|---|---|---|
| JACTB | probe8 | CGCTCGTCGTCGACATTCGTCGGATCGAGGTTATCTGCGTCTATTTAGTGGAGCCATGATGATATCGCCG | SEQ ID NO: 39 |
| | probe9 | CGGTGAGGGGGTCACTTCGTCGGATCGAGGTTATCTGCGTCTATTTAGTGGAGCCCATCGTGATGGACTC | SEQ ID NO: 40 |
| | probe10 | CCGACAGGATGCAGATTCGTCGGATCGAGGTTATCTGCGTCTATTTAGTGGAGCCTGTACCCTGGCATTG | SEQ ID NO: 41 |
| | probe11 | TGTGGATCGGCGGCTTTCGTCGGATCGAGGTTATCTGCGTCTATTTAGTGGAGCCAGCGCAAGTACTCCG | SEQ ID NO: 42 |

TABLE 6-C

| Gene | | padlock probe sequence | |
|---|---|---|---|
| HOXD8 | probe1 | TTGGCGAGGACCCAGCACGGATAACTACTAGACCGTGCGTCTATTTAGTGGAGCCCGTCCAGTGGTAATA | SEQ ID NO: 43 |
| | probe2 | CCTGACAAATTAACTCACGGATAACTACTAGACCGTGCGTCTATTTAGTGGAGCCAGACAGAGCCGAAGG | SEQ ID NO: 44 |
| | probe3 | AATCGCCTTGTAAAACACGGATAACTACTAGACCGTGCGTCTATTTAGTGGAGCCCTGGCAACTCTGGCA | SEQ ID NO: 45 |
| | probe4 | GTTATTTTCAGTAGGCACGGATAACTACTAGACCGTGCGTCTATTTAGTGGAGCCCTGCCGCTGTGTTCC | SEQ ID NO: 46 |
| | probe5 | GCGATAGCCTCACCTCACGGATAACTACTAGACCGTGCGTCTATTTAGTGGAGCCCTGTTCTTCAGGAAA | SEQ ID NO: 47 |
| HOXB1 | probe1 | GACTATAATAGGATGCCTTAGACACATCATACGGATGCGTCTATTTAGTGGAGCCCGGCCTTGACGCATG | SEQ ID NO: 48 |
| | probe2 | CTCGAGCTACGGGGCCCTTAGACACATCATACGGATGCGTCTATTTAGTGGAGCCAGGCTATTTTCATCC | SEQ ID NO: 49 |
| | probe3 | GCACCTCCCCGGAAGCCTTAGACACATCATACGGATGCGTCTATTTAGTGGAGCCCAGACCAGTCGACAT | SEQ ID NO: 50 |
| | probe4 | AGCACCAGCTGCCTTCCTTAGACACATCATACGGATGCGTCTATTTAGTGGAGCCCACAGACCTTACAAT | SEQ ID NO: 51 |
| | probe5 | AGCTGCAAGGATTTACCTTAGACACATCATACGGATGCGTCTATTTAGTGGAGCCATGAGGGCTCCCAAC | SEQ ID NO: 52 |
| HOXC6 | probe1 | TGGAGCGGCCGTTGCGCCTAGATACCGTAGATGACTGCGTCTATTTAGTGGAGCCGCATTTCTCGACCTA | SEQ ID NO: 53 |
| | probe2 | AGTGGGGTCGGCTACGCCTAGATACCGTAGATGACTGCGTCTATTTAGTGGAGCCCGAATGAATTCGCAC | SEQ ID NO: 54 |
| | probe3 | TCGGGGGGCGGCGGAGCCTAGATACCGTAGATGACTGCGTCTATTTAGTGGAGCCCTCACATCCACTCTC | SEQ ID NO: 55 |
| | probe4 | CCACGCGCCTCCTCCGCCTAGATACCGTAGATGACTGCGTCTATTTAGTGGAGCCCACAACTCTCTTTCA | SEQ ID NO: 56 |
| | probe5 | GTCTACAGGCCCTTTGCCTAGATACCGTAGATGACTGCGTCTATTTAGTGGAGCCGCTCGTTCTCGGCTT | SEQ ID NO: 57 |
| HOXC10 | probe1 | GTATTGCTCCTTAAATAGACGTAGTGAGCATGACTTGCGTCTATTTAGTGGAGCCAGCTCCTCCGCTGTA | SEQ ID NO: 58 |
| | probe2 | GTGTCAAGGAGGAGATAGACGTAGTGAGCATGACTTGCGTCTATTTAGTGGAGCCGCTCCTACCCACCTA | SEQ ID NO: 59 |
| | probe3 | CGAAAAGGAGAGGGCTAGACGTAGTGAGCATGACTTGCGTCTATTTAGTGGAGCCGAGCCCCTCGGAGAG | SEQ ID NO: 60 |
| | probe4 | TGCAATGCGACTGCATAGACGTAGTGAGCATGACTTGCGTCTATTTAGTGGAGCCCTCCCTGAGTATAAA | SEQ ID NO: 61 |
| | probe5 | ATATTGTCCTGTCCCTAGACGTAGTGAGCATGACTTGCGTCTATTTAGTGGAGCCTGCCCCCCCCCCCAA | SEQ ID NO: 62 |
| HOXA5 | probe1 | GACTCGGCGAGCATGCGGACATTACCACCTAGAGTTGCGTCTATTTAGTGGAGCCAGCGAGCAATTCAGG | SEQ ID NO: 63 |
| | probe2 | GATCCGCTGCCCTGCCGGACATTACCACCTAGAGTTGCGTCTATTTAGTGGAGCCCACTCTCCTCAGCCC | SEQ ID NO: 64 |
| | probe3 | ACGGCCTACACGCGCCGGACATTACCACCTAGAGTTGCGTCTATTTAGTGGAGCCGGCAAAAGGGCCCGG | SEQ ID NO: 65 |
| | probe4 | GTCAGTACTAAGGTGCGGACATTACCACCTAGAGTTGCGTCTATTTAGTGGAGCCCGGATCCCGCGTAGT | SEQ ID NO: 66 |
| | probe5 | AGCTTCAGTGATGTACGGACATTACCACCTAGAGTTGCGTCTATTTAGTGGAGCCGGCTCTTATAAGCGC | SEQ ID NO: 67 |
| HOXD12 | probe1 | AGGCCGAATGGCGGCCTAGACAGACTGCGACATACTGCGTCTATTTAGTGGAGCCTACTTCTCCAACCTG | SEQ ID NO: 68 |
| | probe2 | CCCGACGGCCTGCCGCTAGACAGACTGCGACATACTGCGTCTATTTAGTGGAGCCCTGCGACCTTCACTG | SEQ ID NO: 69 |
| | probe3 | GGTCTCTGCAGCAGACTAGACAGACTGCGACATACTGCGTCTATTTAGTGGAGCCCTGTGGGTACAATTA | SEQ ID NO: 70 |
| | probe4 | CTTTAAGGCCCTGGGCTAGACAGACTGCGACATACTGCGTCTATTTAGTGGAGCCCTGTCCCAGTGGAGA | SEQ ID NO: 71 |
| | probe5 | ACTGACTTCAGTTGACTAGACAGACTGCGACATACTGCGTCTATTTAGTGGAGCCCTGAGCCTTAGCCCA | SEQ ID NO: 72 |
| HOXD10 | probe1 | AAGAGAAGTGAACCACTAGACATATCTCACAGCGATGCGTCTATTTAGTGGAGCCCCCGTCTCTGGCCAA | SEQ ID NO: 73 |
| | probe2 | CCTGAGGTTCCCGTCCTAGACATATCTCACAGCGATGCGTCTATTTAGTGGAGCCTGTCCCGTTGAGAAC | SEQ ID NO: 74 |
| | probe3 | CGTGAGCGGCCAGGACTAGACATATCTCACAGCGATGCGTCTATTTAGTGGAGCCGAAGATGAACGAGCC | SEQ ID NO: 75 |
| | probe4 | CACCGACAGGCAGGTCTAGACATATCTCACAGCGATGCGTCTATTTAGTGGAGCCTAAGAGCGTTAACCT | SEQ ID NO: 76 |

TABLE 6-D

| | | | |
|---|---|---|---|
| | probe5 | TGCATCTGTGGTTTGCTAGACATATCTCACAGCGATGCGTCTATTTAGTGGAGCCGCCTTTCCCTTGTGG | SEQ ID NO: 77 |
| HOXB9 | probe1 | GGTGTTCGGCGCCTCCTTTCCAATACGGTGTACGATGCGTCTATTTAGTGGAGCCCCAGCCCAAAGCGCC | SEQ ID NO: 78 |
| | probe2 | AGTCAAGCTCCTAGTCTTTCCAATACGGTGTACGATGCGTCTATTTAGTGGAGCCGGAAAGAGAGACCCC | SEQ ID NO: 79 |
| | probe3 | TAGAAAGTACAAGAACTTTCCAATACGGTGTACGATGCGTCTATTTAGTGGAGCCCCCAAGCCGGTGGGC | SEQ ID NO: 80 |
| | probe4 | TCCGAATGAGAATTTCTTTCCAATACGGTGTACGATGCGTCTATTTAGTGGAGCCGCCAGGCCAGGAGAG | SEQ ID NO: 81 |
| | probe5 | CTCTAATTGTTTGTTCTTTCCAATACGGTGTACGATGCGTCTATTTAGTGGAGCCGCGAGGGGCCTAGAG | SEQ ID NO: 82 |
| HOXA10 | probe1 | ACGGACAGACAAGTGCTTTCGACGCTGACACTAAATGCGTCTATTTAGTGGAGCCCGCAGCGTCCACCTC | SEQ ID NO: 83 |
| | probe2 | CCGAGCGCTGGTCCCCTTTCGACGCTGACACTAAATGCGTCTATTTAGTGGAGCCCGGTTTTTTCACTTC | SEQ ID NO: 84 |
| | probe3 | CTTGGGGTCCCACTGCTTTCGACGCTGACACTAAATGCGTCTATTTAGTGGAGCCGGCTGGGTTTGCTGT | SEQ ID NO: 85 |
| | probe4 | TGCCTGGAGTGCTGTCTTTCGACGCTGACACTAAATGCGTCTATTTAGTGGAGCCGCCACCGGGCATGTC | SEQ ID NO: 86 |
| | probe5 | GTGGAAAAAGACGATCTTTCGACGCTGACACTAAATGCGTCTATTTAGTGGAGCCGTATTGCTGCTGTGC | SEQ ID NO: 87 |
| HOXC11 | probe1 | CAAAACCTCCATCCGGCACTATTACGCAGAGCATCTGCGTCTATTTAGTGGAGCCTCGCTAGACCGGGTC | SEQ ID NO: 88 |
| | probe2 | GGCTCCTACGGCGGCGCACTATTACGCAGAGCATCTGCGTCTATTTAGTGGAGCCCTCATGAAAAACGAA | SEQ ID NO: 89 |
| | probe3 | GTGAAGGGAAGTGTCGCACTATTACGCAGAGCATCTGCGTCTATTTAGTGGAGCCGGGCCTGGAAAGGGG | SEQ ID NO: 90 |
| | probe4 | TAGCTACTCTGAAGCGCACTATTACGCAGAGCATCTGCGTCTATTTAGTGGAGCCGCTGAGGACATTCCA | SEQ ID NO: 91 |
| | probe5 | GACAACTCATCTCATGCACTATTACGCAGAGCATCTGCGTCTATTTAGTGGAGCCGGGGCAGAGGTTCAG | SEQ ID NO: 92 |
| HOXC8 | probe1 | GCACCGAGGCGCCCCGGTATAGTACATTCCCGCACTGCGTCTATTTAGTGGAGCCATCCGGCCGAGCTCA | SEQ ID NO: 93 |
| | probe2 | AGCAGAACCCGTGCTGGTATAGTACATTCCCGCACTGCGTCTATTTAGTGGAGCCCCAACTCAGGCTACC | SEQ ID NO: 94 |
| | probe3 | GAAGTCTCTCATGCCGGTATAGTACATTCCCGCACTGCGTCTATTTAGTGGAGCCCGAAAACGTCGGATT | SEQ ID NO: 95 |
| | probe4 | CCTCGAAATGCAGAAGGTATAGTACATTCCCGCACTGCGTCTATTTAGTGGAGCCTTTAGAGGGGAGCCC | SEQ ID NO: 96 |
| | probe5 | TTTTATTGTGCTTCTGGTATAGTACATTCCCGCACTGCGTCTATTTAGTGGAGCCGCCGGTCCTGTGCGC | SEQ ID NO: 97 |
| HOXD3 | probe1 | CTATGAAAACCCAGGGGTATGCGACCTACTCTTACTGCGTCTATTTAGTGGAGCCGCAGAAGGCTGCTTA | SEQ ID NO: 98 |
| | probe2 | AGAACTCCAAGCAGAGGTATGCGACCTACTCTTACTGCGTCTATTTAGTGGAGCCTGAAAGAGTCTCGAC | SEQ ID NO: 99 |
| | probe3 | CACTCAGCAGCTGCCGGTATGCGACCTACTCTTACTGCGTCTATTTAGTGGAGCCCCGCCTACACGGCGC | SEQ ID NO: 100 |
| | probe4 | AATTACCTCTCTTGCGGTATGCGACCTACTCTTACTGCGTCTATTTAGTGGAGCCCCGAACTCGCGGCAA | SEQ ID NO: 101 |
| | probe5 | TGGCACAACACACTTGGTATGCGACCTACTCTTACTGCGTCTATTTAGTGGAGCCGGAAGCCCGGTGGCG | SEQ ID NO: 102 |
| HOXD1 | probe1 | GTACGTGTCATGCAGGTCTGAGAATGTGTTAGCACTGCGTCTATTTAGTGGAGCCGAGCTCCTACCTGGA | SEQ ID NO: 103 |
| | probe2 | CCGGCTTGTCTCAAAGTCTGAGAATGTGTTAGCACTGCGTCTATTTAGTGGAGCCGAACCCGGCCCTTTT | SEQ ID NO: 104 |
| | probe3 | TGGGGCCGCTAGCCCGTCTGAGAATGTGTTAGCACTGCGTCTATTTAGTGGAGCCCAAACTCGCCGAGTA | SEQ ID NO: 105 |
| | probe4 | TCTAGACTTAGGAGCGTCTGAGAATGTGTTAGCACTGCGTCTATTTAGTGGAGCCCGACCCCCATCCCTA | SEQ ID NO: 106 |
| | probe5 | GCTCAGAAGAGCACCGTCTGAGAATGTGTTAGCACTGCGTCTATTTAGTGGAGCCTCAGCACTCTGATGT | SEQ ID NO: 107 |
| HOXA9 | probe1 | CCGGGGACCCTGGGCGTCTGCATAGAGAACAACGTTGCGTCTATTTAGTGGAGCCGTTGGCCGCTATGCG | SEQ ID NO: 108 |
| | probe2 | GAACCCAGTGCACGCGTCTGCATAGAGAACAACGTTGCGTCTATTTAGTGGAGCCGTTTGGCGCCTCGTG | SEQ ID NO: 109 |
| | probe3 | GTACATGCGCTCCTGGTCTGCATAGAGAACAACGTTGCGTCTATTTAGTGGAGCCGGCGCCGGACGGCAG | SEQ ID NO: 110 |
| | probe4 | TAAACCTGAACCGCTGTCTGCATAGAGAACAACGTTGCGTCTATTTAGTGGAGCCCCGGCCTTATGGCAT | SEQ ID NO: 111 |

TABLE 6-E

| | | | |
|---|---|---|---|
| | probe5 | TGACACTCACACTTTGTCTGCATAGAGAACAACGTTGCGTCTATTTAGTGGAGCCTGACTGTCCCACGCT | SEQ ID NO: 112 |
| HOXA2 | probe1 | CCTAGGCCTTGCCCCACCTAATAACACTCACGGGTTGCGTCTATTTAGTGGAGCCTGCGCTCGCCTTTTT | SEQ ID NO: 113 |
| | probe2 | CTCGCCACGGCGCTGACCTAATAACACTCACGGGTTGCGTCTATTTAGTGGAGCCACCCCGGCAGTCACC | SEQ ID NO: 114 |
| | probe3 | AATCGCCGATGGCAGACCTAATAACACTCACGGGTTGCGTCTATTTAGTGGAGCCCAAAGAATCCCTGGA | SEQ ID NO: 115 |
| | probe4 | AACCAGCAATGAGAAACCTAATAACACTCACGGGTTGCGTCTATTTAGTGGAGCCCCCAGTCTCGCCTTT | SEQ ID NO: 116 |
| | probe5 | TTGCAGCATCTGAATACCTAATAACACTCACGGGTTGCGTCTATTTAGTGGAGCCCTCACCACAATCGAC | SEQ ID NO: 117 |
| HOXA11 | probe1 | ATGACATACTCCTACACCTACCTAAAGTGACATCGTGCGTCTATTTAGTGGAGCCCCGTCTTCGCGCCCA | SEQ ID NO: 118 |
| | probe2 | CCGCAAAAAGCGCTGACCTACCTAAAGTGACATCGTGCGTCTATTTAGTGGAGCCCAGTGGCCAACGCAC | SEQ ID NO: 119 |
| | probe3 | GCTGTGTGCTCTCCAACCTACCTAAAGTGACATCGTGCGTCTATTTAGTGGAGCCCCCCTGAGTAAAAAA | SEQ ID NO: 120 |
| | probe4 | GTTTGGCAAACTCTCACCTACCTAAAGTGACATCGTGCGTCTATTTAGTGGAGCCCTGTGGAGTGTGGCA | SEQ ID NO: 121 |
| | probe5 | AGGCAACTGGGCACAACCTACCTAAAGTGACATCGTGCGTCTATTTAGTGGAGCCTTGCAGCACTTATAC | SEQ ID NO: 122 |
| HOXA13 | probe1 | GGCGCACCCGGCGCCAGATACTTAACCATAGCGCCTGCGTCTATTTAGTGGAGCCGTGCCGCAACCTGAT | SEQ ID NO: 123 |
| | probe2 | ATGGAAAGCTACCAGAGATACTTAACCATAGCGCCTGCGTCTATTTAGTGGAGCCCCCTTGGGTCTTCCC | SEQ ID NO: 124 |
| | probe3 | CCACGACGAATCTCTAGATACTTAACCATAGCGCCTGCGTCTATTTAGTGGAGCCGGAGGCGGATATCAG | SEQ ID NO: 125 |
| | probe4 | GCTGTCGTATTTTAAAGATACTTAACCATAGCGCCTGCGTCTATTTAGTGGAGCCAGCAGCAATGCCTAG | SEQ ID NO: 126 |
| | probe5 | CTGCCATCAAGCCAAAGATACTTAACCATAGCGCCTGCGTCTATTTAGTGGAGCCCCATTTCCAAATGAG | SEQ ID NO: 127 |
| HOXA1 | probe1 | ACTCGGGGACCTGCTATAGAGCGAACGATAGTTGCTGCGTCTATTTAGTGGAGCCTACTTAGCAGTGGCG | SEQ ID NO: 128 |
| | probe2 | AATCAGGAAGCAGACATAGAGCGAACGATAGTTGCTGCGTCTATTTAGTGGAGCCAGCCCCTACGCGTTA | SEQ ID NO: 129 |
| | probe3 | GCGTTCCTTCCCCGGATAGAGCGAACGATAGTTGCTGCGTCTATTTAGTGGAGCCCCAGCTCTTCGCCCT | SEQ ID NO: 130 |
| | probe4 | GGGTGCCAGCATACAATAGAGCGAACGATAGTTGCTGCGTCTATTTAGTGGAGCCACCTTCATCCAGATT | SEQ ID NO: 131 |
| | probe5 | TATTTGGCTGGGCTAATAGAGCGAACGATAGTTGCTGCGTCTATTTAGTGGAGCCGCTTAAGCATCCGTG | SEQ ID NO: 132 |
| HOXB2 | probe1 | TCCCCCACCATTGAAATAGAGTCAACGAGCGTACCTGCGTCTATTTAGTGGAGCCCCCCCAGCAGCCCCC | SEQ ID NO: 133 |
| | probe2 | GGCCTGCTCTGCCGCATAGAGTCAACGAGCGTACCTGCGTCTATTTAGTGGAGCCAGCGAGCCGAAGATG | SEQ ID NO: 134 |
| | probe3 | GGAACCCGCGGCCAGATAGAGTCAACGAGCGTACCTGCGTCTATTTAGTGGAGCCCTGCGACCCTGCCGA | SEQ ID NO: 135 |
| | probe4 | CCCTTTTTCCGAGGAATAGAGTCAACGAGCGTACCTGCGTCTATTTAGTGGAGCCTCTCGACAGCCCGGT | SEQ ID NO: 136 |
| | probe5 | GCATAGACTTATGTGATAGAGTCAACGAGCGTACCTGCGTCTATTTAGTGGAGCCCTCCCACCCTCAGTC | SEQ ID NO: 137 |
| HOXD11 | probe1 | GCCTTCGTTCCTTTCCCGTCAGAAGCCATACTAGATGCGTCTATTTAGTGGAGCCTGACTTCGCTAGCAA | SEQ ID NO: 138 |
| | probe2 | ACTACGCGGCGGCGGCCGTCAGAAGCCATACTAGATGCGTCTATTTAGTGGAGCCGGGGCTACGCTCCCT | SEQ ID NO: 139 |
| | probe3 | CAGCGCCCGGGCCCCCCGTCAGAAGCCATACTAGATGCGTCTATTTAGTGGAGCCACCAGTTCTACGAGG | SEQ ID NO: 140 |
| | probe4 | CGGGACCTCCCAGCGCCGTCAGAAGCCATACTAGATGCGTCTATTTAGTGGAGCCTTCCCACGGTCAACT | SEQ ID NO: 141 |
| | probe5 | CGCTGGTGTTTTGCTCCGTCAGAAGCCATACTAGATGCGTCTATTTAGTGGAGCCGGGGTTGCTAGAAGG | SEQ ID NO: 142 |
| HOXA7 | probe1 | GCGGCTACGGGGCGGCCGTCGATACAGACTCAGATTGCGTCTATTTAGTGGAGCCCCAACTCACAGAGAA | SEQ ID NO: 143 |
| | probe2 | CGACTGCCGCCGCAGCCGTCGATACAGACTCAGATTGCGTGTATTTAGTGGAGCCATAAGGACGAAGGTC | SEQ ID NO: 144 |
| | probe3 | CCGACTGCTCCCAGGCCGTCGATACAGACTCAGATTGCGTCTATTTAGTGGAGCCGCCTGCTACCTAGTG | SEQ ID NO: 145 |
| | probe4 | GAATGTTCCTGAGCTCCGTCGATACAGACTCAGATTGCGTCTATTTAGTGGAGCCCCCACTTCTGGAGGG | SEQ ID NO: 146 |

TABLE 6-F

| | | | |
|---|---|---|---|
| | probe5 | ACTTCCCAGCCCAGCCCGTCGATACAGACTCAGATTGCGTCTATTTAGTGGAGCCGTTCAGGGAAGGCAC | SEQ ID NO: 147 |
| HOXA4 | probe1 | CCCAGTTATAACGGACTAGACTGAATCATCACCGATGCGTCTATTTAGTGGAGCCGTCAGCGCCGTTAAC | SEQ ID NO: 148 |
| | probe2 | CCGGCGGCGCCGCATCTAGACTGAATCATCACCGATGCGTCTATTTAGTGGAGCCCAATCGATACCTGAC | SEQ ID NO: 149 |
| | prabe3 | AGAGAGAACAGTTGTCTAGACTGAATCATCACCGATGCGTCTATTTAGTGGAGCCGCTGGTTGCCACCCA | SEQ ID NO: 150 |
| | probe4 | CATACACTTGCATCTCTAGACTGAATCATCACCGATGCGTCTATTTAGTGGAGCCCATGCTGAGTGGGGA | SEQ ID NO: 151 |
| | probe5 | CTGGGGCACGGTCTTCTAGACTGAATCATCACCGATGCGTCTATTTAGTGGAGCCTACTGTTGTCCCCTT | SEQ ID NO: 152 |
| HOXC4 | probe1 | GCATCACCACCAGGACTCTGTGAATACCGAGTACATGCGTCTATTTAGTGGAGCCGGAATCGGGATTCCA | SEQ ID NO: 153 |
| | probe2 | GGCCACGGGCCGGCCCTCTGTGAATACCGAGTACATGCGTCTATTTAGTGGAGCCCCCGGCAATTCGCGA | SEQ ID NO: 154 |
| | probe3 | CTATACCCGGCAGCACTCTGTGAATACCGAGTACATGCGTCTATTTAGTGGAGCCGCGCTCGAGGACAGC | SEQ ID NO: 155 |
| | probe4 | GTACTTCTGAAGACCCTCTGTGAATACCGAGTACATGCGTCTATTTAGTGGAGCCCGGCAGCTACCCCGG | SEQ ID NO: 156 |
| | probe5 | CCTCTCAGAGCTGTTCTCTGTGAATACCGAGTACATGCGTCTATTTAGTGGAGCCAATGCATCTGGAGAG | SEQ ID NO: 157 |
| HOXC5 | probe1 | CCGGTTCCTGTCCCTGGTCAGAGACGTAGCATCAATGCGTCTATTTAGTGGAGCCAGAGCGCGCCCCTAG | SEQ ID NO: 158 |
| | probe2 | AGCTCGGTACCCGGGGGTCAGAGACGTAGCATCAATGCGTCTATTTAGTGGAGCCCCCTCAGCTCGGCTC | SEQ ID NO: 159 |
| | probe3 | CAGCCAAAGGCTGGCGGTCAGAGACGTAGCATCAATGCGTCTATTTAGTGGAGCCGCTGGACGGGTTAGA | SEQ ID NO: 160 |
| | probe4 | GGGGTTTCCTGGCACGGTCAGAGACGTAGCATCAATGCGTCTATTTAGTGGAGCCGCTAGCTCAACTAGT | SEQ ID NO: 161 |
| | probe5 | TCTGTGCCCTCCTGAGGTCAGAGACGTAGCATCAATGCGTCTATTTAGTGGAGCCTCCCTGCCACGAATT | SEQ ID NO: 162 |
| HOXA3 | probe1 | CTACCCCTACCAGGCGTCTGGAGAGATCAGTATCCTGCGTCTATTTAGTGGAGCCGGCGATCTACGGTGG | SEQ ID NO: 163 |
| | probe2 | GGCTTCGTCCAAGCGGTCTGGAGAGATCAGTATCCTGCGTCTATTTAGTGGAGCCGAGCCCGCCGGGGCA | SEQ ID NO: 164 |
| | probe3 | ACGGCGGCAGGGGCGGTCTGGAGAGATCAGTATCCTGCGTCTATTTAGTGGAGCCCCACAGAAGCGCTAC | SEQ ID NO: 165 |
| | probe4 | CACCATCCTTCTCAGGTCTGGAGAGATCAGTATCCTGCGTCTATTTAGTGGAGCCACGGACCTTACCGGC | SEQ ID NO: 166 |
| | probe5 | CGGTCTTCTGCTCCAGTCTGGAGAGATCAGTATCCTGCGTCTATTTAGTGGAGCCGGGGATAACGCAGGG | SEQ ID NO: 167 |
| HOXB5 | probe1 | CGGCTACAATTACAATACTATGCCTACCTGTCGATTGCGTCTATTTAGTGGAGCCGCACACCGGCTCTTA | SEQ ID NO: 168 |
| | probe2 | TCCGACCAGGCGACCTACTATGCCTACCTGTCGATTGCGTCTATTTAGTGGAGCCTCTGCTTCGTCCCCC | SEQ ID NO: 169 |
| | probe3 | CCGCTACCAGACCCTTACTATGCCTACCTGTCGATTGCGTCTATTTAGTGGAGCCCCGGACCGCGTATAC | SEQ ID NO: 170 |
| | probe4 | TGGAGTCCCTTTTCATACTATGCCTACCTGTCGATTGCGTCTATTTAGTGGAGCCAGCTGGCTTCGGAGA | SEQ ID NO: 171 |
| | probe5 | TGCTGTGCTACGTGTTACTATGCCTACCTGTCGATTGCGTCTATTTAGTGGAGCCTAGTGAGCGAGTGGA | SEQ ID NO: 172 |
| HOXB7 | probe1 | GAGCGGGTTCGGGCGTCGCGTCTACCTGACTTTAGTGCGTCTATTTAGTGGAGCCAGCGCCCGGGCTATG | SEQ ID NO: 173 |
| | probe2 | CTTCAACATGCACTGTCGCGTCTACCTGACTTTAGTGCGTCTATTTAGTGGAGCCGCTCGAGCCGAGTTC | SEQ ID NO: 174 |
| | probe3 | GGAACTGACCGCAAATCGCGTCTACCTGACTTTAGTGCGTCTATTTAGTGGAGCCTGGATGCGAAGCTCA | SEQ ID NO: 175 |
| | probe4 | GCAGCGGCACCAAGGTCGCGTCTACCTGACTTTAGTGCGTCTATTTAGTGGAGCCGCCTCCGCCTTCCCA | SEQ ID NO: 176 |
| | probe5 | TTCCCCAAGCGCCTGTCGCGTCTACCTGACTTTAGTGCGTCTATTTAGTGGAGCCCCTACCACTCGCGTG | SEQ ID NO: 177 |
| ROH10 | probe1 | GCGGCAGCCCGCTGGTCGCGTTCACCGTAATCTTATGCGTCTATTTAGTGGAGCCGAGTTGACAACTCCC | SEQ ID NO: 178 |
| | probe2 | CGTTCGTGCTGGCCGTCGCGTTCACCGTAATCTTATGCGTCTATTTAGTGGAGCCTCAAAGTGCTCTGGG | SEQ ID NO: 179 |
| | probe3 | TTCCTACGATGCTGGTCGCGTTCACCGTAATCTTATGCGTCTATTTAGTGGAGCCCCACTAAGGCTTTTC | SEQ ID NO: 180 |
| | probe4 | AGGAGCGGACAAGTGTCGCGTTCACCGTAATGTTATGCGTCTATTTAGTGGAGCCCATGTATCGGTTCCT | SEQ ID NO: 181 |

TABLE 6-G

| | | | |
|---|---|---|---|
| | probe5 | ATGGAGGCACTGGCTTCGCGTTCACCGTAATCTTATGCGTCTATTTAGTGGAGCCATGGCCCTTATAACA | SEQ ID NO: 182 |
| HOXB6 | probe1 | CGGCGGAGCAGCAGCACCTACGAACACTAAGTCGGTGCGTCTATTTAGTGGAGCCAACAAATCATAAACC | SEQ ID NO: 183 |
| | probe2 | CTGCGCACTCTCCGGACCTACGAACACTAAGTCGGTGCGTCTATTTAGTGGAGCCCGAGAAAGAGTCGGC | SEQ ID NO: 184 |
| | probe3 | CAACAGTTCCTCCTTACCTACGAACACTAAGTCGGTGCGTCTATTTAGTGGAGCCGCGGATGAATTCGTG | SEQ ID NO: 185 |
| | probe4 | GCCCACCCAGCATCCACCTACGAACACTAAGTCGGTGCGTCTATTTAGTGGAGCCGGCCTCACTACTCGA | SEQ ID NO: 186 |
| | probe5 | CCGTGCACGGTTCAAACCTACGAACACTAAGTCGGTGCGTCTATTTAGTGGAGCCTTTTGTCCATGTCCC | SEQ ID NO: 187 |
| HOXB3 | probe1 | AGCGCGCAGCTGGTGTAGACTCTCGATCAGCCGTATGCGTCTATTTAGTGGAGCCCGGACGGCGTACACG | SEQ ID NO: 188 |
| | probe2 | GCGCTGCCCTCCAACTAGACTCTCGATCAGCCGTATGCGTCTATTTAGTGGAGCCCACCAGAATGCCTAC | SEQ ID NO: 189 |
| | probe3 | CTGCGAACCCCACCCTAGACTCTCGATCAGCCGTATGCGTCTATTTAGTGGAGCCCCAGGACCACGGACC | SEQ ID NO: 190 |
| | probe4 | TGGGGTAGAGGAAGATAGACTCTCGATCAGCCGTATGCGTCTATTTAGTGGAGCCTTCCACGCCGGGGAG | SEQ ID NO: 191 |
| | probe5 | AAAAGCGTGTGTTCTTAGACTCTCGATCAGCCGTATGCGTCTATTTAGTGGAGCCTCCAGCATTGCTCAA | SEQ ID NO: 192 |
| HOXC12 | probe1 | GCCCTACCTCGGCAGTTGACATAGTGGTCGAGTTCTGCGTCTATTTAGTGGAGCCCAATGGCTACCCGCA | SEQ ID NO: 193 |
| | probe2 | CAACTGGCAGAGCTGTTGACATAGTGGTCGAGTTCTGCGTCTATTTAGTGGAGCCCCCTATTCGAAGTTG | SEQ ID NO: 194 |
| | probe3 | GGTTAGAAGAGGCTGTTGACATAGTGGTCGAGTTCTGCGTCTATTTAGTGGAGCCCCTCACAGTTCCTGG | SEQ ID NO: 195 |
| | probe4 | AATTTACGAGGCGGGTTGACATAGTGGTCGAGTTCTGCGTCTATTTAGTGGAGCCAGGGTGGACCAGCGT | SEQ ID NO: 196 |
| | probe5 | CCTTCCTGTGACCTCTTGACATAGTGGTCGAGTTCTGGGTCTATTTAGTGGAGCCCCCCCTTTATGCAGC | SEQ ID NO: 197 |
| HOXD13 | probe1 | GTTGTAGCGGCGCGCAACGACTAACAACTGTCCGTTGCGTCTATTTAGTGGAGCCTCGTCCTCTTCTGCC | SEQ ID NO: 198 |
| | probe2 | GAGGCCTACATCTCCAACGACTAACAACTGTCCGTTGCGTCTATTTAGTGGAGCCGGGGAGCCTCGGCAC | SEQ ID NO: 199 |
| | probe3 | CCAAAAGGCCTTTGGAACGACTAACAACTGTCCGTTGCGTCTATTTAGTGGAGCCGCCATTCGGTTGTCT | SEQ ID NO: 200 |
| | probe4 | CCGAGAAAGTCTAAAAACGACTAACAACTGTCCGTTGCGTCTATTTAGTGGAGCCAGTGCTCTGGGGTCA | SEQ ID NO: 201 |
| | probe5 | TGGAGCTGTAAAGCAAACGACTAACAACTGTCCGTTGCGTCTATTTAGTGGAGCCCAAGGGGGCCTCGCA | SEQ ID NO: 202 |
| HOXB13 | probe1 | ATGCCACCTTGGATGACGAGGTTAAAGTCAAGTCCTGCGTCTATTTAGTGGAGCCTGGAGCCCGGCAATT | SEQ ID NO: 203 |
| | probe2 | TTCCGTACAGCAAGGACGAGGTTAAAGTCAAGTCCTGCGTCTATTTAGTGGAGCCGCCGCAAGAAACGCA | SEQ ID NO: 204 |
| | probe3 | TAGGTGGACAATTGTACGAGGTTAAAGTCAAGTCCTGCGTCTATTTAGTGGAGCCCAGCTGACAGCTGGG | SEQ ID NO: 205 |
| | probe4 | TGGACAACCCGCAGAACGAGGTTAAAGTCAAGTCCTGCGTCTATTTAGTGGAGCCCACAGGCCTGAAGTC | SEQ ID NO: 206 |
| | probe5 | CCTTTGGGGGTCTGGACGAGGTTAAAGTCAAGTCCTGCGTCTATTTAGTGGAGCCTGGCCCTGGTAGAGA | SEQ ID NO: 207 |
| HOXD9 | probe1 | TTATAGGCCATGAGGACGAGTAGAAATGACGCTCCTGCGTCTATTTAGTGGAGCCACTACGTGGACTCGC | SEQ ID NO: 208 |
| | probe2 | GCTCCGTGGCCCGGGACGAGTAGAAATGACGCTCCTGCGTCTATTTAGTGGAGCCCCAAACGGACTGAGT | SEQ ID NO: 209 |
| | probe3 | GCTGAAGGAGGAGGAACGAGTAGAAATGACGCTCCTGCGTCTATTTAGTGGAGCCGATCCCAGGCTGTTC | SEQ ID NO: 210 |
| | probe4 | CTATCCCACTCCCTCACGAGTAGAAATGACGCTCCTGCGTCTATTTAGTGGAGCCCTTTGGGGTTTCGCC | SEQ ID NO: 211 |
| | probe5 | TTAGAGGAGCCCAGGACGAGTAGAAATGACGCTCCTGCGTCTATTTAGTGGAGCCGATCTCCAGTGAGGC | SEQ ID NO: 212 |
| HOXB4 | probe1 | CTGTCCCCTCGGGCTAGCGGACTAAATATAGGTCGTGCGTCTATTTAGTGGAGCCCCGCCACCGCCCGGT | SEQ ID NO: 213 |
| | probe2 | TATCTGCCCTCCCCCAGCGGACTAAATATAGGTCGTGCGTCTATTTAGTGGAGCCGGCCCCGGAAAAATC | SEQ ID NO: 214 |
| | probe3 | TCTGCCGAGGAGAAGAGCGGACTAAATATAGGTCGTGCGTCTATTTAGTGGAGCCATCTGTCTTGTTTCC | SEQ ID NO: 215 |
| | probe4 | TGACTGCTCACCCACAGCGGACTAAATATAGGTCGTGCGTCTATTTAGTGGAGCCCACTGAGGGCCAGAA | SEQ ID NO: 216 |

TABLE 6-H

| | | | |
|---|---|---|---|
| | probe5 | AGAACCCTTCGTATGAGCGGACTAAATATAGGTCGTGCGTCTATTTAGTGGAGCCGGGCTGCTTGAGTCT | SEQ ID NO: 217 |
| HOXC9 | probe1 | GGACTGTAGCGATTTCACGGTATAGACACCACGTATGCGTCTATTTAGTGGAGCCCAGCGGTTTGGTGCC | SEQ ID NO: 218 |
| | probe2 | TTGGCTCGAGCCGCTCACGGTATAGACACCACGTATGCGTCTATTTAGTGGAGCCGCGCTACATGCGGAC | SEQ ID NO: 219 |
| | probe3 | TCTCACCGAGCGGCACACGGTATAGACACCACGTATGCGTCTATTTAGTGGAGCCGGCCCGGGTTCTCAA | SEQ ID NO: 220 |
| | probe4 | AGATTTTGTACAAAACACGGTATAGACACCACGTATGCGTCTATTTAGTGGAGCCGGGCGCTCTGCGTGC | SEQ ID NO: 221 |
| | probe5 | GTGCTTCTGTTTGTTCACGGTATAGACACCACGTATGCGTCTATTTAGTGGAGCCCGTCCGTCCTGTAAC | SEQ ID NO: 222 |
| HOXB8 | probe1 | TGGAGGCTTCTCTGTCACGGTCAACTAGGTGAGAATGCGTCTATTTAGTGGAGCCGCAAGCTTAGACTTT | SEQ ID NO: 223 |
| | probe2 | TTCGGTGCGCAGGATCACGGTCAACTAGGTGAGAATGCGTCTATTTAGTGGAGCCCAAGGCCAGAGCCTA | SEQ ID NO: 224 |
| | probe3 | AGGGCGACAAGAAGTCACGGTCAACTAGGTGAGAATGCGTCTATTTAGTGGAGCCAGGGCGACGCGCAGA | SEQ ID NO: 225 |
| | probe4 | GTGAGGGCGAGCGGCCACGGTCAACTAGGTGAGAATGCGTCTATTTAGTGGAGCCAGGCTCCCTCGCCCT | SEQ ID NO: 226 |
| | probe5 | AGGGCGGAAGAGTTACACGGTCAACTAGGTGAGAATGCGTCTATTTAGTGGAGCCTGTCTGCGCCTGAAA | SEQ ID NO: 227 |
| HOXA6 | probe1 | TACACCTCACCTTGTCCTTATCGAAGTAGACGACATGCGTCTATTTAGTGGAGCCCTCCCGGACAAGACG | SEQ ID NO: 228 |
| | probe2 | TCGCCCTCGGGCAGTCCTTATCGAAGTAGACGACATGCGTCTATTTAGTGGAGCCGACCTCAGTGGCGCC | SEQ ID NO: 229 |
| | probe3 | TGTGTATGGGAGCCACCTTATCGAAGTAGACGACATGCGTCTATTTAGTGGAGCCCTCCTGCGCGGGTGC | SEQ ID NO: 230 |
| | probe4 | TAGATGCCTGGGCAGCCTTATCGAAGTAGACGACATGCGTCTATTTAGTGGAGCCGCAAAGGGGGGGGAG | SEQ ID NO: 231 |
| | probe5 | GCAAGCGTCTGTGCACCTTATCGAAGTAGACGACATGCGTCTATTTAGTGGAGCCGGAGCCCGGAGCTTT | SEQ ID NO: 232 |
| HOXC13 | probe1 | GACGACTTCGCTGCTCGGTACTAAGGCTGACCTAATGCGTCTATTTAGTGGAGCCAGCAGATCATGTCAT | SEQ ID NO: 233 |
| | probe2 | GGAGTTCGCCTTCTACGGTACTAAGGCTGACCTAATGCGTCTATTTAGTGGAGCCGTCCTCTAGGGCCAA | SEQ ID NO: 234 |
| | probe3 | CGGCTAGCAAGTTCACGGTACTAAGGCTGACCTAATGCGTCTATTTAGTGGAGCCTAGAGAAGGAATACG | SEQ ID NO: 235 |
| | probe4 | ATCATTCCAACCAAACGGTACTAAGGCTGACCTAATGCGTCTATTTAGTGGAGCCGCAGCGCAGAGCCCA | SEQ ID NO: 236 |
| | probe5 | GCCCAGGAATGACTCCGGTACTAAGGCTGACCTAATGCGTCTATTTAGTGGAGCCTCCATCAGGGCACTA | SEQ ID NO: 237 |
| HOXD4 | probe1 | AGCCTTTCGGAGGCAGACATCAGAGTACCAGCTCATGCGTCTATTTAGTGGAGCCCCGACTTCGGTGAGC | SEQ ID NO: 238 |
| | probe2 | CCCAAGCAGCCGCCCGACATCAGAGTACCAGCTCATGCGTCTATTTAGTGGAGCCTACAGTCAGTCCGAC | SEQ ID NO: 239 |
| | probe3 | CGTCGGATTGAAATCGACATCAGAGTACCAGCTCATGCGTCTATTTAGTGGAGCCTATCTGACAAGGCGC | SEQ ID NO: 240 |
| | probe4 | ACTGTGGGGTGGACCGACATCAGAGTACCAGCTCATGCGTCTATTTAGTGGAGCCGCTGGGCTCTAAGGT | SEQ ID NO: 241 |
| | probe5 | CTCTCTCTAAGTATAGACATCAGAGTACCAGCTCATGCGTCTATTTAGTGGAGCCAGGGGGGGCGGGATT | SEO ID NO: 242 |

TABLE 6-I

| Gene | | padlock probe sequence | |
|---|---|---|---|
| FGF9 | probe1 | CTAAGCCAATGGACACCTTAACTCGCGTTCAAGTATGCGTCTATTTAGTGGAGCCCGCTCGCCACCCGTT | SEQ ID NO: 243 |
| | probe2 | AAGAGCTGGGGATCTCCTTAACTCGCGTTCAAGTATGCGTCTATTTAGTGGAGCCACCTTCCTGCCTGCT | SEQ ID NO: 244 |
| | probe3 | CGGTACCGTTTGGGACCTTAACTCGCGTTCAAGTATGCGTCTATTTAGTGGAGCCTCGGTGTGCAGGATG | SEQ ID NO: 245 |
| | probe4 | TTGCTTGATTTATCACCTTAACTCGCGTTCAAGTATGCGTCTATTTAGTGGAGCCGCTGCCTAGGGCCAç | SEQ ID NO: 246 |
| | probe5 | TTGTCTCTTGGGCAGCCTTAACTCGCGTTCAAGTATGCGTCTATTTAGTGGAGCCGTTTCGGTGTGGGCA | SEQ ID NO: 247 |
| FGF8 | probe1 | CAAAGCTCATCGTGGCGGTACGGAAGTGTATATCATGCGTCTATTTAGTGGAGCCACGGCGACCCCTTCG | SEQ ID NO: 248 |
| | probe2 | ACGGAGATTGTGCTGCGGTACGGAAGTGTATATCATGCGTCTATTTAGTGGAGCCAAGGACTGCGTCTTC | SEQ ID NO: 249 |
| | probe3 | ATGAAGCGGCTGCCCCGGTACGGAAGTGTATATCATGCGTCTATTTAGTGGAGCCCGTGAGGTCCACTTC | SEQ ID NO: 250 |
| | probe4 | CCGCAGAGAGGCTCACGGTACGGAAGTGTATATCATGCGTCTATTTAGTGGAGCCCCCCACAATGCCAGA | SEQ ID NO: 251 |
| | probe5 | CTGTGCGCTGCTCTGCGGTACGGAAGTGTATATCATGCGTCTATTTAGTGGAGCCCTCAAGCAAGATGAG | SEQ ID NO: 252 |
| ALDH1A2 | probe1 | CTTGCAACCATGGAAGACAGTTTACACTATTCGCGTGCGTCTATTTAGTGGAGCCCGGGACAGGGCAGTT | SEQ ID NO: 253 |
| | probe2 | CCCACCACTGAGCAGGACAGTTTACACTATTCGCGTGCGTCTATTTAGTGGAGCCGGGAGTCCCTTTGAC | SEQ ID NO: 254 |
| | probe3 | AAGTTAAGACGGTGAGACAGTTTACAGTATTCGCGTGCGTCTATTTAGTGGAGCCTGCGGGAGTACTCAG | SEQ ID NO: 255 |
| | probe4 | GCCTAGGGGTTTCCTGACAGTTTACACTATTCGCGTGCGTCTATTTAGTGGAGCCGGAAAATCTGGCCAA | SEQ ID NO: 256 |
| | probe5 | GTGTCCAAGTGGACAGACAGTTTACACTATTCGCGTGCGTCTATTTAGTGGAGCCGCCCTCCTGTAGCAT | SEQ ID NO: 257 |
| FGF4 | probe1 | CCCAACGGCACGCTGGACATAGACCATACCACGTTTGCGTCTATTTAGTGGAGCCGCCGCACCCACTGCA | SEQ ID NO: 258 |
| | probe2 | CATGAAGGTCACCCAGACATAGACCATACCACGTTTGCGTCTATTTAGTGGAGCCCCGAGTGTCGCCCAC | SEQ ID NO: 259 |
| | probe3 | CCACCCCCAAAACGGGACATAGACCATACCACGTTTGCGTCTATTTAGTGGAGCCATCGAACCCCCCGCC | SEQ ID NO: 260 |
| | probe4 | CCAGAAATGCTCCGAGACATAGACCATACCACGTTTGCGTCTATTTAGTGGAGCCGACCTTAGCTTGCTA | SEQ ID NO: 261 |
| | probe5 | ACATGCTGACCGACAGACATAGACCATACCACGTTTGCGTCTATTTAGTGGAGCCGTGCTGTGCACATTG | SEQ ID NO: 262 |
| FGF18 | probe1 | ACTTGCGTGTGTTTATAGACGTAGTGAGCATGACTTGCGTCTATTTAGTGGAGCCGCGCCCTCCGCCTGC | SEQ ID NO: 263 |
| | probe2 | AGGCAAGCTCGTGGGTAGACGTAGTGAGCATGACTTGCGTCTATTTAGTGGAGCCGTGCATGAACCGCAA | SEQ ID NO: 264 |
| | probe3 | CCCACACACCCTGCCTAGACGTAGTGAGCATGACTTGCGTCTATTTAGTGGAGCCTCCCGTCGGATCCGG | SEQ ID NO: 265 |
| | probe4 | CGCGCCTCGACCTCCTAGACGTAGTGAGCATGACTTGCGTCTATTTAGTGGAGCCCCCCTGGGAATTCTC | SEQ ID NO: 266 |
| | probe5 | CACGTGACCTAGTACTAGACGTAGTGAGCATGACTTGCGTCTATTTAGTGGAGCCCGACCTTCCGTGACT | SEQ ID NO: 267 |
| WNT3A | probe1 | CTACGTGGAGATCATCTCTGCGAAGGTAGTGTAATTGCGTCTATTTAGTGGAGCCCCGCTTCTGCAGGÅA | SEQ ID NO: 268 |
| | probe2 | CCTCTCTGGTGGCTGGTCTGCGAAGGTAGTGTAATTGCGTCTATTTAGTGGAGCCCTGGAGCTAGTGTCT | SEQ ID NO: 269 |
| | probe3 | AACCCGTGCCCCTGGCTCTGCGAAGGTAGTGTAATTGCGTCTATTTAGTGGAGCCGCGAGTCGGGTCCCC | SEQ ID NO: 270 |
| | probe4 | CGGGAGGTTTGGGGGCTCTGCGAAGGTAGTGTAATTGCGTCTATTTAGTGGAGCCTCATTTGGGGGCGTT | SEQ ID NO: 271 |
| | probe5 | GCACACCGCCATCCTCTCTGCGAAGGTAGTGTAATTGCGTCTATTTAGTGGAGCCAGAGCCCAAAAGAGG | SEQ ID NO: 272 |
| SFRP1 | probe1 | CCAGTCGGACATCGGCTTTCCAATACGGTGTACGATGCGTCTATTTAGTGGAGCCCGACTACGTGAGCTT | SEQ ID NO: 273 |
| | probe2 | GTTCTTCGGCTTCTACTTTCCAATAGGGTGTACGATGCGTCTATTTAGTGGAGCCCGAGCCGGTCATGCA | SEQ ID NO: 274 |
| | probe3 | TGACGGCCATCCACACTTTCCAATACGGTGTACGATGCGTCTATTTAGTGGAGCCAGAGCCAGTACTTGC | SEQ ID NO: 275 |
| | probe4 | GCTCCCTTCCCTCCACTTTCCAATACGGTGTACGATGCGTCTATTTAGTGGAGCCTCTTGCAGCATTCCC | SEQ ID NO: 276 |
| | probe5 | GCAACCGGGCCCTCTCTTTCCAATACGGTGTACGATGCGTCTATTTAGTGGAGCCGACAAAGTGCCTCAT | SEQ ID NO: 277 |
| FGFBP3 | probe1 | CGGTCCTGGGGAATGCTTTCGACGCTGACACTAAATGCGTCTATTTAGTGGAGCCGCCTCTCCCAGAACT | SEQ ID NO: 278 |
| | probe2 | GCCCAAAGAAAACCCGTTTCGACGCTGACACTAAATGCGTCTATTTAGTGGAGCCTCCCCAAAGCGCACC | SEQ ID NO: 279 |
| | probe3 | GGGGAGGCGTGGGGACTTTCGACGCTGACACTAAATGCGTCTATTTAGTGGAGCCTAGGGAGGGTTGATG | SEQ ID NO: 280 |

TABLE 6-J

| | | | |
|---|---|---|---|
| | probe4 | TGGTGGGTGTCATAGCTTTCGACGCTGACACTAAATGCGTCTATTTAGTGGAGCCAACTTGGATGCGGCG | SEQ ID NO: 281 |
| | probe5 | GTAGGAGTAGCTGGGCTTTCGACGCTGACACTAAATGCGTCTATTTAGTGGAGCCCCAGAATAGCTAGGA | SEQ ID NO: 282 |
| FGFR1 | probe1 | TTGAAAAATGGCAAAGCACTATTACGCAGAGCATCTGCGTCTATTTAGTGGAGCCCCCACACTGCGCTGG | SEQ ID NO: 283 |
| | probe2 | CAAAACAGTGGCCCTGCACTATTACGCAGAGCATCTGCGTCTATTTAGTGGAGCCAGGGTTGCCCGCCAA | SEQ ID NO: 284 |
| | probe3 | TTCTGGAAGCCCTGGGCACTATTACGCAGAGCATCTGCGTCTATTTAGTGGAGCCCTGCATGGTTGACCG | SEQ ID NO: 285 |
| | probe4 | GGCTATCGGGCTGGAGCACTATTACGCAGAGCATCTGCGTCTATTTAGTGGAGCCGGTGGTGTTGGCAGA | SEQ ID NO: 286 |
| | probe5 | CGCATGGACAAGCCCGCACTATTACGCAGAGCATCTGCGTCTATTTAGTGGAGCCCTGAAGGAGGGTCAC | SEQ ID NO: 287 |
| FGFR2 | probe1 | CGAGTATGAACTTCGGGTATAGTACATTCCCGCACTGCGTCTATTTAGTGGAGCCGCTGGCAGGGGTCTC | SEQ ID NO: 288 |
| | probe2 | GACACTGGGCAAGCCGGTATAGTACATTCCCGCACTGCGTCTATTTAGTGGAGCCTCCAAGAGATAAGCT | SEQ ID NO: 289 |
| | probe3 | GGAGTACTCCTATGAGGTATAGTACATTCCCGCACTGCGTCTATTTAGTGGAGCCGAGGCCACCCGGGAT | SEQ ID NO: 290 |
| | probe4 | ACCTACCAGCTGGCCGGTATAGTACATTCCCGCACTGCGTCTATTTAGTGGAGCCGACTTGGTGTCATGC | SEQ ID NO: 291 |
| | probe5 | ACCAACGAACTGTACGGTATAGTACATTCCCGCACTGCGTCTATTTAGTGGAGCCAAGCCAGCCAACTGC | SEQ ID NO: 292 |
| FGFR3 | probe1 | GGCAGCGGGGATGCTGGTATGCGACCTACTCTTACTGCGTCTATTTAGTGGAGCCGAGCAGTTGGTCTTC | SEQ ID NO: 293 |
| | probe2 | GACGAAGACGGGGAGGGTATGCGACCTACTCTTACTGCGTCTATTTAGTGGAGCCCCATCCTCGGGAGAT | SEQ ID NO: 294 |
| | probe3 | GTGGTGCCCTCGGACGGTATGCGACCTACTCTTACTGCGTCTATTTAGTGGAGCCCTGGTCATGGAAAGC | SEQ ID NO: 295 |
| | probe4 | GTTACCGTGCTCAAGGGTATGCGACCTACTCTTACTGCGTCTATTTAGTGGAGCCGACGGCACACCCTAC | SEQ ID NO: 296 |
| | probe5 | CCTGACTGGTGCTGCGGTATGCGACCTACTCTTACTGCGTCTATTTAGTGGAGCCGCTGTGCAACGGTCT | SEQ ID NO: 297 |
| FGFR4 | probe1 | ATGCTGGCCGCTACCGTCTGAGAATGTGTTAGCACTGGGTCTATTTAGTGGAGCCGCTTCCTACCTGAGG | SEQ ID NO: 298 |
| | probe2 | ACCGTCAAGTTCCGCGTCTGAGAATGTGTTAGCACTGCGTCTATTTAGTGGAGCCGTACCTGGGGGGAAC | SEQ ID NO: 299 |
| | probe3 | TGTCAGCCGAGGACGGTCTGAGAATGTGTTAGCACTGCGTCTATTTAGTGGAGCCTGTACCTGCGGAACG | SEQ ID NO: 300 |
| | probe4 | GGCCGCCTGCCTGTGGTCTGAGAATGTGTTAGCACTGCGTGTATTTAGTGGAGCCAAGAAAACCAGCAAC | SEQ ID NO: 301 |
| | probe5 | GTCCCCATCCCGGGTGTCTGAGAATGTGTTAGCACTGCGTCTATTTAGTGGAGCCGGACCTTGGCGCTTA | SEQ ID NO: 302 |
| SPRY2 | probe1 | CGAGAGACTCCACGGTACGTTTGACGAGGAGTACATGCGTCTATTTAGTGGAGCCCACTCAGCACAAACA | SEQ ID NO: 303 |
| | probe2 | TGCTGATGGCATAATTACGTTTGACGAGGAGTACATGCGTCTATTTAGTGGAGCCCTCCTCCGGGCCTGT | SEQ ID NO: 304 |
| | probe3 | AGGTGTGAGGACTGTTACGTTTGACGAGGAGTACATGCGTCTATTTAGTGGAGCCGGCCTGCACGCCTAC | SEQ ID NO: 305 |
| | probe4 | TCCCTCTTTTTGCCTTAGGTTTGACGAGGAGTACATGCGTCTATTTAGTGGAGCCGCCATGGGTGTCATG | SEQ ID NO: 306 |
| | probe5 | TGCTGTTTGCGGTGATACGTTTGACGAGGAGTACATGCGTCTATTTAGTGGAGCCAGGTGGGATAGTCTT | SEQ ID NO: 307 |
| SPRY4 | probe1 | GCTCCAGCACCCACTACCTACCTAAAGTGACATCGTGCGTCTATTTAGTGGAGCCGATGTCCCACAGCCG | SEQ ID NO: 308 |
| | probe2 | TGTGCGAGGCCTGTGACCTACCTAAAGTGACATCGTGCGTCTATTTAGTGGAGCCACAAGCACTTCTTGC | SEQ ID NO: 309 |
| | probe3 | AGGACGATGAGGGCTACCTACCTAAAGTGACATCGTGCGTCTATTTAGTGGAGCCACCACTGCACGAATG | SEQ ID NO: 310 |
| | probe4 | CAGTGCTCTGCCTGGACCTACCTAAAGTGACATCGTGCGTCTATTTAGTGGAGCCTTTGTGTCGAAGCCC | SEQ ID NO: 311 |
| | probe5 | CAGTGCACTTCTCCCACCTACCTAAAGTGACATCGTGCGTCTATTTAGTGGAGCCTTAGGAGCCACTGCT | SEQ ID NO: 312 |
| WNT5B | probe1 | CAGCTTCCCGGGCTCAGATACGGAAATGGACGTTCTGCGTGTATTTAGTGGAGCCCAGCCCGTGTGCAGT | SEQ ID NO: 313 |
| | probe2 | GCCAAGGAGTTTGTGAGATACGGAAATGGACGTTCTGCGTCTATTTAGTGGAGCCTACGGCTACCGCTTC | SEQ ID NO: 314 |
| | probe3 | GCTGCGGGCGTGGCTAGATACGGAAATGGACGTTCTGCGTCTATTTAGTGGAGCCGCTGTGAGCTCATGT | SEQ ID NO: 315 |
| | probe4 | GGTGCTCTCTTACTCAGATACGGAAATGGACGTTCTGCGTCTATTTAGTGGAGCCTTCCGTAAGAGGCCT | SEQ ID NO: 316 |
| | probe5 | TATACTGTCTTCCCCAGATACGGAAATGGACGTTCTGCGTCTATTTAGTGGAGCCAGGCTGCGTGGACGT | SEQ ID NO: 317 |
| DUSP6 | probe1 | TGAGGTGCAGCCTTGAGATACTTAACCATAGCGCCTGCGTCTATTTAGTGGAGCCACGCCCGGGTAGATT | SEQ ID NO: 318 |
| | probe2 | CAGACCCGTGCCCTTAGATACTTAACCATAGCGCCTGCGTCTATTTAGTGGAGCCCATGATAGATACGCT | SEQ ID NO: 319 |

TABLE 6-K

| | | | |
|---|---|---|---|
| | probe3 | AGTGGTGCTCTACGAAGATACTTAACCATAGCGCCTGCGTCTATTTAGTGGAGCCCTGTGGCACCGACAC | SEQ ID NO: 320 |
| | probe4 | TCTGGCCCTTCAGCAAGATACTTAACCATAGCGCCTGCGTCTATTTAGTGGAGCCCCTTGCTGGAATGTG | SEQ ID NO: 321 |
| | probe5 | CTCACCGTGTCAGGAAGATACTTAACCATAGCGCCTGCGTCTATTTAGTGGAGCCGCATATCTAAGCTTC | SEQ ID NO: 322 |
| WNTSA | probet | TACCGCTTTGCCAAGATAGAGTCAACGAGCGTACCTGCGTCTATTTAGTGGAGCCAACATCGACTATGGC | SEQ ID NO: 323 |
| | probez | CCACACAAGACCTGGATAGAGTCAACGAGCGTACCTGCGTCTATTTAGTGGAGCCGCTTCAACTCGCCCA | SEQ ID NO: 324 |
| | probe3 | GGGCATGGATGGCTGATAGAGTCAACGAGCGTACCTGCGTCTATTTAGTGGAGCCCAACAAGACGTCGGA | SEO ID NO: 325 |
| | probe4 | GGGTGCCACCCAGCAATAGAGTCAACGAGCGTACCTGCGTCTATTTAGTGGAGCCTTGTGTGCAAGTAGT | SEQ ID NO: 326 |
| | probe5 | GAGACTGGCACTGTGATAGAGTCAACGAGCGTACCTGCGTCTATTTAGTGGAGCCAGCTACTGACCTCCT | SEQ ID NO: 327 |
| FGF17 | probe1 | GAGAGTGAGAAGTACCACACTAACTCGAATGACGTTGCGTCTATTTAGTGGAGCCCGCATCAAAGGGGCT | SEQ ID NO: 328 |
| | probe2 | ACCAAGGCCAGCTGCCACACTAACTCGAATGACGTTGCGTCTATTTAGTGGAGCCTCATCAAGCGCCTCT | SEQ ID NO: 329 |
| | probe3 | CCCCCAGCTGGGAAGCACACTAACTCGAATGACGTTGCGTCTATTTAGTGGAGCCCGCGAAGCATCCGAG | SEQ ID NO: 330 |
| | probe4 | CTCCCTCAGTCTGCCCACACTAACTCGAATGACGTTGCGTCTATTTAGTGGAGCCATCTGCTTCTCGGAT | SEQ ID NO: 331 |
| | probe5 | CAGGCCTGCACCCCACACACTAACTCGAATGACGTTGCGTCTATTTAGTGGAGCCTCACATTCCACGACC | SEQ ID NO: 332 |
| WNTBA | probe1 | TTCAGCTCTCCACCCCACACTGTAAGACGATAGCCTGCGTCTATTTAGTGGAGCCGCCCTGAAAATGCTC | SEQ ID NO: 333 |
| | probe2 | AGCCACCATGAAAAGCACACTGTAAGACGATAGCCTGCGTCTATTTAGTGGAGCCCAGACTGGCAGTGAG | SEQ ID NO: 334 |
| | probe3 | AGCTGAGAGCTGGGACACACTGTAAGACGATAGCCTGCGTCTATTTAGTGGAGCCAAATGGATAAGCGGC | SEQ ID NO: 335 |
| | probe4 | TGCCTACAGAACAGCCACACTGTAAGACGATAGCCTGCGTCTATTTAGTGGAGCCACAGAGGGTCGTGAG | SEQ ID NO: 335 |
| | probe5 | TGACCAGTGTAGGCACACACTGTAAGACGATAGCCTGCGTCTATTTAGTGGAGCCCTGTACGGTCAAGTG | SEQ ID NO: 337 |
| RSPO3 | probe1 | CAACATGCTACCTGCCCGTCAGAAGCCATACTAGATGCGTCTATTTAGTGGAGCCGCGGCAGTTGCCGCA | SEQ ID NO: 338 |
| | probe2 | TATCGGATGTGAGAGCCGTCAGAAGCCATACTAGATGCGTCTATTTAGTGGAGCCCCATCACAGCACGCC | SEQ ID NO: 339 |
| | probe3 | AACACGGGTCCGAGACCGTCAGAAGCCATACTAGATGCGTCTATTTAGTGGAGCCCAAAAGAGGGACTGA | SEQ ID NO: 340 |
| | probe4 | TGATTTCACTGCTCCCCGTCAGAAGCCATACTAGATGCGTCTATTTAGTGGAGCCATGGGCCTCCCTAGC | SEQ ID NO: 341 |
| | probe5 | AGTGACTGCGTCCTTCCGTCAGAAGCCATACTAGATGCGTCTATTTAGTGGAGCCCCCTGAGTCCTCATT | SEQ ID NO: 342 |
| RSPO4 | probe1 | GAACCGAAGGAAGAACCGTCGATACAGACTCAGATTGCGTCTATTTAGTGGAGCCGGACATGCTCGCCCT | SEQ ID NO: 343 |
| | probe2 | AGAGGCAGTTTTACTCCGTCGATACAGACTCAGATTGCGTCTATTTAGTGGAGCCTCTGCATCCGGTGCA | SEQ ID NO: 344 |
| | Probe3 | GAATCCTGAAGGCCCCCGTCGATACAGACTCAGATTGCGTCTATTTAGTGGAGCCGGACACAGCACAGGT | SEQ ID NO: 345 |
| | probe4 | CTCGTCCACACCCACCCGTCGATACAGACTCAGATTGCGTCTATTTAGTGGAGCCGGGAAGATCCGTGCA | SEQ ID NO: 346 |
| | probe5 | CGTGACTGGGACAGGCCGTCGATACAGACTCAGATTGCGTCTATTTAGTGGAGCCGAACACGTAGGGCTC | SEQ ID NO: 347 |
| SFRP2 | probs1 | AGCCCGACTTCTCCTCGGACTAAGGGCGACTAATATGCGTCTATTTAGTGGAGCCTCTTCCTCTTTGGCC | SEQ ID NO: 348 |
| | probe2 | CAGTGCCACCCGGACCGGACTAAGGGCGACTAATATGCGTCTATTTAGTGGAGCCCCGCTGGTCATGAAG | SEQ ID NO: 349 |
| | probe3 | GCTGTGGCTCAAAGACGGACTAAGGGCGACTAATATGCGTCTATTTAGTGGABCCCCTGAAGAAATCGGT | SEQ ID NO: 350 |
| | probe4 | CTCCGGGATCTCAGCCGGACTAAGGGCGACTAATATGCGTCTATTTAGTGGAGCCGGCTGACCATTTCTG | SEO ID NO: 351 |
| | probe5 | CACGTTTGCATCCCCCGGACTAAGGGCGACTAATATGCGTCTATTTAGTGGAGCCTCCCCCTGCCTTTTG | SEQ ID NO: 352 |
| DKK1 | probe1 | CGCGATGGTAGCGGCCTAGACTGAATCATCACCGATGCGTCTATTTAGTGGAGCCTACCGGGGTCTTTGT | SEQ ID NO: 353 |
| | probe2 | CCTGCCCCCACCCCTCTAGACTGAATCATCACCGATGCGTCTATTTAGTGGAGCCCAACGCTATCAAGAA | SEQ ID NO: 354 |
| | probe3 | AGCCGTACCCGTGCGCTAGACTGAATCATCACCGATGCGTCTATTTAGTGGAGCCCCATTGACAACTACC | SEQ ID NO: 355 |
| | probe4 | ACCCGCGGAGGGGACCTAGACTGAATCATCACCGATGCGTCTATTTAGTGGAGCCTACTGCGCTAGTCCC | SEQ ID NO: 356 |
| | probe5 | TTACTGCAAAAATGGCTAGACTGAATCATCACCGATGCGTCTATTTAGTGGAGCCGTGCTGCCCCGGGAA | SEQ ID NO: 357 |
| LEF1 | probe1 | CCATCCCATGCGGTCGAACTATGCTGACAGTACCGTGCGTCTATTTAGTGGAGCCGTGCCCGTGGTGCAG | SEO ID NO: 358 |

TABLE 6-L

| | | | |
|---|---|---|---|
| | probe2 | CGTGAAGCCTCAGCAGAACTATGCTGACAGTACCGTGCGTCTATTTAGTGGAGCCCAGTGACCTAATGCA | SEQ ID NO: 359 |
| | probe3 | TTCGGCAAGGACGGTGAACTATGCTGACAGTACCGTGCGTCTATTTAGTGGAGCCCCAAACCTGTAACCT | SEQ ID NO: 360 |
| | probe4 | GGTGTTCAGTAGAGCGAACTATGCTGACAGTACCGTGCGTCTATTTAGTGGAGCCTCTCCGCCCTTGTAA | SEQ ID NO: 361 |
| | probe5 | CTGGTTTGCAGTGAAGAACTATGCTGACAGTACCGTGCGTCTATTTAGTGGAGCCGATGCGTTCAGCAGA | SEQ ID NO: 362 |
| CRABP2 | probe1 | TTTTGACTAAAAGACGTCTGGAGAGATCAGTATCCTGCGTCTATTTAGTGGAGCCCGACGGCGACGTCTC | SEQ ID NO: 363 |
| | probe2 | GAGATTAACTTCAAGGTCTGGAGAGATCAGTATCCTGCGTCTATTTAGTGGAGCCACCGTGCGCACCACA | SEQ ID NO: 364 |
| | probe3 | TACGTCCGAGAGTGAGTCTGGAGAGATCAGTATCCTGCGTCTATTTAGTGGAGCCGTGTGCACCAGGGTC | SEQ ID NO: 365 |
| | probe4 | CCCCTTACCCCAGTCGTCTGGAGAGATCAGTATCCTGCGTCTATTTAGTGGAGCCTTCTAGGATAGCGCT | SEQ ID NO: 366 |
| | probe5 | TTCACTCCCCGCCTCGTCTGGAGAGATCAGTATCCTGCGTCTATTTAGTGGAGCCATCACCCATTCCGGG | SEQ ID NO: 367 |
| DACT2 | probe1 | GCCCAGGCCTGTGTCTCGCGTCTACCTGACTTTAGTGCGTCTATTTAGTGGAGCCGTGGGGCACATTCTG | SEQ ID NO: 368 |
| | probe2 | TCGTTCCCTAGGGAGTCGCGTCTACCTGACTTTAGTGCGTCTATTTAGTGGAGCCCAGAGAGGTGGCCCC | SEQ ID NO: 369 |
| | probe3 | ACAGGCCTGAGGGAGTCGCGTCTACCTGACTTTAGTGCGTCTATTTAGTGGAGCCCGCTGCTTCTACTGG | SEQ ID NO: 370 |
| | probe4 | GGGGCCCCCCAGGCCTCGCGTCTACCTGACTTTAGTGCGTCTATTTAGTGGAGCCTCCTGAGTCTAACCT | SEQ ID NO: 371 |
| | probe5 | GATGGGTTCTGCCCTTCGCGTGTACCTGACTTTAGTGCGTCTATTTAGTGGAGCCACAGGAGTCCCCGCA | SEQ ID NO: 372 |
| RDH10 | probe1 | GCGGCAGCCCGCTGGTCGCGTTCACCGTAATCTTATGCGTCTATTTAGTGGAGCCGAGTTGACAACTCCC | SEQ ID NO: 373 |
| | probe2 | CGTTCGTGCTGGCCGTCGCGTTCACCGTAATCTTATGCGTCTATTTAGTGGAGCCTCAAAGTGCTCTGGG | SEQ ID NO: 374 |
| | probe3 | TTCCTACGATGCTGGTCGCGTTCACCGTAATCTTATGCGTCTATTTAGTGGAGCCCCACTAAGGCTTTTC | SEQ ID NO: 375 |
| | probe4 | AGGAGCGGACAAGTGTCGCGTTCACCGTAATCTTATGCGTCTATTTAGTGGAGCCCATGTATGGGTTCCT | SEQ ID NO: 376 |
| | probe5 | ATGGAGGCACTGGCTTCGCGTTCACCGTAATCTTATGCGTCTATTTAGTGGAGCCATGGCCCTTATAACA | SEQ ID NO: 377 |
| AXIN2 | probe1 | CAGTAACAGCCCGAGACCTACGAACACTAAGTCGGTGCGTCTATTTAGTGGAGCCAAGGTCCTGGCAACT | SEQ ID NO: 378 |
| | probe2 | CGGAACGAAGATGGGACCTACGAACACTAAGTCGGTGCGTCTATTTAGTGGAGCCTCTTCCAACACCAGG | SEQ ID NO: 379 |
| | probe3 | ACCAGCCACCAGCGCACCTACGAACACTAAGTCGGTGCGTCTATTTAGTGGAGCCTGGCTATGTCTTTGC | SEQ IO NO: 380 |
| | probe4 | TCAGACGGGAGCCACACCTACGAACACTAAGTCGGTGCGTCTATTTAGTGGAGCCTCATTCGGCCACTGT | SEQ ID NO: 381 |
| | probe5 | GAACTACCCCCTGCCACCTACGAACACTAAGTCGGTGCGTCTATTTAGTGGAGCCACCAGAGCCATTCAG | SEQ ID NO: 382 |
| RBP1 | probe1 | GCCAGTCGACTTCACTAGACTCTCGATCAGCCGTATGCGTCTATTTAGTGGAGCCGTCACTCCCCGAAAT | SEQ ID NO: 383 |
| | probe2 | GGAGTACCTGCGCGCTAGACTCTCGATCAGCCGTATGCGTCTATTTAGTGGAGCCCAACGAGAATTTCGA | SEQ ID NO: 384 |
| | probe3 | CGCCAACTTGCTGAATAGACTCTCGATCAGCCGTATGGGTCTATTTAGTGGAGCCGGCCTTGCGCAAAA? | SEQ ID NO: 385 |
| | probe4 | TTTAGGAACTACATCTAGACTCTCGATCAGCCGTATGCGTCTATTTAGTGGAGCCCGCACGCTGAGCACT | SEQ ID NO: 386 |
| | probe5 | GACCGCAAGTGCATGTAGACTCTCGATCAGCCGTATGCGTCTATTTAGTGGAGCCCTGACAGGCATAGAT | SEQ ID NO: 387 |
| DHRS3 | probe1 | TCTGGACCACCAAGGTTGACATAGTGGTCGAGTTCTGCGTCTATTTAGTGGAGCCACACCCTGGGCCAGT | SEQ ID NO: 388 |
| | probe2 | CCTTCGCCTTCATGGTTGACATAGTGGTCGAGTTCTGCGTCTATTTAGTGGAGCCCATCCAAAGCGTCAG | SEQ ID NO: 389 |
| | probe3 | GCTCAACCAGGCCCTTTGACATAGTGGTCGAGTTCTGCGTCTATTTAGTGGAGCCAGTGGAAGCTGTGCA | SEQ ID NO: 390 |
| | probe4 | CCTGTCCATTGGCATTTGACATAGTGGTCGAGTTCTGCGTCTATTTAGTGGAGCCGCACACACCCGAGCA | SEQ ID NO: 391 |
| | probe5 | CCACAGGGAGGCAGGTTGACATAGTGGTCGAGTTCTGCGTCTATTTAGTGGAGCCGGGTATAACTGACCC | SEQ ID NO: 392 |
| CYP26A1 | probe1 | CCACTGGCCAGCGTCAACCGTCTAAACCATACGTGTGCGTCTATTTAGTGGAGCCCCGGCTGGTGTCGGT | SEQ ID NO: 393 |
| | probe2 | TTCCGAATCGCCATGAACCGTCTAAACCATACGTGTGCGTCTATTTAGTGGAGCCGTGAAGCGCCTCATG | SEQ ID NO: 394 |
| | probe3 | CTGCGGGCTGCGGGCAACCGTCTAAACCATACGTGTGCGTCTATTTAGTGGAGCCCATTCGCGCCAAGAT | SEQ ID NO: 395 |
| | probe4 | TCTCCAGAAAGTGCGAACCGTCTAAACCATACGTGTGCGTCTATTTAGTGGAGCCGCTCTACCCACATGT | SEQ ID NO: 396 |
| | probe5 | ACAATCTCCCTGCAAAACCGTCTAAACCATACGTGTGCGTCTATTTAGTGGAGCCCCGTGTATCCTGTGG | SEQ ID NO: 397 |

TABLE 6-M

| | | | |
|---|---|---|---|
| RARG | probe1 | AATGACCGGAACAAGACGAGGTTAAAGTCAAGTCCTGCGTCTATTTAGTGGAGCCAAGGAAGCTGTGCGA | SEQ ID NO: 398 |
| | probe2 | GGTACACCCCAGAGCACGAGGTTAAAGTCAAGTCCTGCGTCTATTTAGTGGAGCCTGCGTATCTGCACAA | SEQ ID NO: 399 |
| | probe3 | TCCCTTAATCCGAGAACGAGGTTAAAGTCAAGTCCTGCGTCTATTTAGTGGAGCCTCCAGGCCCGATGCC | SEQ ID NO: 400 |
| | probe4 | GTCCCTCCCCCAGCCACGAGGTTAAAGTCAAGTCCTGCGTCTATTTAGTGGAGCCTGCCCTCCGTTATAA | SEQ ID NO: 401 |
| | probe5 | GTGGGGGAGCCCAGGACGAGGTTAAAGTCAAGTCCTGCGTCTATTTAGTGGAGCCCTCCAGGGGTCTTGG | SEQ ID NO: 402 |
| RXRA | probe1 | TGGAGTCGACCAGCAACGAGTAGAAATGACGCTCCTGCGTCTATTTAGTGGAGCCGGAACGAGAATGAGG | SEQ ID NO: 403 |
| | probe2 | TACTGGTGCCCAGCCACGAGTAGAAATGACGCTCCTGCGTCTATTTAGTGGAGCCTTCATCTGCTCTGAA | SEQ ID NO: 404 |
| | probe3 | GCAGAGTGTTGAGCCACGAGTAGAAATGACGCTCCTGCGTCTATTTAGTGGAGCCATCCACCGTCCTGAG | SEQ ID NO: 405 |
| | probe4 | GGCCTCTGGGAAGGAACGAGTAGAAATGACGCTCCTGCGTCTATTTAGTGGAGCCACCCTGGAAGCACAC | SEQ ID NO: 406 |
| | probe5 | TTTCAAGGGTTTTCTACGAGTAGAAATGACGCTCCTGCGTCTATTTAGTGGAGCCGGGTGGGCCTGAGGC | SEQ ID NO: 407 |
| RXRB | probe1 | AGGGTCCTGGGGGAAAGCGGACTAAATATAGGTCGTGCGTCTATTTAGTGGAGCCGTGACCAGGGCGTTG | SEQ ID NO: 408 |
| | probe2 | ATTGCTGCGGGCAGGAGCGGACTAAATATAGGTCGTGCGTCTATTTAGTGGAGCCGGATGATCAGGTCAT | SEQ ID NO: 409 |
| | probe3 | TAAGTGTCTAGAGCAAGCGGACTAAATATAGGTCGTGCGTCTATTTAGTGGAGCCCCGGTCCATTGGCCT | SEQ ID NO: 410 |
| | probe4 | GTGGTGCTTCTCACAAGCGGACTAAATATAGGTCGTGCGTCTATTTAGTGGAGCCAGCTCAGACCCAGAC | SEQ ID NO: 411 |
| | probe5 | GCCTTCCCAGCTTCCAGCGGACTAAATATAGGTCGTGCGTCTATTTAGTGGAGCCGCTCTCCAAGCACTA | SEQ ID NO: 412 |
| NCCR1 | probe1 | CATGGAGGCATGGGAATATCTTTAACGTCGAGCGCTGCGTCTATTTAGTGGAGCCGCGTTATGATCAGCT | SEQ ID NO: 413 |
| | probe2 | AGGAGTGAGCATGAGATATCTTTAACGTCGAGCGCTGCGTCTATTTAGTGGAGCCTCAGCCACCATTGCT | SEQ ID NO: 414 |
| | probe3 | ATGACAAACGAAGCTATATCTTTAACGTCGAGCGCTGCGTCTATTTAGTGGAGCCCGGATCACCAGGTCC | SEQ ID NO: 415 |
| | probe4 | TCTCGATGGACAGAAATATCTTTAACGTCGAGCGCTGCGTCTATTTAGTGGAGCCGAGCCTGTGGAGACC | SEQ ID NO: 416 |
| | probe5 | CTGCTCAGGAGGATGATATCTTTAACGTCGAGCGCTGCGTCTATTTAGTGGAGCCTCGCTTCCACTGTTT | SEQ ID NO: 417 |
| NCCR2 | probe1 | CTGAGTCTTTGAGGACACGGTATAGACACCACGTATGCGTCTATTTAGTGGAGCCCTCCGCGAGGTCTCC | SEQ ID NO: 418 |
| | probe2 | CTGGAAGGCCTGGGGCACGGTATAGACACCACGTATGCGTCTATTTAGTGGAGCCGCTGCACATCGGATT | SEQ ID NO: 419 |
| | probe3 | GATCTTGCAGCAGCACACGGTATAGACACCACGTATGCGTCTATTTAGTGGAGCCGCAGAACCTCGATGA | SEQ ID NO: 420 |
| | probe4 | CACGACGTACGCTCCCACGGTATAGACACCACGTATGCGTCTATTTAGTGGAGCCACTGGCTCCAAAAAG | SEQ ID NO: 421 |
| | probe5 | ACAGGCCTTATGACCCACGGTATAGACACCACGTATGCGTCTATTTAGTGGAGCCCCCGCCATCACCGGA | SEQ ID NO: 422 |
| WNT3A | probe1 | CTACGTGGAGATCATCTCTGCGAAGGTAGTGTAATTGCGTCTATTTAGTGGAGCCCCGCTTCTGCAGGAA | SEQ ID NO: 423 |
| | probe2 | CCTCTCTGGTGGCTGCTCTGCGAAGGTAGTGTAATTGCGTCTATTTAGTGGAGCCCTGGAGCTAGTGTCT | SEQ ID NO: 424 |
| | probe3 | AACCCGTGCCCCTGGCTCTGCGAAGGTAGTGTAATTGCGTCTATTTAGTGGAGCCGCGAGTCGGGTCCCC | SEQ ID NO: 425 |
| | probe4 | CGGGAGGTTTGGGGGCTCTGCGAAGGTAGTGTAATTGCGTCTATTTAGTGGAGCCTCATTTGGGGGCGTT | SEQ ID NO: 426 |
| | probe5 | GCACACCGCCATCCTCTCTGCGAAGGTAGTGTAATTGCGTCTATTTAGTGGAGCCAGAGCCCAAAAGAGG | SEQ ID NO: 427 |

TABLE 6-N

| ID | DO_seq1 | | DO_seq2 | |
|---|---|---|---|---|
| BpID_0001 | ACGGATAACTACTAGACtt | SEQ ID NO: 428 | ACGGATAACTACTAGACttT | SEQ ID NO: 486 |
| | TGTACCCGGTCACATAGAA | | GTGAGGGATCGTTATCTCC | |
| BpID_0002 | CTTAACTCGCGTTCAAGcc | SEQ ID NO: 429 | CTTAACTCGCGTTCAAGccT | SEQ ID NO: 487 |
| | TGTACCCGGTCACATAGAA | | GTGAGGGATCGTTATCTCC | |
| BpID_0003 | CTTAGACACATCATACGtc | SEQ ID NO: 430 | CTTAGACACATCATACGtcT | SEQ ID NO: 488 |
| | TGTACCCGGTCACATAGAA | | GTGAGGGATCGTTATCTCC | |
| BpID_0004 | GGTACGGAAGTGTATATag | SEQ ID NO: 431 | GGTACGGAAGTGTATATagT | SEQ ID NO: 489 |
| | TGTACCCGGTCACATAGAA | | GTGAGGGATCGTTATCTCC | |
| BpID_0065 | ACAGTTTAGACTATTCGaa | SEQ ID NO: 432 | ACAGTTTACACTATTCGaaT | SEQ ID NO: 490 |
| | TGTACCCGGTCACATAGAA | | GTGAGGGATCGTTATCTCC | |
| BpID_0006 | ACATAGACCATACCACGcc | SEQ ID NO: 433 | ACATAGACCATACCACGccT | SEQ ID NO: 491 |
| | TGTACCCGGTCACATAGAA | | GTGAGGGATCGTTATCTCC | |
| BpID_0012 | CCTAGATACCGTAGATGtg | SEO ID NO: 434 | CCTAGATACCGTAGATGtGT | SEQ ID NO: 492 |
| | TGTACCCGGTCACATAGAA | | GTGAGGGATCGTTATCTCC | |
| BpID_0013 | GTTGCTACGATGACTCAaa | SEQ ID NO: 435 | GTTGCTACGATGACTCAaaT | SEQ ID NO: 493 |
| | TGTACCCGGTCACATAGAA | | GTGAGGGATCGTTATCTCC | |
| BpID_0007 | AGACGTAGTGAGCATGAaa | SEQ ID NO: 436 | AGACGTAGTGAGCATGAgaT | SEQ ID NO: 494 |
| | TGTACCCGGTCACATAGAA | | GTGAGGGATCGTTATCTCC | |
| BpID_0008 | GGACATTACCACCTAGAca | SEQ ID NO: 437 | GGACATTACCACCTAGAcaT | SEQ ID NO: 495 |
| | TGTACCCGGTCACATAGAA | | GTGAGGGATCGTTATCTCC | |
| BpID_0018 | TAGACAGACTGCGACATtg | SEQ ID NO: 438 | TAGACAGACTGCGACATtgT | SEQ ID NO: 496 |
| | TGTACCCGGTCACATAGAA | | GTGAGGGATCGTTATCTCC | |
| BpID_0019 | TAGACATATCTCACAGCct | SEQ ID NO: 439 | TAGACATATCTCACAGCctT | SEQ ID NO: 497 |
| | TGTACCCGGTCACATAGAA | | GTGAGGGATCGTTATCTCC | |
| BpID_0021 | TCTGCGAAGGTAGTGTAta | SEQ ID NO: 440 | TCTGCGAAGGTAGTGTAtaT | SEQ ID NO: 498 |
| | TGTACCCGGTCACATAGAA | | GTGAGGGATCGTTATCTCC | |
| BpID_0022 | TTTCCAATACGGTGTACct | SEQ ID NO: 441 | TTTCCAATACGGTGTACctT | SEQ ID NO: 499 |
| | TGTACCCGGTCACATAGAA | | GTGAGGGATCGTTATCTCC | |
| BpID_0023 | TTTCGACGCTGACACTAtt | SEQ ID NO: 442 | TTTCGACGCTGACACTAttT | SEQ ID NO: 500 |
| | TGTACCCGGTCACATAGAA | | GTGAGGGATCGTTATCTCC | |
| BpID_0024 | TTTGTGAATAGTCTAGCcg | SEQ ID NO: 443 | TTTGTGAATAGTCTAGCcgT | SEQ ID NO: 501 |
| | TGTACCCGGTCACATAGAA | | GTGAGGGATCGTTATCTCC | |
| BpID_0025 | CACTATTACGCAGAGCAag | SEG ID NO: 444 | CACTATTACGCAGAGCAagT | SEQ ID NO: 502 |
| | TGTACCCGGTCACATAGAA | | GTGAGGGATCGTTATCTCC | |
| BpID_0026 | GTATAGTACATTCCCGCtg | SEO ID NO: 445 | GTATAGTACATTCCCGCtgT | SEQ ID NO: 503 |
| | TGTACCCGGTCACATAGAA | | GTGAGGGATCGTTATCTCC | |
| BpID_0027 | GTATECGACCTACTCTTtg | SEQ ID NO: 446 | GTATGCGACCTACTCTTtgT | SEQ ID NO: 504 |
| | TGTACCCGGTCACATAGAA | | GTGAGGGATCGTTATCTCC | |
| BpID_0028 | TCTGAGAATGTGTTAGCtg | SEQ ID NO: 447 | TCTGAGAATGTGTTAGCtgT | SEQ ID NO: 505 |
| | TGTACCCGGTCACATAGAA | | GTGAGGGATCGTTATCTCC | |
| BpID_0029 | TCTGCATAGAGAACAACca | SEQ ID NO: 448 | TCTGCATAGAGAACAACcaT | SEQ ID NO: 506 |
| | TGTACCCGGTCACATAGAA | | GTGAGGGATCGTTATCTCC | |
| BpID_0030 | ACGTTTGACGAGGAGTAgt | SEQ ID NO: 449 | ACGTTTGACGAGGAGTAgtT | SEQ ID NO: 507 |
| | TGTACCCGGTCACATAGAA | | GTGAGGGATCGTTATCTCC | |
| BpID_0033 | CCTAATAACACTCACGGaa | SEQ ID NO: 450 | CCTAATAACACTCACGGaaT | SEQ ID NO: 509 |
| | TGTACCCGGTCACATAGAA | | GTGAGGGATCGTTATCTCG | |
| BpID_0034 | CCTACCTAAAGTGACATaa | SEQ ID NO: 451 | CCTACCTAAAGTGACATaaT | SEQ ID NO: 509 |
| | TGTACCCGGTCACATAGAA | | GTGAGGGATCGTTATCTCC | |
| BpID_0035 | GATACGGAAATGGACGTcg | SEQ ID NO: 452 | GATACGGAAATGGACGTcgT | SEQ ID NO: 510 |
| | TGTACCCGGTCACATAGAA | | GTGAGGGATCGTTATCTCC | |
| BpID_0036 | GATACTTAACCATAGCGtt | SEQ ID NO: 453 | GATACTTAACCATAGCGttT | SEQ ID NO: 511 |
| | TGTACCCGGTCACATAGAA | | GTGAGGGATCGTTATCTCC | |
| BpID_0037 | TAGACTTAACGCACGATtt | SEO ID NO: 454 | TAGACTTAACGCACGATttT | SEQ ID NO: 512 |
| | TGTACCCGGTCACATAGAA | | GTGAGGGATCGTTATCTCC | |
| BpID_0038 | TAGAGCGAAGGATAGTTaa | SEQ ID NO: 455 | TAGAGCGAACGATAGTTaaT | SEQ ID NO: 513 |
| | TGTACCCGGTCACATAGAA | | GTGAGGGATCGTTATCTCC | |
| BpID_0039 | TAGAGTCAACGAGCGTAtt | SEO ID NO: 456 | TAGAGTCAACGAGCGTAttT | SEQ ID NO: 514 |
| | TGTACCCGGTCACATAGAA | | GTGAGGGATCGTTATCTCC | |
| BpID_0040 | ACACTAACTCGAATGACCA | SEQ ID NO: 457 | ACACTAACTCGAATGACCAT | SEQ ID NO: 515 |
| | TGTACCCGGTCACATAGAA | | GTGAGGGATCGTTATCTCC | |
| BpID_0041 | ACACTGTAAGACGATAGGG | SEQ ID NO: 458 | ACACTGTAAGACGATAGGGT | SEQ ID NO: 516 |
| | TGTACCCGGTCACATAGAA | | GTGAGGGATCGTTATCTCC | |
| BpID_0042 | CGTCAGAAGCCATACTACT | SEQ ID NO: 459 | CGTCAGAAGCCATACTACTT | SEQ ID NO: 517 |
| | TGTACCCGGTCACATAGAA | | GTGAGGGATCGTTATCTCC | |
| BpID_0043 | CGTCGATACAGACTCAGTA | SEQ ID NO: 460 | CGTCGATACAGACTCAGTAT | SEQ ID NO: 518 |
| | TGTACCCGGTCACATAGAA | | GTGAGGGATCGTTATCTCC | |
| BpID_0044 | GGACTAAGGGCGACTAAAT | SEQ ID NO: 461 | GGACTAAGGGCGACTAAATT | SEQ ID NO: 519 |
| | TGTACCCGGTCACATAGAA | | GTGAGGGATCGTTATCTCC | |
| BpID_0045 | TAGACTGAATCATCACCCT | SEQ ID NO: 462 | TAGACTGAATCATCACCCTT | SEQ ID NO: 520 |
| | TGTACCCGGTCACATAGAA | | GTGAGGGATCGTTATCTCC | |

TABLE 6-O

| BpID_0046 | TCTGTGAATACCGAGTAGTTGT | SEQ ID NO: 463 | TCTGTGAATACCGAGTAGTTGTG | SEQ ID NO: 521 |
|---|---|---|---|---|
| | ACCCGGTCACATAGAA | | AGGGATCGTTATCTCC | |
| BpID_0047 | AACTATGCTGACAGTACGCTGT | SEQ ID NO: 464 | AACTATGCTGACASTAGGCTGTG | SEQ ID NO: 522 |
| | ACCCGGTCACATAGAA | | AGCGATCGTTATCTCC | |
| BpID_0048 | GTCAGAGACGTAGCATCTTTGT | SEQ ID NO: 465 | GTCAGAGACGTAGCATCTTTGTG | SEQ ID NO: 523 |
| | ACCCGGTCACATAGAA | | AGGGATCGTTATCTCC | |
| BpID_0049 | TCTGGAGAGATCAGTATGGTGT | SEQ ID NO: 465 | TCTGGAGAGATCAGTATGGTGTG | SEQ ID NO: 524 |
| | ACCCGGTCACATAGAA | | AGGGATCGTTATCTCC | |
| BpID_0050 | ACTATGCCTACCTGTCGTATGT | SEQ ID NO: 467 | ACTATGCCTACCTGTCGTATGTG | SEQ ID NO: 525 |
| | ACCCGGTCACATAGAA | | AGGGATCGTTATCTCC | |
| BpID_0051 | CGCGTCTACCTGACTTTTCTGT | SEQ ID NO: 468 | CGCGTCTACCTGACTTTTGTGTG | SEQ ID NO: 526 |
| | ACCCGGTCACATAGAA | | AGGGATCGTTATCTCC | |
| BpID_0062 | CGCGTTCACCGTAATCTATTGT | SEQ ID NO: 469 | CGCGTTCACCGTAATCTATTGTG | SEQ ID NO: 527 |
| | ACCCGGTCACATAGAA | | AGGGATCGTTATCTCC | |
| BpID_0053 | CCTAGGAACACTAAGTCCCTGT | SEQ ID NG: 470 | CCTACGAACACTAAGTCCCTGTG | SEQ ID NO: 528 |
| | ACCCGGTCACATAGAA | | AGGGATCGTTATCTCC | |
| BpID_0G54 | AGACTCTCGATCAGCCGATTGT | SEQ ID NO: 471 | AGACTCTCGATCAGCCGATTGTG | SEQ ID NO: 529 |
| | ACCCGGTCACATAGAA | | AGGGATCGTTATCTCC | |
| BpID_0065 | TGACATAGTGGTCGAGTAGTGT | SEQ ID NO: 472 | TGACATAGTGGTCGAGTAGTGTG | SEQ ID NO: 530 |
| | ACCCGGTCACATAGAA | | AGGGATCGTTATGTCC | |
| BpID_0056 | ACCGTCTAAACCATACGACTGT | SEQ ID NO: 473 | ACGGTCTAAACCATACGACTGTG | SEQ ID NO: 531 |
| | ACCCGGTCACATAGAA | | AGGGATCGTTATGTCC | |
| BpID_0057 | ACGACTAACAACTGTCCCATGT | SEQ ID NO: 474 | ACGACTAACAACTGTCCCATGTG | SEQ ID NO: 532 |
| | ACCCGGTCACATAGAA | | AGGGATCGTTATCTCC | |
| BpID_0058 | CGAGGTTAAAGTCAAGTGGTGT | SEQ ID NO: 475 | CGAGGTTAAAGTCAAGTGGTGTG | SEQ ID NO: 533 |
| | ACCCGGTCACATAGAA | | AGGGATCGTTATCTCC | |
| BpID_0069 | CGAGTAGAAATGACGCTGGTGT | SEQ ID NO: 478 | CCAGTAGAAATGADGETGGTGTG | SEQ ID NO: 534 |
| | ACCCGGTCACATAGAA | | AGGGATCGTTATCTCC | |
| BpID_0080 | GCGGACTAAATATAGGTGCTGT | SEQ ID NO: 477 | GCGGACTAAATATAGGTGCTGTG | SEQ ID NO: 535 |
| | ACCCGGTCACATAGAA | | AGGGATCGTTATCTCC | |
| BpID_0061 | TATCTTTAACGTCGAGCCGTGT | SEQ ID NO: 478 | TATCTTTAACGTCGAGCCGTGTG | SEQ ID NO: 536 |
| | ACCCGGTCACATAGAA | | AGGGATCGTTATCTCC | |
| BpID_0063 | ACGGTATAGACACCACGATTGT | SEQ ID NO: 479 | ACGGTATAGACACCACGATTGTG | SEQ ID NO: 537 |
| | ACCCGGTCACATAGAA | | AGGGATCGTTATCTCC | |
| BpID_0064 | ACGGTCAACTAGGTGAGTTTGT | SEQ ID NO: 480 | ACGGTCAACTAGGTGAGTTGTGA | SEG ID NO: 538 |
| | ACCCGGTCACATAGAA | | GGGATCGTTATCTCC | |
| BpID_0065 | CTTATCGAAGTAGACGAGTTGT | SEQ ID NO: 481 | CTTATCGAAGTAGACGAGTTGTG | SEQ ID NO: 539 |
| | ACCCGGTCACATAGAA | | AGGGATCGTTATCTCC | |
| BpID_0666 | GGTACTAAGGCTGACCTTTTGT | SEQ ID NO: 482 | GGTACTAAGGCTGACCTTTTGTG | SEQ ID NO: 540 |
| | ACCCGGTCACATAGAA | | AGGGATCGTTATCTCC | |
| BpID_0067 | GGTCAACTCTACCTAAGCTTGT | SEQ ID NO: 483 | GGTCAACTCTACCTAAGCTTGTG | SEQ ID NO: 541 |
| | ACCCGGTCACATAGAA | | AGGGATCGTTATCTCC | |
| BpID_0068 | ACATCAGAGTACCAGCTGTTGT | SEQ ID NO: 484 | ACATCAGAGTACCAGCTGTTGTG | SEQ ID NO: 542 |
| | ACCCGGTCACATAGAA | | AGGGATCGTTATCTCC | |
| BpID_0126 | TCGTCGGATCGAGGTTAAGTGT | SEQ ID NO: 485 | TCGTCGGATCGAGGTTAAGTGTG | SEQ ID NO: 543 |
| | ACCCGGTCACATAGAA | | AGGGATCGTTATCTCC | |
| ID | DQ_seq3 | | DQ_seq4 | |
| BpID_0001 | ACGGATAACTACTAGACttACA | SEQ ID NO: 544 | ACGGATAACTACTAGACttGACA | SEQ ID NO: 602 |
| | GTTGGAAAGCCGAGTAC | | GGCAGTCACAGATATG | |
| BpID_0002 | CTTAACTCGCGTTCAAGccACA | SEQ ID NO: 545 | CTTAACTCGCGTTCAAGccGACA | SEQ ID NO: 603 |
| | GTTGGAAAGCCGAGTAC | | GGCAGTCACAGATATG | |
| BpID_0003 | CTTAGACACATCATACGtcACA | SEQ ID NO: 546 | CTTAGACACATCATACGtcGACA | SEQ ID NO: 604 |
| | GTTGGAAAGCCGAGTAC | | GGCAGTCACAGATATG | |
| BpID_0004 | GGTACGGAAGTGTATATagACA | SEQ ID NO: 547 | GGTACGGAAGTGTATATagGACA | SEQ ID NO: 605 |
| | GTTGGAAAGCCGAGTAC | | GGCAGTCACAGATATG | |
| BpID_0005 | ACAGTTTACACTATTCGaaACA | SEQ ID NO: 548 | ACAGTTTACACTATTCGaaGACA | SEQ ID NO: 606 |
| | GTTGGAAAGCCGAGTAC | | GGCAGTCACAGATATG | |
| BpID_0006 | ACATAGACCATACCACGccACA | SEQ ID NO: 549 | ACATAGACCATACCACGccGACA | SEQ ID NO: 607 |
| | GTTGGAAAGCCGAGTAC | | GGCAGTCACAGATATG | |
| BpID_0012 | CCTAGATACCGTAGATGtgACA | SEQ ID NO: 550 | CCTAGATACCGTAGATGtgGACA | SEQ ID NO: 608 |
| | GTTGGAAAGCCGAGTAC | | GGCAGTCACAGATATG | |
| BpID_0013 | GTTGCTACGATGACTCAggACA | SEQ ID NO: 551 | GTTGCTACGATGACTCAaaGACA | SEQ ID NO: 609 |
| | GTTGGAAAGCCGAGTAC | | GGCAGTCACAGATATG | |
| BpID_0007 | AGACGTAGTGAGCATGAaaACA | SEQ ID NO: 552 | AGACGTAGTGAGCATGAaaGACA | SEQ ID NO: 610 |
| | GTTGGAAAGCCGAGTAC | | GGCAGTCACAGATATG | |
| BpID_0008 | GGACATTACCACCTAGAcaACA | SEQ ID NO: 553 | GGACATTACCACCTAGAcaGACA | SEQ ID NO: 611 |
| | GTTGGAAAGCCGAGTAC | | GGCAGTCACAGATATG | |

TABLE 6-P

| | | | | |
|---|---|---|---|---|
| BpID_0018 | TAGACAGACTGCGACATtgA | SEQ ID NO: 554 | TAGACAGACTGCGACATtgG | SEQ ID NO: 612 |
| | CAGTTGGAAAGCCGAGTAC | | ACAGGCAGTCACAGATATG | |
| BpID_0019 | TAGACATATCTCACAGCCTA | SEQ ID NO: 555 | TAGACATATCTCACAGCctG | SEQ ID NO: 613 |
| | CAGTTGGAAAGCCGAGTAC | | ACAGGCAGTCACAGATATG | |
| BpID_0021 | TCTGCGAAGGTAGTGTAtaA | SEQ ID NO: 556 | TCTGCGAAGGTAGTGTAtaG | SEQ ID NO: 614 |
| | CAGTTGGAAAGCCGAGTAC | | ACAGGCAGTCACAGATATG | |
| BpID_0022 | TTTCCAATACGGTGTACctA | SEQ ID NO: 557 | TTTCCAATACGGTGTACctG | SEQ ID NO: 615 |
| | CAGTTGGAAAGCCGAGTAC | | ACAGGCAGTCACAGATATG | |
| BpID_0023 | TTTCGACGCTGACACTAttA | SEQ ID NO: 558 | TTTCGACGCTGACACTAttG | SEQ ID NO: 616 |
| | CAGTTGGAAAGCCGAGTAC | | ACAGGCAGTCACAGATATG | |
| BpID_0024 | TTTGTGAATAGTCTAGCcgA | SEQ ID NO: 559 | TTTGTGAATAGTCTAGCcgG | SEQ ID NO: 617 |
| | CAGTTGGAAAGCCGAGTAC | | ACAGGCAGTCACAGATATG | |
| BpID_0025 | CACTATTACGCAGAGCAagA | SEQ ID NO: 560 | CACTATTACGCAGAGCAagG | SEQ ID NO: 618 |
| | CAGTTGGAAAGCCGAGTAC | | ACAGGCAGTCACAGATATG | |
| BpID_0026 | GTATAGTACATTCCCGCtgA | SEQ ID NO: 561 | GTATAGTACATTCCCGCtgG | SEQ ID NO: 619 |
| | CAGTTGGAAAGCCGAGTAC | | ACAGGCAGTCACAGATATG | |
| BpID_0027 | GTATGCGACCTACTCTTtgA | SEQ ID NO: 562 | GTATGCGACCTACTCTTtgG | SEQ ID NO: 620 |
| | CAGTTGGAAAGCCGAGTAC | | ACAGGCAGTCACAGATATG | |
| BpID_0028 | TCTGAGAATGTGTTAGCtgA | SEQ ID NO: 563 | TCTGAGAATGTGTTAGGTgG | SEQ ID NO: 621 |
| | CAGTTGGAAAGCCGAGTAC | | ACAGGCAGTCACAGATATG | |
| BpID_0029 | TCTGCATAGAGAACAAGcaA | SEQ ID NO: 564 | TTCGCATAGAGAACAACcaG | SEQ ID NO: 622 |
| | CAGTTGGAAAGCCGAGTAC | | ACAGGCAGTCACAGATATG | |
| BpID_0030 | ACGTTTGACGAGGAGTAGTA | SEQ ID NO: 565 | ACGTTTGACGAGGAGTAgtG | SEQ ID NO: 623 |
| | CAGTTGGAAAGCCGAGTAC | | ACAGGCAGTCACAGATATG | |
| BpID_0033 | CCTAATAACACTCACGGaaA | SEQ ID NO: 566 | CCTAATAACACTCACGGaaG | SEQ ID NO: 624 |
| | CAGTTGGAAAGCCGAGTAC | | ACAGGCAGTCACAGATATG | |
| BpID_0034 | CCTACCTAAAGTGACATaaA | SEQ ID NO: 567 | CCTACCTAAAGTGACATaaG | SEQ ID NO: 625 |
| | CAGTTGGAAAGCCGAGTAC | | ACAGGCAGTCACAGATATG | |
| BpID_0035 | GATACGGAAATGGACGTcgA | SEQ ID NO: 568 | GATACGGAAATGGACGTcgG | SEQ ID NO: 626 |
| | CAGTTGGAAAGCCGAGTAC | | ACAGGCAGTCACAGATATG | |
| BpID_0036 | GATACTTAACCATAGCGttA | SEQ ID NO: 569 | GATACTTAACCATAGCGttG | SEQ ID NO: 627 |
| | CAGTTGGAAAGCCGAGTAC | | ACAGGCAGTCACAGATATG | |
| BpID_0037 | TAGACTTAACGCACGATttA | SEQ ID NO: 570 | TAGACTTAACGCACGATttG | SEQ ID NO: 628 |
| | CAGTTGGAAAGCCGAGTAC | | ACAGGCAGTCACAGATATG | |
| BpID_0038 | TAGAGCGAACGATAGTTaaA | SEQ ID NO: 571 | TAGAGCGAACGATAGTTaaG | SEQ ID NO: 629 |
| | CAGTTGGAAAGCCGAGTAC | | ACAGGCAGTCACAGATATG | |
| BpID_0039 | TAGAGTCAACGAGCGTAttA | SEQ ID NO: 572 | TAGAGTCAACGAGCGTAttG | SEQ ID NO: 630 |
| | CAGTTGGAAAGCCGAGTAC | | ACAGGCAGTCACAGATATG | |
| BpID_0040 | ACACTAACTCGAATGACCAA | SEQ ID NO: 573 | ACACTAACTCGAATGACCAG | SEQ ID NO: 631 |
| | CAGTTGGAAAGCCGAGTAC | | ACAGGCAGTCACAGATATG | |
| BpID_0041 | ACACTGTAAGACGATAGGGA | SEQ ID NO: 574 | ACACTGTAAGACGATAGGGG | SEQ ID NO: 632 |
| | CAGTTGGAAAGCCGAGTAC | | ACAGGCAGTCACAGATATG | |
| BpID_0042 | CGTCAGAAGCCATAGTACTA | SEQ ID NO: 575 | CGTCAGAAGCCATACTACTG | SEQ ID NO: 633 |
| | CAGTTGGAAAGCCGAGTAC | | ACAGGCAGTCACAGATATG | |
| BpID_0043 | CGTCGATACAGACTCAGTAA | SEQ ID NO: 576 | CGTCGATACAGACTCAGTAG | SEQ ID NO: 634 |
| | CAGTTGGAAAGCCGAGTAC | | ACAGGCAGTCACAGATATG | |
| BpID_0044 | GGACTAAGGGCGACTAAATA | SEQ ID NO: 577 | GGACTAAGGGCGACTAAATG | SEQ ID NO: 635 |
| | CAGTTGGAAAGCCGAGTAC | | ACAGGCAGTCACAGATATG | |
| BpID_0045 | TAGACTGAATCATCACCCTA | SEQ ID NO: 578 | TAGACTGAATCATCACCCTG | SEQ ID NO: 636 |
| | CAGTTGGAAAGCCGAGTAC | | ACAGGCAGTCACAGATATG | |
| BpID_0046 | TCTGTGAATACCGAGTAGTA | SEQ ID NO: 579 | TCTGTGAATACCGAGTAGTG | SEQ ID NO: 637 |
| | CAGTTGGAAAGCCGAGTAC | | ACAGGCAGTCACAGATATG | |
| BpID_0047 | AACTATGCTGACAGTACGCA | SEQ ID NO: 580 | AACTATGCTGACAGTACGCG | SEQ ID NO: 638 |
| | CAGTTGGAAAGCCGAGTAC | | ACAGGCAGTCACAGATATG | |
| BpID_0048 | GTCAGAGACGTAGCATCTTA | SEQ ID NO: 551 | GTCAGAGACGTAGCATCTTG | SEQ ID NO: 639 |
| | CAGTTGGAAAGGCGAGTAC | | ACAGGCAGTCACAGATATG | |
| BpID_0649 | TCTGGAGAGATCAGTATGGA | SEQ ID NO: 582 | TCTGGAGAGATCAGTATGGG | SEQ ID NO: 640 |
| | CAGTTGGAAAGCCGAGTAC | | ACAGGCAGTCACAGATATG | |
| BpID_0050 | ACTATGCCTACCTGTCGTAA | SEQ ID NO: 583 | ACTATGCCTACCTGTCGTAG | SEQ ID NO: 641 |
| | CAGTTGGAAAGCCGAGTAG | | ACAGGCAGTCACAGATATG | |
| BpID_0051 | CGCGTCTACCTGACTTTTCA | SEQ ID NO: 584 | CGCGTCTACCTGACTTTTCG | SEQ ID NO: 642 |
| | CAGTTGGAAAGCCGAGTAC | | ACAGGCAGTCACAGATATG | |
| BpID_0052 | CGCGTTCACCGTAATCTATA | SEQ ID NO: 585 | CGCGTTCACCGTAATCTATG | SEQ ID NO: 643 |
| | CAGTTGGAAAGCCGAGTAC | | ACAGGCAGTCAçAGATATG | |
| BpID_0053 | CCTACGAACACTAAGTCCCA | SEQ ID NO: 586 | CCTACGAACACTAAGTCCCG | SEQ ID NO: 644 |
| | CAGTTGGAAAGCCGAGTAC | | ACAGGCAGTCACAGATATG | |
| BpID_0054 | AGACTCTCGATCAGCCGATA | SEQ ID NO: 587 | AGACTCTCGATCAGCCGATG | SEQ ID NO: 645 |
| | CAGTTGGAAAGCCGAGTAC | | ACAGGGAGTCACAGATATG | |
| BpID_0055 | TGACATAGTGGTCGAGTAGA | SEQ ID NO: 588 | TGACATAGTGGTCGAGTAGG | SEQ ID NO: 646 |
| | CAGTTGGAAAGCCGAGTAC | | ACAGGCAGTCACAGATATG | |
| BpID_0056 | ACCGTCTAAACCATACGACA | SEQ ID NO: 589 | ACCGTCTAAACCATACGACG | SEQ ID NO: 647 |
| | CAGTTGGAAAGCCGAGTAC | | ACAGGCAGTCACAGATATG | |

TABLE 6-Q

| | | | | |
|---|---|---|---|---|
| BpID_0057 | ACGACTAACAACTGTCCCAAC | SEQ ID NO: 560 | ACGACTAACAACTGTCCCAGAC | SEQ ID NO: 648 |
| | AGTTGGAAAGCCGAGTAC | | AGGCAGTCACAGATATG | |
| BpID_0058 | CGAGGTTAAAGTCAAGTGGAC | SEQ ID NO: 591 | CGAGGTTAAAGTCAAGTGGGAC | SEQ ID NO: 649 |
| | ACTTCGAAAGCCGAGTAC | | AGGCAGTCACAGATATG | |
| BpID_0059 | CGAGTAGAAATGACGCTGGAC | SEQ ID NO: 592 | CGAGTAGAAATGACGCTGGGAC | SEQ ID NO: 650 |
| | AGTTGGAAAGCCGAGTAC | | AGGCAGTCACAGATATG | |
| BpID_0060 | GCGGACTAAATATAGGTGCAC | SEQ ID NO: 593 | GCGGACTAAATATAGGTGCGAC | SEQ ID NO: 651 |
| | AGTTGGAAAGCCGAGTAC | | AGGCAGTCACAGATATG | |
| BpID_0061 | TATCTTTAACGTCGAGCCGAC | SEQ ID NO: 594 | TATCTTTAACGTCGAGCCGGAC | SEQ ID NO: 652 |
| | AGTTGGAAAGCCGAGTAC | | AGGCAGTCACAGATATG | |
| BpID_0063 | ACGGTATAGACACCACGATAC | SEQ ID NO: 595 | AGGGTATAGACACCACGATGAC | SEQ ID NO: 653 |
| | AGTTGGAAAGCCGAGTAC | | AGGCACTCACAGATATG | |
| BpID_0064 | ACGGTCAACTAGGTGAGTTAC | SEQ ID NO: 596 | ACGGTCAACTAGGTCAGTTGAC | SEQ ID NO: 654 |
| | AGTTGGAAAGCCGAGTAC | | AGGCAGTCACAGATATG | |
| BpID_0065 | CTTATCGAAGTAGACGAGTAC | SEQ ID NO: 597 | CTTATCGAAGTAGACGAGTGAC | SEQ ID NO: 655 |
| | AGTTGGAAAGCCGAGTAC | | AGGCAGTCACAGATATG | |
| BpID_0068 | GGTACTAAGGCTGACCTTTAC | SEQ ID NO: 598 | GGTACTAAGGCTGACCTTTGAC | SEQ ID NO: 656 |
| | AGTTGGAAAGCCGAGTAC | | AGGCAGTCACAGATATG | |
| BpID_0067 | GGTCAACTCTACCTAAGCTAC | SEQ ID NO: 599 | GGTCAACTCTACCTAAGCTGAC | SEQ ID NO: 657 |
| | AGTTGGAAAGCCGAGTAC | | AGGCAGTCACAGATATG | |
| BpID_0068 | ACATCAGAGTACCAGCTGTAC | SEQ ID NO: 800 | ACATCAGAGTACCAGCTGTGAC | SEQ ID NO: 658 |
| | AGTTGGAAAGCCGAGTAC | | AGGCAGTCACAGATATG | |
| BpID_0126 | TCGTCGGATCGAGGTTAAGAC | SEQ ID NO: 601 | TCGTCGGATCGAGGTTAAGGAC | SEQ ID NO: 659 |
| | AGTTGGAAAGCCGAGTAC | | AGGCAGTCACAGATATG | |

TABLE 6-R

| | | |
|---|---|---|
| DO_seq1_ | TTCTATGTGACCGGGTACA | SEQ ID NO: 660 |
| AlexaFluor750 | | |
| DO_seq2_ | GGAGATAACGATCCCTCACA | SEQ ID NO: 661 |
| AlexaFluor488 | | |
| DO_seq3_Cy3 | GTACTCGGCTTTCCAACTGT | SEQ ID NO: 662 |
| DO_seq4_Cy5 | CATATCTGTGACTGCCTGTC | SEQ ID NO: 663 |

Supplementary Table 7 (Table 7): List of primers used for CUT&Tag experiments. List of utilized primers used for CUT&Tag-based analysis of human axioloids.

TABLE 7
Primer1

| | | |
|---|---|---|
| H3K4me3_rep1 | AATGATACGGCGACCACCGAGATCTACACACATGTGCT | SEQ ID NO: 664 |
| | CGTCGGCAGCGTCAGATGTGTAT | |
| H3K27me3_rep1 | AATGATACGGCGACCACCGAGATCTACACTTGTGTGCT | SEQ ID NO: 665 |
| | CGTCGGCAGCGTCAGATGTGTAT | |
| H3K4me3_rep2 | AATGATACGGCGACCACCGAGATCTACACTGGTTCAAT | SEQ ID NO: 666 |
| | CGTCGGCAGCGTCAGATGTGTAT | |
| H3K27me3_rep2 | AATGATACGGCGACCACCGAGATCTACACAAGTAGAGT | SEQ ID NO: 667 |
| | CGTCGGCAGCGTCAGATGTGTAT | |

Primer2

| | | |
|---|---|---|
| H3K4me3_rep1 | CAAGCAGAAGACGGCATACGAGATCTTGCACTGTCTCG | SEQ ID NO: 668 |
| | TGGGCTCGGAGATGTG | |
| H3K27me3_rep1 | CAAGCAGAAGACGGCATACGAGATGAAGCAACGTCTCG | SEQ ID NO: 669 |
| | TGGGCTCGGAGATGTG | |
| H3K4me3_rep2 | CAAGCAGAAGACGGCATACGAGATGAGAGAAGGTCTCG | SEQ ID NO: 670 |
| | TGGGCTCGGAGATGTG | |
| H3K27me3_rep2 | CAAGCAGAAGACGGCATACGAGATTGTCGTCTGTCTCG | SEQ ID NO: 671 |
| | TGGGCTCGGAGATGTG | |

### Supplementary Videos

### Supplementary Video 1: Symmetry Breaking and Initial Elongation of Axioloids.

Live imaging of axioloids derived from 409B2 (upper) and 201B7 Luc (lower) between 24 h and 72 h of culture. Data shown is representative of at least three independent experiments. Scale bar is 200 m.

### Supplementary Video 2: Effect of Matrigel on Axioloid Morphology.

Live imaging of axioloids derived from 409B2 (upper) and 201B7 Luc (lower) with +MG (right) or without −MG (left) embedding into Matrigel (MG) between 72 h and 120 h of culture. Data shown is representative of at least three independent experiments. Scale bar is 200 m.

### Supplementary Video 3: HES7 Gene Expression Dynamics in MG Embedded Axioloids.

Live imaging of the spatiotemporal morphogenetic expression of the HES7 gene in a 201B7 Luc-derived axioloid embedded in MG from 72 h to 120 h of culture. BF video (left) and HES7:Luciferase signal (right). Data shown is representative of at least three independent experiments. Scale bar is 200 m.

### Supplementary Video 4: Effect of Retinoid Signaling on Axioloid Growth and Morphology.

Live imaging of axioloids derived from 409B2 (top panels) and 201B7 Luc (bottom panels) after embedding in MG only (left) or in MG supplemented with retinol (ROL), retinal (RAL) or retinoic acid (RA) (right) from 72 h to 120 h of culture. Data shown is representative of at least three independent experiments. Scale bar is 200 m.

### Supplementary Video 5: 3D Reconstruction of Retinoid Treated Axioloids.

3D reconstruction of 409B2-derived axioloids embedded in MG only (upper left) or in MG supplemented with retinol (ROL) (lower left), retinal (RAL) (lower right) or retinoic acid (RA) (upper right) at 120 h of culture stained for Factin (Phalloidin) in gray, TBXT (BRA) in blue, Fibronectin (FN1) in green and MEOX1 in red.

### Supplementary Video 6: Effect of RA Pathway Inhibition on Axioloid Growth and Morphology

Live imaging of axioloids derived from 201B7 Luc after embedding in +MG+RAL supplemented with (from left to right) DMSO, BMS493, AGN193109 or ER50891 from 72 h to 120 h of culture. Data shown is representative of at least three independent experiments. Scale bar is 200 m.

Supplementary Video 7: 3D Visualization of Axioloids Treated with RA Pathway Inhibitors.

3D reconstruction of axioloids derived from 201B7 Luc embedded in +MG+RAL (upper left) or in +MG+RAL supplemented with three different RA inhibitors, including BMS493 (upper right), AGN193109 (lower left) or ER50891 (lower right) at 120 h of culture stained for F-actin (Phalloidin) in gray, TBXT (BRA) in blue, Fibronectin (FN1) in green and MEOX1 in red.

### Supplementary Video 8: Midline and Bilateral Somite Formation in Axioloids.

Visualization of midline formation in a 409B2-derived axioloid embedded in +MG +RAL (top) and of formation of a single bilateral somite in 409B2-derived axioloid embedded in +MG+ROL (bottom). Live imaging was performed between 72 and 120 h of culture. Scale bar is 200 m

### Supplementary Video 9: 3D Reconstruction of Somites in Axioloids and Human CS9-11 Embryos.

3D reconstruction of somites formed in 409B2-derived MG embedded axioloids (top) treated retinol (ROL) (left), retinal (RAL) (middle) and retinoic acid (RA); and 3D reconstructions of somites in human embryos (bottom) found in CS9 (left), CS10 (middle) and CS11 (right) human embryos. Each somite-like structure is highlighted by a different color depending on its position along the antero-posterior axis.

### Supplementary Video 10: Effect of ROL, RAL or RA on HES7 Gene Expression Dynamics.

Live imaging of the spatiotemporal morphogenetic expression of the HES7 gene in 201B7 Luc-derived axioloids embedded in MG only (upper left pair) or in MG supplemented with retinol (ROL) (lower left pair), retinal (RAL) (lower right pair) or retinoic acid (RA) (upper right pair) from 72 h to 120 h of culture. BF video (left) and HES7:Luciferase signal (right) for each condition. Data shown is representative of at least three independent experiments. Scale bar is 200 m.

### Supplementary Video 11: Effect of BMS493 Supplementation on HES7 Gene Expression Dynamics.

Live imaging of the spatiotemporal morphogenetic expression of the HES7 gene in 201B7 Luc-derived axioloids embedded in MG supplemented with +RAL+DMSO (left pair) or +RAL+BMS493 (right pair), from 72 h to 120 h of culture. BF video (left) and HES7:Luciferase signal (right) for each condition. Data shown is representative of at least three independent experiments. Scale bar is 200 m.

### Supplementary Video 12: Effect of NOTCH, FGF and WNT Pathway Inhibition on Axioloids.

Live imaging of axioloids derived from 201B7 Luc after embedding in +MG+RAL supplemented with DMSO (top left), DAPT (top right), PD173074 (middle left), PD0325901 (middle right), XAV939 (bottom left) or IWP2 (bottom right) from 72 h to 120 h of culture. Data shown is representative of at least three independent experiments. Scale bar is 200 m.

### Supplementary Video 13: Effect of HES7 Gene KO on Axioloid Growth and Morphology.

Top panel, live imaging of axioloids embedded in +MG+RAL derived from 201B7 Luc (top left), HES7 KO1 (top middle) and HES7 KO2 (top right) cell lines. Data shown is representative of at least three independent experiments. Scale bar is 200 m. Bottom panel, 3D reconstruction of axioloids embedded in +MG+RAL derived from HES7 KO1 (middle) and HES7 KO2 (bottom) stained with F-actin (Phalloidin) in gray, TBXT (BRA) in blue, Fibronectin (FN1) in green and MEOX1 in red.

### Supplementary Video 14: Effect of HES7 Gene KO on HES7 Gene Expression Dynamics.

Live imaging of the spatiotemporal expression of the HES7 gene in 201B7 Luc, (top pair), HES7 KO1 (middle pair) and HES7 KO2 (bottom pair)-derived axioloids embedded in +MG+RAL. BF video (left) and HES7:Luciferase signal (right) for each condition. Data shown is representative of at least three independent experiments. Scale bar is 200 m.

### Supplementary Video 15: Effect of HES7 Point Mutation (Rs113994160: c.73C>T; HES7R25W) on Axioloid Growth and Morphology.

Top panel, live imaging of axioloids embedded in +MG+RAL derived from 201B7 Luc (top left), HES7R25W MT1 (top middle) and HES7R25W MT2 (top right) cell lines. Data shown is representative of at least three independent experiments. Scale bar is 200 m. Bottom panel, 3D reconstruction of axioloids embedded in +MG+RAL derived from HES7R25W MT1 (middle) and HES7R25W MT2 (bottom) stained with F-actin (Phalloidin) in gray, TBXT (BRA) in blue, Fibronectin (FN1) in green and MEOX1 in red.

### Supplementary Video 16: Effect of HES7 Point Mutation (Rs113994160: c.73C>T; HES7R25W) on HES7 Gene Expression Dynamics.

Live imaging of the spatiotemporal expression of the HES7 gene in 201B7 Luc, (top pair), HES7R25W MT1 (middle pair) and HES7R25W MT2 (bottom pair)-derived axioloids. BF video (left) and HES7:Luciferase signal (right) for each condition. Data shown is representative of at least three independent experiments. Scale bar is 200 m.

### Supplementary Video 17: Effect of MESP2 Gene KO on Axioloid Growth and Morphology.

Top panel, live imaging of axioloids embedded in +MG+RAL derived from 201B7 Luc (top left), MESP2 KO1 (top middle) and MESP2 KO2 (top right) cell lines. Data shown is representative of at least three independent experiments. Scale bar is 200 m. Bottom panel, 3D reconstruction of axioloids embedded in +MG+RAL derived from MESP2 KO1 (middle) and MESP2 KO2 (bottom) stained with F-actin (Phalloidin) in gray, TBXT (BRA) in blue, Fibronectin (FN1) in green and MEOX1 in red.

### Supplementary Video 18: Effect of MESP2 Gene KO on HES7 Gene Expression Dynamics.

Live imaging of the spatiotemporal expression of the HES7 gene in 201B7 Luc (top pair), MESP2 KO1 (middle pair) and MESP2 KO2 (bottom pair)-derived axioloids embedded in +MG+RAL. BF video (left) and HES7:Luciferase signal (right) for each condition. Data shown is representative of at least three independent experiments. Scale bar is 200 m.

### Appendix for Patent Application ‘Axioloids: A Stem Cell-Based Model of Human Axial Development’

### Introduction

Somitogenesis is a core developmental event during which the metameric body plan is laid out in vertebrates. It is well studied in model organisms such as mouse, zebrafish or chick but remains poorly understood in human and other primates. Despite recent progress with pluripotent stem cell (PSC)-based in vitro models of organogenesis and embryonic development, an experimental model system that can robustly recapitulate core features of somitogenesis in vitro remained out of reach. Using in vitro-derived presomitic mesoderm (PSM), it has been previously succeeded to reconstitute and quantify oscillatory activity of the segmentation clock, a molecular oscillator believed to control somite formation. Extending on these earlier findings it was then asked whether not only the segmentation clock but also the actual process of segmentation and epithelial somite formation in vitro could be recapitulated.

To this end “axioloids” was generated, a self-organizing 3D in vitro model of human somitogenesis which shares morphological and molecular features of the emerging vertebrate embryonic tail and axis including presence of somitogenesis associated major cell populations and opposing morphogen gradients and signaling activities as well as periodic formation of properly patterned epithelial somites in synchrony with the segmentation clock. Using this model, an unknown function of retinoic acid (RA) signaling in the stabilization and epithelialization of the newly formed somite-like structures within axioloids was unveiled.

Here the effect on initial and final axioloid morphogenesis of variations in the concentration or nature of the molecules and reagents used during axioloid derivation was assessed and is reported, including effect of different CHIR mediated WNT activation levels, bFGF mediated FGF pathways activation levels, and different TGF-β inhibitor mediated TGF-β pathway activation levels. Moreover, the effect of various extracellular matrix compounds and a synthetic analog of the retinoic acid pathway was also evaluated.

### Material and Methods

Culture of Human Induced Pluripotent Stem Cells (iPSCs)

Human iPS cell lines derived from healthy donors, e.g. 409B2 were used in this study. Human iPS cells were maintained in StemFit AK02N (Reprocell) medium supplemented with 50 U penicillin and 50 g ml-1 streptomycin (Gibco) on iMatrix-511 silk-coated plates or dishes (Nippi). StemFit AK02N (Reprocell) medium contains three components, A, B and C, all three of which were mixed and used for standard maintenance culture of human iPS cells in humidified incubators at 37° C. and 5% CO₂. Used iPS cells were regularly tested and reported negative for mycoplasma contamination.

Based on the axioloid protocol it was explored if alternative compounds, molecules or inhibitors could be used and would support the generation of axioloids from PSCs. It was started by testing if NDiff227 (Takara, Cat: Y40002) medium or RPMI 1640 (Nacalai, Cat: 30264-85) supplemented with B27 either with (Gibco, Cat: 17504-044) or without (in house) retinol could be used as an alternative to AK02N-C induction media. The effects of different concentrations of bFGF (from 5 to 250 ng ml-1), or CHIR99021 (from 2.5 to 10 μM), or SB431542 (from 1.25 to 15 μM) were then tested on axioloid morphology. It was also tested if another member of the FGF superfamily: FGF8b (PeproTech, Cat: 100-25) (from 20 to 250 ng ml⁻¹) or another TGFβ inhibitor A83-01 (Selleck Chemicals, Cat: S7692) (from 1.25 to 15 μM) could have similar results as described for bFGF and SB431542. Finally, during the embedding phase it was tested if the alternative ECM containing compounds Geltrex (Gibco, Cat: A14132-0), Cultrex (R&D Systems, Cat: 3433-005-01), and ECMgel (Sigma-Aldrich, Cat: E6909) could be used instead of Matrigel and would support axioloid morphogenesis. It was also assessed whether a synthetic RA selective agonist, TTNPB (4-[(E)-2-(5,6,7,8-Tetrahydro-5,5,8,8-tetramethyl-2-naphthalenyl)-1-propenyl]benzoic acid) (Selleck Chemicals, Cat: S4627) could replace vitamin A derivatives such as retinol or retinal.

### Results

Basic Strategy for Axioloid Induction from Human Pluripotent Stem Cells (PSCs)

The generation of a mesoderm-based human induced pluripotent stem cell (iPSC)-derived 3D model of human axial development was recently reported, which is termed ‘axioloids’. This was achieved through an initial induction step in 2D, using both bFGF (20 ng/ml) and CHIR99421 (5 M) which efficiently and reproducibly promoted the formation of primitive streak-like cells from human PSCs. This was then followed by a 3D aggregation step in the presence of bFGF and CHIR (same concentrations as during 1^{st} step) in addition to 10 μM of SB431542 (TGFβ inhibitor) and 10 μM of Y-27632 (ROCK inhibitor). This step aimed at promoting the commitment of the initial primitive streak cells to a presomitic or paraxial mesoderm (PSM) fate. Formed mesodermal aggregates spontaneously broke symmetry and started elongating and adopted an initial ‘bean-like’ morphology. At this point, embedding into 10% Matrigel (MG)-containing medium supplemented with retinoids led both to further elongation and the sequential emergence of somite-like epithelialized segments along the anterior-posterior axis of these structures, which is termed ‘axioloids’.

### Alternative Culture Media can Also Support Axioloid Induction & Morphogenesis

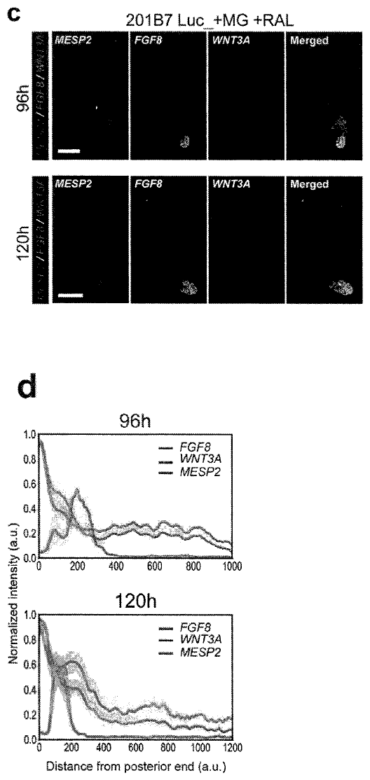

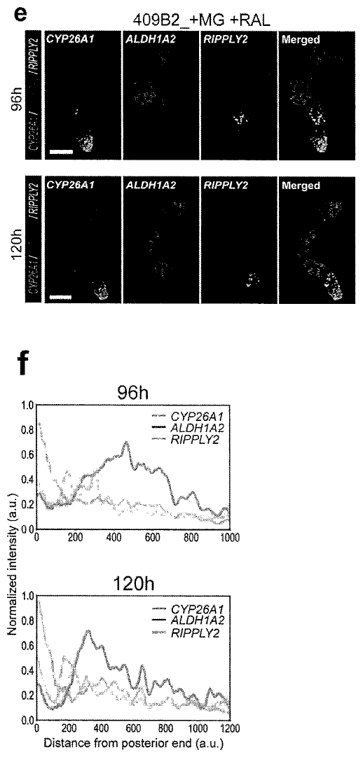

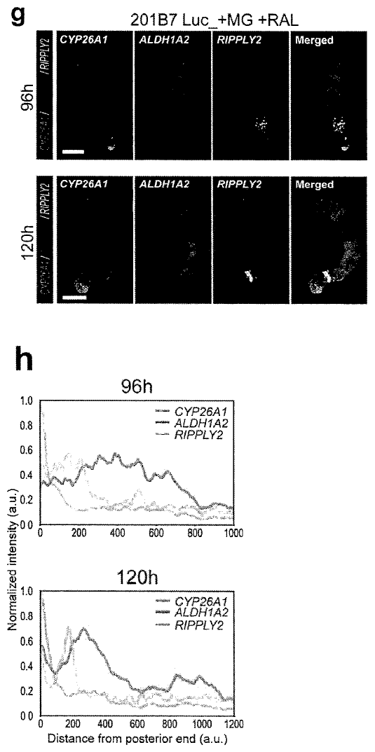

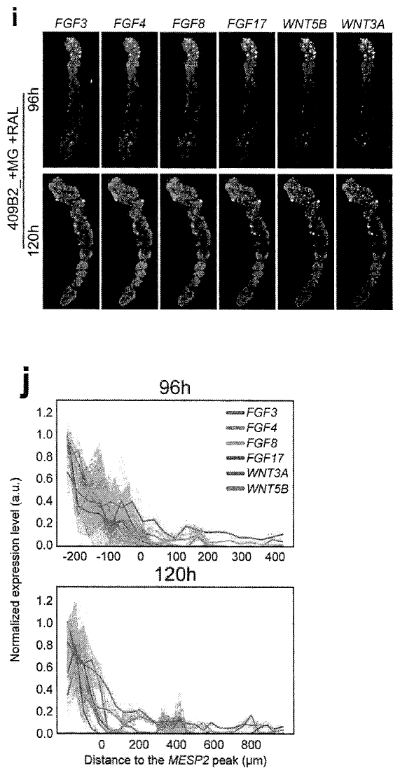

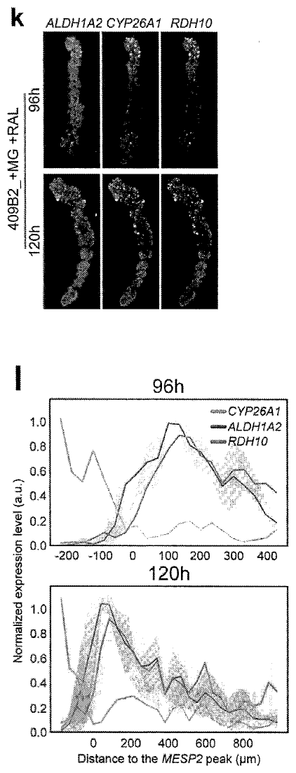

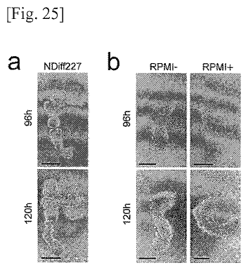

Different culture media in addition to the standard AK02N medium was tested. Several groups in the field have reported the successful generation of mouse and human PSC-derived gastruloids and similar structures using N2B27 media (PMID: 30283134). A quality controlled commercial version of the N2B27 medium called NDiff227 was initially tested, and it was shown that it can support axioloid formation (FIG. 25 a). RPMI was then tested as a possible alternative base medium supplemented with B27 supplement either containing retinol or not. It was shown that although it could support initial axioloid elongation, generated structures were morphologically different from normal axioloids, with no visible segments or somite-like structures forming (FIG. 25 b).

Effective Range of Concentrations of bFGF, CHIR (WNT Agonist) & SB431542 (TGFβ Inhibitor)

It was shown in the corresponding publication (Yamanaka, Hamidi et al., Nature 2023) that for proper axioloid induction bFGF, CHIR99421 (a WNT agonist) and SB431542 (TGFβ-inhibitor) are necessary and sufficient. Here different concentrations of all three components were tested and their impact was assessed on axioloid induction and morphology. The results indicate that higher concentrations of bFGF do not largely affect overall axioloid morphology with regards to both elongation and segmentation observed at 96 h and 120 h, while structures at 120 h appear to get thinner at higher bFGF concentrations. Total absence of bFGF during axioloid induction results in loss of polarization, elongation and failure to generate axioloid structures from PSCs (FIG. 26 a). The effect of various concentrations of CHIR on axioloid induction and morphology was also tested and it was observed that lower concentrations of e.g. 2 μM or 2.5 μM of CHIR99421 resulted in round cellular aggregates with no sign of symmetry breaking or elongation. Intermediate levels of CHIR e.g. 3 μM of CHIR99421 resulted in the formation of elongated neural tube-like structures. Higher than usual (5 uM) concentrations of CHIR e.g. 7.5 μM or 10 μM CHIR99421 had no dramatic effect on overall axioloid morphology albeit some negative impact on overall axioloid length (FIG. 26 b). The results also suggest that CHIR concentrations used during axioloid induction may show variable outcomes depending on the utilized cell line.

Different concentrations of the TGFβ pathway inhibitor SB431542 used for axioloid induction were furthermore assessed. The results indicate that SB431542 is active and effective over a large range of concentrations (FIG. 26 c). Taken together the utilized CHIR99421 concentration appears to be the most critical factor determining the overall success rate of axioloid generation, while bFGF and TGFβ-inhibitor concentrations show wider ranges of activity. For most PSC lines CHIR concentrations around 5 μM might work but will have to be determined and optimized for each cell line. The data overall suggests the presence of ‘ranges of activity’ rather than discrete single working concentrations for signaling molecules, which are conducive and sufficient for proper and efficient axioloid induction and morphogenesis. FGF8b can not substitute bFGF but A-83-01 can be used as an alternative to SB431542.

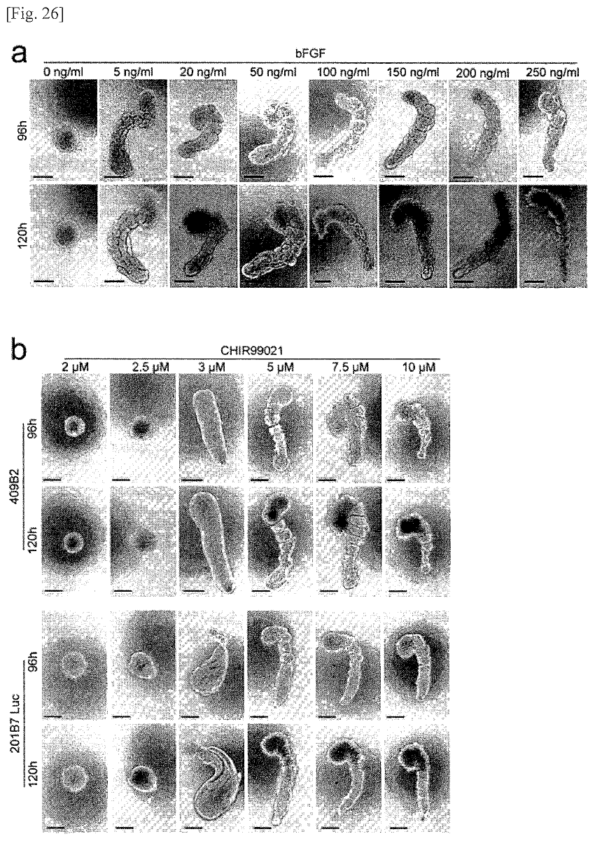

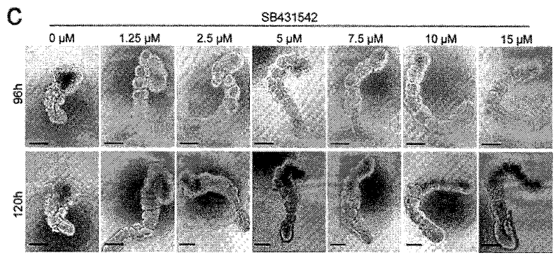

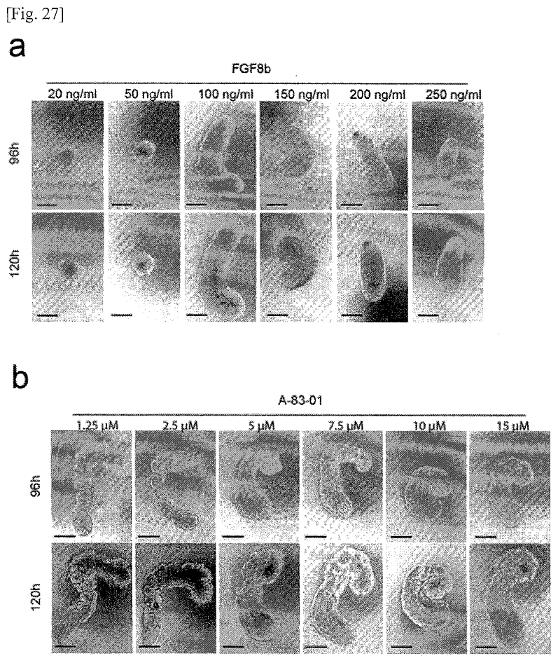

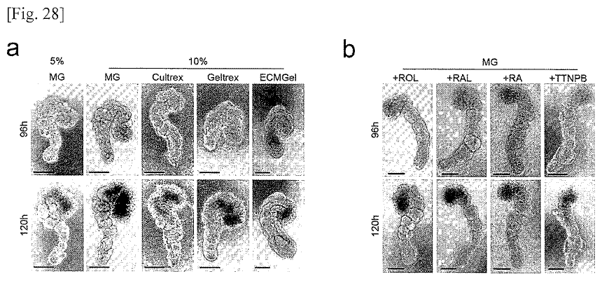

Basic FGF (bFGF) is a member of the fibroblast growth factor family that includes 23 heparin-binding peptides widely expressed during embryo development. A large number of recombinant FGFs were tested for a putative effect on axioloid induction and morphogenesis. An effect for recombinant FGF8b which behaved differently from bFGF was observed. At higher FGF8b concentrations (100 ng/ml) elongated structures were obtained but somites were not discernable within the forming structures (FIG. 27 a).

Similarly, it was tested if A-83-01, an alternative TGFβ pathway inhibitor could be used to replace SB431542. The results indicate that axioloids generated in different concentrations of A-83-01 recapitulate hallmarks of proper axioloids morphogenesis, similar to what it has been described for SB431542, with best results obtained for smaller concentrations of A-83-01 e.g. 1.25 uM or 2.5 uM (FIG. 27 b).

### Alternative Extracellular Matrix (ECM)-Rich Components can be Used for Axioloid Induction

Matrigel (MG) is an extracellular matrix (ECM)-rich solubilized basement membrane secreted by Engelbreth-Holm-Swarm (EHS) mouse sarcoma cells that can support the formation of complex tissue architectures and help mimic morphogenetic processes in vitro. In the experimental system addition of both 5% or 10% MG to the culture medium (during the embedding phase after initial symmetry breaking) is sufficient and leads to effective axioloid induction in the presence of retinoids. Here supplementation of the culture medium with alternative ECM-rich compounds (10%) such as Cultrex, Geltrex and ECMgel was assessed. All three tested ECM-rich compounds lead to variable morphological axioloid phenotypes, with axial elongation observed in all three compounds albeit to a lesser extent than Matrigel. It is believed that other ECM containing compounds can likely be used as well and may be able to replace MG. It is furthermore envisioned that distinct mixtures of defined recombinant matrix proteins and basement membrane components may be also able to replace MG or similar complex ECM-rich compounds in order to achieve reproducible and efficient axioloid induction and morphogenesis in the presence of active retinoid signaling which appears to be essential and working in synergy with MG.

In addition to assessing ECM-rich compounds it has been also investigated and confirmed that TTNPB, a synthetic analog of retinoic acid, selective for the retinoic acid receptor (RAR) subtype, could be used in replacement of retinoids. TTNPB showed overall similar effect on axioloid morphology and somite epithelialization than the other retinoids including retinol (ROL), retinal (RAL) and retinoic acid (RA).

### REFERENCES

1 Matsuda, M. et al. Recapitulating the human segmentation clock with pluripotent stem cells. Nature 580, 124-129, doi:10.1038/s41586-020-2144-9 (2020).
2 van den Brink, S. C. et al. Single-cell and spatial transcriptomics reveal somitogenesis in gastruloids. Nature 582, 405-409, doi:10.1038/s41586-020-2024-3 (2020).
3 Veenvliet, J. V. et al. Mouse embryonic stem cells self-organize into trunk-like structures with neural tube and somites. Science 370, doi:10.1126/science.aba4937 (2020).
4 Diaz-Cuadros, M. et al. In vitro characterization of the human segmentation clock. Nature 580, 113-118, doi:10.1038/s41586-019-1885-9 (2020).
5 Moris, N. et al. An in vitro model of early anteroposterior organization during human development. Nature 582, 410-415, doi:10.1038/s41586-020-2383-9 (2020).
6 Chu, L. F. et al. An In Vitro Human Segmentation Clock Model Derived from Embryonic Stem Cells. Cell Rep 28, 2247-2255 e2245, doi:10.1016/j.celrep.2019.07.090 (2019).
7 Wilson, V., Rashbass, P. & Beddington, R. S. Chimeric analysis of T (Brachyury) gene function. Development 117, 1321-1331, doi:10.1242/dev.117.4.1321 (1993).
8 McGrew, M. J., Dale, J. K., Fraboulet, S. & Pourquie, O. The lunatic fringe gene is a target of the molecular clock linked to somite segmentation in avian embryos. Curr Biol 8, 979-982, doi:10.1016/s0960-9822(98)70401-4 (1998).
9 Prince, V. E. et al. Zebrafish lunatic fringe demarcates segmental boundaries. Mech Dev 105, 175-180, doi:10.1016/s0925-4773(01)00398-7 (2001).
10 Evrard, Y. A., Lun, Y., Aulehla, A., Gan, L. & Johnson, R. L. lunatic fringe is an essential mediator of somite segmentation and patterning. Nature 394, 377-381, doi:10.1038/28632 (1998).
11 Neidhardt, L. M., Kispert, A. & Herrmann, B. G. A mouse gene of the paired-related homeobox class expressed in the caudal somite compartment and in the developing vertebral column, kidney and nervous system. Dev Genes Evol 207, 330-339, doi:10.1007/s004270050120 (1997).
12 Kraus, F., Haenig, B. & Kispert, A. Cloning and expression analysis of the mouse T-box gene Tbx18. Mech Dev 100, 83-86, doi:10.1016/s0925-4773(00)00494-9 (2001).
13 Pourquie, O. The segmentation clock: converting embryonic time into spatial pattern. Science 301, 328-330, doi:10.1126/science.1085887 (2003).
14 Holley, S. A., Geisler, R. & Nusslein-Volhard, C. Control of her1 expression during zebrafish somitogenesis by a delta-dependent oscillator and an independent wave-front activity. Genes Dev 14, 1678-1690 (2000).
15 Saga, Y. & Takeda, H. The making of the somite: molecular events in vertebrate segmentation. Nat Rev Genet 2, 835-845, doi:10.1038/35098552 (2001).
16 Bessho, Y. et al. Dynamic expression and essential functions of Hes7 in somite segmentation. Genes Dev 15, 2642-2647, doi:10.1101/gad.930601 (2001).
17 Palmeirim, I., Henrique, D., Ish-Horowicz, D. & Pourquie, O. Avian hairy gene expression identifies a molecular clock linked to vertebrate segmentation and somitogenesis. Cell 91, 639-648, doi:10.1016/s0092-8674(00)80451-1 (1997).
18 Ishimatsu, K., Takamatsu, A. & Takeda, H. Emergence of traveling waves in the zebrafish segmentation clock. Development 137, 1595-1599, doi:10.1242/dev.046888 (2010).
19 Cooke, J. & Zeeman, E. C. A clock and wavefront model for control of the number of repeated structures during animal morphogenesis. J Theor Biol 58, 455-476, doi:10.1016/s0022-5193(76)80131-2 (1976).
20 Kondrashov, N. et al. Ribosome-mediated specificity in Hox mRNA translation and vertebrate tissue patterning. Cell 145, 383-397, doi:10.1016/j.cell.2011.03.028 (2011).
21 Diez del Corral, R. et al. Opposing FGF and retinoid pathways control ventral neural pattern, neuronal differentiation, and segmentation during body axis extension. Neuron 40, 65-79, doi:10.1016/s0896-6273(03)00565-8 (2003).
22 Olivera-Martinez, I. & Storey, K. G. Wnt signals provide a timing mechanism for the FGF-retinoid differentiation switch during vertebrate body axis extension. Development 134, 2125-2135, doi:10.1242/dev.000216 (2007).
23 Moreno, T. A. & Kintner, C. Regulation of segmental patterning by retinoic acid signaling during Xenopus somitogenesis. Dev Cell 6, 205-218, doi:10.1016/s1534-5807(04)00026-7 (2004).
24 Dubrulle, J., McGrew, M. J. & Pourquie, O. FGF signaling controls somite boundary position and regulates segmentation clock control of spatiotemporal Hox gene activation. Cell 106, 219-232, doi:10.1016/s0092-8674(01)00437-8 (2001).
25 Aulehla, A. et al. Wnt3a plays a major role in the segmentation clock controlling somitogenesis. Dev Cell 4, 395-406, doi:10.1016/s1534-5807(03)00055-8 (2003).
26 Aulehla, A. et al. A beta-catenin gradient links the clock and wavefront systems in mouse embryo segmentation. Nat Cell Biol 10, 186-193, doi:10.1038/ncb1679 (2008).
27 Vermot, J. et al. Retinoic acid controls the bilateral symmetry of somite formation in the mouse embryo. Science 308, 563-566, doi:10.1126/science.1108363 (2005).
28 Sato, Y. et al. Notch mediates the segmental specification of angioblasts in somites and their directed migration toward the dorsal aorta in avian embryos. Dev Cell 14, 890-901, doi:10.1016/j.devcel.2008.03.024 (2008).
29 Jacob, M., Christ, B., Jacob, H. J. & Poelmann, R. E. The role of fibronectin and laminin in development and migration of the avian Wolffian duct with reference to somitogenesis. Anat Embryol (Berl) 183, 385-395, doi:10.1007/BF00196840 (1991).
30 Mills, C. L., Ariyo, O., Yamada, K. M., Lash, J. W. & Bellairs, R. Evidence for the involvement of receptors for fibronectin in the promotion of chick tail segmentation. Anat Embryol (Berl) 182, 425-434, doi:10.1007/BF00178907 (1990).
31 Page, M. Changing patterns of cytokeratins and vimentin in the early chick embryo. Development 105, 97-107, doi:10.1242/dev.105.1.97 (1989).
32 Burgess, R., Rawls, A., Brown, D., Bradley, A. & Olson, E. N. Requirement of the paraxis gene for somite formation and musculoskeletal patterning. Nature 384, 570-573, doi:10.1038/384570a0 (1996).
33 Wiggan, O., Fadel, M. P. & Hamel, P. A. Pax3 induces cell aggregation and regulates phenotypic mesenchymal-epithelial interconversion. J Cell Sci 115, 517-529, doi:10.1242/jcs.115.3.517 (2002).
34 Vermot, J. & Pourquie, O. Retinoic acid coordinates somitogenesis and left-right patterning in vertebrate embryos. Nature 435, 215-220, doi:10.1038/nature03488 (2005).
35 Xu, Y. et al. A single-cell transcriptome atlas of human early embryogenesis. bioRxiv, 2021.2011.2030.470583, doi:10.1101/2021.11.30.470583 (2021).
36 Stuart, T. et al. Comprehensive Integration of Single-Cell Data. Cell 177, 1888-1902 e1821, doi:10.1016/j.cell.2019.05.031 (2019).
37 Olivera-Martinez, I., Harada, H., Halley, P. A. & Storey, K. G. Loss of FGF-dependent mesoderm identity and rise of endogenous retinoid signalling determine cessation of body axis elongation. PLoS Biol 10, e1001415, doi:10.1371/journal.pbio.1001415 (2012).
38 Gyllborg, D. et al. Hybridization-based in situ sequencing (HybISS) for spatially resolved transcriptomics in human and mouse brain tissue. Nucleic Acids Res 48, e112, doi:10.1093/nar/gkaa792 (2020).
39 Beccari, L. et al. Multi-axial self-organization properties of mouse embryonic stem cells into gastruloids. Nature 562, 272-276, doi:10.1038/s41586-018-0578-0 (2018).
40 Matsumiya, M., Tomita, T., Yoshioka-Kobayashi, K., Isomura, A. & Kageyama, R. ES cell-derived presomitic mesoderm-like tissues for analysis of synchronized oscillations in the segmentation clock. Development 145, doi:10.1242/dev.156836 (2018).
41 Wahl, M. B., Deng, C., Lewandoski, M. & Pourquie, O. FGF signaling acts upstream of the NOTCH and WNT signaling pathways to control segmentation clock oscillations in mouse somitogenesis. Development 134, 4033-4041, doi:10.1242/dev.009167 (2007).
42 Dubrulle, J. & Pourquie, O. fgf8 mRNA decay establishes a gradient that couples axial elongation to patterning in the vertebrate embryo. Nature 427, 419-422, doi:10.1038/nature02216 (2004).
43 Boulet, A. M. & Capecchi, M. R. Signaling by FGF4 and FGF8 is required for axial elongation of the mouse embryo. Dev Biol 371, 235-245, doi:10.1016/j.ydbio.2012.08.017 (2012).
44 Sparrow, D. B., Guillen-Navarro, E., Fatkin, D. & Dunwoodie, S. L. Mutation of Hairy-and-Enhancer-of-Split-7 in humans causes spondylocostal dysostosis. Hum Mol Genet 17, 3761-3766, doi:10.1093/hmg/ddn272 (2008).
45 Whittock, N. V. et al. Mutated MESP2 causes spondylocostal dysostosis in humans. Am J Hum Genet 74, 1249-1254, doi:10.1086/421053 (2004).
46 Takahashi, Y. et al. Mesp2 initiates somite segmentation through the Notch signalling pathway. Nat Genet 25, 390-396, doi:10.1038/78062 (2000).
47 Takahashi, Y. et al. Transcription factors Mesp2 and Paraxis have critical roles in axial musculoskeletal formation. Dev Dyn 236, 1484-1494, doi:10.1002/dvdy.21178 (2007).
48 Okita, K. et al. A more efficient method to generate integration-free human iPS cells. Nat Methods 8, 409-412, doi:10.1038/nmeth.1591 (2011).
49 Takahashi, K. et al. Induction of pluripotent stem cells from adult human fibroblasts by defined factors. Cell 131, 861-872, doi:10.1016/j.cell.2007.11.019 (2007).
50 Sharpe, J. et al. Optical projection tomography as a tool for 3D microscopy and gene expression studies. Science 296, 541-545, doi:10.1126/science.1068206 (2002).
51 Kerwin, J. et al. 3 dimensional modelling of early human brain development using optical projection tomography. BMC Neurosci 5, 27, doi:10.1186/1471-2202-5-27 (2004).
52 Kerwin, J. et al. The HUDSEN Atlas: a three-dimensional (3D) spatial framework for studying gene expression in the developing human brain. J Anat 217, 289-299, doi:10.1111/j.1469-7580.2010.01290.x (2010).
53 Hama, H. et al. ScaleS: an optical clearing palette for biological imaging. Nat Neurosci 18, 1518-1529, doi:10.1038/nn.4107 (2015).
54 Choi, H. M. T. et al. Third-generation in situ hybridization chain reaction: multiplexed, quantitative, sensitive, versatile, robust. Development 145, doi:10.1242/dev.165753 (2018).
55 Masamizu, Y. et al. Real-time imaging of the somite segmentation clock: revelation of unstable oscillators in the individual presomitic mesoderm cells. Proc Natl Acad Sci USA 103, 1313-1318, doi:10.1073/pnas.0508658103 (2006).
56 Isomura, A., Ogushi, F., Kori, H. & Kageyama, R. Optogenetic perturbation and bioluminescence imaging to analyze cell-to-cell transfer of oscillatory information. Genes Dev 31, 524-535, doi:10.1101/gad.294546.116 (2017).
57 Schindelin, J. et al. Fiji: an open-source platform for biological-image analysis. Nat Methods 9, 676-682, doi:10.1038/nmeth.2019 (2012).
58 Kaya-Okur, H. S. et al. CUT&Tag for efficient epigenomic profiling of small samples and single cells. Nat Commun 10, 1930, doi:10.1038/s41467-019-09982-5 (2019).
59 Martin, M. Cutadapt Removes Adapter Sequences from High-Throughput Sequencing Reads. EMBnet.journal 17 (1), 10-12 (2011).
60 Langmead, B. & Salzberg, S. L. Fast gapped-read alignment with Bowtie 2. Nat Methods 9, 357-359, doi:10.1038/nmeth.1923 (2012).
61 Li, H. et al. The Sequence Alignment/Map format and SAMtools. Bioinformatics 25, 2078-2079, doi:10.1093/bioinformatics/btp352 (2009).
62 Quinlan, A. R. & Hall, I. M. BEDTools: a flexible suite of utilities for comparing genomic features. Bioinformatics 26, 841-842, doi:10.1093/bioinformatics/btq033 (2010).
63 Ramirez, F. et al. deepTools2: a next generation web server for deep-sequencing data analysis. Nucleic Acids Research 44, W160-W165, doi:10.1093/nar/gkw257 (2016).
64 Wolock, S. L., Lopez, R. & Klein, A. M. Scrublet: Computational Identification of Cell Doublets in Single-Cell Transcriptomic Data. Cell Syst 8, 281-291 e289, doi:10.1016/j.cels.2018.11.005 (2019).
65 Hao, Y. et al. Integrated analysis of multimodal single-cell data. Cell 184, 3573-3587 e3529, doi:10.1016/j.cell.2021.04.048 (2021).
66 Butler, A., Hoffman, P., Smibert, P., Papalexi, E. & Satija, R. Integrating single-cell transcriptomic data across different conditions, technologies, and species. Nat Biotechnol 36, 411-420, doi:10.1038/nbt.4096 (2018).
67 Hafemeister, C. & Satija, R. Normalization and variance stabilization of single-cell RNA-seq data using regularized negative binomial regression. Genome Biol 20, 296, doi:10.1186/s13059-019-1874-1 (2019).
68 Chen, E. Y. et al. Enrichr: interactive and collaborative HTML5 gene list enrichment analysis tool. BMC Bioinformatics 14, 128, doi:10.1186/1471-2105-14-128 (2013).
69 Bray, N. L., Pimentel, H., Melsted, P. & Pachter, L. Near-optimal probabilistic RNA-seq quantification. Nat Biotechnol 34, 525-527, doi:10.1038/nbt.3519 (2016).
70 Bergen, V., Lange, M., Peidli, S., Wolf, F. A. & Theis, F. J. Generalizing RNA velocity to transient cell states through dynamical modeling. Nat Biotechnol 38, 1408-1414, doi:10.1038/s41587-020-0591-3 (2020).

While the present disclosure has been described above with reference to exemplary embodiments and example, the present disclosure is by no means limited thereto. Various changes and modifications that may become apparent to those skilled in the art may be made in the configuration and specifics of the present disclosure without departing from the scope of the present disclosure.

This application claims priority from U.S. Provisional Patent Application No. 63/326,611 filed on Apr. 1, 2022. The entire disclosure of this US provisional patent application is incorporated herein by reference.

Patents, patent applications, and references cited in the present specification are incorporated herein in their entirety by reference, as if fully and specifically set forth herein.

### SUPPLEMENTARY NOTES

The whole or part of the exemplary embodiments and example disclosed above can be described as, but not limited to, the following Supplementary Notes.

### (Supplementary Note 1)

A three-dimensional cellular aggregate generated in vitro from a pluripotent stem cell, comprising:
a mesodermal cell, wherein
the cellular aggregate has a polarity in an antero-posterior axis or a rostro-caudal axis and an apical-basolateral axis, and
the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition.

### (Supplementary Note 2)

A three-dimensional cellular aggregate generated in vitro from a pluripotent stem cell, comprising:
a mesodermal cell, wherein
the cellular aggregate has a polarity in an antero-posterior axis or a rostro-caudal axis and an apical-basolateral axis, and
a proportion of the mesodermal cell in the cellular aggregate is at least 50%, at least 55%, at least 60%, at least 65%, at least 70%, at least 75%, at least 80%, at least 85%, at least 90%, at least 95%, at least 96%, at least 97%, at least 98%, or at least 99%, based on the number of cells.

### (Supplementary Note 3)

The cellular aggregate according to Supplementary Note 1 or 2, wherein
the antero-posterior axis is defined by an anterior region and a posterior region, and an anterior region cell has a higher or lower expression of one or more markers as compared to a posterior region cell.

### (Supplementary Note 4)

The cellular aggregate according to Supplementary Note 3, wherein
the anterior region cell has a lower expression of one or more markers as compared to the posterior region cell, and
the one or more markers are selected from the group consisting of TBXT, MIXL1, SOX2, TBX6, HES7, MSGN1, MEOX1, TCF15, CYP26 A1, FGF3, FGF4, FGF8, FGF17, WNT3a, WNT5a, WNT5 b, HOXD13, HOXB, HOXA9, HOXA10, and CDX2.

### (Supplementary Note 5)

The cellular aggregate according to Supplementary Note 3 or 4, wherein
the anterior region cell has a higher expression of one or more markers as compared to the posterior region cell, and
the one or more markers comprise LFNG, MEOX1, TCF15, UNCX, TBX18, ALDH1 A2, and RDH10.

### (Supplementary Note 6)

The cellular aggregate according to Supplementary Note 5, wherein
the posterior region comprises a tailbud (TB) like-cell.

### (Supplementary Note 7)

The cellular aggregate according to any one of Supplementary Notes 1 to 6, wherein
the apical-basolateral axis is defined by an apical region and a basolateral region, and an apical region of a cell has a higher or lower expression of one or more markers as compared to a basolateral region of a cell.

### (Supplementary Note 8)

The cellular aggregate according to Supplementary Note 3, wherein
the apical region of a cell has a lower expression of one or more markers as compared to the basolateral region of a cell, and
the one or more markers are selected from the group consisting of Fibronectin, Collagen V, and Laminin.

### (Supplementary Note 9)

The cellular aggregate according to Supplementary Note 3 or 4, wherein
the apical region of a cell has a higher expression of one or more markers as compared to the basolateral region of a cell, and
the one or more markers are selected from the group consisting of aPKC, CDH2, Ezrin, ZO1, and F-Actin.

### (Supplementary Note 10)

The cellular aggregate according to any one of Supplementary Notes 1 and 3 to 9, wherein
a proportion of the mesodermal cell in the cellular aggregate is at least 50%, at least 55%, at least 60%, at least 65%, at least 70%, at least 75%, at least 80%, at least 85%, at least 90%, at least 95%, at least 96%, at least 97%, at least 98%, or at least 99%, based on the number of cells.

### (Supplementary Note 11)

The cellular aggregate according to any one of Supplementary Notes 1 to 10, wherein
the mesodermal cell expresses a marker selected from the group consisting of TBXT, SOX2, NODAL, WNT3a, WNT5a, DLL1, TCF15, MEOX1, TBX18, UNCX, ALDH1 A2, RDH10, RIPPLY1, RIPPLY2, MESP1, MESP2, HES7, TBX6, MSGN1, and FLK1/KDR.

### (Supplementary Note 12)

The cellular aggregate according to any one of Supplementary Notes 2 to 11, wherein
the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition.

### (Supplementary Note 13)

The cellular aggregate according to Supplementary Note 1 or 12, wherein
the somitogenic culture condition is a presence of a gel or a matrix and a retinoic acid, a retinoic acid precursor or derivative, and/or a retinoic acid receptor (RAR) agonist; or
The cellular aggregate according to Supplementary Note 1 or 12, wherein
the somitogenic culture condition is a presence of a gel or a matrix and a retinoid, including retinoic acid, a retinoic acid precursor or derivative, and/or a retinoic acid receptor (RAR) agonist.

### (Supplementary Note 14)

The cellular aggregate according to Supplementary Note 13, wherein
the RAR agonist comprises retinal or retinol; or
The cellular aggregate according to Supplementary Note 13, wherein
the retinoid, comprises, retinoic acid, a retinoic acid precursor such as retinal or retinol or a RAR agonist; or
The cellular aggregate according to Supplementary Note 13, wherein
the retinoid comprises retinal or retinol; or
The cellular aggregate according to Supplementary Note 13, wherein
the retinoid, comprises, retinoic acid, a retinoic acid precursor such as retinal or retinol or a RAR agonist.

### (Supplementary Note 15)

The cellular aggregate according to Supplementary Note 13 or 14, wherein
the cellular aggregate is embedded in the gel or the matrix or disposed inside the gel or the matrix.

### (Supplementary Note 16)

The cellular aggregate according to any one of Supplementary Notes 13 to 15, wherein
the matrix includes an extracellular matrix.

### (Supplementary Note 17)

The cellular aggregate according to any one of Supplementary Notes 13 to 16, wherein
the extracellular matrix includes collagen, laminin, fibronectin, vitronectin, gelatin, and/or entactin.

### (Supplementary Note 18)

The cellular aggregate according to any one of Supplementary Notes 13 to 17, wherein
the gel includes a hydrogel.

### (Supplementary Note 19)

The cellular aggregate according to any one of Supplementary Notes 13 to 18, wherein
the gel includes a basement membrane matrix.

### (Supplementary Note 20)

The cellular aggregate according to Supplementary Note 19, wherein
the basement membrane matrix includes laminin, collagen, heparan sulphate proteoglycan and/or entactin.

### (Supplementary Note 21)

The cellular aggregate according to any one of Supplementary Notes 13 to 17, wherein
the gel includes an acrylamide gel, an arginine gel, an agarose gel, and/or a polyethylene glycol hydrogel.

### (Supplementary Note 22)

The cellular aggregate according to any one of Supplementary Notes 1 and 12 to 21, wherein
the somite or the somite-like structure comprises an anterior portion and a posterior portion in the antero-posterior axis, and
an anterior portion cell has a higher or lower expression of one or more markers as compared to a posterior portion cell.

### (Supplementary Note 23)

The cellular aggregate according to Supplementary Note 22, wherein
the anterior portion cell has a higher expression of one or more markers than the posterior portion cell, and
the one or more markers are selected from the group consisting of TBX18 and ALDH1 A2.

### (Supplementary Note 24)

The cellular aggregate according to Supplementary Note 22 or 23, wherein
the anterior portion cell has a lower expression of one or more markers as compared to the posterior portion cell, and
the one or more markers are selected from the group consisting of UNCX and LNFG.

### (Supplementary Note 25)

The cellular aggregate according to any one of Supplementary Notes 1 and 12 to 24, comprising:
anterior paraxial/presomitic mesoderm (PSM), wherein
the anterior PSM has an expression of one or more makers selected from group consisting of MESP1, MESP2, RIPPLY1, RIPPLY2, and PCDH8, preferably MESP2.

### (Supplementary Note 26)

The cellular aggregate according to any one of Supplementary Notes 1 and 12 to 25, wherein
the somite is formed in a cycle of 3 to 7 hours, a cycle of 3.5 to 6.6 hours, or a cycle of 4 to 6 hours.

### (Supplementary Note 27)

The cellular aggregate according to any one of Supplementary Notes 1 to 26, comprising:
the pluripotent stem cell.

### (Supplementary Note 28)

The cellular aggregate according to Supplementary Note 27, wherein
a proportion of the pluripotent stem cell in the cellular aggregate is 10% or less, 9% or less, 8% or less, 7% or less, 6% or less, 5% or less, 4% or less, 3% or less, 2% or less, or 1% or less, based on the number of cells.

### (Supplementary Note 29)

The cellular aggregate according to any one of Supplementary Notes 1 to 28, substantially not comprising an endodermal cell and/or an ectodermal cell.

### (Supplementary Note 30)

The cellular aggregate according to Supplementary Note 29, wherein
a proportion of the endodermal cell in the cellular aggregate is 10% or less, 9% or less, 8% or less, 7% or less, 6% or less, 5% or less, 4% or less, 3% or less, 2% or less, or 1% or less, based on the number of cells.

### (Supplementary Note 31)

The cellular aggregate according to Supplementary Note 29 or 30, wherein
the endodermal cell expresses a marker selected from the group consisting of GATA6, GSC, CDX2, NEDD9, PYY, SHH, SORCS2, CER1, SOX17, FOXA2, TRH1, and FOXA1.

### (Supplementary Note 32)

The cellular aggregate according to any one of Supplementary Notes 29 to 31, wherein
a proportion of the ectodermal cell in the cellular aggregate is 10% or less, 9% or less, 8% or less, 7% or less, 6% or less, 5% or less, 4% or less, 3% or less, 2% or less, or 1% or less, based on the number of cells.

### (Supplementary Note 33)

The cellular aggregate according to any one of Supplementary Notes 29 to 32, wherein
the ectodermal cell expresses a marker selected from the group consisting of OTX2, GBX2, SIX1, SIX3, SOX1, SOX2, SOX3, DLXS, EYA2, and BARX1; or
The cellular aggregate according to any one of Supplementary Notes 29 to 32, wherein the ectodermal cell expresses a marker selected from the group consisting of OTX2, GBX2, SIX1, SIX3, SOX1, SOX3, DLXS, EYA2, and BARX1.

### (Supplementary Note 34)

The cellular aggregate according to any one of Supplementary Notes 1 to 33, wherein
an expression of a segmentation clock gene is subjected to gene oscillation.

### (Supplementary Note 35)

The cellular aggregate according to Supplementary Note 34, wherein
the segmentation clock gene is a gene selected from the group consisting of LFNG, DKK1, DLL1, DLL3, and HES7.

### (Supplementary Note 36)

The cellular aggregate according to Supplementary Note 34 or 35, wherein
a cycle of the gene oscillation is a cycle of 3 to 7 hours, a cycle of 3.5 to 6.6 hours, or a cycle of 4 to 6 hours.

### (Supplementary Note 37)

The cellular aggregate according to any one of Supplementary Notes 1 to 36, wherein
the pluripotent stem cell is a human pluripotent stem cell.

### (Supplementary Note 38)

The cellular aggregate according to any one of Supplementary Notes 1 to 37, wherein
the pluripotent stem cell is an embryonic stem cell or an artificial pluripotent stem cell.

### (Supplementary Note 39)

The cellular aggregate according to any one of Supplementary Notes 1 to 38, comprising:
at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000 cells, at least 2500 cells, at least 5000 cells, at least 10000 cells, at least 15000 cells, at least 20000 cells, at least 30000 cells, at least 40000 cells, or at least 50000 cells.

### (Supplementary Note 40)

The cellular aggregate according to any one of Supplementary Notes 1 to 39, which has a length of at least 0, 05 mm, at least 0, 1 mm, at least 0.2 mm, at least 0.3 mm, at least 0.4 mm, at least 0.5 mm, at least 0.6 mm, at least 0.7 mm, at least 0.8 mm, at least 0.9 mm, or at least 1 mm.

(Supplementary Note 41) A method for producing a three-dimensional cellular aggregate generated in vitro from a pluripotent stem cell, comprising the steps of:
(a) culturing a pluripotent stem cell to induce a three-dimensional cellular aggregate comprising a mesodermal cell; and
(b) culturing the cellular aggregate comprising the mesodermal cell to induce a three-dimensional cellular aggregate, wherein
the three-dimensional cellular aggregate is the cellular aggregate according to any one of Supplementary Notes 1 to 40.

### (Supplementary Note 42)

The method according to Supplementary Note 41, comprising the steps of:
(a1) culturing the pluripotent stem cell in a medium containing a GSK3β inhibitor and FGF to initiate their commitment toward a primitive streak and mesodermal fate, and/or induce the mesodermal cell;
(a2) culturing the cells derived, obtained, or obtainable from step (a1) in a medium containing the Wnt agonist (GSK3β inhibitor), the FGF agonist, a TGFβ inhibitor, and a ROCK inhibitor to induce the three-dimensional cellular aggregate including the mesodermal cell; and optionally
(b2) culturing the three-dimensional cellular aggregate including the mesodermal cell in a medium containing a retinoic acid, a retinoic acid derivative, and/or a retinoic acid receptor (RAR) agonist in the presence of a gel or a matrix to induce the three-dimensional cellular aggregate, and/or, a morphogenesis and/or a self-organization of the three-dimensional cellular aggregate.

### (Supplementary Note 43)

The method according to Supplementary Note 41 or 42, comprising the steps of:
(a1) culturing the pluripotent stem cell in a medium containing a GSK3β inhibitor and FGF to initiate their commitment toward a mesodermal fate, and/or induce the mesodermal cell;
(a2) culturing the cells derived, obtained, or obtainable from step (a1) in a medium containing the GSK3B inhibitor, the FGF, a TGFβ inhibitor, and a ROCK inhibitor to induce the three-dimensional cellular aggregate including the mesodermal cell;
(b1) culturing the three-dimensional cellular aggregate comprising the mesodermal cell in a medium not containing the GSK3β inhibitor, the FGF, the TGFβ inhibitor, and the ROCK inhibitor; and optionally
(b2) culturing the three-dimensional cellular aggregate including the mesodermal cell in a medium containing a retinoic acid, a retinoic acid derivative, and/or a retinoic acid receptor (RAR) agonist in the presence of a gel or a matrix to induce the three-dimensional cellular aggregate, and/or, a morphogenesis and/or a self-organization of the three-dimensional cellular aggregate.

### (Supplementary Note 44)

The method according to Supplementary Note 42 or 43, comprising the step of:
culturing the three-dimensional cellular aggregate comprising the mesodermal cell, which is embedded in the gel or the matrix or disposed inside the gel or the matrix, in a medium containing a retinoic acid, a retinoic acid derivative, and/or a retinoic acid receptor (RAR) agonist to induce the three-dimensional cellular aggregate; or
The method according to Supplementary Note 42 or 43, comprising the step of: culturing the three-dimensional cellular aggregate comprising the mesodermal cell, which is embedded in the gel or the matrix or disposed inside the gel or the matrix, in a medium containing a retinoic acid, a retinoic acid derivative, and/or a retinoid to induce the three-dimensional cellular aggregate.

### (Supplementary Note 45)

The method according to Supplementary Note 43 or 44, wherein
the RAR agonist is retinal or retinol; or
The method according to Supplementary Note 43 or 44, wherein
the retinoid, comprises, retinoic acid, a retinoic acid precursor such as retinal or retinol or a RAR agonist; or
The method according to Supplementary Note 43 or 44, wherein
the retinoid is retinal or retinol; or
The method according to Supplementary Note 43 or 44, wherein
the retinoid, comprises, retinoic acid, a retinoic acid precursor such as retinal or retinol or a RAR agonist.

### (Supplementary Note 46)

The method according to any one of Supplementary Notes 42 to 45, wherein
the GSK3β inhibitor is CHIR99021,
the FGF is bFGF,
the TGFβ inhibitor is SB431542, and/or
the ROCK inhibitor is Y-27632.

### (Supplementary Note 47)

The method according to any one of Supplementary Notes 42 to 46, wherein
the matrix includes an extracellular matrix.

### (Supplementary Note 48)

The method according to any one of Supplementary Notes 42 to 47, wherein
the extracellular matrix includes collagen, laminin, fibronectin, vitronectin, gelatin, and/or entactin.

### (Supplementary Note 49)

The method according to any one of Supplementary Notes 42 to 48, wherein
the gel includes a hydrogel.

### (Supplementary Note 50)

The method according to any one of Supplementary Notes 42 to 49, wherein
the gel includes a basement membrane matrix.

### (Supplementary Note 51)

The method according to Supplementary Note 50, wherein
the basement membrane matrix includes laminin, collagen, heparan sulphate proteoglycan, and/or entactin.

### (Supplementary Note 52)

The method according to any one of Supplementary Notes 42 to 51, wherein
the gel includes an acrylamide gel, an arginine gel, an agarose gel, and/or a polyethylene glycol hydrogel.

### (Supplementary Note 53)

The method according to any one of Supplementary Notes 41 to 52, wherein
a proportion of the mesodermal cell in the cellular aggregate comprising the mesodermal cell is at least 50%, at least 55%, at least 60%, at least 65%, at least 70%, at least 75%, at least 80%, at least 85%, at least 90%, at least 95%, at least 96%, at least 97%, at least 98%, or at least 99%, based on the number of cells.

### (Supplementary Note 54)

The method according to any one of Supplementary Notes 41 to 53, wherein
the mesodermal cell expresses a marker selected from the group consisting of TBXT, MIXL1, LEFTY1, LEFTY2, AXIN2, TRH1, NODAL, WNT3a, WNT5a, DLL1, MEOX1, OSR1, PAX2, ALDH1 A2, MESP1, MESP2, TBX6, HES7, MSGN1, TCF15, MEOX1, and FLK1/KDR; or
The method according to any one of Supplementary Notes 41 to 53, wherein
the mesodermal cell expresses a marker selected from the group consisting of TBXT, SOX2, MIXL1, LEFTY1, LEFTY2, AXIN2, TRH1, NODAL, WNT3a, WNT5a, DLL1, MEOX1, OSR1, PAX2, ALDH1 A2, MESP1, MESP2, TBX6, HES7, MSGN1, TCF15, MEOX1, and FLK1/KDR.

### (Supplementary Note 55)

The method according to any one of Supplementary Notes 41 to 54, wherein
the cellular aggregate comprising the mesodermal cell substantially not comprises an endodermal cell and/or an ectodermal cell.

### (Supplementary Note 56)

The method according to Supplementary Note 55, wherein
a proportion of the endodermal cell in the cellular aggregate comprising the mesodermal cell is 10% or less, 9% or less, 8% or less, 7% or less, 6% or less, 5% or less, 4% or less, 3% or less, 2% or less, or 1% or less, based on the number of cells.

### (Supplementary Note 57)

The method according to Supplementary Note 55 or 56, wherein
the endodermal cell expresses a marker selected from the group consisting of GATA6, GSC, CDX2, NEDD9, PYY, SHH, SORCS2, CER1, SOX17, FOXA2, TRH1, and FOXAL.

### (Supplementary Note 58)

The method according to any one of Supplementary Notes 55 to 57, wherein
a proportion of the ectodermal cell in the cellular aggregate comprising the mesodermal cell is 10% or less, 9% or less, 8% or less, 7% or less, 6% or less, 5% or less, 4% or less, 3% or less, 2% or less, or 1% or less, based on the number of cells.

### (Supplementary Note 59)

The method according to any one of Supplementary Notes 55 to 58, wherein
the ectodermal cell expresses a marker selected from the group consisting of OTX2, GBX2, SIX1, SIX3, SOX1, SOX2, SOX3, DLXS, EYA2, and BARX1; or
The method according to any one of Supplementary Notes 55 to 58, wherein the ectodermal cell expresses a marker selected from the group consisting of OTX2, GBX2, SIX1, SIX3, SOX1, SOX3, DLXS, EYA2, and BARX1.

### (Supplementary Note 60)

The method according to any one of Supplementary Notes 41 to 59, wherein
the pluripotent stem cell is a dissociated pluripotent stem cell or a cell suspension containing a pluripotent stem cell.

### (Supplementary Note 61)

The method according to any one of Supplementary Notes 41 to 60, wherein
the pluripotent stem cell is a human pluripotent stem cell.

### (Supplementary Note 62)

The method according to any one of Supplementary Notes 41 to 61, wherein
the pluripotent stem cell is an embryonic stem cell or an artificial pluripotent stem cell.

### (Supplementary Note 63)

The method according to any one of Supplementary Notes 41 to 62, wherein
the cellular aggregate comprising the mesodermal cell comprises at least 50 cells, at least 100 cells, at least 200 cells, at least 300 cells, at least 400 cells, at least 500 cells, at least 600 cells, at least 800 cells, at least 900 cells, at least 1000 cells, at least 1500 cells, at least 2000 cells, at least 2500 cells, at least 5000 cells, at least 10000 cells, at least 15000 cells, at least 20000 cells, at least 30000 cells, at least 40000 cells, or at least 50000 cells.

### (Supplementary Note 64)

The method according to any one of Supplementary Notes 41 to 63, wherein
the cellular aggregate comprising the mesodermal cell has a length of at least 0.05 mm, at least 0.1 mm, at least 0.2 mm, at least 0.3 mm, at least 0.4 mm, at least 0.5 mm, at least 0.6 mm, at least 0.7 mm, at least 0.8 mm, at least 0.9 mm or at least 1 mm.

### (Supplementary Note 65)

A cell obtained from the cellular aggregate according to any one of Supplementary Notes 1 to 40.

### (Supplementary Note 66)

A method for producing a progenitor cell or a differentiated cell, comprising the step of:
culturing the cellular aggregate according to any one of Supplementary Notes 1 to 40 to induce the progenitor cell or the differentiated cell selected from the group consisting of the following (a) to (f):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endothelial or hemogenic cell or a progenitor cell thereof; or
A method for producing a progenitor cell or a differentiated cell, comprising the step of:
culturing the cellular aggregate according to any one of Supplementary Notes 1 to 40 to induce the progenitor cell or the differentiated cell selected from the group consisting of the following (a) to (i):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endotome or endothelial or hemogenic cell or a progenitor cell thereof;
(g) an adipocyte cell including white, beige and brown cell or a progenitor cell thereof;
(h) a dermis cell or a progenitor cell thereof; and
(i) a neural tube cell or a progenitor cell thereof.

### (Supplementary Note 67)

The method according to Supplementary Note 66, comprising the step of:
inducing a three-dimensional cellular aggregate from a pluripotent stem cell prior to the inducing, wherein
the three-dimensional cellular aggregate inducing is performed by the method according to any one of Supplementary Notes 41 to 64.

### (Supplementary Note 68)

A method for evaluating a test substance, comprising the steps of:
culturing a test substance in the presence of a three-dimensional cellular aggregate; and
evaluating the three-dimensional cellular aggregate after the culturing, wherein
the three-dimensional cellular aggregate is the cellular aggregate according to any one of Supplementary Notes 1 to 40.

### (Supplementary Note 69)

The method according to Supplementary Note 68, wherein
during the evaluation, a test substance that changes a polarity of the cellular aggregate, a shape of the cellular aggregate, and/or a size of the cellular aggregate is selected as a candidate substance that modifies, promotes, or suppresses the polarity of the cellular aggregate, the shape of the cellular aggregate, and/or the size of the cellular aggregate.

### (Supplementary Note 70)

The method according to Supplementary Note 68, wherein
the culture is a culture under a segmentation culture condition.

### (Supplementary Note 71)

The method according to Supplementary Note 70, wherein
during the evaluation, a test substance that changes somitogenesis of the cellular aggregate is selected as a candidate substance that modifies, promotes, or suppresses the somitogenesis of the cellular aggregate.

### (Supplementary Note 72)

The method according to Supplementary Note 68, wherein
the culture is a culture inducing the progenitor cell or the differentiated cell selected from the group consisting of the following (a) to (f):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endothelial or hemogenic cell or a progenitor cell thereof; or
The method according to Supplementary Note 68, wherein
the culture is a culture inducing the progenitor cell or the differentiated cell selected from the group consisting of the following (a) to (i):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endotome or endothelial or hemogenic cell or a progenitor cell thereof;
(g) an adipocyte cell including white, beige and brown cell or a progenitor cell thereof;
(h) a dermis cell or a progenitor cell thereof; and
(i) a neural tube cell or a progenitor cell thereof.

### (Supplementary Note 73)

The method according to Supplementary Note 72, wherein
during the evaluation, a test substance that promotes or suppresses induction of the progenitor cell or the differentiated cell selected from the group consisting of (a) to (f) is selected as a candidate substance that promotes or suppresses induction of the progenitor cell or the differentiated cell selected from the group consisting of (a) to (f); or
The method according to Supplementary Note 72, wherein
during the evaluation, a test substance that promotes or suppresses induction of the progenitor cell or the differentiated cell selected from the group consisting of (a) to (i) is selected as a candidate substance that promotes or suppresses induction of the progenitor cell or the differentiated cell selected from the group consisting of (a) to (i).

### (Supplementary Note 74)

The method according to any one of Supplementary Notes 68 to 73, wherein
the evaluation is an evaluation using a control in which the test substance is not present as a reference.

### (Supplementary Note 75)

The method according to Supplementary Note 68, wherein
the culture is generation of a three-dimensional cellular aggregate from a pluripotent stem cell, and
the generation of the three-dimensional cellular aggregate is carried out by the method according to any one of Supplementary Notes 41 to 64.

### (Supplementary Note 76)

A method for evaluating gene function or genome function, comprising the steps of:
preparing a pluripotent stem cell in which a test gene or a test genome is modified;
generating a three-dimensional cellular aggregate from the pluripotent stem cell; and
evaluating a three-dimensional cellular aggregate after the culturing, wherein
the generation of the three-dimensional cellular aggregate is carried out by the method according to any one of Supplementary Notes 41 to 64: or
A method for evaluating gene function or genomic sequence function, comprising the steps of:
preparing a pluripotent stem cell in which a test gene or a test genomic sequence is modified;
generating a three-dimensional cellular aggregate from the pluripotent stem cell; and
evaluating a three-dimensional cellular aggregate after the culturing, wherein
the generation of the three-dimensional cellular aggregate is carried out by the method according to any one of Supplementary Notes 41 to 64.

### (Supplementary Note 77)

The method according to Supplementary Note 75, wherein
during the evaluation, a test gene or a test genome that changes a polarity of the cellular aggregate, a shape of the cellular aggregate, and/or a size of the cellular aggregate is evaluated as a candidate gene or a candidate genome that modifies, promotes, or suppresses the polarity of the cellular aggregate, the shape of the cellular aggregate, and/or the size of the cellular aggregate; or
The method according to Supplementary Note 75, wherein
during the evaluation, a test gene or a test genomic sequence that changes a polarity of the cellular aggregate, a shape of the cellular aggregate, and/or a size of the cellular aggregate is evaluated as a candidate gene or a candidate genomic sequence that modifies, promotes, or suppresses the polarity of the cellular aggregate, the shape of the cellular aggregate, and/or the size of the cellular aggregate.

### (Supplementary Note 78)

The method according to Supplementary Note 75, comprising the step of:
culturing in the presence of a three-dimensional cellular aggregate under a somitogenic culture condition, wherein
during the evaluation, a test gene or a test genome that changes somitogenesis of the cellular aggregate is evaluated as a candidate gene or a candidate genome that modifies, promotes, or suppresses the somitogenesis of the cellular aggregate; or
The method according to Supplementary Note 75, comprising the step of:
culturing in the presence of a three-dimensional cellular aggregate under a somitogenic culture condition, wherein
during the evaluation, a test gene or a test genomic sequence that changes somitogenesis of the cellular aggregate is evaluated as a candidate gene or a candidate genomic sequence that modifies, promotes, or suppresses the somitogenesis of the cellular aggregate.

### (Supplementary Note 79)

The method according to any one of Supplementary Notes 75 to 78, wherein
the genome is an exon region, an intron region, a promoter region, an enhancer region, and/or
a non-coding region of the genome; or
The method according to any one of Supplementary Notes 75 to 78, wherein
the genomic sequence is an exon region, an intron region, a promoter region, an enhancer region, and/or a non-coding region of the genome.

1. A three-dimensional cellular aggregate generated in vitro from a pluripotent stem cell, comprising:
a mesodermal cell, wherein the cellular aggregate has a polarity in an antero-posterior axis or a rostro-caudal axis and an apical-basolateral axis, and
the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition.

2. A three-dimensional cellular aggregate generated in vitro from a pluripotent stem cell, comprising:
a mesodermal cell, wherein the cellular aggregate has a polarity in an antero-posterior axis or a rostro-caudal axis
and an apical-basolateral axis, and
a proportion of the mesodermal cell in the cellular aggregate is at least 50%, at least 55%, at least 60%, at least 65%, at least 70%, at least 75%, at least 80%, at least 85%, at least 90%, at least 95%, at least 96%, at least 97%, at least 98% or at least 99%, based on the number of cells.

3. The cellular aggregate according to claim 1,
wherein the antero-posterior axis is defined by an anterior region and a posterior region, and
an anterior region cell has a lower expression of one or more markers as compared to the posterior region cell,
the one or more markers are selected from the group consisting of TBXT, SOX2, MIXL1, TBX6, HES7, MSGN1, MEOX1, TCF15, CYP26 A1, FGF3, FGF4, FGF8, FGF17, WNT3a, WNT5a, WNT5 b, HOXD13, HOXB, HOXA9, HOXA10 and CDX2, or
the anterior region cell has a higher expression of one or more markers as compared to the posterior region cell,
the one or more markers comprise LFNG, MEOX1, TCF15, UNCX, TBX18, ALDH1 A2 and RDH10.

4. The cellular aggregate according to claim 3, wherein the posterior region comprises a tailbud (TB) like-cell.

5. The cellular aggregate according to claim 1, wherein the apical-basolateral axis is defined by an apical region and a basolateral region,
an apical region of a cell has a lower expression of one or more markers as compared to the basolateral region of a cell,
the one or more markers are selected from the group consisting of Fibronectin, Collagen V and Laminin, or
the apical region of a cell has a higher expression of one or more markers as compared to the basolateral region of a cell,
the one or more markers are selected from the group consisting of aPKC, CDH2, Ezrin, ZO1 and F-Actin.

6. The cellular aggregate according to claim 1, wherein the mesodermal cell expresses a marker selected from the group consisting of TBXT, SOX2, NODAL, WNT3a, WNT5a, DLL1, TCF15, MEOX1, TBX18, UNCX, ALDH1 A2, RDH10, RIPPLY1, RIPPLY2, MESP1, MESP2, HES7, TBX6, MSGN1 and FLK1/KDR.

7. The cellular aggregate according to claim 2, wherein the cellular aggregate can reconstitute various aspects of somitogenesis and axial development, including axial elongation, segmentation, epithelial somite formation and patterning (formation of one or more somite like structures), and oscillation of the segmentation clock under a somitogenic culture condition.

8. The cellular aggregate according to claim 1, wherein the somitogenic culture condition is a presence of a gel or a matrix and a retinoid, retinoic acid, a retinoic acid precursor or derivative, and/or a retinoic acid receptor (RAR) agonist.

9. The cellular aggregate according to claim 8 wherein the cellular aggregate is embedded in the gel or the matrix or disposed inside the gel or the matrix.

10. The cellular aggregate according to claim 1, substantially not comprising an endodermal cell and/or an ectodermal cell.

11. The cellular aggregate according to claim 1, wherein an expression of a segmentation clock gene is subjected to gene oscillation.

12. The cellular aggregate according to claim 11, wherein the segmentation clock gene is a gene selected from the group consisting of LFNG, DKK1, DLL1, DLL3 and HES7.

13. A method for producing a three-dimensional cellular aggregate generated in vitro from a pluripotent stem cell, comprising:
culturing a pluripotent stem cell to induce a three-dimensional cellular aggregate comprising a mesodermal cell; and
culturing the cellular aggregate comprising the mesodermal cell to induce a three-dimensional cellular aggregate, wherein the three-dimensional cellular aggregate is the cellular aggregate of claim 1.

14. The method according to claim 13, comprising:
culturing the pluripotent stem cell in a medium containing a GSK33 inhibitor and FGF to initiate their commitment toward a primitive streak and mesodermal fate, and/or induce the mesodermal cell;
culturing the cells derived, obtained or obtainable from the culturing of the pluripotent stem cell in a medium containing the WNT agonist (GSK3β inhibitor), the FGF agonist, a TGFβ inhibitor and a ROCK inhibitor to induce the three-dimensional cellular aggregate including the mesodermal cell; and optionally
culturing the three-dimensional cellular aggregate including the mesodermal cell in a medium containing a retinoid, retinoic acid, a retinoic acid derivative and/or a retinoic acid receptor (RAR) agonist in the presence of a gel or a matrix to induce the three-dimensional cellular aggregate, and/or, a morphogenesis and/or a self-organization of the three-dimensional cellular aggregate.

15. The method according to claim 13, wherein a proportion of the mesodermal cell in the cellular aggregate comprising the mesodermal cell is at least 50%, at least 55%, at least 60%, at least 65%, at least 70%, at least 75%, at least 80%, at least 85%, at least 90%, at least 95%, at least 96%, at least 97%, at least 98% or at least 99%, based on the number of cells.

16. The method according to claim 13, wherein the mesodermal cell expresses a marker selected from the group consisting of TBXT, SOX2, MIXL1, LEFTY1, LEFTY2, AXIN2, TRH1, NODAL, WNT3a, WNT5a, DLL1, MEOX1, OSR1, PAX2, ALDH1 A2, MESP1, MESP2, TBX6, HES7, MSGN1, TCF15, MEOX1 and FLK1/KDR.

17. A cell obtained from the cellular aggregate of claim 1.

18. A method for producing a progenitor cell or a differentiated cell, comprising:
culturing the cellular aggregate according to claim 1 to induce the progenitor cell or the differentiated cell selected from the group consisting of the following
(a) to (i):
(a) neuro-mesodermal cell or a progenitor cell thereof;
(b) a muscle cell or a progenitor cell thereof;
(c) an osteocyte or a progenitor cell thereof;
(d) a chondrocyte or a progenitor cell thereof;
(e) a tenocyte or a progenitor cell thereof; and
(f) an endotome or endothelial or hemogenic cell or a progenitor cell thereof;
(g) an adipocyte cell including white, beige and brown cell or a progenitor cell thereof;
(h) a dermis cell or a progenitor cell thereof; and
(i) a neural tube cell or a progenitor cell thereof.

19. A method for evaluating a test substance, comprising:
culturing a test substance in the presence of a three-dimensional cellular aggregate; and evaluating the three-dimensional cellular aggregate after the culturing, wherein the three-dimensional cellular aggregate is the cellular aggregate of claim 1,
wherein during the evaluation, a test substance that changes a polarity of the cellular aggregate, a shape of the cellular aggregate and/or a size of the cellular aggregate is selected as a candidate substance that modifies, promotes or suppresses the polarity of the cellular aggregate, the shape of the cellular aggregate and/or the size of the cellular aggregate.

20. A method for evaluating gene function or genomic sequence function, comprising:
preparing a pluripotent stem cell in which a test gene or a test genomic sequence is modified; generating a three-dimensional cellular aggregate from the pluripotent stem cell; and
evaluating a three-dimensional cellular aggregate after the culturing, wherein the generation of the three-dimensional cellular aggregate is carried out by the method of claim 13, wherein during the evaluation, a test gene or a test genomic sequence that changes a polarity of the cellular aggregate, a shape of the cellular aggregate and/or a size of the cellular aggregate is evaluated as a candidate gene or a candidate genomic sequence that modifies, promotes or suppresses the polarity of the cellular aggregate, the shape of the cellular aggregate and/or the size of the cellular aggregate.


SEQUENCE LISTING (671 sequences)

SEQ ID NO: 1 - DNA, 33 bp, synthetic construct
  source 1..33: mol_type = other DNA
tcgtcggcagcgtcagatgtgtataagagacag

SEQ ID NO: 2 - DNA, 34 bp, synthetic construct
  source 1..34: mol_type = other DNA
gtctcgtgggctcggagatgtgtataagagacag

SEQ ID NO: 3 - DNA, 19 bp, synthetic construct
  source 1..19: mol_type = other DNA
ctgtctcttatacacatct

SEQ ID NO: 4 - DNA, 70 bp, synthetic construct
  source 1..70: mol_type = other DNA
ggctgctgcgagggcccttaactcgcgttcaagtatgcgtctatttagtggagcctcggaccagtcgttc

SEQ ID NO: 5 - DNA, 70 bp, synthetic construct
  source 1..70: mol_type = other DNA
gtgaacacggccttcccttaactcgcgttcaagtatgcgtctatttagtggagccgaccgcactcagagc

SEQ ID NO: 6 - DNA, 70 bp, synthetic construct
  source 1..70: mol_type = other DNA
gccgtcgtgacctggccttaactcgcgttcaagtatgcgtctatttagtggagccagcgcaaggggggtg

SEQ ID NO: 7 - DNA, 70 bp, synthetic construct
  source 1..70: mol_type = other DNA
atccattcagcctgcccttaactcgcgttcaagtatgcgtctatttagtggagcctggagagctgtgagg

SEQ ID NO: 8 - DNA, 70 bp, synthetic construct
  source 1..70: mol_type = other DNA
caaactttcctcagtccttaactcgcgttcaagtatgcgtctatttagtggagcctccgggcgccataac

SEQ ID NO: 9 - DNA, 70 bp, synthetic construct
  source 1..70: mol_type = other DNA
tccctgggggccggcggttgctacgatgactcagttgcgtctatttagtggagcccgagaattgtacccg

SEQ ID NO: 10 - DNA, 70 bp, synthetic construct
  source 1..70: mol_type = other DNA
acattcaccccgactggttgctacgatgactcagttgcgtctatttagtggagcctgcctgaccgtgtct

SEQ ID NO: 11 - DNA, 70 bp, synthetic construct
  source 1..70: mol_type = other DNA
tggagacattcgtgaggttgctacgatgactcagttgcgtctatttagtggagcccgactccaccctggg

SEQ ID NO: 12 - DNA, 70 bp, synthetic construct
  source 1..70: mol_type = other DNA
actgaaccactgctgggttgctacgatgactcagttgcgtctatttagtggagccgctccaaacccatgt

SEQ ID NO: 13 - DNA, 70 bp, synthetic construct
  source 1..70: mol_type = other DNA
ctgttgttctgggacggttgctacgatgactcagttgcgtctatttagtggagccgcaggccacaccagt

SEQ ID NO: 14 - DNA, 70 bp, synthetic construct
  source 1..70: mol_type = other DNA
gaccacctgctgagcatagacttaacgcacgatcctgcgtctatttagtggagccctgcagtaccgagtg

SEQ ID NO: 15 - DNA, 70 bp, synthetic construct
  source 1..70: mol_type = other DNA
cccgtctccttcagcatagacttaacgcacgatcctgcgtctatttagtggagcccactggatgaaggct

SEQ ID NO: 16 - DNA, 70 bp, synthetic construct
  source 1..70: mol_type = other DNA
caacggcgccgtcacatagacttaacgcacgatcctgcgtctatttagtggagcccctgtggtctgtgag

SEQ ID NO: 17 - DNA, 70 bp, synthetic construct
  source 1..70: mol_type = other DNA
tgtcgtggcagccagatagacttaacgcacgatcctgcgtctatttagtggagccgatgcagtgactttt

SEQ ID NO: 18 - DNA, 70 bp, synthetic construct
  source 1..70: mol_type = other DNA
ctagaatgtgtgtatatagacttaacgcacgatcctgcgtctatttagtggagcctgactgctctgcccc

SEQ ID NO: 19 - DNA, 70 bp, synthetic construct
  source 1..70: mol_type = other DNA
ctgcgacggcgcccgcggactaagggcgactaatatgcgtctatttagtggagccgtcgggttcgtgccc

SEQ ID NO: 20 - DNA, 70 bp, synthetic construct
  source 1..70: mol_type = other DNA
gtccccttggggctgcggactaagggcgactaatatgcgtctatttagtggagccgcgcggggacgcggg

SEQ ID NO: 21 - DNA, 70 bp, synthetic construct
  source 1..70: mol_type = other DNA
tcgtccccagagccccggactaagggcgactaatatgcgtctatttagtggagcctgtccctggacgcag

SEQ ID NO: 22 - DNA, 70 bp, synthetic construct
  source 1..70: mol_type = other DNA
ccctggatggagtcacggactaagggcgactaatatgcgtctatttagtggagccgatagcgggtacctt

SEQ ID NO: 23 - DNA, 70 bp, synthetic construct
  source 1..70: mol_type = other DNA
gttggccagagccctcggactaagggcgactaatatgcgtctatttagtggagcctgcttcttggtgaca

SEQ ID NO: 24 - DNA, 70 bp, synthetic construct
  source 1..70: mol_type = other DNA
ccggcgcaggcccaagaactatgctgacagtaccgtgcgtctatttagtggagccccctgtctcagacgc

SEQ ID NO: 25 - DNA, 70 bp, synthetic construct
  source 1..70: mol_type = other DNA
acccaactacccccagaactatgctgacagtaccgtgcgtctatttagtggagccaggactgagccccct

SEQ ID NO: 26 - DNA, 70 bp, synthetic construct
  source 1..70: mol_type = other DNA
cgactcggaaagtaagaactatgctgacagtaccgtgcgtctatttagtggagcctttggaaccagctgg

SEQ ID NO: 27 - DNA, 70 bp, synthetic construct
  source 1..70: mol_type = other DNA
ggcttgactgggtgggaactatgctgacagtaccgtgcgtctatttagtggagccaggcccgtccacaca

SEQ ID NO: 28 - DNA, 70 bp, synthetic construct
  source 1..70: mol_type = other DNA
gaaggttctccagtggaactatgctgacagtaccgtgcgtctatttagtggagcctggaaccctggagtg

SEQ ID NO: 29 - DNA, 70 bp, synthetic construct
  source 1..70: mol_type = other DNA
ggcgatgcccgatggatatctttaacgtcgagcgctgcgtctatttagtggagccgaaccacgccgcgga

SEQ ID NO: 30 - DNA, 70 bp, synthetic construct
  source 1..70: mol_type = other DNA
ttaccaattcaggcaatatctttaacgtcgagcgctgcgtctatttagtggagccagcctcaggaaagct

SEQ ID NO: 31 - DNA, 97 bp, synthetic construct
  source 1..97: mol_type = other DNA
gaggcaagaaagaagaggagatatctttaacgtcgagcgctgcgtctatttagtggagccagccgtgactatcgactgagaccctgggtggacgccg

SEQ ID NO: 32 - DNA, 97 bp, synthetic construct
  source 1..97: mol_type = other DNA
ctgaaaaattttccaattcaatatctttaacgtcgagcgctgcgtctatttagtggagccagccgtgactatcgactatcaagaagcagaagctctt

SEQ ID NO: 33 - DNA, 97 bp, synthetic construct
  source 1..97: mol_type = other DNA
atccaaagcttgtggggtttatatctttaacgtcgagcgctgcgtctatttagtggagccagccgtgactatcgactgattagctacttttgattat

SEQ ID NO: 34 - DNA, 70 bp, synthetic construct
  source 1..70: mol_type = other DNA
agcgaagccgggtggcggtcaactctacctaaggatgcgtctatttagtggagcctgggctacttgaggg

SEQ ID NO: 35 - DNA, 70 bp, synthetic construct
  source 1..70: mol_type = other DNA
cgagcaggtgttgggcggtcaactctacctaaggatgcgtctatttagtggagccacccgggcgtccggg

SEQ ID NO: 36 - DNA, 70 bp, synthetic construct
  source 1..70: mol_type = other DNA
tccctagtgcatggacggtcaactctacctaaggatgcgtctatttagtggagcccagtcccttcccgag

SEQ ID NO: 37 - DNA, 70 bp, synthetic construct
  source 1..70: mol_type = other DNA
agagccacccgacgacggtcaactctacctaaggatgcgtctatttagtggagcctatccattgcgccac

SEQ ID NO: 38 - DNA, 70 bp, synthetic construct
  source 1..70: mol_type = other DNA
atatagtcccacccacggtcaactctacctaaggatgcgtctatttagtggagccgctccccactcccag

SEQ ID NO: 39 - DNA, 70 bp, synthetic construct
  source 1..70: mol_type = other DNA
cgctcgtcgtcgacattcgtcggatcgaggttatctgcgtctatttagtggagccatgatgatatcgccg

SEQ ID NO: 40 - DNA, 70 bp, synthetic construct
  source 1..70: mol_type = other DNA
cggtgacggggtcacttcgtcggatcgaggttatctgcgtctatttagtggagcccatcgtgatggactc

SEQ ID NO: 41 - DNA, 70 bp, synthetic construct
  source 1..70: mol_type = other DNA
ccgacaggatgcagattcgtcggatcgaggttatctgcgtctatttagtggagcctgtaccctggcattg

SEQ ID NO: 42 - DNA, 70 bp, synthetic construct
  source 1..70: mol_type = other DNA
tgtggatcggcggctttcgtcggatcgaggttatctgcgtctatttagtggagccagcgcaagtactccg

SEQ ID NO: 43 - DNA, 70 bp, synthetic construct
  source 1..70: mol_type = other DNA
ttggcgaggacccagcacggataactactagaccgtgcgtctatttagtggagcccgtccagtggtaata

SEQ ID NO: 44 - DNA, 70 bp, synthetic construct
  source 1..70: mol_type = other DNA
cctgacaaattaactcacggataactactagaccgtgcgtctatttagtggagccagacagagccgaagg

SEQ ID NO: 45 - DNA, 70 bp, synthetic construct
  source 1..70: mol_type = other DNA
aatcgccttgtaaaacacggataactactagaccgtgcgtctatttagtggagccctggcaactctggca

SEQ ID NO: 46 - DNA, 70 bp, synthetic construct
  source 1..70: mol_type = other DNA
gttattttcagtaggcacggataactactagaccgtgcgtctatttagtggagccctgccgctgtgttcc

SEQ ID NO: 47 - DNA, 70 bp, synthetic construct
  source 1..70: mol_type = other DNA
gcgatagcctcacctcacggataactactagaccgtgcgtctatttagtggagccctgttcttcaggaaa

SEQ ID NO: 48 - DNA, 70 bp, synthetic construct
  source 1..70: mol_type = other DNA
gactataataggatgccttagacacatcatacggatgcgtctatttagtggagcccggccttgacgcatg

SEQ ID NO: 49 - DNA, 70 bp, synthetic construct
  source 1..70: mol_type = other DNA
ctcgagctacggggcccttagacacatcatacggatgcgtctatttagtggagccaggctattttcatcc

SEQ ID NO: 50 - DNA, 70 bp, synthetic construct
  source 1..70: mol_type = other DNA
gcacctccccggaagccttagacacatcatacggatgcgtctatttagtggagcccagaccagtcgacat

SEQ ID NO: 51 - DNA, 70 bp, synthetic construct
  source 1..70: mol_type = other DNA
agcaccagctgccttccttagacacatcatacggatgcgtctatttagtggagcccacagaccttacaat

SEQ ID NO: 52 - DNA, 70 bp, synthetic construct
  source 1..70: mol_type = other DNA
agctgcaaggatttaccttagacacatcatacggatgcgtctatttagtggagccatgagggctcccaac

SEQ ID NO: 53 - DNA, 70 bp, synthetic construct
  source 1..70: mol_type = other DNA
tggagcggccgttgcgcctagataccgtagatgactgcgtctatttagtggagccgcatttctcgaccta

SEQ ID NO: 54 - DNA, 70 bp, synthetic construct
  source 1..70: mol_type = other DNA
agtggggtcggctacgcctagataccgtagatgactgcgtctatttagtggagcccgaatgaattcgcac

SEQ ID NO: 55 - DNA, 70 bp, synthetic construct
  source 1..70: mol_type = other DNA
tcggggggcggcggagcctagataccgtagatgactgcgtctatttagtggagccctcacatccactctc

SEQ ID NO: 56 - DNA, 70 bp, synthetic construct
  source 1..70: mol_type = other DNA
ccacgcgcctcctccgcctagataccgtagatgactgcgtctatttagtggagcccacaactctctttca

SEQ ID NO: 57 - DNA, 70 bp, synthetic construct
  source 1..70: mol_type = other DNA
gtctacaggccctttgcctagataccgtagatgactgcgtctatttagtggagccgctcgttctcggctt

SEQ ID NO: 58 - DNA, 70 bp, synthetic construct
  source 1..70: mol_type = other DNA
gtattgctccttaaatagacgtagtgagcatgacttgcgtctatttagtggagccagctcctccgctgta

SEQ ID NO: 59 - DNA, 70 bp, synthetic construct
  source 1..70: mol_type = other DNA
gtgtcaaggaggagatagacgtagtgagcatgacttgcgtctatttagtggagccgctcctacccaccta

SEQ ID NO: 60 - DNA, 70 bp, synthetic construct
  source 1..70: mol_type = other DNA
cgaaaaggagagggctagacgtagtgagcatgacttgcgtctatttagtggagccgagcccctcggagag

SEQ ID NO: 61 - DNA, 70 bp, synthetic construct
  source 1..70: mol_type = other DNA
tgcaatgcgactgcatagacgtagtgagcatgacttgcgtctatttagtggagccctccctgagtataaa

SEQ ID NO: 62 - DNA, 70 bp, synthetic construct
  source 1..70: mol_type = other DNA
atattgtcctgtccctagacgtagtgagcatgacttgcgtctatttagtggagcctgcccccccccccaa

SEQ ID NO: 63 - DNA, 70 bp, synthetic construct
  source 1..70: mol_type = other DNA
gactcggcgagcatgcggacattaccacctagagttgcgtctatttagtggagccagcgagcaattcagg

SEQ ID NO: 64 - DNA, 70 bp, synthetic construct
  source 1..70: mol_type = other DNA
gatccgctgccctgccggacattaccacctagagttgcgtctatttagtggagcccactctcctcagccc

SEQ ID NO: 65 - DNA, 70 bp, synthetic construct
  source 1..70: mol_type = other DNA
acggcctacacgcgccggacattaccacctagagttgcgtctatttagtggagccggcaaaagggcccgg

SEQ ID NO: 66 - DNA, 70 bp, synthetic construct
  source 1..70: mol_type = other DNA
gtcagtactaaggtgcggacattaccacctagagttgcgtctatttagtggagcccggatcccgcgtagt

SEQ ID NO: 67 - DNA, 70 bp, synthetic construct
  source 1..70: mol_type = other DNA
agcttcagtgatgtacggacattaccacctagagttgcgtctatttagtggagccggctcttataagcgc

SEQ ID NO: 68 - DNA, 70 bp, synthetic construct
  source 1..70: mol_type = other DNA
aggccgaatggcggcctagacagactgcgacatactgcgtctatttagtggagcctacttctccaacctg

SEQ ID NO: 69 - DNA, 70 bp, synthetic construct
  source 1..70: mol_type = other DNA
cccgacggcctgccgctagacagactgcgacatactgcgtctatttagtggagccctgcgaccttcactg

SEQ ID NO: 70 - DNA, 70 bp, synthetic construct
  source 1..70: mol_type = other DNA
ggtctctgcagcagactagacagactgcgacatactgcgtctatttagtggagccctgtgggtacaatta

SEQ ID NO: 71 - DNA, 70 bp, synthetic construct
  source 1..70: mol_type = other DNA
ctttaaggccctgggctagacagactgcgacatactgcgtctatttagtggagccctgtcccagtggaga

SEQ ID NO: 72 - DNA, 70 bp, synthetic construct
  source 1..70: mol_type = other DNA
actgacttcagttgactagacagactgcgacatactgcgtctatttagtggagccctgagccttagccca

SEQ ID NO: 73 - DNA, 70 bp, synthetic construct
  source 1..70: mol_type = other DNA
aagagaagtgaaccactagacatatctcacagcgatgcgtctatttagtggagcccccgtctctggccaa

SEQ ID NO: 74 - DNA, 70 bp, synthetic construct
  source 1..70: mol_type = other DNA
cctgaggttcccgtcctagacatatctcacagcgatgcgtctatttagtggagcctgtcccgttgagaac

SEQ ID NO: 75 - DNA, 70 bp, synthetic construct
  source 1..70: mol_type = other DNA
cgtgagcggccaggactagacatatctcacagcgatgcgtctatttagtggagccgaagatgaacgagcc

SEQ ID NO: 76 - DNA, 70 bp, synthetic construct
  source 1..70: mol_type = other DNA
caccgacaggcaggtctagacatatctcacagcgatgcgtctatttagtggagcctaagagcgttaacct

SEQ ID NO: 77 - DNA, 70 bp, synthetic construct
  source 1..70: mol_type = other DNA
tgcatctgtggtttgctagacatatctcacagcgatgcgtctatttagtggagccgcctttcccttgtgg

SEQ ID NO: 78 - DNA, 70 bp, synthetic construct
  source 1..70: mol_type = other DNA
ggtgttcggcgcctcctttccaatacggtgtacgatgcgtctatttagtggagccccagcccaaagcgcc

SEQ ID NO: 79 - DNA, 70 bp, synthetic construct
  source 1..70: mol_type = other DNA
agtcaagctcctagtctttccaatacggtgtacgatgcgtctatttagtggagccggaaagagagacccc

SEQ ID NO: 80 - DNA, 70 bp, synthetic construct
  source 1..70: mol_type = other DNA
tagaaagtacaagaactttccaatacggtgtacgatgcgtctatttagtggagcccccaagccggtgggc

SEQ ID NO: 81 - DNA, 70 bp, synthetic construct
  source 1..70: mol_type = other DNA
tccgaatgagaatttctttccaatacggtgtacgatgcgtctatttagtggagccgccaggccaggagag

SEQ ID NO: 82 - DNA, 70 bp, synthetic construct
  source 1..70: mol_type = other DNA
ctctaattgtttgttctttccaatacggtgtacgatgcgtctatttagtggagccgcgaggggcctagag

SEQ ID NO: 83 - DNA, 70 bp, synthetic construct
  source 1..70: mol_type = other DNA
acggacagacaagtgctttcgacgctgacactaaatgcgtctatttagtggagcccgcagcgtccacctc

SEQ ID NO: 84 - DNA, 70 bp, synthetic construct
  source 1..70: mol_type = other DNA
ccgagcgctggtcccctttcgacgctgacactaaatgcgtctatttagtggagcccggttttttcacttc

SEQ ID NO: 85 - DNA, 70 bp, synthetic construct
  source 1..70: mol_type = other DNA
cttggggtcccactgctttcgacgctgacactaaatgcgtctatttagtggagccggctgggtttgctgt

SEQ ID NO: 86 - DNA, 70 bp, synthetic construct
  source 1..70: mol_type = other DNA
tgcctggagtgctgtctttcgacgctgacactaaatgcgtctatttagtggagccgccaccgggcatgtc

SEQ ID NO: 87 - DNA, 70 bp, synthetic construct
  source 1..70: mol_type = other DNA
gtggaaaaagacgatctttcgacgctgacactaaatgcgtctatttagtggagccgtattgctgctgtgc

SEQ ID NO: 88 - DNA, 70 bp, synthetic construct
  source 1..70: mol_type = other DNA
caaaacctccatccggcactattacgcagagcatctgcgtctatttagtggagcctcgctagaccgggtc

SEQ ID NO: 89 - DNA, 70 bp, synthetic construct
  source 1..70: mol_type = other DNA
ggctcctacggcggcgcactattacgcagagcatctgcgtctatttagtggagccctcatgaaaaacgaa

SEQ ID NO: 90 - DNA, 70 bp, synthetic construct
  source 1..70: mol_type = other DNA
gtgaagggaagtgtcgcactattacgcagagcatctgcgtctatttagtggagccgggcctggaaagggg

SEQ ID NO: 91 - DNA, 70 bp, synthetic construct
  source 1..70: mol_type = other DNA
tagctactctgaagcgcactattacgcagagcatctgcgtctatttagtggagccgctgaggacattcca

SEQ ID NO: 92 - DNA, 70 bp, synthetic construct
  source 1..70: mol_type = other DNA
gacaactcatctcatgcactattacgcagagcatctgcgtctatttagtggagccggggcagaggttcag

SEQ ID NO: 93 - DNA, 70 bp, synthetic construct
  source 1..70: mol_type = other DNA
gcaccgaggcgccccggtatagtacattcccgcactgcgtctatttagtggagccatccggccgagctca

SEQ ID NO: 94 - DNA, 70 bp, synthetic construct
  source 1..70: mol_type = other DNA
agcagaacccgtgctggtatagtacattcccgcactgcgtctatttagtggagccccaactcaggctacc

SEQ ID NO: 95 - DNA, 70 bp, synthetic construct
  source 1..70: mol_type = other DNA
gaagtctctcatgccggtatagtacattcccgcactgcgtctatttagtggagcccgaaaacgtcggatt

SEQ ID NO: 96 - DNA, 70 bp, synthetic construct
  source 1..70: mol_type = other DNA
cctcgaaatgcagaaggtatagtacattcccgcactgcgtctatttagtggagcctttagaggggagccc

SEQ ID NO: 97 - DNA, 70 bp, synthetic construct
  source 1..70: mol_type = other DNA
ttttattgtgcttctggtatagtacattcccgcactgcgtctatttagtggagccgccggtcctgtgcgc

SEQ ID NO: 98 - DNA, 70 bp, synthetic construct
  source 1..70: mol_type = other DNA
ctatgaaaacccaggggtatgcgacctactcttactgcgtctatttagtggagccgcagaaggctgctta

SEQ ID NO: 99 - DNA, 70 bp, synthetic construct
  source 1..70: mol_type = other DNA
agaactccaagcagaggtatgcgacctactcttactgcgtctatttagtggagcctgaaagagtctcgac

SEQ ID NO: 100 - DNA, 70 bp, synthetic construct
  source 1..70: mol_type = other DNA
cactcagcagctgccggtatgcgacctactcttactgcgtctatttagtggagccccgcctacacggcgc

SEQ ID NO: 101 - DNA, 70 bp, synthetic construct
  source 1..70: mol_type = other DNA
aattacctctcttgcggtatgcgacctactcttactgcgtctatttagtggagccccgaactcgcggcaa

SEQ ID NO: 102 - DNA, 70 bp, synthetic construct
  source 1..70: mol_type = other DNA
tggcacaacacacttggtatgcgacctactcttactgcgtctatttagtggagccggaagcccggtggcg

SEQ ID NO: 103 - DNA, 70 bp, synthetic construct
  source 1..70: mol_type = other DNA
gtacgtgtcatgcaggtctgagaatgtgttagcactgcgtctatttagtggagccgagctcctacctgga

SEQ ID NO: 104 - DNA, 70 bp, synthetic construct
  source 1..70: mol_type = other DNA
ccggcttgtctcaaagtctgagaatgtgttagcactgcgtctatttagtggagccgaacccggccctttt

SEQ ID NO: 105 - DNA, 70 bp, synthetic construct
  source 1..70: mol_type = other DNA
tggggccgctagcccgtctgagaatgtgttagcactgcgtctatttagtggagcccaaactcgccgagta

SEQ ID NO: 106 - DNA, 70 bp, synthetic construct
  source 1..70: mol_type = other DNA
tctagacttaggagcgtctgagaatgtgttagcactgcgtctatttagtggagcccgacccccatcccta

SEQ ID NO: 107 - DNA, 70 bp, synthetic construct
  source 1..70: mol_type = other DNA
gctcagaagagcaccgtctgagaatgtgttagcactgcgtctatttagtggagcctcagcactctgatgt

SEQ ID NO: 108 - DNA, 70 bp, synthetic construct
  source 1..70: mol_type = other DNA
ccggggaccctgggcgtctgcatagagaacaacgttgcgtctatttagtggagccgttggccgctatgcg

SEQ ID NO: 109 - DNA, 70 bp, synthetic construct
  source 1..70: mol_type = other DNA
gaacccagtgcacgcgtctgcatagagaacaacgttgcgtctatttagtggagccgtttggcgcctcgtg

SEQ ID NO: 110 - DNA, 70 bp, synthetic construct
  source 1..70: mol_type = other DNA
gtacatgcgctcctggtctgcatagagaacaacgttgcgtctatttagtggagccggcgccggacggcag

SEQ ID NO: 111 - DNA, 70 bp, synthetic construct
  source 1..70: mol_type = other DNA
taaacctgaaccgctgtctgcatagagaacaacgttgcgtctatttagtggagccccggccttatggcat

SEQ ID NO: 112 - DNA, 70 bp, synthetic construct
  source 1..70: mol_type = other DNA
tgacactcacactttgtctgcatagagaacaacgttgcgtctatttagtggagcctgactgtcccacgct

SEQ ID NO: 113 - DNA, 70 bp, synthetic construct
  source 1..70: mol_type = other DNA
cctaggccttgccccacctaataacactcacgggttgcgtctatttagtggagcctgcgctcgccttttt

SEQ ID NO: 114 - DNA, 70 bp, synthetic construct
  source 1..70: mol_type = other DNA
ctcgccacggcgctgacctaataacactcacgggttgcgtctatttagtggagccaccccggcagtcacc

SEQ ID NO: 115 - DNA, 70 bp, synthetic construct
  source 1..70: mol_type = other DNA
aatcgccgatggcagacctaataacactcacgggttgcgtctatttagtggagcccaaagaatccctgga

SEQ ID NO: 116 - DNA, 70 bp, synthetic construct
  source 1..70: mol_type = other DNA
aaccagcaatgagaaacctaataacactcacgggttgcgtctatttagtggagcccccagtctcgccttt

SEQ ID NO: 117 - DNA, 70 bp, synthetic construct
  source 1..70: mol_type = other DNA
ttgcagcatctgaatacctaataacactcacgggttgcgtctatttagtggagccctcaccacaatcgac

SEQ ID NO: 118 - DNA, 70 bp, synthetic construct
  source 1..70: mol_type = other DNA
atgacatactcctacacctacctaaagtgacatcgtgcgtctatttagtggagccccgtcttcgcgccca

SEQ ID NO: 119 - DNA, 70 bp, synthetic construct
  source 1..70: mol_type = other DNA
ccgcaaaaagcgctgacctacctaaagtgacatcgtgcgtctatttagtggagcccagtggccaacgcac

SEQ ID NO: 120 - DNA, 70 bp, synthetic construct
  source 1..70: mol_type = other DNA
gctgtgtgctctccaacctacctaaagtgacatcgtgcgtctatttagtggagcccccctgagtaaaaaa

SEQ ID NO: 121 - DNA, 70 bp, synthetic construct
  source 1..70: mol_type = other DNA
gtttggcaaactctcacctacctaaagtgacatcgtgcgtctatttagtggagccctgtggagtgtggca

SEQ ID NO: 122 - DNA, 70 bp, synthetic construct
  source 1..70: mol_type = other DNA
aggcaactgggcacaacctacctaaagtgacatcgtgcgtctatttagtggagccttgcagcacttatac

SEQ ID NO: 123 - DNA, 70 bp, synthetic construct
  source 1..70: mol_type = other DNA
ggcgcacccggcgccagatacttaaccatagcgcctgcgtctatttagtggagccgtgccgcaacctgat

SEQ ID NO: 124 - DNA, 70 bp, synthetic construct
  source 1..70: mol_type = other DNA
atggaaagctaccagagatacttaaccatagcgcctgcgtctatttagtggagcccccttgggtcttccc

SEQ ID NO: 125 - DNA, 70 bp, synthetic construct
  source 1..70: mol_type = other DNA
ccacgacgaatctctagatacttaaccatagcgcctgcgtctatttagtggagccggaggcggatatcag

SEQ ID NO: 126 - DNA, 70 bp, synthetic construct
  source 1..70: mol_type = other DNA
gctgtcgtattttaaagatacttaaccatagcgcctgcgtctatttagtggagccagcagcaatgcctag

SEQ ID NO: 127 - DNA, 70 bp, synthetic construct
  source 1..70: mol_type = other DNA
ctgccatcaagccaaagatacttaaccatagcgcctgcgtctatttagtggagccccatttccaaatgag

SEQ ID NO: 128 - DNA, 70 bp, synthetic construct
  source 1..70: mol_type = other DNA
actcggggacctgctatagagcgaacgatagttgctgcgtctatttagtggagcctacttagcagtggcg

SEQ ID NO: 129 - DNA, 70 bp, synthetic construct
  source 1..70: mol_type = other DNA
aatcaggaagcagacatagagcgaacgatagttgctgcgtctatttagtggagccagcccctacgcgtta

SEQ ID NO: 130 - DNA, 70 bp, synthetic construct
  source 1..70: mol_type = other DNA
gcgttccttccccggatagagcgaacgatagttgctgcgtctatttagtggagccccagctcttcgccct

SEQ ID NO: 131 - DNA, 70 bp, synthetic construct
  source 1..70: mol_type = other DNA
gggtgccagcatacaatagagcgaacgatagttgctgcgtctatttagtggagccaccttcatccagatt

SEQ ID NO: 132 - DNA, 70 bp, synthetic construct
  source 1..70: mol_type = other DNA
tatttggctgggctaatagagcgaacgatagttgctgcgtctatttagtggagccgcttaagcatccgtg

SEQ ID NO: 133 - DNA, 70 bp, synthetic construct
  source 1..70: mol_type = other DNA
tcccccaccattgaaatagagtcaacgagcgtacctgcgtctatttagtggagcccccccagcagccccc

SEQ ID NO: 134 - DNA, 70 bp, synthetic construct
  source 1..70: mol_type = other DNA
ggcctgctctgccgcatagagtcaacgagcgtacctgcgtctatttagtggagccagcgagccgaagatg

SEQ ID NO: 135 - DNA, 70 bp, synthetic construct
  source 1..70: mol_type = other DNA
ggaacccgcggccagatagagtcaacgagcgtacctgcgtctatttagtggagccctgcgaccctgccga

SEQ ID NO: 136 - DNA, 70 bp, synthetic construct
  source 1..70: mol_type = other DNA
ccctttttccgaggaatagagtcaacgagcgtacctgcgtctatttagtggagcctctcgacagcccggt

SEQ ID NO: 137 - DNA, 70 bp, synthetic construct
  source 1..70: mol_type = other DNA
gcatagacttatgtgatagagtcaacgagcgtacctgcgtctatttagtggagccctcccaccctcagtc

SEQ ID NO: 138 - DNA, 70 bp, synthetic construct
  source 1..70: mol_type = other DNA
gccttcgttcctttcccgtcagaagccatactagatgcgtctatttagtggagcctgacttcgctagcaa

SEQ ID NO: 139 - DNA, 70 bp, synthetic construct
  source 1..70: mol_type = other DNA
actacgcggcggcggccgtcagaagccatactagatgcgtctatttagtggagccggggctacgctccct

SEQ ID NO: 140 - DNA, 70 bp, synthetic construct
  source 1..70: mol_type = other DNA
cagcgcccgggccccccgtcagaagccatactagatgcgtctatttagtggagccaccagttctacgagg

SEQ ID NO: 141 - DNA, 70 bp, synthetic construct
  source 1..70: mol_type = other DNA
cgggacctcccagcgccgtcagaagccatactagatgcgtctatttagtggagccttcccacggtcaact

SEQ ID NO: 142 - DNA, 70 bp, synthetic construct
  source 1..70: mol_type = other DNA
cgctggtgttttgctccgtcagaagccatactagatgcgtctatttagtggagccggggttgctagaagg

SEQ ID NO: 143 - DNA, 70 bp, synthetic construct
  source 1..70: mol_type = other DNA
gcggctacggggcggccgtcgatacagactcagattgcgtctatttagtggagccccaactcacagagaa

SEQ ID NO: 144 - DNA, 70 bp, synthetic construct
  source 1..70: mol_type = other DNA
cgactgccgccgcagccgtcgatacagactcagattgcgtctatttagtggagccataaggacgaaggtc

SEQ ID NO: 145 - DNA, 70 bp, synthetic construct
  source 1..70: mol_type = other DNA
ccgactgctcccaggccgtcgatacagactcagattgcgtctatttagtggagccgcctgctacctagtg

SEQ ID NO: 146 - DNA, 70 bp, synthetic construct
  source 1..70: mol_type = other DNA
gaatgttcctgagctccgtcgatacagactcagattgcgtctatttagtggagcccccacttctggaggg

SEQ ID NO: 147 - DNA, 70 bp, synthetic construct
  source 1..70: mol_type = other DNA
acttcccagcccagcccgtcgatacagactcagattgcgtctatttagtggagccgttcagggaaggcac

SEQ ID NO: 148 - DNA, 70 bp, synthetic construct
  source 1..70: mol_type = other DNA
cccagttataacggactagactgaatcatcaccgatgcgtctatttagtggagccgtcagcgccgttaac

SEQ ID NO: 149 - DNA, 70 bp, synthetic construct
  source 1..70: mol_type = other DNA
ccggcggcgccgcatctagactgaatcatcaccgatgcgtctatttagtggagcccaatcgatacctgac

SEQ ID NO: 150 - DNA, 70 bp, synthetic construct
  source 1..70: mol_type = other DNA
agagagaacagttgtctagactgaatcatcaccgatgcgtctatttagtggagccgctggttgccaccca

SEQ ID NO: 151 - DNA, 70 bp, synthetic construct
  source 1..70: mol_type = other DNA
catacacttgcatctctagactgaatcatcaccgatgcgtctatttagtggagcccatgctgagtgggga

SEQ ID NO: 152 - DNA, 70 bp, synthetic construct
  source 1..70: mol_type = other DNA
ctggggcacggtcttctagactgaatcatcaccgatgcgtctatttagtggagcctactgttgtcccctt

SEQ ID NO: 153 - DNA, 70 bp, synthetic construct
  source 1..70: mol_type = other DNA
gcatcaccaccaggactctgtgaataccgagtacatgcgtctatttagtggagccggaatcgggattcca

SEQ ID NO: 154 - DNA, 70 bp, synthetic construct
  source 1..70: mol_type = other DNA
ggccacgggccggccctctgtgaataccgagtacatgcgtctatttagtggagcccccggcaattcgcga

SEQ ID NO: 155 - DNA, 70 bp, synthetic construct
  source 1..70: mol_type = other DNA
ctatacccggcagcactctgtgaataccgagtacatgcgtctatttagtggagccgcgctcgaggacagc

SEQ ID NO: 156 - DNA, 70 bp, synthetic construct
  source 1..70: mol_type = other DNA
gtacttctgaagaccctctgtgaataccgagtacatgcgtctatttagtggagcccggcagctaccccgg

SEQ ID NO: 157 - DNA, 70 bp, synthetic construct
  source 1..70: mol_type = other DNA
cctctcagagctgttctctgtgaataccgagtacatgcgtctatttagtggagccaatgcatctggagag

SEQ ID NO: 158 - DNA, 70 bp, synthetic construct
  source 1..70: mol_type = other DNA
ccggttcctgtccctggtcagagacgtagcatcaatgcgtctatttagtggagccagagcgcgcccctag

SEQ ID NO: 159 - DNA, 70 bp, synthetic construct
  source 1..70: mol_type = other DNA
agctcggtacccgggggtcagagacgtagcatcaatgcgtctatttagtggagccccctcagctcggctc

SEQ ID NO: 160 - DNA, 70 bp, synthetic construct
  source 1..70: mol_type = other DNA
cagccaaaggctggcggtcagagacgtagcatcaatgcgtctatttagtggagccgctggacgggttaga

SEQ ID NO: 161 - DNA, 70 bp, synthetic construct
  source 1..70: mol_type = other DNA
ggggtttcctggcacggtcagagacgtagcatcaatgcgtctatttagtggagccgctagctcaactagt

SEQ ID NO: 162 - DNA, 70 bp, synthetic construct
  source 1..70: mol_type = other DNA
tctgtgccctcctgaggtcagagacgtagcatcaatgcgtctatttagtggagcctccctgccacgaatt

SEQ ID NO: 163 - DNA, 70 bp, synthetic construct
  source 1..70: mol_type = other DNA
ctacccctaccaggcgtctggagagatcagtatcctgcgtctatttagtggagccggcgatctacggtgg

SEQ ID NO: 164 - DNA, 70 bp, synthetic construct
  source 1..70: mol_type = other DNA
ggcttcgtccaagcggtctggagagatcagtatcctgcgtctatttagtggagccgagcccgccggggca

SEQ ID NO: 165 - DNA, 70 bp, synthetic construct
  source 1..70: mol_type = other DNA
acggcggcaggggcggtctggagagatcagtatcctgcgtctatttagtggagccccacagaagcgctac

SEQ ID NO: 166 - DNA, 70 bp, synthetic construct
  source 1..70: mol_type = other DNA
caccatccttctcaggtctggagagatcagtatcctgcgtctatttagtggagccacggaccttaccggc

SEQ ID NO: 167 - DNA, 70 bp, synthetic construct
  source 1..70: mol_type = other DNA
cggtcttctgctccagtctggagagatcagtatcctgcgtctatttagtggagccggggataacgcaggg

SEQ ID NO: 168 - DNA, 70 bp, synthetic construct
  source 1..70: mol_type = other DNA
cggctacaattacaatactatgcctacctgtcgattgcgtctatttagtggagccgcacaccggctctta

SEQ ID NO: 169 - DNA, 70 bp, synthetic construct
  source 1..70: mol_type = other DNA
tccgaccaggcgacctactatgcctacctgtcgattgcgtctatttagtggagcctctgcttcgtccccc

SEQ ID NO: 170 - DNA, 70 bp, synthetic construct
  source 1..70: mol_type = other DNA
ccgctaccagacccttactatgcctacctgtcgattgcgtctatttagtggagccccggaccgcgtatac

SEQ ID NO: 171 - DNA, 70 bp, synthetic construct
  source 1..70: mol_type = other DNA
tggagtcccttttcatactatgcctacctgtcgattgcgtctatttagtggagccagctggcttcggaga

SEQ ID NO: 172 - DNA, 70 bp, synthetic construct
  source 1..70: mol_type = other DNA
tgctgtgctacgtgttactatgcctacctgtcgattgcgtctatttagtggagcctagtgagcgagtgga

SEQ ID NO: 173 - DNA, 70 bp, synthetic construct
  source 1..70: mol_type = other DNA
gagcgggttcgggcgtcgcgtctacctgactttagtgcgtctatttagtggagccagcgcccgggctatg

SEQ ID NO: 174 - DNA, 70 bp, synthetic construct
  source 1..70: mol_type = other DNA
cttcaacatgcactgtcgcgtctacctgactttagtgcgtctatttagtggagccgctcgagccgagttc

SEQ ID NO: 175 - DNA, 70 bp, synthetic construct
  source 1..70: mol_type = other DNA
ggaactgaccgcaaatcgcgtctacctgactttagtgcgtctatttagtggagcctggatgcgaagctca

SEQ ID NO: 176 - DNA, 70 bp, synthetic construct
  source 1..70: mol_type = other DNA
gcagcggcaccaaggtcgcgtctacctgactttagtgcgtctatttagtggagccgcctccgccttccca

SEQ ID NO: 177 - DNA, 70 bp, synthetic construct
  source 1..70: mol_type = other DNA
ttccccaagcgcctgtcgcgtctacctgactttagtgcgtctatttagtggagcccctaccactcgcgtg

SEQ ID NO: 178 - DNA, 70 bp, synthetic construct
  source 1..70: mol_type = other DNA
gcggcagcccgctggtcgcgttcaccgtaatcttatgcgtctatttagtggagccgagttgacaactccc

SEQ ID NO: 179 - DNA, 70 bp, synthetic construct
  source 1..70: mol_type = other DNA
cgttcgtgctggccgtcgcgttcaccgtaatcttatgcgtctatttagtggagcctcaaagtgctctggg

SEQ ID NO: 180 - DNA, 70 bp, synthetic construct
  source 1..70: mol_type = other DNA
ttcctacgatgctggtcgcgttcaccgtaatcttatgcgtctatttagtggagccccactaaggcttttc

SEQ ID NO: 181 - DNA, 70 bp, synthetic construct
  source 1..70: mol_type = other DNA
aggagcggacaagtgtcgcgttcaccgtaatcttatgcgtctatttagtggagcccatgtatcggttcct

SEQ ID NO: 182 - DNA, 70 bp, synthetic construct
  source 1..70: mol_type = other DNA
atggaggcactggcttcgcgttcaccgtaatcttatgcgtctatttagtggagccatggcccttataaca

SEQ ID NO: 183 - DNA, 70 bp, synthetic construct
  source 1..70: mol_type = other DNA
cggcggagcagcagcacctacgaacactaagtcggtgcgtctatttagtggagccaacaaatcataaacc

SEQ ID NO: 184 - DNA, 70 bp, synthetic construct
  source 1..70: mol_type = other DNA
ctgcgcactctccggacctacgaacactaagtcggtgcgtctatttagtggagcccgagaaagagtcggc

SEQ ID NO: 185 - DNA, 70 bp, synthetic construct
  source 1..70: mol_type = other DNA
caacagttcctccttacctacgaacactaagtcggtgcgtctatttagtggagccgcggatgaattcgtg

SEQ ID NO: 186 - DNA, 70 bp, synthetic construct
  source 1..70: mol_type = other DNA
gcccacccagcatccacctacgaacactaagtcggtgcgtctatttagtggagccggcctcactactcga

SEQ ID NO: 187 - DNA, 70 bp, synthetic construct
  source 1..70: mol_type = other DNA
ccgtgcacggttcaaacctacgaacactaagtcggtgcgtctatttagtggagccttttgtccatgtccc

SEQ ID NO: 188 - DNA, 70 bp, synthetic construct
  source 1..70: mol_type = other DNA
agcgcgcagctggtgtagactctcgatcagccgtatgcgtctatttagtggagcccggacggcgtacacg

SEQ ID NO: 189 - DNA, 70 bp, synthetic construct
  source 1..70: mol_type = other DNA
gcgctgccctccaactagactctcgatcagccgtatgcgtctatttagtggagcccaccagaatgcctac

SEQ ID NO: 190 - DNA, 70 bp, synthetic construct
  source 1..70: mol_type = other DNA
ctgcgaaccccaccctagactctcgatcagccgtatgcgtctatttagtggagccccagcaccacggacc

SEQ ID NO: 191 - DNA, 70 bp, synthetic construct
  source 1..70: mol_type = other DNA
tggggtagaggaagatagactctcgatcagccgtatgcgtctatttagtggagccttccacgcccgggag

SEQ ID NO: 192 - DNA, 70 bp, synthetic construct
  source 1..70: mol_type = other DNA
aaaagcgtgtgttcttagactctcgatcagccgtatgcgtctatttagtggagcctccagcattgctcaa

SEQ ID NO: 193 - DNA, 70 bp, synthetic construct
  source 1..70: mol_type = other DNA
gccctacctcggcagttgacatagtggtcgagttctgcgtctatttagtggagcccaatggctacccgca

SEQ ID NO: 194 - DNA, 70 bp, synthetic construct
  source 1..70: mol_type = other DNA
caactggcagagctgttgacatagtggtcgagttctgcgtctatttagtggagccccctattcgaagttg

SEQ ID NO: 195 - DNA, 70 bp, synthetic construct
  source 1..70: mol_type = other DNA
ggttagaagaggctgttgacatagtggtcgagttctgcgtctatttagtggagcccctcacagttcctgg

SEQ ID NO: 196 - DNA, 70 bp, synthetic construct
  source 1..70: mol_type = other DNA
aatttacgaggcgggttgacatagtggtcgagttctgcgtctatttagtggagccagggtggaccagcgt

SEQ ID NO: 197 - DNA, 70 bp, synthetic construct
  source 1..70: mol_type = other DNA
ccttcctgtgacctcttgacatagtggtcgagttctgcgtctatttagtggagccccccctttatgcagc

SEQ ID NO: 198 - DNA, 70 bp, synthetic construct
  source 1..70: mol_type = other DNA
gttgtagcggcgcgcaacgactaacaactgtccgttgcgtctatttagtggagcctcgtcctcttctgcc

SEQ ID NO: 199 - DNA, 70 bp, synthetic construct
  source 1..70: mol_type = other DNA
gaggcctacatctccaacgactaacaactgtccgttgcgtctatttagtggagccggggagcctcggcac

SEQ ID NO: 200 - DNA, 70 bp, synthetic construct
  source 1..70: mol_type = other DNA
ccaaaaggcctttggaacgactaacaactgtccgttgcgtctatttagtggagccgccattcggttgtct

SEQ ID NO: 201 - DNA, 70 bp, synthetic construct
  source 1..70: mol_type = other DNA
ccgagaaagtctaaaaacgactaacaactgtccgttgcgtctatttagtggagccagtgctctggggtca

SEQ ID NO: 202 - DNA, 70 bp, synthetic construct
  source 1..70: mol_type = other DNA
tggagctgtaaagcaaacgactaacaactgtccgttgcgtctatttagtggagcccaagggggcctcgca

SEQ ID NO: 203 - DNA, 70 bp, synthetic construct
  source 1..70: mol_type = other DNA
atgccaccttggatgacgaggttaaagtcaagtcctgcgtctatttagtggagcctggagcccggcaatt

SEQ ID NO: 204 - DNA, 70 bp, synthetic construct
  source 1..70: mol_type = other DNA
ttccgtacagcaaggacgaggttaaagtcaagtcctgcgtctatttagtggagccgccgcaagaaacgca

SEQ ID NO: 205 - DNA, 70 bp, synthetic construct
  source 1..70: mol_type = other DNA
taggtggacaattgtacgaggttaaagtcaagtcctgcgtctatttagtggagcccagctgacagctggg

SEQ ID NO: 206 - DNA, 70 bp, synthetic construct
  source 1..70: mol_type = other DNA
tggacaacccgcagaacgaggttaaagtcaagtcctgcgtctatttagtggagcccacaggcctgaagtc

SEQ ID NO: 207 - DNA, 70 bp, synthetic construct
  source 1..70: mol_type = other DNA
cctttgggggtctggacgaggttaaagtcaagtcctgcgtctatttagtggagcctggccctggtagaga

SEQ ID NO: 208 - DNA, 70 bp, synthetic construct
  source 1..70: mol_type = other DNA
ttataggccatgaggacgagtagaaatgacgctcctgcgtctatttagtggagccactacgtggactcgc

SEQ ID NO: 209 - DNA, 70 bp, synthetic construct
  source 1..70: mol_type = other DNA
gctccgtggcccgggacgagtagaaatgacgctcctgcgtctatttagtggagccccaaacggactgagt

SEQ ID NO: 210 - DNA, 70 bp, synthetic construct
  source 1..70: mol_type = other DNA
gctgaaggaggaggaacgagtagaaatgacgctcctgcgtctatttagtggagccgatcccaggctgttc

SEQ ID NO: 211 - DNA, 70 bp, synthetic construct
  source 1..70: mol_type = other DNA
ctatcccactccctcacgagtagaaatgacgctcctgcgtctatttagtggagccctttggggtttcgcc

SEQ ID NO: 212 - DNA, 70 bp, synthetic construct
  source 1..70: mol_type = other DNA
ttagaggagcccaggacgagtagaaatgacgctcctgcgtctatttagtggagccgatctccagtgaggc

SEQ ID NO: 213 - DNA, 70 bp, synthetic construct
  source 1..70: mol_type = other DNA
ctgtcccctcgggctagcggactaaatataggtcgtgcgtctatttagtggagccccgccaccgcccggt

SEQ ID NO: 214 - DNA, 70 bp, synthetic construct
  source 1..70: mol_type = other DNA
tatctgccctcccccagcggactaaatataggtcgtgcgtctatttagtggagccggccccggaaaaatc

SEQ ID NO: 215 - DNA, 70 bp, synthetic construct
  source 1..70: mol_type = other DNA
tctgccgaggagaagagcggactaaatataggtcgtgcgtctatttagtggagccatctgtcttgtttcc

SEQ ID NO: 216 - DNA, 70 bp, synthetic construct
  source 1..70: mol_type = other DNA
tgactgctcacccacagcggactaaatataggtcgtgcgtctatttagtggagcccactgagggccagaa

SEQ ID NO: 217 - DNA, 70 bp, synthetic construct
  source 1..70: mol_type = other DNA
agaacccttcgtatgagcggactaaatataggtcgtgcgtctatttagtggagccgggctgcttgagtct

SEQ ID NO: 218 - DNA, 70 bp, synthetic construct
  source 1..70: mol_type = other DNA
ggactgtagcgatttcacggtatagacaccacgtatgcgtctatttagtggagcccagcggtttggtgcc

SEQ ID NO: 219 - DNA, 70 bp, synthetic construct
  source 1..70: mol_type = other DNA
ttggctcgagccgctcacggtatagacaccacgtatgcgtctatttagtggagccgcgctacatgcggac

SEQ ID NO: 220 - DNA, 70 bp, synthetic construct
  source 1..70: mol_type = other DNA
tctcaccgagcggcacacggtatagacaccacgtatgcgtctatttagtggagccggcccgggttctcaa

SEQ ID NO: 221 - DNA, 70 bp, synthetic construct
  source 1..70: mol_type = other DNA
agattttgtacaaaacacggtatagacaccacgtatgcgtctatttagtggagccgggcgctctgcgtgc

SEQ ID NO: 222 - DNA, 70 bp, synthetic construct
  source 1..70: mol_type = other DNA
gtgcttctgtttgttcacggtatagacaccacgtatgcgtctatttagtggagcccgtccgtcctgtaac

SEQ ID NO: 223 - DNA, 70 bp, synthetic construct
  source 1..70: mol_type = other DNA
tggaggcttctctgtcacggtcaactaggtgagaatgcgtctatttagtggagccgcaagcttagacttt

SEQ ID NO: 224 - DNA, 70 bp, synthetic construct
  source 1..70: mol_type = other DNA
ttcggtgcgcaggatcacggtcaactaggtgagaatgcgtctatttagtggagcccaacgccagagccta

SEQ ID NO: 225 - DNA, 70 bp, synthetic construct
  source 1..70: mol_type = other DNA
agggcgacaagaagtcacggtcaactaggtgagaatgcgtctatttagtggagccagggcgacgcgcaga

SEQ ID NO: 226 - DNA, 70 bp, synthetic construct
  source 1..70: mol_type = other DNA
gtgagggcgagcggccacggtcaactaggtgagaatgcgtctatttagtggagccaggctccctcgccct

SEQ ID NO: 227 - DNA, 70 bp, synthetic construct
  source 1..70: mol_type = other DNA
agggcggaagagttacacggtcaactaggtgagaatgcgtctatttagtggagcctgtctgcgcctgaaa

SEQ ID NO: 228 - DNA, 70 bp, synthetic construct
  source 1..70: mol_type = other DNA
tacacctcaccttgtccttatcgaagtagacgacatgcgtctatttagtggagccctcccggacaagacg

SEQ ID NO: 229 - DNA, 70 bp, synthetic construct
  source 1..70: mol_type = other DNA
tcgccctcgggcagtccttatcgaagtagacgacatgcgtctatttagtggagccgacctcagtggcgcc

SEQ ID NO: 230 - DNA, 70 bp, synthetic construct
  source 1..70: mol_type = other DNA
tgtgtatgggagccaccttatcgaagtagacgacatgcgtctatttagtggagccctcctgcgcgggtgc

SEQ ID NO: 231 - DNA, 70 bp, synthetic construct
  source 1..70: mol_type = other DNA
tagatgcctgggcagccttatcgaagtagacgacatgcgtctatttagtggagccgcaaaggcgggcgag

SEQ ID NO: 232 - DNA, 70 bp, synthetic construct
  source 1..70: mol_type = other DNA
gcaagcgtctgtgcaccttatcgaagtagacgacatgcgtctatttagtggagccggagcccggagcttt

SEQ ID NO: 233 - DNA, 70 bp, synthetic construct
  source 1..70: mol_type = other DNA
gacgacttcgctgctcggtactaaggctgacctaatgcgtctatttagtggagccagcagatcatgtcat

SEQ ID NO: 234 - DNA, 70 bp, synthetic construct
  source 1..70: mol_type = other DNA
ggagttcgccttctacggtactaaggctgacctaatgcgtctatttagtggagccgtcctctagggccaa

SEQ ID NO: 235 - DNA, 70 bp, synthetic construct
  source 1..70: mol_type = other DNA
cggctagcaagttcacggtactaaggctgacctaatgcgtctatttagtggagcctagagaaggaatacg

SEQ ID NO: 236 - DNA, 70 bp, synthetic construct
  source 1..70: mol_type = other DNA
atcattccaaccaaacggtactaaggctgacctaatgcgtctatttagtggagccgcagcgcagagccca

SEQ ID NO: 237 - DNA, 70 bp, synthetic construct
  source 1..70: mol_type = other DNA
gcccaggaatgactccggtactaaggctgacctaatgcgtctatttagtggagcctccatcagggcacta

SEQ ID NO: 238 - DNA, 70 bp, synthetic construct
  source 1..70: mol_type = other DNA
agcctttcggaggcagacatcagagtaccagctcatgcgtctatttagtggagccccgacttcggtgagc

SEQ ID NO: 239 - DNA, 70 bp, synthetic construct
  source 1..70: mol_type = other DNA
cccaagcagccgcccgacatcagagtaccagctcatgcgtctatttagtggagcctacagtcagtccgac

SEQ ID NO: 240 - DNA, 70 bp, synthetic construct
  source 1..70: mol_type = other DNA
cgtcggattgaaatcgacatcagagtaccagctcatgcgtctatttagtggagcctatctgacaaggcgc

SEQ ID NO: 241 - DNA, 70 bp, synthetic construct
  source 1..70: mol_type = other DNA
actgtggggtggaccgacatcagagtaccagctcatgcgtctatttagtggagccgctgggctctaaggt

SEQ ID NO: 242 - DNA, 70 bp, synthetic construct
  source 1..70: mol_type = other DNA
ctctctctaagtatagacatcagagtaccagctcatgcgtctatttagtggagccagggggggcgggatt

SEQ ID NO: 243 - DNA, 70 bp, synthetic construct
  source 1..70: mol_type = other DNA
ctaagccaatggacaccttaactcgcgttcaagtatgcgtctatttagtggagcccgctcgccacccgtt

SEQ ID NO: 244 - DNA, 70 bp, synthetic construct
  source 1..70: mol_type = other DNA
aagagctggggatctccttaactcgcgttcaagtatgcgtctatttagtggagccaccttcctgcctgct

SEQ ID NO: 245 - DNA, 70 bp, synthetic construct
  source 1..70: mol_type = other DNA
cggtaccgtttgggaccttaactcgcgttcaagtatgcgtctatttagtggagcctcggtgtgcaggatg

SEQ ID NO: 246 - DNA, 70 bp, synthetic construct
  source 1..70: mol_type = other DNA
ttgcttgatttatcaccttaactcgcgttcaagtatgcgtctatttagtggagccgctgcctagggccac

SEQ ID NO: 247 - DNA, 70 bp, synthetic construct
  source 1..70: mol_type = other DNA
ttgtctcttgggcagccttaactcgcgttcaagtatgcgtctatttagtggagccgtttcggtgtgggca

SEQ ID NO: 248 - DNA, 70 bp, synthetic construct
  source 1..70: mol_type = other DNA
caaagctcatcgtggcggtacggaagtgtatatcatgcgtctatttagtggagccacggcgaccccttcg

SEQ ID NO: 249 - DNA, 70 bp, synthetic construct
  source 1..70: mol_type = other DNA
acggagattgtgctgcggtacggaagtgtatatcatgcgtctatttagtggagccaaggactgcgtcttc

SEQ ID NO: 250 - DNA, 70 bp, synthetic construct
  source 1..70: mol_type = other DNA
atgaagcggctgccccggtacggaagtgtatatcatgcgtctatttagtggagcccgtgaggtccacttc

SEQ ID NO: 251 - DNA, 70 bp, synthetic construct
  source 1..70: mol_type = other DNA
ccgcagagaggctcacggtacggaagtgtatatcatgcgtctatttagtggagccccccacaatgccaga

SEQ ID NO: 252 - DNA, 70 bp, synthetic construct
  source 1..70: mol_type = other DNA
ctgtgcgctgctctgcggtacggaagtgtatatcatgcgtctatttagtggagccctcaagcaagatgag

SEQ ID NO: 253 - DNA, 70 bp, synthetic construct
  source 1..70: mol_type = other DNA
cttgcaaccatggaagacagtttacactattcgcgtgcgtctatttagtggagcccgggacagggcagtt

SEQ ID NO: 254 - DNA, 70 bp, synthetic construct
  source 1..70: mol_type = other DNA
cccaccactgagcaggacagtttacactattcgcgtgcgtctatttagtggagccgggagtccctttgac

SEQ ID NO: 255 - DNA, 70 bp, synthetic construct
  source 1..70: mol_type = other DNA
aagttaagacggtgagacagtttacactattcgcgtgcgtctatttagtggagcctgcgggagtactcag

SEQ ID NO: 256 - DNA, 70 bp, synthetic construct
  source 1..70: mol_type = other DNA
gcctaggggtttcctgacagtttacactattcgcgtgcgtctatttagtggagccggaaaatctggccaa

SEQ ID NO: 257 - DNA, 70 bp, synthetic construct
  source 1..70: mol_type = other DNA
gtgtccaagtggacagacagtttacactattcgcgtgcgtctatttagtggagccgccctcctgtagcat

SEQ ID NO: 258 - DNA, 70 bp, synthetic construct
  source 1..70: mol_type = other DNA
cccaacggcacgctggacatagaccataccacgtttgcgtctatttagtggagccgccgcacccactgca

SEQ ID NO: 259 - DNA, 70 bp, synthetic construct
  source 1..70: mol_type = other DNA
catgaaggtcacccagacatagaccataccacgtttgcgtctatttagtggagccccgagtgtcgcccac

SEQ ID NO: 260 - DNA, 70 bp, synthetic construct
  source 1..70: mol_type = other DNA
ccacccccaaaacgggacatagaccataccacgtttgcgtctatttagtggagccatcgaaccccccgcc

SEQ ID NO: 261 - DNA, 70 bp, synthetic construct
  source 1..70: mol_type = other DNA
ccagaaatgctccgagacatagaccataccacgtttgcgtctatttagtggagccgaccttagcttgcta

SEQ ID NO: 262 - DNA, 70 bp, synthetic construct
  source 1..70: mol_type = other DNA
acatgctgaccgacagacatagaccataccacgtttgcgtctatttagtggagccgtgctgtgcacattg

SEQ ID NO: 263 - DNA, 70 bp, synthetic construct
  source 1..70: mol_type = other DNA
acttgcctgtgtttatagacgtagtgagcatgacttgcgtctatttagtggagccgcgccctccgcctgc

SEQ ID NO: 264 - DNA, 70 bp, synthetic construct
  source 1..70: mol_type = other DNA
aggcaagctcgtgggtagacgtagtgagcatgacttgcgtctatttagtggagccgtgcatgaaccgcaa

SEQ ID NO: 265 - DNA, 70 bp, synthetic construct
  source 1..70: mol_type = other DNA
cccacacaccctgcctagacgtagtgagcatgacttgcgtctatttagtggagcctcccgtcggatccgg

SEQ ID NO: 266 - DNA, 70 bp, synthetic construct
  source 1..70: mol_type = other DNA
cgcgcctcgacctcctagacgtagtgagcatgacttgcgtctatttagtggagcccccctgggaattctc

SEQ ID NO: 267 - DNA, 70 bp, synthetic construct
  source 1..70: mol_type = other DNA
cacgtgacctagtactagacgtagtgagcatgacttgcgtctatttagtggagcccgaccttccgtgact

SEQ ID NO: 268 - DNA, 70 bp, synthetic construct
  source 1..70: mol_type = other DNA
ctacgtggagatcatctctgcgaaggtagtgtaattgcgtctatttagtggagccccgcttctgcaggaa

SEQ ID NO: 269 - DNA, 70 bp, synthetic construct
  source 1..70: mol_type = other DNA
cctctctggtggctgctctgcgaaggtagtgtaattgcgtctatttagtggagccctggagctagtgtct

SEQ ID NO: 270 - DNA, 70 bp, synthetic construct
  source 1..70: mol_type = other DNA
aacccgtgcccctggctctgcgaaggtagtgtaattgcgtctatttagtggagccgcgagtcgggtcccc

SEQ ID NO: 271 - DNA, 70 bp, synthetic construct
  source 1..70: mol_type = other DNA
cgggaggtttgggggctctgcgaaggtagtgtaattgcgtctatttagtggagcctcatttgggggcgtt

SEQ ID NO: 272 - DNA, 70 bp, synthetic construct
  source 1..70: mol_type = other DNA
gcacaccgccatcctctctgcgaaggtagtgtaattgcgtctatttagtggagccagagcccaaaagagg

SEQ ID NO: 273 - DNA, 70 bp, synthetic construct
  source 1..70: mol_type = other DNA
ccagtcggacatcggctttccaatacggtgtacgatgcgtctatttagtggagcccgactacgtgagctt

SEQ ID NO: 274 - DNA, 70 bp, synthetic construct
  source 1..70: mol_type = other DNA
gttcttcggcttctactttccaatacggtgtacgatgcgtctatttagtggagcccgagccggtcatgca

SEQ ID NO: 275 - DNA, 70 bp, synthetic construct
  source 1..70: mol_type = other DNA
tgacggccatccacactttccaatacggtgtacgatgcgtctatttagtggagccagagccagtacttgc

SEQ ID NO: 276 - DNA, 70 bp, synthetic construct
  source 1..70: mol_type = other DNA
gctcccttccctccactttccaatacggtgtacgatgcgtctatttagtggagcctcttgcagcattccc

SEQ ID NO: 277 - DNA, 70 bp, synthetic construct
  source 1..70: mol_type = other DNA
gcaaccgggccctctctttccaatacggtgtacgatgcgtctatttagtggagccgacaaagtgcctcat

SEQ ID NO: 278 - DNA, 70 bp, synthetic construct
  source 1..70: mol_type = other DNA
cggtcctggggaatgctttcgacgctgacactaaatgcgtctatttagtggagccgcctctcccagaact

SEQ ID NO: 279 - DNA, 70 bp, synthetic construct
  source 1..70: mol_type = other DNA
gcccaaagaaaacccctttcgacgctgacactaaatgcgtctatttagtggagcctccccaaagcgcacc

SEQ ID NO: 280 - DNA, 70 bp, synthetic construct
  source 1..70: mol_type = other DNA
ggggaggcgtggggactttcgacgctgacactaaatgcgtctatttagtggagcctagggagggttgatg

SEQ ID NO: 281 - DNA, 70 bp, synthetic construct
  source 1..70: mol_type = other DNA
tggtgggtgtcatagctttcgacgctgacactaaatgcgtctatttagtggagccaacttggatgcggcg

SEQ ID NO: 282 - DNA, 70 bp, synthetic construct
  source 1..70: mol_type = other DNA
gtaggagtagctgggctttcgacgctgacactaaatgcgtctatttagtggagccccagaatagctagga

SEQ ID NO: 283 - DNA, 70 bp, synthetic construct
  source 1..70: mol_type = other DNA
ttgaaaaatggcaaagcactattacgcagagcatctgcgtctatttagtggagcccccacactgcgctgg

SEQ ID NO: 284 - DNA, 70 bp, synthetic construct
  source 1..70: mol_type = other DNA
caaaacagtggccctgcactattacgcagagcatctgcgtctatttagtggagccagggttgcccgccaa

SEQ ID NO: 285 - DNA, 70 bp, synthetic construct
  source 1..70: mol_type = other DNA
ttctggaagccctgggcactattacgcagagcatctgcgtctatttagtggagccctgcatggttgaccg

SEQ ID NO: 286 - DNA, 70 bp, synthetic construct
  source 1..70: mol_type = other DNA
ggctatcgggctggagcactattacgcagagcatctgcgtctatttagtggagccggtggtgttggcaga

SEQ ID NO: 287 - DNA, 70 bp, synthetic construct
  source 1..70: mol_type = other DNA
cgcatggacaagcccgcactattacgcagagcatctgcgtctatttagtggagccctgaaggagggtcac

SEQ ID NO: 288 - DNA, 70 bp, synthetic construct
  source 1..70: mol_type = other DNA
cgagtatgaacttccggtatagtacattcccgcactgcgtctatttagtggagccgctggcaggggtctc

SEQ ID NO: 289 - DNA, 70 bp, synthetic construct
  source 1..70: mol_type = other DNA
gacactgggcaagccggtatagtacattcccgcactgcgtctatttagtggagcctccaagagataagct

SEQ ID NO: 290 - DNA, 70 bp, synthetic construct
  source 1..70: mol_type = other DNA
ggagtactcctatgaggtatagtacattcccgcactgcgtctatttagtggagccgaggccacccgggat

SEQ ID NO: 291 - DNA, 70 bp, synthetic construct
  source 1..70: mol_type = other DNA
acctaccagctggccggtatagtacattcccgcactgcgtctatttagtggagccgacttggtgtcatgc

SEQ ID NO: 292 - DNA, 70 bp, synthetic construct
  source 1..70: mol_type = other DNA
accaacgaactgtacggtatagtacattcccgcactgcgtctatttagtggagccaagccagccaactgc

SEQ ID NO: 293 - DNA, 70 bp, synthetic construct
  source 1..70: mol_type = other DNA
ggcagcggggatgctggtatgcgacctactcttactgcgtctatttagtggagccgagcagttggtcttc

SEQ ID NO: 294 - DNA, 70 bp, synthetic construct
  source 1..70: mol_type = other DNA
gacgaagacggggagggtatgcgacctactcttactgcgtctatttagtggagccccatcctcgggagat

SEQ ID NO: 295 - DNA, 70 bp, synthetic construct
  source 1..70: mol_type = other DNA
gtggtgccctcggacggtatgcgacctactcttactgcgtctatttagtggagccctggtcatggaaagc

SEQ ID NO: 296 - DNA, 70 bp, synthetic construct
  source 1..70: mol_type = other DNA
gttaccgtgctcaagggtatgcgacctactcttactgcgtctatttagtggagccgacggcacaccctac

SEQ ID NO: 297 - DNA, 70 bp, synthetic construct
  source 1..70: mol_type = other DNA
cctgactggtgctgcggtatgcgacctactcttactgcgtctatttagtggagccgctgtgcaacggtct

SEQ ID NO: 298 - DNA, 70 bp, synthetic construct
  source 1..70: mol_type = other DNA
atgctggccgctaccgtctgagaatgtgttagcactgcgtctatttagtggagccgcttcctacctgagg

SEQ ID NO: 299 - DNA, 70 bp, synthetic construct
  source 1..70: mol_type = other DNA
accgtcaagttccgcgtctgagaatgtgttagcactgcgtctatttagtggagccgtacctgcggggaac

SEQ ID NO: 300 - DNA, 70 bp, synthetic construct
  source 1..70: mol_type = other DNA
tgtcagccgaggacggtctgagaatgtgttagcactgcgtctatttagtggagcctgtacctgcggaacg

SEQ ID NO: 301 - DNA, 70 bp, synthetic construct
  source 1..70: mol_type = other DNA
ggccgcctgcctgtggtctgagaatgtgttagcactgcgtctatttagtggagccaagaaaaccagcaac

SEQ ID NO: 302 - DNA, 70 bp, synthetic construct
  source 1..70: mol_type = other DNA
gtccccatcccgggtgtctgagaatgtgttagcactgcgtctatttagtggagccggaccttggcgctta

SEQ ID NO: 303 - DNA, 70 bp, synthetic construct
  source 1..70: mol_type = other DNA
cgagagactccacggtacgtttgacgaggagtacatgcgtctatttagtggagcccactcagcacaaaca

SEQ ID NO: 304 - DNA, 70 bp, synthetic construct
  source 1..70: mol_type = other DNA
tgctgatggcataattacgtttgacgaggagtacatgcgtctatttagtggagccctcctccgggcctgt

SEQ ID NO: 305 - DNA, 70 bp, synthetic construct
  source 1..70: mol_type = other DNA
aggtgtgaggactgttacgtttgacgaggagtacatgcgtctatttagtggagccggcctgcacgcctac

SEQ ID NO: 306 - DNA, 70 bp, synthetic construct
  source 1..70: mol_type = other DNA
tccctctttttgccttacgtttgacgaggagtacatgcgtctatttagtggagccgccatgggtgtcatg

SEQ ID NO: 307 - DNA, 70 bp, synthetic construct
  source 1..70: mol_type = other DNA
tgctgtttgcggtgatacgtttgacgaggagtacatgcgtctatttagtggagccaggtgggatagtctt

SEQ ID NO: 308 - DNA, 70 bp, synthetic construct
  source 1..70: mol_type = other DNA
gctccagcacccactacctacctaaagtgacatcgtgcgtctatttagtggagccgatgtcccacagccg

SEQ ID NO: 309 - DNA, 70 bp, synthetic construct
  source 1..70: mol_type = other DNA
tgtgcgaggcctgtgacctacctaaagtgacatcgtgcgtctatttagtggagccacaagcacttcttgc

SEQ ID NO: 310 - DNA, 70 bp, synthetic construct
  source 1..70: mol_type = other DNA
aggacgatgagggctacctacctaaagtgacatcgtgcgtctatttagtggagccaccactgcacgaatg

SEQ ID NO: 311 - DNA, 70 bp, synthetic construct
  source 1..70: mol_type = other DNA
cagtgctctgcctggacctacctaaagtgacatcgtgcgtctatttagtggagcctttgtgtcgaagccc

SEQ ID NO: 312 - DNA, 70 bp, synthetic construct
  source 1..70: mol_type = other DNA
cagtgcacttctcccacctacctaaagtgacatcgtgcgtctatttagtggagccttaggagccactgct

SEQ ID NO: 313 - DNA, 70 bp, synthetic construct
  source 1..70: mol_type = other DNA
cagcttcccgggctcagatacggaaatggacgttctgcgtctatttagtggagcccagcccgtgtgcagt

SEQ ID NO: 314 - DNA, 70 bp, synthetic construct
  source 1..70: mol_type = other DNA
gccaaggagtttgtgagatacggaaatggacgttctgcgtctatttagtggagcctacggctaccgcttc

SEQ ID NO: 315 - DNA, 70 bp, synthetic construct
  source 1..70: mol_type = other DNA
gctgcgggcgtggctagatacggaaatggacgttctgcgtctatttagtggagccgctgtgagctcatgt

SEQ ID NO: 316 - DNA, 70 bp, synthetic construct
  source 1..70: mol_type = other DNA
ggtgctctcttactcagatacggaaatggacgttctgcgtctatttagtggagccttccgtaagaggcct

SEQ ID NO: 317 - DNA, 70 bp, synthetic construct
  source 1..70: mol_type = other DNA
tatactgtcttccccagatacggaaatggacgttctgcgtctatttagtggagccaggctgcgtggacgt

SEQ ID NO: 318 - DNA, 70 bp, synthetic construct
  source 1..70: mol_type = other DNA
tgaggtgcagccttgagatacttaaccatagcgcctgcgtctatttagtggagccacgcccgggtagatt

SEQ ID NO: 319 - DNA, 70 bp, synthetic construct
  source 1..70: mol_type = other DNA
cagacccgtgcccttagatacttaaccatagcgcctgcgtctatttagtggagcccatgatagatacgct

SEQ ID NO: 320 - DNA, 70 bp, synthetic construct
  source 1..70: mol_type = other DNA
agtggtgctctacgaagatacttaaccatagcgcctgcgtctatttagtggagccctgtggcaccgacac

SEQ ID NO: 321 - DNA, 70 bp, synthetic construct
  source 1..70: mol_type = other DNA
tctggcccttcagcaagatacttaaccatagcgcctgcgtctatttagtggagccccttgctggaatgtg

SEQ ID NO: 322 - DNA, 70 bp, synthetic construct
  source 1..70: mol_type = other DNA
ctcaccgtgtcaggaagatacttaaccatagcgcctgcgtctatttagtggagccgcatatctaagcttc

SEQ ID NO: 323 - DNA, 70 bp, synthetic construct
  source 1..70: mol_type = other DNA
taccgctttgccaagatagagtcaacgagcgtacctgcgtctatttagtggagccaacatcgactatggc

SEQ ID NO: 324 - DNA, 70 bp, synthetic construct
  source 1..70: mol_type = other DNA
ccacacaagacctggatagagtcaacgagcgtacctgcgtctatttagtggagccgcttcaactcgccca

SEQ ID NO: 325 - DNA, 70 bp, synthetic construct
  source 1..70: mol_type = other DNA
gggcatggatggctgatagagtcaacgagcgtacctgcgtctatttagtggagcccaacaagacgtcgga

SEQ ID NO: 326 - DNA, 70 bp, synthetic construct
  source 1..70: mol_type = other DNA
gggtgccacccagcaatagagtcaacgagcgtacctgcgtctatttagtggagccttgtgtgcaagtagt

SEQ ID NO: 327 - DNA, 70 bp, synthetic construct
  source 1..70: mol_type = other DNA
gagactggcactgtgatagagtcaacgagcgtacctgcgtctatttagtggagccagctactgacctcct

SEQ ID NO: 328 - DNA, 70 bp, synthetic construct
  source 1..70: mol_type = other DNA
gagagtgagaagtaccacactaactcgaatgacgttgcgtctatttagtggagcccgcatcaaaggggct

SEQ ID NO: 329 - DNA, 70 bp, synthetic construct
  source 1..70: mol_type = other DNA
accaaggccagctgccacactaactcgaatgacgttgcgtctatttagtggagcctcatcaagcgcctct

SEQ ID NO: 330 - DNA, 70 bp, synthetic construct
  source 1..70: mol_type = other DNA
cccccagctgggaagcacactaactcgaatgacgttgcgtctatttagtggagcccgcgaagcatccgag

SEQ ID NO: 331 - DNA, 70 bp, synthetic construct
  source 1..70: mol_type = other DNA
ctccctcagtctgcccacactaactcgaatgacgttgcgtctatttagtggagccatctgcttctcggat

SEQ ID NO: 332 - DNA, 70 bp, synthetic construct
  source 1..70: mol_type = other DNA
caggcctgcaccccacacactaactcgaatgacgttgcgtctatttagtggagcctcacattccacgacc

SEQ ID NO: 333 - DNA, 70 bp, synthetic construct
  source 1..70: mol_type = other DNA
ttcagctctccaccccacactgtaagacgatagcctgcgtctatttagtggagccgccctgaaaatgctc

SEQ ID NO: 334 - DNA, 70 bp, synthetic construct
  source 1..70: mol_type = other DNA
agccaccatgaaaagcacactgtaagacgatagcctgcgtctatttagtggagcccagactggcagtgag

SEQ ID NO: 335 - DNA, 70 bp, synthetic construct
  source 1..70: mol_type = other DNA
agctgagagctgggacacactgtaagacgatagcctgcgtctatttagtggagccaaatggataagcggc

SEQ ID NO: 336 - DNA, 70 bp, synthetic construct
  source 1..70: mol_type = other DNA
tgcctacagaacagccacactgtaagacgatagcctgcgtctatttagtggagccacagagggtcgtgag

SEQ ID NO: 337 - DNA, 70 bp, synthetic construct
  source 1..70: mol_type = other DNA
tgaccagtgtaggcacacactgtaagacgatagcctgcgtctatttagtggagccctgtacggtcaagtg

SEQ ID NO: 338 - DNA, 70 bp, synthetic construct
  source 1..70: mol_type = other DNA
caacatgctacctgcccgtcagaagccatactagatgcgtctatttagtggagccgcggcagttgccgca

SEQ ID NO: 339 - DNA, 70 bp, synthetic construct
  source 1..70: mol_type = other DNA
tatcggatgtgagagccgtcagaagccatactagatgcgtctatttagtggagccccatcacagcacgcc

SEQ ID NO: 340 - DNA, 70 bp, synthetic construct
  source 1..70: mol_type = other DNA
aacacgggtccgagaccgtcagaagccatactagatgcgtctatttagtggagcccaaaagagggactga

SEQ ID NO: 341 - DNA, 70 bp, synthetic construct
  source 1..70: mol_type = other DNA
tgatttcactgctccccgtcagaagccatactagatgcgtctatttagtggagccatgggcctccctagc

SEQ ID NO: 342 - DNA, 70 bp, synthetic construct
  source 1..70: mol_type = other DNA
agtgactgcgtccttccgtcagaagccatactagatgcgtctatttagtggagccccctgagtcctcatt

SEQ ID NO: 343 - DNA, 70 bp, synthetic construct
  source 1..70: mol_type = other DNA
gaaccgaaggaagaaccgtcgatacagactcagattgcgtctatttagtggagccggacatgctcgccct

SEQ ID NO: 344 - DNA, 70 bp, synthetic construct
  source 1..70: mol_type = other DNA
agaggcagttttactccgtcgatacagactcagattgcgtctatttagtggagcctctgcatccggtgca

SEQ ID NO: 345 - DNA, 70 bp, synthetic construct
  source 1..70: mol_type = other DNA
gaatcctgaaggcccccgtcgatacagactcagattgcgtctatttagtggagccggacacagcacaggt

SEQ ID NO: 346 - DNA, 70 bp, synthetic construct
  source 1..70: mol_type = other DNA
ctcgtccacacccacccgtcgatacagactcagattgcgtctatttagtggagccgggaagatccgtgca

SEQ ID NO: 347 - DNA, 70 bp, synthetic construct
  source 1..70: mol_type = other DNA
cgtgactgggacaggccgtcgatacagactcagattgcgtctatttagtggagccgaacacgtagggctc

SEQ ID NO: 348 - DNA, 70 bp, synthetic construct
  source 1..70: mol_type = other DNA
agcccgacttctcctcggactaagggcgactaatatgcgtctatttagtggagcctcttcctctttggcc

SEQ ID NO: 349 - DNA, 70 bp, synthetic construct
  source 1..70: mol_type = other DNA
cagtgccacccggaccggactaagggcgactaatatgcgtctatttagtggagccccgctggtcatgaag

SEQ ID NO: 350 - DNA, 70 bp, synthetic construct
  source 1..70: mol_type = other DNA
gctgtggctcaaagacggactaagggcgactaatatgcgtctatttagtggagcccctgaagaaatcggt

SEQ ID NO: 351 - DNA, 70 bp, synthetic construct
  source 1..70: mol_type = other DNA
ctccgggatctcagccggactaagggcgactaatatgcgtctatttagtggagccggctgaccatttctg

SEQ ID NO: 352 - DNA, 70 bp, synthetic construct
  source 1..70: mol_type = other DNA
cacgtttgcatcccccggactaagggcgactaatatgcgtctatttagtggagcctccccctgccttttg

SEQ ID NO: 353 - DNA, 70 bp, synthetic construct
  source 1..70: mol_type = other DNA
cgcgatggtagcggcctagactgaatcatcaccgatgcgtctatttagtggagcctacccgggtctttgt

SEQ ID NO: 354 - DNA, 70 bp, synthetic construct
  source 1..70: mol_type = other DNA
cctgcccccaccgctctagactgaatcatcaccgatgcgtctatttagtggagcccaacgctatcaagaa

SEQ ID NO: 355 - DNA, 70 bp, synthetic construct
  source 1..70: mol_type = other DNA
agccgtacccgtgcgctagactgaatcatcaccgatgcgtctatttagtggagccccattgacaactacc

SEQ ID NO: 356 - DNA, 70 bp, synthetic construct
  source 1..70: mol_type = other DNA
acccgcggaggggacctagactgaatcatcaccgatgcgtctatttagtggagcctactgcgctagtccc

SEQ ID NO: 357 - DNA, 70 bp, synthetic construct
  source 1..70: mol_type = other DNA
ttactgcaaaaatggctagactgaatcatcaccgatgcgtctatttagtggagccgtgctgccccgggaa

SEQ ID NO: 358 - DNA, 70 bp, synthetic construct
  source 1..70: mol_type = other DNA
ccatcccatgcggtcgaactatgctgacagtaccgtgcgtctatttagtggagccgtgcccgtggtgcag

SEQ ID NO: 359 - DNA, 70 bp, synthetic construct
  source 1..70: mol_type = other DNA
cgtgaagcctcagcagaactatgctgacagtaccgtgcgtctatttagtggagcccagtgacctaatgca

SEQ ID NO: 360 - DNA, 70 bp, synthetic construct
  source 1..70: mol_type = other DNA
ttcggcaaggacggtgaactatgctgacagtaccgtgcgtctatttagtggagccccaaacctgtaacct

SEQ ID NO: 361 - DNA, 70 bp, synthetic construct
  source 1..70: mol_type = other DNA
ggtgttcagtagagcgaactatgctgacagtaccgtgcgtctatttagtggagcctctccgcccttgtaa

SEQ ID NO: 362 - DNA, 70 bp, synthetic construct
  source 1..70: mol_type = other DNA
ctggtttgcagtgaagaactatgctgacagtaccgtgcgtctatttagtggagccgatgcgttcagcaga

SEQ ID NO: 363 - DNA, 70 bp, synthetic construct
  source 1..70: mol_type = other DNA
ttttgactaaaagacgtctggagagatcagtatcctgcgtctatttagtggagcccgacggcgacgtctc

SEQ ID NO: 364 - DNA, 70 bp, synthetic construct
  source 1..70: mol_type = other DNA
gagattaacttcaaggtctggagagatcagtatcctgcgtctatttagtggagccaccgtgcgcaccaca

SEQ ID NO: 365 - DNA, 70 bp, synthetic construct
  source 1..70: mol_type = other DNA
tacgtccgagagtgagtctggagagatcagtatcctgcgtctatttagtggagccgtgtgcaccagggtc

SEQ ID NO: 366 - DNA, 70 bp, synthetic construct
  source 1..70: mol_type = other DNA
ccccttaccccagtcgtctggagagatcagtatcctgcgtctatttagtggagccttctaggatagcgct

SEQ ID NO: 367 - DNA, 70 bp, synthetic construct
  source 1..70: mol_type = other DNA
ttcactccccgcctcgtctggagagatcagtatcctgcgtctatttagtggagccatcacccattccggg

SEQ ID NO: 368 - DNA, 70 bp, synthetic construct
  source 1..70: mol_type = other DNA
gcccaggcctgtgtctcgcgtctacctgactttagtgcgtctatttagtggagccgtggggcacattctg

SEQ ID NO: 369 - DNA, 70 bp, synthetic construct
  source 1..70: mol_type = other DNA
tcgttccctagggagtcgcgtctacctgactttagtgcgtctatttagtggagcccagagaggtggcccc

SEQ ID NO: 370 - DNA, 70 bp, synthetic construct
  source 1..70: mol_type = other DNA
acaggcctgagggagtcgcgtctacctgactttagtgcgtctatttagtggagcccgctgcttctactgg

SEQ ID NO: 371 - DNA, 70 bp, synthetic construct
  source 1..70: mol_type = other DNA
ggggccccccaggcctcgcgtctacctgactttagtgcgtctatttagtggagcctcctgagtctaacct

SEQ ID NO: 372 - DNA, 70 bp, synthetic construct
  source 1..70: mol_type = other DNA
gatgggttctgcccttcgcgtctacctgactttagtgcgtctatttagtggagccacaggagtccccgca

SEQ ID NO: 373 - DNA, 70 bp, synthetic construct; identical to SEQ ID NO: 178
  source 1..70: mol_type = other DNA

SEQ ID NO: 374 - DNA, 70 bp, synthetic construct; identical to SEQ ID NO: 179
  source 1..70: mol_type = other DNA

SEQ ID NO: 375 - DNA, 70 bp, synthetic construct; identical to SEQ ID NO: 180
  source 1..70: mol_type = other DNA

SEQ ID NO: 376 - DNA, 70 bp, synthetic construct; identical to SEQ ID NO: 181
  source 1..70: mol_type = other DNA

SEQ ID NO: 377 - DNA, 70 bp, synthetic construct; identical to SEQ ID NO: 182
  source 1..70: mol_type = other DNA

SEQ ID NO: 378 - DNA, 70 bp, synthetic construct
  source 1..70: mol_type = other DNA
cagtaacagcccgagacctacgaacactaagtcggtgcgtctatttagtggagccaaggtcctggcaact

SEQ ID NO: 379 - DNA, 70 bp, synthetic construct
  source 1..70: mol_type = other DNA
cggaacgaagatgggacctacgaacactaagtcggtgcgtctatttagtggagcctcttccaacaccagg

SEQ ID NO: 380 - DNA, 70 bp, synthetic construct
  source 1..70: mol_type = other DNA
accagccaccagcgcacctacgaacactaagtcggtgcgtctatttagtggagcctggctatgtctttgc

SEQ ID NO: 381 - DNA, 70 bp, synthetic construct
  source 1..70: mol_type = other DNA
tcagacgggagccacacctacgaacactaagtcggtgcgtctatttagtggagcctcattcggccactgt

SEQ ID NO: 382 - DNA, 70 bp, synthetic construct
  source 1..70: mol_type = other DNA
gaactaccccctgccacctacgaacactaagtcggtgcgtctatttagtggagccaccacagccattcag

SEQ ID NO: 383 - DNA, 70 bp, synthetic construct
  source 1..70: mol_type = other DNA
gccagtcgacttcactagactctcgatcagccgtatgcgtctatttagtggagccgtcactccccgaaat

SEQ ID NO: 384 - DNA, 70 bp, synthetic construct
  source 1..70: mol_type = other DNA
ggagtacctgcgcgctagactctcgatcagccgtatgcgtctatttagtggagcccaacgagaatttcga

SEQ ID NO: 385 - DNA, 70 bp, synthetic construct
  source 1..70: mol_type = other DNA
cgccaacttgctgaatagactctcgatcagccgtatgcgtctatttagtggagccggccttgcgcaaaat

SEQ ID NO: 386 - DNA, 70 bp, synthetic construct
  source 1..70: mol_type = other DNA
tttaggaactacatctagactctcgatcagccgtatgcgtctatttagtggagcccgcacgctgagcact

SEQ ID NO: 387 - DNA, 70 bp, synthetic construct
  source 1..70: mol_type = other DNA
gaccgcaagtgcatgtagactctcgatcagccgtatgcgtctatttagtggagccctgacaggcatagat

SEQ ID NO: 388 - DNA, 70 bp, synthetic construct
  source 1..70: mol_type = other DNA
tctggaccaccaaggttgacatagtggtcgagttctgcgtctatttagtggagccacaccctgggccagt

SEQ ID NO: 389 - DNA, 70 bp, synthetic construct
  source 1..70: mol_type = other DNA
ccttcgccttcatggttgacatagtggtcgagttctgcgtctatttagtggagcccatccaaagcgtcag

SEQ ID NO: 390 - DNA, 70 bp, synthetic construct
  source 1..70: mol_type = other DNA
gctcaaccaggccctttgacatagtggtcgagttctgcgtctatttagtggagccagtggaagctgtgca

SEQ ID NO: 391 - DNA, 70 bp, synthetic construct
  source 1..70: mol_type = other DNA
cctgtccattggcatttgacatagtggtcgagttctgcgtctatttagtggagccgcacacacccgagca

SEQ ID NO: 392 - DNA, 70 bp, synthetic construct
  source 1..70: mol_type = other DNA
ccacagggaggcaggttgacatagtggtcgagttctgcgtctatttagtggagccgggtataactgaccc

SEQ ID NO: 393 - DNA, 70 bp, synthetic construct
  source 1..70: mol_type = other DNA
ccactggccagcgtcaaccgtctaaaccatacgtgtgcgtctatttagtggagccccggctggtgtcggt

SEQ ID NO: 394 - DNA, 70 bp, synthetic construct
  source 1..70: mol_type = other DNA
ttccgaatcgccatgaaccgtctaaaccatacgtgtgcgtctatttagtggagccgtgaagcgcctcatg

SEQ ID NO: 395 - DNA, 70 bp, synthetic construct
  source 1..70: mol_type = other DNA
ctgcgggctgcgggcaaccgtctaaaccatacgtgtgcgtctatttagtggagcccattcgcgccaagat

SEQ ID NO: 396 - DNA, 70 bp, synthetic construct
  source 1..70: mol_type = other DNA
tctccagaaagtgcgaaccgtctaaaccatacgtgtgcgtctatttagtggagccgctctacccacatgt

SEQ ID NO: 397 - DNA, 70 bp, synthetic construct
  source 1..70: mol_type = other DNA
acaatctccctgcaaaaccgtctaaaccatacgtgtgcgtctatttagtggagccccgtgtatcctgtgg

SEQ ID NO: 398 - DNA, 70 bp, synthetic construct
  source 1..70: mol_type = other DNA
aatgaccggaacaagacgaggttaaagtcaagtcctgcgtctatttagtggagccaaggaagctgtgcga

SEQ ID NO: 399 - DNA, 70 bp, synthetic construct
  source 1..70: mol_type = other DNA
ggtacaccccagagcacgaggttaaagtcaagtcctgcgtctatttagtggagcctgcgtatctgcacaa

SEQ ID NO: 400 - DNA, 70 bp, synthetic construct
  source 1..70: mol_type = other DNA
tcccttaatccgagaacgaggttaaagtcaagtcctgcgtctatttagtggagcctccaggcccgatgcc

SEQ ID NO: 401 - DNA, 70 bp, synthetic construct; identical to SEQ ID NO: 400
  source 1..70: mol_type = other DNA

SEQ ID NO: 402 - DNA, 70 bp, synthetic construct
  source 1..70: mol_type = other DNA
gtgggggagcccaggacgaggttaaagtcaagtcctgcgtctatttagtggagccctccaggggtcttgg

SEQ ID NO: 403 - DNA, 70 bp, synthetic construct
  source 1..70: mol_type = other DNA
tggagtcgaccagcaacgagtagaaatgacgctcctgcgtctatttagtggagccggaacgagaatgagg

SEQ ID NO: 404 - DNA, 70 bp, synthetic construct
  source 1..70: mol_type = other DNA
tactggtgcccagccacgagtagaaatgacgctcctgcgtctatttagtggagccttcatctgctctgaa

SEQ ID NO: 405 - DNA, 70 bp, synthetic construct
  source 1..70: mol_type = other DNA
gcagagtgttgagccacgagtagaaatgacgctcctgcgtctatttagtggagccatccaccgtcctgag

SEQ ID NO: 406 - DNA, 70 bp, synthetic construct
  source 1..70: mol_type = other DNA
ggcctctgggaaggaacgagtagaaatgacgctcctgcgtctatttagtggagccaccctggaagcacac

SEQ ID NO: 407 - DNA, 70 bp, synthetic construct
  source 1..70: mol_type = other DNA
tttcaagggttttctacgagtagaaatgacgctcctgcgtctatttagtggagccgggtgggcctgaggc

SEQ ID NO: 408 - DNA, 70 bp, synthetic construct
  source 1..70: mol_type = other DNA
agggtcctgggggaaagcggactaaatataggtcgtgcgtctatttagtggagccgtgaccagggcgttg

SEQ ID NO: 409 - DNA, 70 bp, synthetic construct
  source 1..70: mol_type = other DNA
attgctgcgggcaggagcggactaaatataggtcgtgcgtctatttagtggagccggatgatcaggtcat

SEQ ID NO: 410 - DNA, 70 bp, synthetic construct
  source 1..70: mol_type = other DNA
taagtgtctagagcaagcggactaaatataggtcgtgcgtctatttagtggagccccggtccattggcct

SEQ ID NO: 411 - DNA, 70 bp, synthetic construct
  source 1..70: mol_type = other DNA
gtggtgcttctcacaagcggactaaatataggtcgtgcgtctatttagtggagccagctcagacccagac

SEQ ID NO: 412 - DNA, 70 bp, synthetic construct
  source 1..70: mol_type = other DNA
gccttcccagcttccagcggactaaatataggtcgtgcgtctatttagtggagccgctctccaagcacta

SEQ ID NO: 413 - DNA, 70 bp, synthetic construct
  source 1..70: mol_type = other DNA
catggaggcatgggaatatctttaacgtcgagcgctgcgtctatttagtggagccgcgttatgatcagct

SEQ ID NO: 414 - DNA, 70 bp, synthetic construct
  source 1..70: mol_type = other DNA
aggagtgagcatgagatatctttaacgtcgagcgctgcgtctatttagtggagcctcagccaccattgct

SEQ ID NO: 415 - DNA, 70 bp, synthetic construct
  source 1..70: mol_type = other DNA
atgacaaacgaagctatatctttaacgtcgagcgctgcgtctatttagtggagcccggatcaccaggtcc

SEQ ID NO: 416 - DNA, 70 bp, synthetic construct
  source 1..70: mol_type = other DNA
tctcgatggacagaaatatctttaacgtcgagcgctgcgtctatttagtggagccgagcctgtggagacc

SEQ ID NO: 417 - DNA, 70 bp, synthetic construct
  source 1..70: mol_type = other DNA
ctgctcaggaggatgatatctttaacgtcgagcgctgcgtctatttagtggagcctcgcttccactgttt

SEQ ID NO: 418 - DNA, 70 bp, synthetic construct
  source 1..70: mol_type = other DNA
ctgagtctttgaggacacggtatagacaccacgtatgcgtctatttagtggagccctccgcgaggtctcc

SEQ ID NO: 419 - DNA, 70 bp, synthetic construct
  source 1..70: mol_type = other DNA
ctggaaggcctggggcacggtatagacaccacgtatgcgtctatttagtggagccgctgcacatcggatt

SEQ ID NO: 420 - DNA, 70 bp, synthetic construct
  source 1..70: mol_type = other DNA
gatcttgcagcagcacacggtatagacaccacgtatgcgtctatttagtggagccgcagaacctcgatga

SEQ ID NO: 421 - DNA, 70 bp, synthetic construct
  source 1..70: mol_type = other DNA
cacgacgtacgctcccacggtatagacaccacgtatgcgtctatttagtggagccactggctccaaaaag

SEQ ID NO: 422 - DNA, 70 bp, synthetic construct
  source 1..70: mol_type = other DNA
acaggccttatgacccacggtatagacaccacgtatgcgtctatttagtggagcccccgccatcaccgga

SEQ ID NO: 423 - DNA, 70 bp, synthetic construct; identical to SEQ ID NO: 268
  source 1..70: mol_type = other DNA

SEQ ID NO: 424 - DNA, 70 bp, synthetic construct; identical to SEQ ID NO: 269
  source 1..70: mol_type = other DNA

SEQ ID NO: 425 - DNA, 70 bp, synthetic construct; identical to SEQ ID NO: 270
  source 1..70: mol_type = other DNA

SEQ ID NO: 426 - DNA, 70 bp, synthetic construct; identical to SEQ ID NO: 271
  source 1..70: mol_type = other DNA

SEQ ID NO: 427 - DNA, 70 bp, synthetic construct; identical to SEQ ID NO: 272
  source 1..70: mol_type = other DNA

SEQ ID NO: 428 - DNA, 38 bp, synthetic construct
  source 1..38: mol_type = other DNA
acggataactactagactttgtacccggtcacatagaa

SEQ ID NO: 429 - DNA, 38 bp, synthetic construct
  source 1..38: mol_type = other DNA
cttaactcgcgttcaagcctgtacccggtcacatagaa

SEQ ID NO: 430 - DNA, 38 bp, synthetic construct
  source 1..38: mol_type = other DNA
cttagacacatcatacgtctgtacccggtcacatagaa

SEQ ID NO: 431 - DNA, 38 bp, synthetic construct
  source 1..38: mol_type = other DNA
ggtacggaagtgtatatagtgtacccggtcacatagaa

SEQ ID NO: 432 - DNA, 38 bp, synthetic construct
  source 1..38: mol_type = other DNA
acagtttacactattcgaatgtacccggtcacatagaa

SEQ ID NO: 433 - DNA, 38 bp, synthetic construct
  source 1..38: mol_type = other DNA
acatagaccataccacgcctgtacccggtcacatagaa

SEQ ID NO: 434 - DNA, 38 bp, synthetic construct
  source 1..38: mol_type = other DNA
cctagataccgtagatgtgtgtacccggtcacatagaa

SEQ ID NO: 435 - DNA, 38 bp, synthetic construct
  source 1..38: mol_type = other DNA
gttgctacgatgactcaaatgtacccggtcacatagaa

SEQ ID NO: 436 - DNA, 38 bp, synthetic construct
  source 1..38: mol_type = other DNA
agacgtagtgagcatgaaatgtacccggtcacatagaa

SEQ ID NO: 437 - DNA, 38 bp, synthetic construct
  source 1..38: mol_type = other DNA
ggacattaccacctagacatgtacccggtcacatagaa

SEQ ID NO: 438 - DNA, 38 bp, synthetic construct
  source 1..38: mol_type = other DNA
tagacagactgcgacattgtgtacccggtcacatagaa

SEQ ID NO: 439 - DNA, 38 bp, synthetic construct
  source 1..38: mol_type = other DNA
tagacatatctcacagccttgtacccggtcacatagaa

SEQ ID NO: 440 - DNA, 38 bp, synthetic construct
  source 1..38: mol_type = other DNA
tctgcgaaggtagtgtatatgtacccggtcacatagaa

SEQ ID NO: 441 - DNA, 38 bp, synthetic construct
  source 1..38: mol_type = other DNA
tttccaatacggtgtaccttgtacccggtcacatagaa

SEQ ID NO: 442 - DNA, 38 bp, synthetic construct
  source 1..38: mol_type = other DNA
tttcgacgctgacactatttgtacccggtcacatagaa

SEQ ID NO: 443 - DNA, 38 bp, synthetic construct
  source 1..38: mol_type = other DNA
tttgtgaatagtctagccgtgtacccggtcacatagaa

SEQ ID NO: 444 - DNA, 38 bp, synthetic construct
  source 1..38: mol_type = other DNA
cactattacgcagagcaagtgtacccggtcacatagaa

SEQ ID NO: 445 - DNA, 38 bp, synthetic construct
  source 1..38: mol_type = other DNA
gtatagtacattcccgctgtgtacccggtcacatagaa

SEQ ID NO: 446 - DNA, 38 bp, synthetic construct
  source 1..38: mol_type = other DNA
gtatgcgacctactctttgtgtacccggtcacatagaa

SEQ ID NO: 447 - DNA, 38 bp, synthetic construct
  source 1..38: mol_type = other DNA
tctgagaatgtgttagctgtgtacccggtcacatagaa

SEQ ID NO: 448 - DNA, 38 bp, synthetic construct
  source 1..38: mol_type = other DNA
tctgcatagagaacaaccatgtacccggtcacatagaa

SEQ ID NO: 449 - DNA, 38 bp, synthetic construct
  source 1..38: mol_type = other DNA
acgtttgacgaggagtagttgtacccggtcacatagaa

SEQ ID NO: 450 - DNA, 38 bp, synthetic construct
  source 1..38: mol_type = other DNA
cctaataacactcacggaatgtacccggtcacatagaa

SEQ ID NO: 451 - DNA, 38 bp, synthetic construct
  source 1..38: mol_type = other DNA
cctacctaaagtgacataatgtacccggtcacatagaa

SEQ ID NO: 452 - DNA, 38 bp, synthetic construct
  source 1..38: mol_type = other DNA
gatacggaaatggacgtcgtgtacccggtcacatagaa

SEQ ID NO: 453 - DNA, 38 bp, synthetic construct
  source 1..38: mol_type = other DNA
gatacttaaccatagcgtttgtacccggtcacatagaa

SEQ ID NO: 454 - DNA, 38 bp, synthetic construct
  source 1..38: mol_type = other DNA
tagacttaacgcacgattttgtacccggtcacatagaa

SEQ ID NO: 455 - DNA, 38 bp, synthetic construct
  source 1..38: mol_type = other DNA
tagagcgaacgatagttaatgtacccggtcacatagaa

SEQ ID NO: 456 - DNA, 38 bp, synthetic construct
  source 1..38: mol_type = other DNA
tagagtcaacgagcgtatttgtacccggtcacatagaa

SEQ ID NO: 457 - DNA, 38 bp, synthetic construct
  source 1..38: mol_type = other DNA
acactaactcgaatgaccatgtacccggtcacatagaa

SEQ ID NO: 458 - DNA, 38 bp, synthetic construct
  source 1..38: mol_type = other DNA
acactgtaagacgatagggtgtacccggtcacatagaa

SEQ ID NO: 459 - DNA, 38 bp, synthetic construct
  source 1..38: mol_type = other DNA
cgtcagaagccatactacttgtacccggtcacatagaa

SEQ ID NO: 460 - DNA, 38 bp, synthetic construct
  source 1..38: mol_type = other DNA
cgtcgatacagactcagtatgtacccggtcacatagaa

SEQ ID NO: 461 - DNA, 38 bp, synthetic construct
  source 1..38: mol_type = other DNA
ggactaagggcgactaaattgtacccggtcacatagaa

SEQ ID NO: 462 - DNA, 38 bp, synthetic construct
  source 1..38: mol_type = other DNA
tagactgaatcatcacccttgtacccggtcacatagaa

SEQ ID NO: 463 - DNA, 38 bp, synthetic construct
  source 1..38: mol_type = other DNA
tctgtgaataccgagtagttgtacccggtcacatagaa

SEQ ID NO: 464 - DNA, 38 bp, synthetic construct
  source 1..38: mol_type = other DNA
aactatgctgacagtacgctgtacccggtcacatagaa

SEQ ID NO: 465 - DNA, 38 bp, synthetic construct
  source 1..38: mol_type = other DNA
gtcagagacgtagcatctttgtacccggtcacatagaa

SEQ ID NO: 466 - DNA, 38 bp, synthetic construct
  source 1..38: mol_type = other DNA
tctggagagatcagtatggtgtacccggtcacatagaa

SEQ ID NO: 467 - DNA, 38 bp, synthetic construct
  source 1..38: mol_type = other DNA
actatgcctacctgtcgtatgtacccggtcacatagaa

SEQ ID NO: 468 - DNA, 38 bp, synthetic construct
  source 1..38: mol_type = other DNA
cgcgtctacctgacttttctgtacccggtcacatagaa

SEQ ID NO: 469 - DNA, 38 bp, synthetic construct
  source 1..38: mol_type = other DNA
cgcgttcaccgtaatctattgtacccggtcacatagaa

SEQ ID NO: 470 - DNA, 38 bp, synthetic construct
  source 1..38: mol_type = other DNA
cctacgaacactaagtccctgtacccggtcacatagaa

SEQ ID NO: 471 - DNA, 38 bp, synthetic construct
  source 1..38: mol_type = other DNA
agactctcgatcagccgattgtacccggtcacatagaa

SEQ ID NO: 472 - DNA, 38 bp, synthetic construct
  source 1..38: mol_type = other DNA
tgacatagtggtcgagtagtgtacccggtcacatagaa

SEQ ID NO: 473 - DNA, 38 bp, synthetic construct
  source 1..38: mol_type = other DNA
accgtctaaaccatacgactgtacccggtcacatagaa

SEQ ID NO: 474 - DNA, 38 bp, synthetic construct
  source 1..38: mol_type = other DNA
acgactaacaactgtcccatgtacccggtcacatagaa

SEQ ID NO: 475 - DNA, 38 bp, synthetic construct
  source 1..38: mol_type = other DNA
cgaggttaaagtcaagtggtgtacccggtcacatagaa

SEQ ID NO: 476 - DNA, 38 bp, synthetic construct
  source 1..38: mol_type = other DNA
cgagtagaaatgacgctggtgtacccggtcacatagaa

SEQ ID NO: 477 - DNA, 38 bp, synthetic construct
  source 1..38: mol_type = other DNA
gcggactaaatataggtgctgtacccggtcacatagaa

SEQ ID NO: 478 - DNA, 38 bp, synthetic construct
  source 1..38: mol_type = other DNA
tatctttaacgtcgagccgtgtacccggtcacatagaa

SEQ ID NO: 479 - DNA, 38 bp, synthetic construct
  source 1..38: mol_type = other DNA
acggtatagacaccacgattgtacccggtcacatagaa

SEQ ID NO: 480 - DNA, 38 bp, synthetic construct
  source 1..38: mol_type = other DNA
acggtcaactaggtgagtttgtacccggtcacatagaa

SEQ ID NO: 481 - DNA, 38 bp, synthetic construct
  source 1..38: mol_type = other DNA
cttatcgaagtagacgagttgtacccggtcacatagaa

SEQ ID NO: 482 - DNA, 38 bp, synthetic construct
  source 1..38: mol_type = other DNA
ggtactaaggctgaccttttgtacccggtcacatagaa

SEQ ID NO: 483 - DNA, 38 bp, synthetic construct
  source 1..38: mol_type = other DNA
ggtcaactctacctaagcttgtacccggtcacatagaa

SEQ ID NO: 484 - DNA, 38 bp, synthetic construct
  source 1..38: mol_type = other DNA
acatcagagtaccagctgttgtacccggtcacatagaa

SEQ ID NO: 485 - DNA, 38 bp, synthetic construct
  source 1..38: mol_type = other DNA
tcgtcggatcgaggttaagtgtacccggtcacatagaa

SEQ ID NO: 486 - DNA, 39 bp, synthetic construct
  source 1..39: mol_type = other DNA
acggataactactagactttgtgagggatcgttatctcc

SEQ ID NO: 487 - DNA, 39 bp, synthetic construct
  source 1..39: mol_type = other DNA
cttaactcgcgttcaagcctgtgagggatcgttatctcc

SEQ ID NO: 488 - DNA, 39 bp, synthetic construct
  source 1..39: mol_type = other DNA
cttagacacatcatacgtctgtgagggatcgttatctcc

SEQ ID NO: 489 - DNA, 39 bp, synthetic construct
  source 1..39: mol_type = other DNA
ggtacggaagtgtatatagtgtgagggatcgttatctcc

SEQ ID NO: 490 - DNA, 39 bp, synthetic construct
  source 1..39: mol_type = other DNA
acagtttacactattcgaatgtgagggatcgttatctcc

SEQ ID NO: 491 - DNA, 39 bp, synthetic construct
  source 1..39: mol_type = other DNA
acatagaccataccacgcctgtgagggatcgttatctcc

SEQ ID NO: 492 - DNA, 39 bp, synthetic construct
  source 1..39: mol_type = other DNA
cctagataccgtagatgtgtgtgagggatcgttatctcc

SEQ ID NO: 493 - DNA, 39 bp, synthetic construct
  source 1..39: mol_type = other DNA
gttgctacgatgactcaaatgtgagggatcgttatctcc

SEQ ID NO: 494 - DNA, 39 bp, synthetic construct; identical to SEQ ID NO: 493
  source 1..39: mol_type = other DNA

SEQ ID NO: 495 - DNA, 39 bp, synthetic construct
  source 1..39: mol_type = other DNA
ggacattaccacctagacatgtgagggatcgttatctcc

SEQ ID NO: 496 - DNA, 39 bp, synthetic construct
  source 1..39: mol_type = other DNA
tagacagactgcgacattgtgtgagggatcgttatctcc

SEQ ID NO: 497 - DNA, 39 bp, synthetic construct
  source 1..39: mol_type = other DNA
tagacatatctcacagccttgtgagggatcgttatctcc

SEQ ID NO: 498 - DNA, 39 bp, synthetic construct
  source 1..39: mol_type = other DNA
tctgcgaaggtagtgtatatgtgagggatcgttatctcc

SEQ ID NO: 499 - DNA, 39 bp, synthetic construct
  source 1..39: mol_type = other DNA
tttccaatacggtgtaccttgtgagggatcgttatctcc

SEQ ID NO: 500 - DNA, 39 bp, synthetic construct
  source 1..39: mol_type = other DNA
tttcgacgctgacactatttgtgagggatcgttatctcc

SEQ ID NO: 501 - DNA, 39 bp, synthetic construct
  source 1..39: mol_type = other DNA
tttgtgaatagtctagccgtgtgagggatcgttatctcc

SEQ ID NO: 502 - DNA, 39 bp, synthetic construct
  source 1..39: mol_type = other DNA
cactattacgcagagcaagtgtgagggatcgttatctcc

SEQ ID NO: 503 - DNA, 39 bp, synthetic construct
  source 1..39: mol_type = other DNA
gtatagtacattcccgctgtgtgagggatcgttatctcc

SEQ ID NO: 504 - DNA, 39 bp, synthetic construct
  source 1..39: mol_type = other DNA
gtatgcgacctactctttgtgtgagggatcgttatctcc

SEQ ID NO: 505 - DNA, 39 bp, synthetic construct
  source 1..39: mol_type = other DNA
tctgagaatgtgttagctgtgtgagggatcgttatctcc

SEQ ID NO: 506 - DNA, 39 bp, synthetic construct
  source 1..39: mol_type = other DNA
tctgcatagagaacaaccatgtgagggatcgttatctcc

SEQ ID NO: 507 - DNA, 39 bp, synthetic construct
  source 1..39: mol_type = other DNA
acgtttgacgaggagtagttgtgagggatcgttatctcc

SEQ ID NO: 508 - DNA, 39 bp, synthetic construct
  source 1..39: mol_type = other DNA
cctaataacactcacggaatgtgagggatcgttatctcc

SEQ ID NO: 509 - DNA, 39 bp, synthetic construct
  source 1..39: mol_type = other DNA
cctacctaaagtgacataatgtgagggatcgttatctcc

SEQ ID NO: 510 - DNA, 39 bp, synthetic construct
  source 1..39: mol_type = other DNA
gatacggaaatggacgtcgtgtgagggatcgttatctcc

SEQ ID NO: 511 - DNA, 39 bp, synthetic construct; identical to SEQ ID NO: 510
  source 1..39: mol_type = other DNA

SEQ ID NO: 512 - DNA, 39 bp, synthetic construct
  source 1..39: mol_type = other DNA
tagacttaacgcacgattttgtgagggatcgttatctcc

SEQ ID NO: 513 - DNA, 39 bp, synthetic construct
  source 1..39: mol_type = other DNA
tagagcgaacgatagttaatgtgagggatcgttatctcc

SEQ ID NO: 514 - DNA, 39 bp, synthetic construct
  source 1..39: mol_type = other DNA
tagagtcaacgagcgtatttgtgagggatcgttatctcc

SEQ ID NO: 515 - DNA, 39 bp, synthetic construct
  source 1..39: mol_type = other DNA
acactaactcgaatgaccatgtgagggatcgttatctcc

SEQ ID NO: 516 - DNA, 39 bp, synthetic construct
  source 1..39: mol_type = other DNA
acactgtaagacgatagggtgtgagggatcgttatctcc

SEQ ID NO: 517 - DNA, 39 bp, synthetic construct
  source 1..39: mol_type = other DNA
cgtcagaagccatactacttgtgagggatcgttatctcc

SEQ ID NO: 518 - DNA, 39 bp, synthetic construct
  source 1..39: mol_type = other DNA
cgtcgatacagactcagtatgtgagggatcgttatctcc

SEQ ID NO: 519 - DNA, 39 bp, synthetic construct
  source 1..39: mol_type = other DNA
ggactaagggcgactaaattgtgagggatcgttatctcc

SEQ ID NO: 520 - DNA, 39 bp, synthetic construct
  source 1..39: mol_type = other DNA
tagactgaatcatcacccttgtgagggatcgttatctcc

SEQ ID NO: 521 - DNA, 39 bp, synthetic construct
  source 1..39: mol_type = other DNA
tctgtgaataccgagtagttgtgagggatcgttatctcc

SEQ ID NO: 522 - DNA, 39 bp, synthetic construct
  source 1..39: mol_type = other DNA
aactatgctgacagtacgctgtgagggatcgttatctcc

SEQ ID NO: 523 - DNA, 39 bp, synthetic construct
  source 1..39: mol_type = other DNA
gtcagagacgtagcatctttgtgagggatcgttatctcc

SEQ ID NO: 524 - DNA, 39 bp, synthetic construct
  source 1..39: mol_type = other DNA
tctggagagatcagtatggtgtgagggatcgttatctcc

SEQ ID NO: 525 - DNA, 39 bp, synthetic construct
  source 1..39: mol_type = other DNA
actatgcctacctgtcgtatgtgagggatcgttatctcc

SEQ ID NO: 526 - DNA, 39 bp, synthetic construct
  source 1..39: mol_type = other DNA
cgcgtctacctgacttttctgtgagggatcgttatctcc

SEQ ID NO: 527 - DNA, 39 bp, synthetic construct
  source 1..39: mol_type = other DNA
cgcgttcaccgtaatctattgtgagggatcgttatctcc

SEQ ID NO: 528 - DNA, 39 bp, synthetic construct
  source 1..39: mol_type = other DNA
cctacgaacactaagtccctgtgagggatcgttatctcc

SEQ ID NO: 529 - DNA, 39 bp, synthetic construct
  source 1..39: mol_type = other DNA
agactctcgatcagccgattgtgagggatcgttatctcc

SEQ ID NO: 530 - DNA, 39 bp, synthetic construct
  source 1..39: mol_type = other DNA
tgacatagtggtcgagtagtgtgagggatcgttatctcc

SEQ ID NO: 531 - DNA, 39 bp, synthetic construct
  source 1..39: mol_type = other DNA
accgtctaaaccatacgactgtgagggatcgttatctcc

SEQ ID NO: 532 - DNA, 39 bp, synthetic construct
  source 1..39: mol_type = other DNA
acgactaacaactgtcccatgtgagggatcgttatctcc

SEQ ID NO: 533 - DNA, 39 bp, synthetic construct
  source 1..39: mol_type = other DNA
cgaggttaaagtcaagtggtgtgagggatcgttatctcc

SEQ ID NO: 534 - DNA, 39 bp, synthetic construct
  source 1..39: mol_type = other DNA
cgagtagaaatgacgctggtgtgagggatcgttatctcc

SEQ ID NO: 535 - DNA, 39 bp, synthetic construct
  source 1..39: mol_type = other DNA
gcggactaaatataggtgctgtgagggatcgttatctcc

SEQ ID NO: 536 - DNA, 39 bp, synthetic construct
  source 1..39: mol_type = other DNA
tatctttaacgtcgagccgtgtgagggatcgttatctcc

SEQ ID NO: 537 - DNA, 39 bp, synthetic construct
  source 1..39: mol_type = other DNA
acggtatagacaccacgattgtgagggatcgttatctcc

SEQ ID NO: 538 - DNA, 39 bp, synthetic construct
  source 1..39: mol_type = other DNA
acggtcaactaggtgagtttgtgagggatcgttatctcc

SEQ ID NO: 539 - DNA, 39 bp, synthetic construct
  source 1..39: mol_type = other DNA
cttatcgaagtagacgagttgtgagggatcgttatctcc

SEQ ID NO: 540 - DNA, 39 bp, synthetic construct
  source 1..39: mol_type = other DNA
ggtactaaggctgaccttttgtgagggatcgttatctcc

SEQ ID NO: 541 - DNA, 39 bp, synthetic construct
  source 1..39: mol_type = other DNA
ggtcaactctacctaagcttgtgagggatcgttatctcc

SEQ ID NO: 542 - DNA, 39 bp, synthetic construct
  source 1..39: mol_type = other DNA
acatcagagtaccagctgttgtgagggatcgttatctcc

SEQ ID NO: 543 - DNA, 39 bp, synthetic construct
  source 1..39: mol_type = other DNA
tcgtcggatcgaggttaagtgtgagggatcgttatctcc

SEQ ID NO: 544 - DNA, 39 bp, synthetic construct
  source 1..39: mol_type = other DNA
acggataactactagacttacagttggaaagccgagtac

SEQ ID NO: 545 - DNA, 39 bp, synthetic construct
  source 1..39: mol_type = other DNA
cttaactcgcgttcaagccacagttggaaagccgagtac

SEQ ID NO: 546 - DNA, 39 bp, synthetic construct
  source 1..39: mol_type = other DNA
cttagacacatcatacgtcacagttggaaagccgagtac

SEQ ID NO: 547 - DNA, 39 bp, synthetic construct
  source 1..39: mol_type = other DNA
ggtacggaagtgtatatagacagttggaaagccgagtac

SEQ ID NO: 548 - DNA, 39 bp, synthetic construct
  source 1..39: mol_type = other DNA
acagtttacactattcgaaacagttggaaagccgagtac

SEQ ID NO: 549 - DNA, 39 bp, synthetic construct
  source 1..39: mol_type = other DNA
acatagaccataccacgccacagttggaaagccgagtac

SEQ ID NO: 550 - DNA, 39 bp, synthetic construct
  source 1..39: mol_type = other DNA
cctagataccgtagatgtgacagttggaaagccgagtac

SEQ ID NO: 551 - DNA, 39 bp, synthetic construct
  source 1..39: mol_type = other DNA
gttgctacgatgactcaaaacagttggaaagccgagtac

SEQ ID NO: 552 - DNA, 39 bp, synthetic construct
  source 1..39: mol_type = other DNA
agacgtagtgagcatgaaaacagttggaaagccgagtac

SEQ ID NO: 553 - DNA, 39 bp, synthetic construct
  source 1..39: mol_type = other DNA
ggacattaccacctagacaacagttggaaagccgagtac

SEQ ID NO: 554 - DNA, 39 bp, synthetic construct
  source 1..39: mol_type = other DNA
tagacagactgcgacattgacagttggaaagccgagtac

SEQ ID NO: 555 - DNA, 39 bp, synthetic construct
  source 1..39: mol_type = other DNA
tagacatatctcacagcctacagttggaaagccgagtac

SEQ ID NO: 556 - DNA, 39 bp, synthetic construct
  source 1..39: mol_type = other DNA
tctgcgaaggtagtgtataacagttggaaagccgagtac

SEQ ID NO: 557 - DNA, 39 bp, synthetic construct
  source 1..39: mol_type = other DNA
tttccaatacggtgtacctacagttggaaagccgagtac

SEQ ID NO: 558 - DNA, 39 bp, synthetic construct
  source 1..39: mol_type = other DNA
tttcgacgctgacactattacagttggaaagccgagtac

SEQ ID NO: 559 - DNA, 39 bp, synthetic construct
  source 1..39: mol_type = other DNA
tttgtgaatagtctagccgacagttggaaagccgagtac

SEQ ID NO: 560 - DNA, 39 bp, synthetic construct
  source 1..39: mol_type = other DNA
cactattacgcagagcaagacagttggaaagccgagtac

SEQ ID NO: 561 - DNA, 39 bp, synthetic construct
  source 1..39: mol_type = other DNA
gtatagtacattcccgctgacagttggaaagccgagtac

SEQ ID NO: 562 - DNA, 39 bp, synthetic construct
  source 1..39: mol_type = other DNA
gtatgcgacctactctttgacagttggaaagccgagtac

SEQ ID NO: 563 - DNA, 39 bp, synthetic construct
  source 1..39: mol_type = other DNA
tctgagaatgtgttagctgacagttggaaagccgagtac

SEQ ID NO: 564 - DNA, 39 bp, synthetic construct
  source 1..39: mol_type = other DNA
tctgcatagagaacaaccaacagttggaaagccgagtac

SEQ ID NO: 565 - DNA, 39 bp, synthetic construct
  source 1..39: mol_type = other DNA
acgtttgacgaggagtagtacagttggaaagccgagtac

SEQ ID NO: 566 - DNA, 39 bp, synthetic construct
  source 1..39: mol_type = other DNA
cctaataacactcacggaaacagttggaaagccgagtac

SEQ ID NO: 567 - DNA, 39 bp, synthetic construct
  source 1..39: mol_type = other DNA
cctacctaaagtgacataaacagttggaaagccgagtac

SEQ ID NO: 568 - DNA, 39 bp, synthetic construct
  source 1..39: mol_type = other DNA
gatacggaaatggacgtcgacagttggaaagccgagtac

SEQ ID NO: 569 - DNA, 39 bp, synthetic construct
  source 1..39: mol_type = other DNA
gatacttaaccatagcgttacagttggaaagccgagtac

SEQ ID NO: 570 - DNA, 39 bp, synthetic construct
  source 1..39: mol_type = other DNA
tagacttaacgcacgatttacagttggaaagccgagtac

SEQ ID NO: 571 - DNA, 39 bp, synthetic construct
  source 1..39: mol_type = other DNA
tagagcgaacgatagttaaacagttggaaagccgagtac

SEQ ID NO: 572 - DNA, 39 bp, synthetic construct
  source 1..39: mol_type = other DNA
tagagtcaacgagcgtattacagttggaaagccgagtac

SEQ ID NO: 573 - DNA, 39 bp, synthetic construct
  source 1..39: mol_type = other DNA
acactaactcgaatgaccaacagttggaaagccgagtac

SEQ ID NO: 574 - DNA, 39 bp, synthetic construct
  source 1..39: mol_type = other DNA
acactgtaagacgatagggacagttggaaagccgagtac

SEQ ID NO: 575 - DNA, 39 bp, synthetic construct
  source 1..39: mol_type = other DNA
cgtcagaagccatactactacagttggaaagccgagtac

SEQ ID NO: 576 - DNA, 39 bp, synthetic construct
  source 1..39: mol_type = other DNA
cgtcgatacagactcagtaacagttggaaagccgagtac

SEQ ID NO: 577 - DNA, 39 bp, synthetic construct
  source 1..39: mol_type = other DNA
ggactaagggcgactaaatacagttggaaagccgagtac

SEQ ID NO: 578 - DNA, 39 bp, synthetic construct
  source 1..39: mol_type = other DNA
tagactgaatcatcaccctacagttggaaagccgagtac

SEQ ID NO: 579 - DNA, 39 bp, synthetic construct
  source 1..39: mol_type = other DNA
tctgtgaataccgagtagtacagttggaaagccgagtac

SEQ ID NO: 580 - DNA, 39 bp, synthetic construct
  source 1..39: mol_type = other DNA
aactatgctgacagtacgcacagttggaaagccgagtac

SEQ ID NO: 581 - DNA, 39 bp, synthetic construct
  source 1..39: mol_type = other DNA
gtcagagacgtagcatcttacagttggaaagccgagtac

SEQ ID NO: 582 - DNA, 39 bp, synthetic construct
  source 1..39: mol_type = other DNA
tctggagagatcagtatggacagttggaaagccgagtac

SEQ ID NO: 583 - DNA, 39 bp, synthetic construct
  source 1..39: mol_type = other DNA
actatgcctacctgtcgtaacagttggaaagccgagtac

SEQ ID NO: 584 - DNA, 39 bp, synthetic construct
  source 1..39: mol_type = other DNA
cgcgtctacctgacttttcacagttggaaagccgagtac

SEQ ID NO: 585 - DNA, 39 bp, synthetic construct
  source 1..39: mol_type = other DNA
cgcgttcaccgtaatctatacagttggaaagccgagtac

SEQ ID NO: 586 - DNA, 39 bp, synthetic construct
  source 1..39: mol_type = other DNA
cctacgaacactaagtcccacagttggaaagccgagtac

SEQ ID NO: 587 - DNA, 39 bp, synthetic construct
  source 1..39: mol_type = other DNA
agactctcgatcagccgatacagttggaaagccgagtac

SEQ ID NO: 588 - DNA, 39 bp, synthetic construct
  source 1..39: mol_type = other DNA
tgacatagtggtcgagtagacagttggaaagccgagtac

SEQ ID NO: 589 - DNA, 39 bp, synthetic construct
  source 1..39: mol_type = other DNA
accgtctaaaccatacgacacagttggaaagccgagtac

SEQ ID NO: 590 - DNA, 39 bp, synthetic construct
  source 1..39: mol_type = other DNA
acgactaacaactgtcccaacagttggaaagccgagtac

SEQ ID NO: 591 - DNA, 39 bp, synthetic construct
  source 1..39: mol_type = other DNA
cgaggttaaagtcaagtggacagttggaaagccgagtac

SEQ ID NO: 592 - DNA, 39 bp, synthetic construct
  source 1..39: mol_type = other DNA
cgagtagaaatgacgctggacagttggaaagccgagtac

SEQ ID NO: 593 - DNA, 39 bp, synthetic construct
  source 1..39: mol_type = other DNA
gcggactaaatataggtgcacagttggaaagccgagtac

SEQ ID NO: 594 - DNA, 39 bp, synthetic construct
  source 1..39: mol_type = other DNA
tatctttaacgtcgagccgacagttggaaagccgagtac

SEQ ID NO: 595 - DNA, 39 bp, synthetic construct
  source 1..39: mol_type = other DNA
acggtatagacaccacgatacagttggaaagccgagtac

SEQ ID NO: 596 - DNA, 39 bp, synthetic construct
  source 1..39: mol_type = other DNA
acggtcaactaggtgagttacagttggaaagccgagtac

SEQ ID NO: 597 - DNA, 39 bp, synthetic construct
  source 1..39: mol_type = other DNA
cttatcgaagtagacgagtacagttggaaagccgagtac

SEQ ID NO: 598 - DNA, 39 bp, synthetic construct
  source 1..39: mol_type = other DNA
ggtactaaggctgacctttacagttggaaagccgagtac

SEQ ID NO: 599 - DNA, 39 bp, synthetic construct
  source 1..39: mol_type = other DNA
ggtcaactctacctaagctacagttggaaagccgagtac

SEQ ID NO: 600 - DNA, 39 bp, synthetic construct
  source 1..39: mol_type = other DNA
acatcagagtaccagctgtacagttggaaagccgagtac

SEQ ID NO: 601 - DNA, 39 bp, synthetic construct
  source 1..39: mol_type = other DNA
tcgtcggatcgaggttaagacagttggaaagccgagtac

SEQ ID NO: 602 - DNA, 39 bp, synthetic construct
  source 1..39: mol_type = other DNA
acggataactactagacttgacaggcagtcacagatatg

SEQ ID NO: 603 - DNA, 39 bp, synthetic construct
  source 1..39: mol_type = other DNA
cttaactcgcgttcaagccgacaggcagtcacagatatg

SEQ ID NO: 604 - DNA, 39 bp, synthetic construct
  source 1..39: mol_type = other DNA
cttagacacatcatacgtcgacaggcagtcacagatatg

SEQ ID NO: 605 - DNA, 39 bp, synthetic construct
  source 1..39: mol_type = other DNA
ggtacggaagtgtatataggacaggcagtcacagatatg

SEQ ID NO: 606 - DNA, 39 bp, synthetic construct
  source 1..39: mol_type = other DNA
acagtttacactattcgaagacaggcagtcacagatatg

SEQ ID NO: 607 - DNA, 39 bp, synthetic construct
  source 1..39: mol_type = other DNA
acatagaccataccacgccgacaggcagtcacagatatg

SEQ ID NO: 608 - DNA, 39 bp, synthetic construct
  source 1..39: mol_type = other DNA
cctagataccgtagatgtggacaggcagtcacagatatg

SEQ ID NO: 609 - DNA, 39 bp, synthetic construct
  source 1..39: mol_type = other DNA
gttgctacgatgactcaaagacaggcagtcacagatatg

SEQ ID NO: 610 - DNA, 39 bp, synthetic construct
  source 1..39: mol_type = other DNA
agacgtagtgagcatgaaagacaggcagtcacagatatg

SEQ ID NO: 611 - DNA, 39 bp, synthetic construct
  source 1..39: mol_type = other DNA
ggacattaccacctagacagacaggcagtcacagatatg

SEQ ID NO: 612 - DNA, 39 bp, synthetic construct
  source 1..39: mol_type = other DNA
tagacagactgcgacattggacaggcagtcacagatatg

SEQ ID NO: 613 - DNA, 39 bp, synthetic construct
  source 1..39: mol_type = other DNA
tagacatatctcacagcctgacaggcagtcacagatatg

SEQ ID NO: 614 - DNA, 39 bp, synthetic construct
  source 1..39: mol_type = other DNA
tctgcgaaggtagtgtatagacaggcagtcacagatatg

SEQ ID NO: 615 - DNA, 39 bp, synthetic construct
  source 1..39: mol_type = other DNA
tttccaatacggtgtacctgacaggcagtcacagatatg

SEQ ID NO: 616 - DNA, 39 bp, synthetic construct
  source 1..39: mol_type = other DNA
tttcgacgctgacactattgacaggcagtcacagatatg

SEQ ID NO: 617 - DNA, 39 bp, synthetic construct
  source 1..39: mol_type = other DNA
tttgtgaatagtctagccggacaggcagtcacagatatg

SEQ ID NO: 618 - DNA, 39 bp, synthetic construct
  source 1..39: mol_type = other DNA
cactattacgcagagcaaggacaggcagtcacagatatg

SEQ ID NO: 619 - DNA, 39 bp, synthetic construct
  source 1..39: mol_type = other DNA
gtatagtacattcccgctggacaggcagtcacagatatg

SEQ ID NO: 620 - DNA, 39 bp, synthetic construct
  source 1..39: mol_type = other DNA
gtatgcgacctactctttggacaggcagtcacagatatg

SEQ ID NO: 621 - DNA, 39 bp, synthetic construct
  source 1..39: mol_type = other DNA
tctgagaatgtgttagctggacaggcagtcacagatatg

SEQ ID NO: 622 - DNA, 39 bp, synthetic construct
  source 1..39: mol_type = other DNA
tctgcatagagaacaaccagacaggcagtcacagatatg

SEQ ID NO: 623 - DNA, 39 bp, synthetic construct
  source 1..39: mol_type = other DNA
acgtttgacgaggagtagtgacaggcagtcacagatatg

SEQ ID NO: 624 - DNA, 39 bp, synthetic construct
  source 1..39: mol_type = other DNA
cctaataacactcacggaagacaggcagtcacagatatg

SEQ ID NO: 625 - DNA, 39 bp, synthetic construct
  source 1..39: mol_type = other DNA
cctacctaaagtgacataagacaggcagtcacagatatg

SEQ ID NO: 626 - DNA, 39 bp, synthetic construct
  source 1..39: mol_type = other DNA
gatacggaaatggacgtcggacaggcagtcacagatatg

SEQ ID NO: 627 - DNA, 39 bp, synthetic construct
  source 1..39: mol_type = other DNA
gatacttaaccatagcgttgacaggcagtcacagatatg

SEQ ID NO: 628 - DNA, 39 bp, synthetic construct
  source 1..39: mol_type = other DNA
tagacttaacgcacgatttgacaggcagtcacagatatg

SEQ ID NO: 629 - DNA, 39 bp, synthetic construct
  source 1..39: mol_type = other DNA
tagagcgaacgatagttaagacaggcagtcacagatatg

SEQ ID NO: 630 - DNA, 39 bp, synthetic construct
  source 1..39: mol_type = other DNA
tagagtcaacgagcgtattgacaggcagtcacagatatg

SEQ ID NO: 631 - DNA, 39 bp, synthetic construct
  source 1..39: mol_type = other DNA
acactaactcgaatgaccagacaggcagtcacagatatg

SEQ ID NO: 632 - DNA, 39 bp, synthetic construct
  source 1..39: mol_type = other DNA
acactgtaagacgataggggacaggcagtcacagatatg

SEQ ID NO: 633 - DNA, 39 bp, synthetic construct
  source 1..39: mol_type = other DNA
cgtcagaagccatactactgacaggcagtcacagatatg

SEQ ID NO: 634 - DNA, 39 bp, synthetic construct
  source 1..39: mol_type = other DNA
cgtcgatacagactcagtagacaggcagtcacagatatg

SEQ ID NO: 635 - DNA, 39 bp, synthetic construct
  source 1..39: mol_type = other DNA
ggactaagggcgactaaatgacaggcagtcacagatatg

SEQ ID NO: 636 - DNA, 39 bp, synthetic construct
  source 1..39: mol_type = other DNA
tagactgaatcatcaccctgacaggcagtcacagatatg

SEQ ID NO: 637 - DNA, 39 bp, synthetic construct
  source 1..39: mol_type = other DNA
tctgtgaataccgagtagtgacaggcagtcacagatatg

SEQ ID NO: 638 - DNA, 39 bp, synthetic construct
  source 1..39: mol_type = other DNA
aactatgctgacagtacgcgacaggcagtcacagatatg

SEQ ID NO: 639 - DNA, 39 bp, synthetic construct
  source 1..39: mol_type = other DNA
gtcagagacgtagcatcttgacaggcagtcacagatatg

SEQ ID NO: 640 - DNA, 39 bp, synthetic construct
  source 1..39: mol_type = other DNA
tctggagagatcagtatgggacaggcagtcacagatatg

SEQ ID NO: 641 - DNA, 39 bp, synthetic construct
  source 1..39: mol_type = other DNA
actatgcctacctgtcgtagacaggcagtcacagatatg

SEQ ID NO: 642 - DNA, 39 bp, synthetic construct
  source 1..39: mol_type = other DNA
cgcgtctacctgacttttcgacaggcagtcacagatatg

SEQ ID NO: 643 - DNA, 39 bp, synthetic construct
  source 1..39: mol_type = other DNA
cgcgttcaccgtaatctatgacaggcagtcacagatatg

SEQ ID NO: 644 - DNA, 39 bp, synthetic construct
  source 1..39: mol_type = other DNA
cctacgaacactaagtcccgacaggcagtcacagatatg

SEQ ID NO: 645 - DNA, 39 bp, synthetic construct
  source 1..39: mol_type = other DNA
agactctcgatcagccgatgacaggcagtcacagatatg

SEQ ID NO: 646 - DNA, 39 bp, synthetic construct
  source 1..39: mol_type = other DNA
tgacatagtggtcgagtaggacaggcagtcacagatatg

SEQ ID NO: 647 - DNA, 39 bp, synthetic construct
  source 1..39: mol_type = other DNA
accgtctaaaccatacgacgacaggcagtcacagatatg

SEQ ID NO: 648 - DNA, 39 bp, synthetic construct
  source 1..39: mol_type = other DNA
acgactaacaactgtcccagacaggcagtcacagatatg

SEQ ID NO: 649 - DNA, 39 bp, synthetic construct
  source 1..39: mol_type = other DNA
cgaggttaaagtcaagtgggacaggcagtcacagatatg

SEQ ID NO: 650 - DNA, 39 bp, synthetic construct
  source 1..39: mol_type = other DNA
cgagtagaaatgacgctgggacaggcagtcacagatatg

SEQ ID NO: 651 - DNA, 39 bp, synthetic construct
  source 1..39: mol_type = other DNA
gcggactaaatataggtgcgacaggcagtcacagatatg

SEQ ID NO: 652 - DNA, 39 bp, synthetic construct
  source 1..39: mol_type = other DNA
tatctttaacgtcgagccggacaggcagtcacagatatg

SEQ ID NO: 653 - DNA, 39 bp, synthetic construct
  source 1..39: mol_type = other DNA
acggtatagacaccacgatgacaggcagtcacagatatg

SEQ ID NO: 654 - DNA, 39 bp, synthetic construct
  source 1..39: mol_type = other DNA
acggtcaactaggtgagttgacaggcagtcacagatatg

SEQ ID NO: 655 - DNA, 39 bp, synthetic construct
  source 1..39: mol_type = other DNA
cttatcgaagtagacgagtgacaggcagtcacagatatg

SEQ ID NO: 656 - DNA, 39 bp, synthetic construct
  source 1..39: mol_type = other DNA
ggtactaaggctgacctttgacaggcagtcacagatatg

SEQ ID NO: 657 - DNA, 39 bp, synthetic construct
  source 1..39: mol_type = other DNA
ggtcaactctacctaagctgacaggcagtcacagatatg

SEQ ID NO: 658 - DNA, 39 bp, synthetic construct
  source 1..39: mol_type = other DNA
acatcagagtaccagctgtgacaggcagtcacagatatg

SEQ ID NO: 659 - DNA, 39 bp, synthetic construct
  source 1..39: mol_type = other DNA
tcgtcggatcgaggttaaggacaggcagtcacagatatg

SEQ ID NO: 660 - DNA, 19 bp, synthetic construct
  source 1..19: mol_type = other DNA
ttctatgtgaccgggtaca

SEQ ID NO: 661 - DNA, 20 bp, synthetic construct
  source 1..20: mol_type = other DNA
ggagataacgatccctcaca

SEQ ID NO: 662 - DNA, 20 bp, synthetic construct
  source 1..20: mol_type = other DNA
gtactcggctttccaactgt

SEQ ID NO: 663 - DNA, 20 bp, synthetic construct
  source 1..20: mol_type = other DNA
catatctgtgactgcctgtc

SEQ ID NO: 664 - DNA, 61 bp, synthetic construct
  source 1..61: mol_type = other DNA
aatgatacggcgaccaccgagatctacacacatgtgctcgtcggcagcgtcagatgtgtat

SEQ ID NO: 665 - DNA, 61 bp, synthetic construct
  source 1..61: mol_type = other DNA
aatgatacggcgaccaccgagatctacacttgtgtgctcgtcggcagcgtcagatgtgtat

SEQ ID NO: 666 - DNA, 61 bp, synthetic construct
  source 1..61: mol_type = other DNA
aatgatacggcgaccaccgagatctacactggttcaatcgtcggcagcgtcagatgtgtat

SEQ ID NO: 667 - DNA, 61 bp, synthetic construct
  source 1..61: mol_type = other DNA
aatgatacggcgaccaccgagatctacacaagtagagtcgtcggcagcgtcagatgtgtat

SEQ ID NO: 668 - DNA, 54 bp, synthetic construct
  source 1..54: mol_type = other DNA
caagcagaagacggcatacgagatcttgcactgtctcgtgggctcggagatgtg

SEQ ID NO: 669 - DNA, 54 bp, synthetic construct
  source 1..54: mol_type = other DNA
caagcagaagacggcatacgagatgaagcaacgtctcgtgggctcggagatgtg

SEQ ID NO: 670 - DNA, 54 bp, synthetic construct
  source 1..54: mol_type = other DNA
caagcagaagacggcatacgagatgagagaaggtctcgtgggctcggagatgtg

SEQ ID NO: 671 - DNA, 54 bp, synthetic construct
  source 1..54: mol_type = other DNA
caagcagaagacggcatacgagattgtcgtctgtctcgtgggctcggagatgtg